(19) United States
(12) Patent Application Publication
(10) Pub. No.: US 2020/0354321 A1
(43) Pub. Date: Nov. 12, 2020

(54) NEW PYRIDINE CARBOXAMIDES

(71) Applicant: BASF SE, Ludwigshafen (DE)

(72) Inventors: Michael Seet, Ludwigshafen (DE); Antje Wolf, Ludwigshafen (DE); Bernd Mueller, Ludwigshafen (DE); Nadine Riediger, Limburgerhof (DE); Marcus Fehr, Limburgerhof (DE); Tobias Mentzel, Mannheim (DE); Thomas Grote, Ludwigshafen (DE); Georg Christoph Rudolf, Ludwigshafen (DE); Jan Klaas Lohmann, Ludwigshafen (DE); Christian Harald Winter, Ludwigshafen (DE); Wassilios Grammenos, Ludwigshafen (DE); Christine Wiebe, Ludwigshafen (DE); Violeta Terteryan-Seiser, Ludwigshafen (DE); Ana Escribano Cuesta, Ludwigshafen (DE)

(21) Appl. No.: 16/966,057

(22) PCT Filed: Jan. 29, 2019
(86) PCT No.: PCT/EP2019/052031

§ 371 Date: Jul. 30, 2020

(30) Foreign Application Priority Data
Feb. 7, 2018 (EP) 18155526.9

(51) Int. Cl. C07D 213/16; A01N 43/40

(52) U.S. Cl. CPC C07D 213/16; A01N 43/40

(57) ABSTRACT

The present invention relates to the use of compounds of formula I, wherein the variables are defined as given in the description and claims. The invention further relates to the compounds I and composition for compounds of formula I.

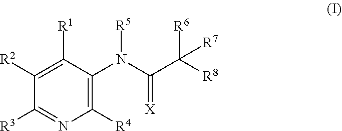

The present invention relates to pyridine compounds and the N-oxides and the salts thereof for combating phytopathogenic fungi, and to the use and methods for combating phytopathogenic fungi and to seeds coated with at least one such compound. The invention also relates to processes for preparing these compounds, intermediates, processes for preparing such intermediates, and to compositions comprising at least one compound I.

In many cases, in particular at low application rates, the fungicidal activity of the known fungicidal compounds is unsatisfactory. Based on this, it was an object of the present invention to provide compounds having improved activity and/or a broader activity spectrum against phytopathogenic harmful fungi.

Surprisingly, this object is achieved by the use of the inventive pyridine compounds of formula I having favorable fungicidal activity against phytopathogenic fungi.

Accordingly, the present invention relates to use of the compounds of formula I

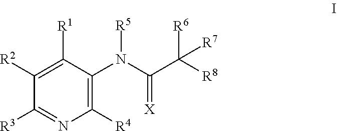

wherein

wherein
X is O, S, NH
R¹ is in each case independently selected from hydrogen, halogen, OH, CN, COOH, CONH₂, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
   R^{x} is C₁-C₄-alkyl, C₁-C₄-halogenalkyl, unsubstituted aryl or aryl that is substituted by 1, 2, 3, 4 or 5 substituents R^{x1} independently selected from C₁-C₄-alkyl, halogen, OH, CN, C₁-C₄-halogenalkyl, C₁-C₄-alkoxy and C₁-C₄-halogenalkoxy;
   wherein the acyclic moieties of R¹ are unsubstituted or substituted with identical or different groups R^{1a} which independently of one another are selected from:
R^{1a} halogen, OH, CN, COOH, CONH₂, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{11a} selected from the group consisting of halogen, OH, C₁-C₄-alkyl, C₁-C₄-halogenalkyl, C₁-C₄-alkoxy and C₁-C₄-halogenalkoxy;
   wherein the carbocyclic, heteroaryl and aryl moieties of R¹ are not further substituted or carry 1, 2, 3, 4, 5 or up to the maximum number of identical or different groups R¹ which independently of one another are selected from:
   R^{1b} halogen, OH, CN, COOH, CONH₂, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R² is in each case independently selected from halogen, OH, CN, COOH, CONH₂, NO₂, SH, NH₂, NH(C₁-C₄-alkyl), N(C₁-C₄-alkyl)₂, NH(C₂-C₄-alkenyl), N(C₂-C₄-alkenyl)₂, NH(C₂-C₄-alkynyl), N(C₂-C₄-alkynyl)₂, NH(C₃-C₆-cycloalkyl), N(C₃-C₆-cycloalkyl)₂, N(C₁-C₄-alkyl)(C₂-C₄-alkenyl), N(C₁-C₄-alkyl)(C₂-C₄-alkynyl), N(C₁-C₄-alkyl)(C₃-C₆-cycloalkyl), N(C₂-C₄-alkenyl)(C₂-C₄-alkynyl), N(C₂-C₄-alkenyl)(C₃-C₆-cycloalkyl), N(C₂-C₄-alkynyl)(C₃-C₆-cycloalkyl), NH(C(═O)C₁-C₄-alkyl), N(C(═O)C₁-C₄-alkyl)₂, NH—SO₂—R^{x}, S(O)ₙ—C₁-C₆-alkyl, S(O)ₙ-aryl, C₁-C₆-cycloalkylthio, S(O)ₙ—C₂-C₆-alkenyl, S(O)ₙ—C₂-C₆-alkynyl, CH(═O), C(═O)C₁-C₆-alkyl, C(═O)C₂-C₆-alkenyl, C(═O)C₂-C₆-alkynyl, C(═O)C₃-C₆-cycloalkyl, C(═O)NH(C₁-C₆-alkyl), CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₂-C₆-alkenyl), C(═S)O(C₂-C₆-alkynyl), C(═S)O(C₃-C₇-cycloalkyl), C(═S)NH(C₁-C₆-alkyl), C(═S)NH(C₂-C₆-alkenyl), C(═S)NH(C₂-C₆-alkynyl), C(═S)NH(C₃-C₇-cycloalkyl),C(═S)N(C₁-C₆-alkyl)₂, C(═S)N(C₂-C₆-alkenyl)₂, C(═S)N(C₂-C₆-alkynyl)₂, C(═S)N(C₃-C₇-cycloalkyl)₂, C₁-C₆-alkyl, C₁-C₆-halogenalkyl, C₂-C₆-alkenyl, C₂-C₆-alkynyl, OR^{Y}, C₃-C₆-cycloalkyl, five- or six-membered heteroaryl and aryl; wherein the heteroaryl contains one, two or three heteroatoms selected from N, O and S; wherein
   R^{x} is as defined above;
   R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, phenyl and phenyl-C₁-C₆-alkyl; wherein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
   wherein the acyclic moieties of R² are unsubstituted or substituted by groups R^{2a} which independently of one another are selected from:
   R^{2a} halogen, OH, CN, COOH, CONH₂, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or substituted by substituents R^{91a} selected from the group consisting of halogen, OH, C₁-C₄-alkyl, C₁-C₄-halogenalkyl, C₁-C₄-alkoxy and C₁-C₄-halogenalkoxy;
   wherein the carbocyclic, heteroaryl and aryl moieties of R² are unsubstituted or substituted by groups R^{3b} which independently of one another are selected from:
   R^{2b} halogen, OH, CN, COOH, CONH₂, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
   and wherein n is defined as above;
R³ is in each case independently selected from the substituents as defined for R², wherein the possible substituents for R³ are R^{3a} and R^{3b}, respectively, which correspond to R^{2a} and R^{2b}, respectively;
R², R³ together with the carbon atoms to which they are bound form a five-, six-, or seven-membered carbo-, heterocyclic or heteroaromatic ring; wherein the heterocyclic or heteroaromatic ring contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted or substituted by substituents selected from C₁-C₄-alkyl, halogen, C₁-C₄-halogenalkyl, C₁-C₄-alkoxy, C₁-C₄-halogenalkoxy, and CN; and wherein S may be in the form of its oxide SO or SO₂; and wherein in each case one or two CH₂ groups of the carbo- or heterocycle may be replaced by a group independently selected from C(═O) and C(═S); and wherein the carbo-, heterocyclic or heteroaromatic ring is substituent by (R²³)ₘ, wherein m is 0, 1, 2, 3 or 4;
R²³ is in each case independently selected from halogen, OH, CN, COOH, CONH₂, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, saturated or partially unsaturated three-, four-, five-, six-, seven-, eight-, nine-, or ten-membered carbo- and heterocycle, five- or six-membered heteroaryl and aryl; wherein the heterocycle and heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
   R^{x} is as defined above;

wherein the acyclic moieties of R²³ are unsubstituted or carry 1, 2, 3 or up to the maximum possible number of identical or different groups R^{23a} which independently of one another are selected from:
R^{23a} halogen, OH, CN, COOH, CONH₂, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or unsubstituted or substituted with R^{91a} selected from the group consisting of halogen, OH, C₁-C₄-alkyl, C₁-C₄-halogenalkyl, C₁-C₄-alkoxy, C₁-C₄-halogenalkoxy, CN, C₃-C₆-cycloalkyl, C₃-C₆-halogencycloalkyl, C₁-C₄-alkylthio;

wherein the carbocyclic, heterocyclic, heteroaryl and aryl moieties of R²³ are unsubstituted or substituted with identical or different groups R^{23b} which independently of one another are selected from:
R^{23b} halogen, OH, CN, COOH, CONH₂, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio;
R⁴ is in each case independently selected from hydrogen, halogen, OH, CN, COOH, CONH₂, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
   R^{x} is as defined above
   wherein the aliphatic moieties of R⁴ are unsubstituted or substituted with identical or different groups R^{2a} which independently of one another are selected from:
   R^{4a} halogen, OH, CN, COOH, CONH₂, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{41a} selected from the group consisting of halogen, OH, C₁-C₄-alkyl, C₁-C₄-halogenalkyl, C₁-C₄-alkoxy and C₁-C₄-halogenalkoxy;
   wherein the cycloalkyl, heteroaryl and aryl moieties of R⁴ are not further substituted or carry 1, 2, 3, 4, 5 or up to the maximum number of identical or different groups R^{4b} which independently of one another are selected from:
   R^{4b} halogen, OH, CN, COOH, CONH₂, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R⁵ is in each case independently selected from hydrogen, OH, CH(═O), C(═O)C₁-C₆-alkyl, C(═O)C₂-C₆-alkenyl, C(═O)C₂-C₆-alkynyl, C(═O)C₃-C₆-cycloalkyl, C₁-C₆-alkyl, C₁-C₄-halogenalkyl, C₃-C₆-cycloalkyl, C₃-C₆-halogencycloalkyl, C₁-C₄-alkoxy, C₁-C₄-halogenalkoxy, OR^{Y}, C₂-C₆-alkenyl, C₂-C₆-halogenalkenyl C₂-C₆-alkynyl, C₂-C₆-halogenalkynyl, five- or six-membered heteroaryl and aryl; wherein the heteroaryl contains one, two or three heteroatoms selected from N, O and S; wherein the aryl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
   R^{Y} is defined as above;
   wherein the acyclic moieties of R⁵ are not further substituted or carry one, two, three or up to the maximum possible number of identical or different groups R^{5a} which independently of one another are selected from:
   R^{5a} halogen, OH, CN, COOH, CONH₂, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl, phenyl and phenoxy group is unsubstituted or carries one, two, three, four or five substituents R^{78a′} selected from the group consisting of halogen, OH, C₁-C₄-alkyl, C₁-C₄-halogenalkyl, C₁-C₄-alkoxy and C₁-C₄-halogenalkoxy;
      wherein the alicyclic, phenyl, heterocyclic and heteroaryl moieties of R¹² are not further substituted or carry one, two, three, four, five or up to the maximum number of identical or different groups R^{12b} which independently of one another are selected from:
   R^{5b} halogen, OH, CN, COOH, CONH₂, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R⁶ is independently selected from hydrogen, halogen, OH, CN, COOH, CONH₂, NO₂, SH, C₁-C₆-alkylthio, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, aryloxy, heteroaryloxy, arylamino, heteroarylamino, arylthio, heteroarylthio, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or wherein the aliphatic moieties of and R⁶ are independently not further substituted or carry 1, 2, 3 or up to the maximum possible number of identical or different groups R^{6a}, respectively, which independently of one another are selected from:
   R^{6a} halogen, OH, CN, COOH, CONH₂, NO₂, SH, NH₂, NH(C₁-C₄-alkyl), N(C₁-C₄-alkyl)₂, NH-aryl, N(aryl)₂, NH(C(═O)C₁-C₄-alkyl), N(C(═O)C₁-C₄-alkyl)₂, NH—SO₂—R^{x}, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S), five- or six-membered heteroaryl and aryl; wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′, R″ and R″ are as defined above
   wherein the carbocyclic, heterocyclic, heteroaryl and aryl moieties of R⁶ are independently not further substituted or carry 1, 2, 3, 4, 5 or up to the maximum number of identical or different groups R^{6b}, respectively, which independently of one another are selected from:
   R^{6b} halogen, OH, CN, COOH, CONH₂, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy;
   and wherein R^{x} is as defined above; or
   n is 0, 1, 2
R⁷ is independently selected from
   halogen, OH, CN, COOH, CONH₂, NO₂, SH, C₁-C₆-alkylthio, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, aryloxy, heteroaryloxy, arylamino, heteroarylamino, arylthio, heteroarylthio, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or
   wherein the aliphatic moieties of and R⁷ are independently not further substituted or carry 1, 2, 3 or up to the maximum possible number of identical or different groups R^{7a}, respectively, which independently of one another are selected from:
   R^{7a}
   halogen, OH, CN, COOH, CONH₂, NO₂, SH, NH₂, NH(C₁-C₄-alkyl), N(C₁-C₄-alkyl)₂, NH-aryl, N(aryl)₂, NH(C(═O)C₁-C₄-alkyl), N(C(═O)C₁-C₄-alkyl)₂, NH—SO₂—R^{x}, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S), five- or six-membered heteroaryl and aryl; wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′, R″ and R″ are as defined above
   wherein the carbocyclic, heterocyclic, heteroaryl and aryl moieties of R⁷ are independently not further substituted or carry 1, 2, 3, 4, 5 or up to the maximum number of identical or different groups R^{7b}, respectively, which independently of one another are selected from:
   R^{7b} halogen, OH, CN, COOH, CONH₂, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy;
   and wherein R^{x} is as defined above;
   n is 0, 1, 2
R⁸ is independently selected from CN, COOH, CONH₂, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S, and wherein heterocycle and heteroaryl are connected via C atom; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or
   wherein the aliphatic moieties of and R⁸ are independently not further substituted or carry 1, 2, 3 or up to the maximum possible number of identical or different groups R^{8a}, respectively, which independently of one another are selected from:
   R^{8a} halogen, OH, CN, COOH, CONH₂, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S), five- or six-membered heteroaryl and aryl; wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′, R″ and R″ are as defined above
   wherein the carbocyclic, heterocyclic, heteroaryl and aryl moieties of R⁸ are independently not further substituted or carry 1, 2, 3, 4, 5 or up to the maximum number of identical or different groups R^{8b}, respectively, which independently of one another are selected from:
   R^{8b} halogen, OH, CN, COOH, CONH₂, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy;
   and wherein R^{x} is as defined above;
   n is 0, 1, 2

and the N-oxides and the agriculturally acceptable salts thereof as fungicides.

The numbering of the ring members and substituents in the compounds of the present invention is as given in formula I above.

A skilled person will realize that compounds of formula I can be accessed via an amide coupling reaction of 3-aminopyridines of type II with carboxylic acids of type III. Among various reported methods for such amide coupling reactions, a robust method involves the treatment of carboxylic acids of type III with thionyl chloride or oxalyl chloride or dicyclohexylcarbodiimide in solvents like tetrahydrofurane, dimethylformamide or dichloromethane at room temperature. Subsequent addition of amines of type II in the presence of a base like triethylamine at room temperature gives the target compounds of type I (see: Chem. Soc. Rev. 2009, 606-631, or Tetrahedron 2005, 10827-10852).

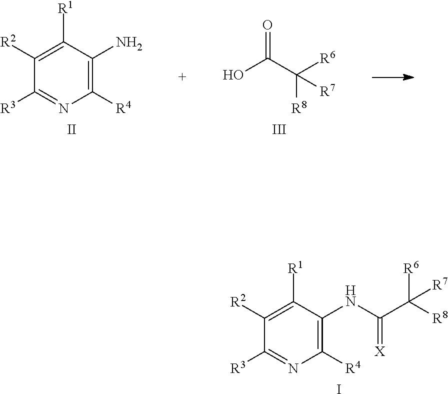

The compounds of the formula and III are commercial available.

The N-oxides may be prepared from the inventive compounds according to conventional oxidation methods, e. g. by treating compounds I with an organic peracid such as metachloroperbenzoic acid (cf. WO 03/64572 or J. Med. Chem. 38(11), 1892-903, 1995); or with inorganic oxidizing agents such as hydrogen peroxide (cf. J. Heterocyc. Chem. 18(7), 1305-8, 1981) or oxone (cf. J. Am. Chem. Soc. 123(25), 5962-5973, 2001). The oxidation may lead to pure mono-N-oxides or to a mixture of different N-oxides, which can be separated by conventional methods such as chromatography.

In the following, the intermediate compounds are further described. A skilled person will readily understand that the preferences for the substituents, also in particular the ones given in the tables below for the respective substituents, given herein in connection with compounds I apply for the intermediates accordingly. Thereby, the substituents in each case have independently of each other or more preferably in combination the meanings as defined herein.

If the synthesis yields mixtures of isomers, a separation is generally not necessarily required since in some cases the individual isomers can be interconverted during work-up for use or during application (e. g. under the action of light, acids or bases). Such conversions may also take place after use, e. g. in the treatment of plants in the treated plant, or in the harmful fungus to be controlled.

In the definitions of the variables given above, collective terms are used which are generally representative for the substituents in question. The term “Cₙ-Cₘ” indicates the number of carbon atoms possible in each case in the substituent or substituent moiety in question.

The term “halogen” refers to fluorine, chlorine, bromine and iodine.

The term “C₁-C₆-alkyl” refers to a straight-chained or branched saturated hydrocarbon group having 1 to 6 carbon atoms, e.g. methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Likewise, the term “C₂-C₄-alkyl” refers to a straight-chained or branched alkyl group having 2 to 4 carbon atoms, such as ethyl, propyl (n-propyl), 1-methylethyl (iso-propoyl), butyl, 1-methylpropyl (sec.-butyl), 2-methylpropyl (iso-butyl), 1,1-dimethylethyl (tert.-butyl).

The term “C₁-C₆-halogenalkyl” refers to an alkyl group having 1 or 6 carbon atoms as defined above, wherein some or all of the hydrogen atoms in these groups may be replaced by halogen atoms as mentioned above. Examples are “C₁-C₂-halogenalkyl” groups such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl or pentafluoroethyl.

The term “C₁-C₆-hydroxyalkyl” refers to an alkyl group having 1 or 6 carbon atoms as defined above, wherein some or all of the hydrogen atoms in these groups may be replaced by OH groups.

The term “C₁-C₄-alkoxy-C₁-C₄-alkyl” refers to alkyl having 1 to 4 carbon atoms (as defined above), where According to one hydrogen atom of the alkyl radical is replaced by a C₁-C₄-alkoxy group (as defined above). Likewise, the term “C₁-C₆-alkoxy-C₁-C₄-alkyl” refers to alkyl having 1 to 4 carbon atoms (as defined above), where According to one hydrogen atom of the alkyl radical is replaced by a C₁-C₆-alkoxy group (as defined above).

The term “C₂-C₆-alkenyl” refers to a straight-chain or branched unsaturated hydrocarbon radical having 2 to 6 carbon atoms and a double bond in any position. Examples are “C₂-C₄-alkenyl” groups, such as ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₂-C₆-alkynyl” refers to a straight-chain or branched unsaturated hydrocarbon radical having 2 to 6 carbon atoms and containing at least one triple bond. Examples are “C₂-C₄-alkynyl” groups, such as ethynyl, prop-1-ynyl, prop-2-ynyl (propargyl), but-1-ynyl, but-2-ynyl, but-3-ynyl, 1-methyl-prop-2-ynyl.

The term “C₁-C₆-alkoxy” refers to a straight-chain or branched alkyl group having 1 to 6 carbon atoms which is bonded via an oxygen, at any position in the alkyl group. Examples are “C₁-C₄-alkoxy” groups, such as methoxy, ethoxy, n-propoxy, 1-methylethoxy, butoxy, 1-methyl, propoxy, 2-methylpropoxy or 1,1-dimethylethoxy.

The term “C₁-C₆-halogenalkoxy” refers to a C₁-C₆-alkoxy radical as defined above, wherein some or all of the hydrogen atoms in these groups may be replaced by halogen atoms as mentioned above. Examples are “C₁-C₄-halogenalkoxy” groups, such as OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chlorothoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoro¬propoxy, 2 chloropropoxy, 3-chloropropoxy, 2,3-dichloropropoxy, 2-bromopropoxy, 3 bromopropoxy, 3,3,3-trifluoropropoxy, 3,3,3-trichloropropoxy, OCH₂—C₂F₅, OCF₂—C₂F₅, 1-fluoromethyl-2-fluoroethoxy, 1-chloromethyl-2-chloroethoxy, 1-bromomethyl-2-bromoethoxy, 4-fluorobutoxy, 4-chlorobutoxy, 4-bromobutoxy or nonafluorobutoxy.

The term “C₂-C₆-alkenyloxy” refers to a straight-chain or branched alkenyl group having 2 to 6 carbon atoms which is bonded via an oxygen, at any position in the alkenyl group. Examples are “C₂-C₄-alkenyloxy” groups.

The term “C₂-C₆-alkynyloxy” refers to a straight-chain or branched alkynyl group having 2 to 6 carbon atoms which is bonded via an oxygen, at any position in the alkynyl group. Examples are “C₂-C₄-alkynyloxy” groups.

The term “C₃-C₆-cycloalkyl” refers to monocyclic saturated hydrocarbon radicals having 3 to 6 carbon ring members, such as cyclopropyl, cyclobutyl, cyclopentyl, cyclohexyl. Accordingly, a saturated three-, four-, five-, six-, seven-, eight-, nine or ten-membered carbocyclyl or carbocycle is a “C₃-C₁₀-cycloalkyl”.

The term “C₃-C₆-cycloalkenyl” refers to a monocyclic partially unsaturated 3-, 4- 5- or 6-membered carbocycle having 3 to 6 carbon ring members and at least one double bond, such as cyclopentenyl, cyclopentadienyl, cyclohexadienyl. Accordingly, a partially unsaturated three-, four, five-, six-, seven-, eight-, nine or ten-membered carbocyclyl or carbocycle is a “C₃-C₁₀-cycloalkenyl”.

The term “C₃-C₈-cycloalkyl-C₁-C₄-alkyl” refers to alkyl having 1 to 4 carbon atoms (as defined above), where According to one hydrogen atom of the alkyl radical is replaced by a cycloalkyl radical having 3 to 8 carbon atoms (as defined above).

The term “C₁-C₆-alkylthio” as used herein refers to straight-chain or branched alkyl groups having 1 to 6 carbon atoms (as defined above) bonded via a sulfur atom. Accordingly, the term “C₁-C₆-halogenalkylthio” as used herein refers to straight-chain or branched halogenalkyl group having 1 to 6 carbon atoms (as defined above) bonded through a sulfur atom, at any position in the halogenalkyl group.

The term “C(═O)—C₁-C₆-alkyl” refers to a radical which is attached through the carbon atom of the group C(═O) as indicated by the number valence of the carbon atom. The number of valence of carbon is 4, that of nitrogen is 3. Likewise the following terms are to be construed: NH(C₁-C₄-alkyl), N(C₁-C₄-alkyl)₂, NH(C₃-C₆-cycloalkyl), N(C₃-C₆-cycloalkyl)₂, C(═O)—NH(C₁-C₆-alkyl), C(═O)—N(C₁-C₆-alkyl)₂.

The term “saturated or partially unsaturated three-, four-, five-, six-, seven-, eight-, nine or ten-membered heterocyclyl or heterocycle, wherein the heterocyclyl or heterocycle contains 1, 2, 3 or 4 heteroatoms selected from N, O and S” is to be understood as meaning both saturated and partially unsaturated heterocycles, wherein the ring member atoms of the heterocycle include besides carbon atoms 1, 2, 3 or 4 heteroatoms independently selected from the group of O, N and S. For example:

a 3- or 4-membered saturated heterocycle which contains 1 or 2 heteroatoms from the group consisting of O, N and S as ring members such as oxirane, aziridine, thiirane, oxetane, azetidine, thiethane, [1,2]dioxetane, [1,2]dithietane, [1,2]diazetidine; and a 5- or 6-membered saturated or partially unsaturated heterocycle which contains 1, 2 or 3 heteroatoms from the group consisting of O, N and S as ring members such as 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals; and

a 7-membered saturated or partially unsaturated heterocycle such as tetra- and hexahydroazepinyl, such as 2,3,4,5-tetrahydro[1H]azepin-1-,-2-,-3-,-4-,-5-,-6- or-7-yl, 3,4,5,6-tetrahydro[2H]azepin-2-,-3-,-4-,-5-,-6- or-7-yl, 2,3,4,7-tetrahydro[1H]azepin-1-,-2-,-3-,-4-,-5-,-6- or-7-yl, 2,3,6,7-tetrahydro[1H]azepin-1-,-2-,-3-,-4-,-5-,-6- or-7-yl, hexahydroazepin-1-,-2-,-3- or-4-yl, tetra- and hexahydrooxepinyl such as 2,3,4,5-tetrahydro[1H]oxepin-2-,-3-,-4-,-5-,-6- or-7-yl, 2,3,4,7-tetrahydro[1H]oxepin-2-,-3-,-4-,-5-,-6- or-7-yl, 2,3,6,7-tetrahydro[1H]oxepin-2-, -3-,-4-,-5-,-6- or-7-yl, hexahydroazepin-1-,-2-,-3- or-4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals.

The term “substituted” refers to substitued with 1, 2, 3 or up to the maximum possible number of substituents.

The term “5-or 6-membered heteroaryl” or “5-or 6-membered heteroaromatic” refers to aromatic ring systems including besides carbon atoms, 1, 2, 3 or 4 heteroatoms independently selected from the group consisting of N, O and S, for example, a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl; or a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

Agriculturally acceptable salts of the inventive compounds encompass especially the salts of those cations or the acid addition salts of those acids whose cations and anions, respectively, have no adverse effect on the fungicidal action of said compounds. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium. Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting such inventive compound with an acid of the corresponding anion, preferably of hydrochloric acid, hydrobromic acid, sulfuric acid, phosphoric acid or nitric acid.

The inventive compounds can be present in atropisomers arising from restricted rotation about a single bond of asymmetric groups. They also form part of the subject matter of the present invention.

Depending on the substitution pattern, the compounds of formula I and their N-oxides may have one or more centers of chirality, in which case they are present as pure enantiomers or pure diastereomers or as enantiomer or diastereomer mixtures. Both, the pure enantiomers or diastereomers and their mixtures are subject matter of the present invention.

In the following, particular embodiments of the inventive compounds are described. Therein, specific meanings of the respective substituents are further detailed, wherein the meanings are in each case on their own but also in any combination with one another, particular embodiments of the present invention.

Furthermore, in respect of the variables, generally, the embodiments of the compounds I also apply to the intermediates.

R¹ according to the invention is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl;

wherein the heteroaryl contains one, two or three heteroatoms selected from N, O and S; and

wherein

R^{x} is C₁-C₄-alkyl, C₁-C₄-halogenalkyl, unsubstituted aryl or aryl that is substituted with one, two, three, four or five substituents R^{x1} independently selected from C₁-C₄-alkyl, halogen, OH, CN, C₁-C₄-halogenalkyl, C₁-C₄-alkoxy and C₁-C₄-halogenalkoxy;

wherein the acyclic moieties of R¹ are unsubstituted or substituted with identical or different groups R^{1a} which independently of one another are selected from:

R^{1a} halogen, OH, CN, C₁-C₆-alkoxy, C₃-C₆-cycloalkyl, C₃-C₆-halogencycloalky, C₁-C₄-halogenalkoxy, C₁-C₆-alkylthio, aryl and phenoxy, wherein the aryl and phenoxy group is unsubstituted or unsubstituted or substituted with R^{11a} selected from the group consisting of halogen, OH, C₁-C₄-alkyl, C₁-C₄-halogenalkyl, C₁-C₄-alkoxy and C₁-C₄-halogenalkoxy;

wherein the carbocyclic, heteroaryl and aryl moieties of R¹ are unsubstituted or substituted with identical or different groups R^{1b} which independently of one another are selected from:

R^{1b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

For every R¹ that is present in the inventive compounds, the following embodiments and preferences apply independently of the meaning of any other R¹ that may be present in the ring.

According to one embodiment of formula I, R¹ is H, halogen or C₁-C₆-alkyl, in particular H, CH₃, Et, F, Cl, more specifically H, CH₃, F or Cl most preferred H, F or Cl.

According to another embodiment of formula I, R¹ is hydrogen.

According to still another embodiment of formula I, R¹ is halogen, in particular Br, F or Cl, more specifically F or Cl.

According to another embodiment of formula I, R¹ is F

According to another embodiment of formula I, R¹ is Cl

According to another embodiment of formula I, R¹ is Br.

According to still another embodiment of formula I, R¹ is OH.

According to still another embodiment of formula I, R¹ is COOH.

According to still another embodiment of formula I, R¹ is CONH₂.

According to still another embodiment of formula I, R¹ is CN.

According to still another embodiment of formula I, R¹ is NO₂.

According to still another embodiment of formula I, R¹ is SH.

According to still another embodiment of formula I R¹ is NH₂, NH(C₁-C₄-alkyl), N(C₁-C₄-alkyl)₂ or NH—SO₂—R^{x}, wherein R^{x} is C₁-C₄-alkyl, C₁-C₄-halogenalkyl, unsubstituted aryl or aryl that is substituted with one, two, three, four or five substituents R^{x1} independently selected from C₁-C₄-alkyl, halogen, OH, CN, C₁-C₄-halogenalkyl, C₁-C₄-alkoxy, or C₁-C₄-halogenalkoxy. In particular C₁-C₄-alkyl, such as NHCH₃ and N(CH₃)₂. In particular R^{x} is C₁-C₄-alkyl, and phenyl that is substituted with one CH₃, more specifically SO₂—R^{x} is CH₃ and tosyl group (“Ts”).

According to still another embodiment of formula I, R¹ is C₁-C₆-alkyl, in particular C₁-C₄-alkyl, such as CH₃ or CH₂CH₃.

According to still another embodiment of formula I, R¹ is C₁-C₆-halogenalkyl, in particular C₁-C₄-halogenalkyl, such as CF₃, CHF₂, CH₂F, CCl₃, CHCl₂, CH₂Cl, CF₃CH₂, CCl₃CH₂ or CF₂CHF₂.

According to still another embodiment of formula I, R¹ is C₂-C₆-alkenyl or C₂-C₆-halogenalkenyl, in particular C₂-C₄-alkenyl or C₂-C₄-halogenalkenyl, such as CH═CH₂, C(CH₃)═CH₂, CH═CCl₂, CH═CF₂, CCl═CCl₂, CF═CF₂, CH═CH₂, CH₂CH═CCl₂, CH₂CH═CF₂, CH₂CCl═CCl₂, CH₂CF═CF₂, CCl₂CH═CCl₂, CF₂CH═CF₂, CCl₂CCl═CCl₂, or CF₂CF═CF₂.

According to still another embodiment of formula I, R¹ is C₂-C₆-alkynyl or C₂-C₆-halogenalkynyl, in particular C₂-C₄-alkynyl or C₂-C₄-halogenalkynyl, such as C≡CH, C≡CCl, C≡CF. CH₂C≡CH, CH₂C≡CCl, or CH₂C≡CF.

According to still another embodiment of formula I, R¹ is C₁-C₆-alkoxy, in particular C₁-C₄-alkoxy, more specifically C₁-C₂-alkoxy such as OCH₃ or OCH₂CH₃.

According to still another embodiment of formula I, R¹ is C₁-C₆-halogenalkoxy, in particular C₁-C₄-halogenalkoxy, more specifically C₁-C₂-halogenalkoxy such as OCF₃, OCHF₂, OCH₂F, OCCl₃, OCHCl₂ or OCH₂Cl, in particular OCF₃, OCHF₂, OCCl₃ or OCHCl₂.

According to still another embodiment of formula I R¹ is C₃-C₆-cycloalkyl, in particular cyclopropyl.

According to still another embodiment of formula I, R¹ is C₃-C₆-cycloalkyl, for example cyclopropyl, substituted with one, two, three or up to the maximum possible number of identical or different groups R^{1b} as defined and preferably herein.

According to still another embodiment of formula I, R¹ is C₃-C₆-halogencycloalkyl. In a special embodiment R¹ is fully or partially halogenated cyclopropyl.

According to still another embodiment of formula I, R¹ is unsubstituted aryl or aryl that is substituted with one, two, three or four R^{b}, as defined herein. In particular, R¹ is unsubstituted phenyl or phenyl that is substituted with one, two, three or four R^{b}, as defined herein.

According to still another embodiment of formula, R¹ is unsubstituted 5- or 6-membered heteroaryl. According to still a further embodiment, R¹ is 5- or 6-membered heteroaryl that is substituted with one, two or three R^{1b}, as defined herein.

According to still another embodiment of formula I, R¹ is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy and C₃-C₆-cycloalkyl; wherein the acyclic moieties of R¹ are not further substituted or carry one, two, three, four or five identical or different groups R^{1a} as defined below and wherein the carbocyclic, heteroaryl and aryl moieties of R¹ are not further substituted or carry one, two, three, four or five identical or different groups R^{1b} as defined below.

According to still another embodiment of formula I, R¹ is independently selected from hydrogen, halogen, CN, OH, C₁-C₆-alkyl, C₁-C₆-alkoxy, C₆-alkenyl, C₂-C₆-alkynyl, C₃-C₆-cycloalkyl, wherein the acyclic and cyclic moieties of R¹ are unsubstituted or substituted by halogen.

According to still another embodiment of formula I, R¹ is independently selected from hydrogen, halogen, CN, OH, C₁-C₆-alkyl, C₁-C₆-halogenalkyl, C₁-C₆-alkoxy and C₁-C₆-halogenalkoxy, in particular independently selected from H, F, Cl, Br, CN, OH, C₁-C₄-alkyl, C₁-C₄-halogenalkyl, C₁-C₄-alkoxy and C₁-C₄-halogenalkoxy.

According to still another embodiment of formula I, R¹ is independently selected from H, CN, halogen or C₁-C₆-alkyl, in particular H, CN, CH₃, Et, F, Cl, more specifically H, CN, CH₃, F or Cl most preferred H, CH₃, F or Cl.

R^{1a} are the possible substituents for the acyclic moieties of R¹.

R^{1a} according to the invention is independently selected from halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or unsubstituted or substituted with R^{11a} selected from the group consisting of halogen, OH, C₁-C₄-alkyl, C₁-C₄-halogenalkyl, C₁-C₄-alkoxy and C₁-C₄-halogenalkoxy, in particular selected from halogen, C₁-C₂-alkyl, C₁-C₂-halogenalkyl, C₁-C₂-alkoxy and C₁-C₂-halogenalkoxy, more specifically selected from halogen, such as F, Cl and Br.

In to one embodiment R^{1a} is independently selected from halogen, OH, CN, C₁-C₂-alkoxy, C₃-C₆-cycloalkyl, C₃-C₆-halogencycloalkyl and C₁-C₂-halogenalkoxy. Specifically, R^{1a} is independently selected from F, Cl, OH, CN, C₁-C₂-alkoxy, cyclopropyl, 1-F-cyclopropyl, 1-Cl-cyclopropyl, 1,1-F₂-cyclopropyl, 1,1-Cl₂-cyclopropyl and C₁-C₂-halogenalkoxy.

According to one embodiment R^{1a} is independently selected from halogen, such as F, Cl, Br and I, more specifically F, Cl and Br.

According to still another embodiment of formula I, R^{1a} is independently selected from OH, C₃-C₆-cycloalkyl, C₃-C₆-halogencycloalkyl and C₁-C₂-halogenalkoxy. Specifically, R^{1a} is independently selected from OH, cyclopropyl and C₁-C₂-halogenalkoxy.

According to still another embodiment of formula I, R^{1a} is independently selected from aryl and phenoxy, wherein the aryl group is unsubstituted or substituted with R^{11a} selected from the group consisting of halogen, OH, C₁-C₄-alkyl, C₁-C₄-halogenalkyl, C₁-C₄-alkoxy and C₁-C₄-halogenalkoxy, in particular selected from halogen, C₁-C₂-alkyl, C₁-C₂-halogenalkyl, C₁-C₂-alkoxy and C₁-C₂-halogenalkoxy, more specifically selected from halogen, such as F, Cl and Br.

R^{1b} are the possible substituents for the carbocyclic, heteroaryl and aryl moieties of R¹.

R^{1b} according to the invention is independently selected from halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;

According to one embodiment thereof R¹ is independently selected from halogen, CN, C₁-C₂-alkyl, C₁-C₂-alkoxy, C₁-C₂-halogenalkyl, C₃-C₆-cycloalkyl, C₃-C₆-halogencycloalkyl and C₁-C₂-halogenalkoxy. Specifically, R^{1b} is independently selected from F, Cl, Br, OH, CN, CH₃, OCH₃, CHF₂, OCHF₂, cyclopropyl, 1-F-cyclopropyl, 1-Cl-cyclopropyl, 1,1-F₂-cyclopropyl, 1,1-Cl₂-cyclopropyl, OCF₃, and OCHF₂.

According to still another embodiment thereof R^{1b} is independently selected from halogen, C₁-C₂-alkyl, C₁-C₂-alkoxy, C₁-C₂-halogenalkyl, C₃-C₆-cycloalkyl, C₃-C₆-halogencycloalkyl and C₁-C₂-halogenalkoxy. Specifically, R^{1b} is independently selected from halogen, CN, OH, CH₃, CHF₂, OCHF₂, OCF₃, OCH₃, cyclopropyl, 1-F-cyclopropyl, 1-Cl-cyclopropyl, 1,1-F₂-cyclopropyl, 1,1-Cl₂-cyclopropyl and halogenmethoxy, more specifically independently selected from F, Cl, OH, CH₃, OCH₃, CHF₂, OCH₃, cyclopropyl, 1-F-cyclopropyl, 1-Cl-cyclopropyl, 1,1-F₂-cyclopropyl, 1,1-Cl₂-cyclopropyl, OCHF₂ and OCF₃.

R^{x} in the substituent NH—SO₂—R^{x} is in each case independently selected from C₁-C₄-alkyl, C₁-C₄-halogenalkyl, unsubstituted aryl and aryl that is substituted with one, two, three, four or five substituents R^{x1} independently selected from C₁-C₄-alkyl, halogen, OH, CN, C₁-C₄-halogenalkyl, C₁-C₄-alkoxy and C₁-C₄-halogenalkoxy. In particular, R^{x} is in each case independently selected from C₁-C₄-alkyl, halogen, OH, CN and phenyl that is substituted with one, two or three R^{x1} independently selected from C₁-C₂-alkyl, more specifically R^{x} is in each case independently selected from C₁-C₄-alkyl and phenyl that is substituted with one CH₃, more specifically SO₂—R^{x} is the tosyl group (“Ts”).

Particularly preferred embodiments of R¹ according to the invention are in Table P1 below, wherein each line of lines P1-1 to P1-16 corresponds to one particular embodiment of the invention. Thereby, for every R¹ that is present in the inventive compounds, these specific embodiments and preferences apply independently of the meaning of any other R¹ that may be present in the ring:

TABLE P1

| No. | R¹ |
|---|---|
| P4-1 | H |
| P4-2 | Cl |
| P4-3 | F |
| P4-4 | Br |
| P4-5 | OH |
| P4-6 | ON |
| P4-7 | NO₂ |
| P4-8 | CH₃ |
| P4-9 | CH₂CH₃ |
| P4-10 | CF₃ |
| P4-11 | CHF₂ |
| P4-12 | OCH₃ |
| P4-13 | OCH₂CH₃ |
| P4-14 | OCF₃ |
| P4-15 | OCHF₂ |
| P4-16 | NH-Ts |

“Ts” in the table stands for the tosylgroup SO₂-(p-CH₃)phenyl.

R² is in each case independently selected from H, halogen, COOH, CONH₂, OH, CN, NO₂, SH, NH₂, NH(C₁-C₄-alkyl), N(C₁-C₄-alkyl)₂, NH(C₂-C₄-alkenyl), N(C₂-C₄-alkenyl)₂, NH(C₂-C₄-alkynyl), N(C₂-C₄-alkynyl)₂, NH(C₃-C₆-cycloalkyl), N(C₃-C₆-cycloalkyl)₂, N(C₂-C₄-alkyl)(C₂-C₄-alkenyl), N(C₂-C₄-alkyl)(C₂-C₄-alkynyl), N(C₂-C₄-alkyl)(C₃-C₆-cycloalkyl), N(C₂-C₄-alkenyl)(C₂-C₄-alkynyl), N(C₂-C₄-alkenyl)(C₃-C₆-cycloalkyl), N(C₂-C₄-alkynyl)(C₃-C₆-cycloalkyl), NH(C(═O)C₁-C₄-alkyl), N(C(═O)C₁-C₄-alkyl)₂, NH—SO₂—R^{x}, S(O)ₙ—C₁-C₆-alkyl, S(O)ₙ-aryl, C₁-C₆-cycloalkylthio, S(O)ₙ—C₂-C₆-alkenyl, S(O)ₙ—C₂-C₆-alkynyl, CH(═O), C(═O)C₁-C₆-alkyl, C(═O)C₂-C₆-alkenyl, C(═O)C₂-C₆-alkynyl, C(═O)C₃-C₆-cycloalkyl, C(═O)NH(C₁-C₆-alkyl), CH(═S), C(═S)C₁-C₆-alkyl, C(═S)C₂-C₆-alkenyl, C(═S)C₂-C₆-alkynyl, C(═S)C₃-C₆-cycloalkyl, (═S)O(C₂-C₆-alkenyl), C(═S)O(C₂-C₆-alkynyl), C(═S)O(C₃-C₇-cycloalkyl), C(═S)NH(C₁-C₆-alkyl), C(═S)NH(C₂-C₆-alkenyl), C(═S)NH(C₂-C₆-alkynyl), C(═S)NH(C₃-C₇-cycloalkyl),C(═S)N(C₁-C₆-alkyl)₂, C(═S)N(C₂-C₆-alkenyl)₂, C(═S)N(C₂-C₆-alkynyl)₂, C(═S)N(C₃-C₇-cycloalkyl)₂, C₁-C₆-alkyl, C₂-C₆-alkenyl, C₂-C₆-alkynyl, OR^{Y}, C₃-C₆-cycloalkyl, five- or six-membered heteroaryl and aryl; wherein the heteroaryl contains one, two or three heteroatoms selected from N, O and S; wherein
R^{x} is as defined above;
R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl; phenyl and phenyl-C₁-C₆-alkyl; wherein the phenyl group is unsubstituted or substituted with substituents selected from the group consisting of halogen, CN, OH, C₁-C₆-alkyl, C₁-C₆-halogenalkyl, C₁-C₆-alkoxy and C₁-C₆-halogenalkoxy;
wherein the acyclic moieties of R² are unsubstituted or substituted with groups R^{2a} which independently of one another are selected from:
R^{2a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl and phenoxy group is unsubstituted or substituted with substituents R^{21a} selected from the group consisting of halogen, OH, C₁-C₄-alkyl, C₁-C₄-halogenalkyl, C₁-C₄-alkoxy and C₁-C₄-halogenalkoxy;
wherein the carbocycle, heteroaryl and aryl moieties of R² are unsubstituted or substituted with groups R^{2b} which independently of one another are selected from:
R^{2b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

According to one embodiment of formula I, R² is selected from the group consisting of halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl, and OR^{Y}.

According to still another embodiment of formula I, R² is halogen, in particular F, Cl, Br or I, more specifically F, Cl or Br, in particular F or Cl.

According to still another embodiment of formula I, R² is F.

According to still another embodiment of formula I, R² is Cl.

According to still another embodiment of formula I, R² is Br.

According to still another embodiment of formula I, R² is COOH.

According to still another embodiment of formula I, R² is CONH₂.

According to still another embodiment of formula I, R² is OH.

According to still another embodiment of formula I, R² is CN.

According to still another embodiment of formula I, R² is NO₂.

According to still another embodiment of formula I, R² is SH.

According to still another embodiment of formula I, R² is NH₂.

According to still another embodiment of formula I, R² is, NH(C₁-C₄-alkyl), in particular NH(CH₃), NH(C₂H₅).

According to still another embodiment of formula I, R² is, N(C₁-C₄-alkyl)₂, in particular NH(CH₃)₂, NH(C₂H₅)₂.

According to still another embodiment of formula I, R² is, NH(C₂-C₄-alkenyl), in particular NH(CH═CH₂), NH(CH₂CH═CH₂).

According to still another embodiment of formula I, R² is N(C₂-C₄-alkenyl)₂, in particular N(CH═CH₂)₂, N(CH₂CH═CH₂)₂.

According to still another embodiment of formula I, R² is, NH(C₂-C₄-alkynyl), in particular NH(C≡CH), NH(CH₂C≡CH).

According to still another embodiment of formula I, R² is, N(C₂-C₄-alkynyl)₂, in particular N(C≡CH)₂, N(CH₂C≡CH)₂.

According to still another embodiment of formula I, R² is, NH(C₃-C₆-cycloalkyl), in particular NH(C₃H₇), NH(C₄H₂).

According to still another embodiment of formula I, R² is N(C₃-C₆-cycloalkyl)₂, in particular N(C₃H₇)₂, N(C₄H₂)₂.

According to still another embodiment of formula I, R² is N(C₁-C₄-alkyl)(C₂-C₄-alkenyl), in particular N(CH₃)(CH═CH₂), N(CH₃)(CH₂CH═CH₂), N(C₂H₅)(CH═CH₂), N(C₂H₅)(CH₂CH═CH₂).

According to still another embodiment of formula I, R² is N(C₁-C₄-alkyl)(C₂-C₄-alkynyl), in particular N(CH₃)(C≡CH), N(CH₃)(CH₂C≡CH), N(C₂H₅)(C≡CH), N(C₂H₅)(CH₂C≡CH).

According to still another embodiment of formula I, R² is N(C₁-C₄-alkyl)(C₃-C₆-cycloalkyl), in particular N(CH₃)(C₃H₇), N(CH₃)(C₄H₂), N(C₂H₅)(C₃H₇), N(CH₃)(C₄H₂).

According to still another embodiment of formula I, R² is N(C₂-C₄-alkenyl)(C₂-C₄-alkynyl), in particular N(CH═CH₂)(C≡CH), N(CH₂CH═CH₂)(CH₂C≡CH), N(CH═CH₂)(C≡CH), N(CH₂CH═CH₂)(CH₂C≡CH).

According to still another embodiment of formula I, R² is N(C₂-C₄-alkenyl)(C₃-C₆-cycloalkyl), in particular N(CH═CH₂)(C₃H₇), N(CH₂CH═CH₂)(C₄H₂), N(CH═CH₂)(C₃H₇), N(CH₂CH═CH₂)(C₄H₂).

According to still another embodiment of formula I, R² is N(C₂-C₄-alkynyl)(C₃-C₆-cycloalkyl), in particular N(C≡CH)(C₃H₇), N(CH₂C≡CH)(C₄H₂), N(C≡CH)(C₃H₇), N(CH₂C≡CH)(C₄H₂).

According to still another embodiment of formula I, R² is, NH(C(═O)(C₁-C₄-alkyl), in particular NH(C(═O)(CH₃), NH(C(═O)(C₂H₅).

According to still another embodiment of formula I, R² is N(C(═O)(C₁-C₄-alkyl)₂, in particular N(C(═O)(CH₃)₂, N(C(═O)(C₂H₅)₂.

According to a further specific embodiment of formula I, R² is NH—SO₂—R^{x} such as NH—SO₂—CH₃, NH—SO₂—CH₂—CH₃, NH—SO₂—CF₃, NH—SO₂-Ts.

According to still another embodiment of formula I, R² is S(O)ₙ—C₁-C₆-alkyl such as SCH₃, S(═O) CH₃, S(O)₂CH₃.

According to still another embodiment of formula I, R² is S(O)ₙ-aryl such as S-phenyl, S(═O) phenyl, S(O)₂phenyl.

According to still another embodiment of formula I, R² is S(O)ₙ—C₂-C₆-alkenyl such as SCH═CH₂, S(═O)CH═CH₂, S(O)₂CH═CH₂, SCH₂CH═CH₂, S(═O)CH₂CH═CH₂, S(O)₂CH₂CH═CH₂.

According to still another embodiment of formula I, R² is S(O)ₙ—C₂-C₆-alkynyl such as SC≡CH, S(═O)C≡CH, S(O)₂C≡CH, SCH₂C≡CH, S(═O)CH₂C≡CH, S(O)₂CH₂C≡CH.

According to a further specific embodiment of formula I, R² is CH(═O).

According to a further specific embodiment of formula I, R² is C(═O)C₁-C₆-alkyl, C(═O)O(C₁-C₆-alkyl) or C(═O)NH(C₁-C₆-alkyl), wherein alkyl is CH₃, C₂H₅, n-propyl, i-propyl, n-butyl, i-butyl, tert-butyl, n-pentyl or i-pentyl.

According to a further specific embodiment of formula I, R² is C(═O)C₂-C₆-alkenyl, C(═O)O(C₂-C₆-alkenyl) or (═O)NH(C₂-C₆-alkenyl) wherein alkenyl is CH═CH₂, CH₂CH═CH₂.

According to a further specific embodiment of formula I, R² is C(═O)C₂-C₆-alkynyl, C(═O)O(C₂-C₆-alkynyl) or C(═O)NH(C₂-C₆-alkynyl), wherein alkynyl is C≡CH, CH₂C≡CH.

According to a further specific embodiment of formula I, R² is C(═O)C₃-C₆-cycloalkyl, C(═O)O(C₃-C₆-cycloalkyl) or C(═O)NH(C₃-C₆-cycloalkyl), wherein cycloalkyl is cyclopropyl (C₃H₇) or cyclobutyl (C₄H₂).

According to a further specific embodiment of formula I, R² is CH(═S).

According to a further specific embodiment of formula I, R² is C(═S)C₁-C₆-alkyl, C(═S)OC₁-C₆-alkyl, C(═S)NH(C₁-C₆-alkyl) or C(═S)NH(C₁-C₆-alkyl), wherein alkyl is CH₃, C₂H₅, n-propyl, i-propyl, n-butyl, i-butyl, tert-butyl, n-pentyl or i-pentyl.

According to a further specific embodiment of formula I, R² is C(═S)C₂-C₆-alkenyl, C(═S)OC₂-C₆-alkenyl, C(═S)NH(C₂-C₆-alkenyl) or C(═S)N(C₂-C₆-alkenyl)₂, wherein alkenyl is CH═CH₂, CH₂CH═CH₂.

According to a further specific embodiment of formula I, R² is C(═S)C₂-C₆-alkynyl, C(═S)O(C₂-C₆-alkynyl), C(═S)NH(C₂-C₆-alkynyl) or C(═S)N(C₂-C₆-alkynyl)₂, wherein alkynyl is C≡CH, CH₂C≡CH.

According to a further specific embodiment of formula I, R² is C(═S)C₃-C₆-cycloalkyl, C(═S)O(C₃-C₇-cycloalkyl), C(═S)NH(C₃-C₇-cycloalkyl) or, C(═S)N(C₃-C₇-cycloalkyl)₂, wherein cycloalkyl is cyclopropyl (C₃H₇) or cyclobutyl (C₄H₂).

According to still another embodiment of formula I, R² is C₁-C₆-alkyl, in particular C₁-C₄-alkyl, such as CH₃. or C₂H₅, in particular CH₃ or CH₂CH₃.

According to still another embodiment of formula I, R² is C₁-C₆-halogenalkyl, in particular C₁-C₄-halogenalkyl, such as CF₃, CCl₃, FCH₂, ClCH₂, F₂CH, Cl₂CH, CF₃CH₂, CCl₃CH₂ or CF₂CHF₂. According to still a further embodiment of formula I, R² is C₂-C₆-alkenyl, in particular C₂-C₄-alkenyl, such as CH═CH₂, C(CH₃)═CH₂, CH₂CH═CH₂.

According to a further specific embodiment of formula I, R² is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CH₂CH═CHF, CH₂CH═CHCl, CH₂CH═CF₂, CH₂CH═CCl₂, CF₂CH═CF₂, CCl₂CH═CCl₂, CF₂CF═CF₂, CCl₂CCl═CCl₂.

According to still a further embodiment of formula I, R² is C₂-C₆-alkynyl or C₂-C₆-halogenalkynyl, in particular C₂-C₄-alkynyl or C₂-C₄-halogenalkynyl, such as C≡CH, CH₂C≡CH, C≡CCl, CH₂C≡CCl, or CCl₂C≡CCl.

According to a further specific embodiment of formula I, R² is OR^{Y}, wherein R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl.

According to a further specific embodiment of formula I, R² is OR^{Y}, wherein R^{Y} is C₁-C₆-alkyl, in particular C₁-C₄-alkyl, more specifically C₁-C₂-alkoxy. R² is such as OCH₃ or OCH₂CH₃.

According to a further specific embodiment of formula I, R² is OR^{Y}, wherein R^{Y} is C₁-C₆-halogenalkyl, in particular C₁-C₄-halogenalkyl, more specifically C₁-C₂-halogenalkyl. R² is such as OCF₃, OCHF₂, OCH₂F, OCCl₃, OCHCl₂ or OCH₂Cl, in particular OCF₃, OCHF₂, OCCl₃ or OCHCl₂.

According to a further specific embodiment of formula I, R² is OR^{Y}, wherein R^{Y} C₂-C₆-alkenyl, in particular C₂-C₄-alkenyl, more specifically C₁-C₂-alkenyl. R² is such as OCH═CH₂, OCH₂CH═CH₂.

According to a further specific embodiment of formula I, R² is OR^{Y}, wherein R^{Y} C₂-C₆-halogenalkenyl, in particular C₂-C₄-halogenalkenyl, more specifically C₁-C₂-halogenalkenyl.

According to a further specific embodiment of formula I, R² is OR^{Y}, wherein R^{Y} C₂-C₆-alkynyl, in particular C₂-C₆-alkynyl, in particular C₂-C₄-alkynyl, more specifically C₁-C₂-alkynyl. R² is such as OC≡CH,

According to a further specific embodiment of formula I, R² is OR^{Y}, wherein R^{Y} C₂-C₆-halogenalkynyl, in particular C₂-C₆-halogenalkynyl, in particular C₂-C₄-halogenalkynyl, more specifically C₁-C₂-halogenalkynyl. R² is such as OC≡CCl, OCH₂C≡CCl, or OCCl₂C≡CCl.

According to still another embodiment of formula I, R² is is OR^{Y}, wherein R^{Y} C₃-C₆-cycloalkenyl, in particular cyclopropenyl.

According to still another embodiment of formula I, R² is C₃-C₆-cycloalkyl, in particular cyclopropyl.

According to still another embodiment of formula I, R² is C₃-C₆-halogencycloalkyl. In a special embodiment R^{2b} is fully or partially halogenated cyclopropyl, such as 1-F-cyclopropyl, 1-Cl-cyclopropyl, 1,1-F₂-cyclopropyl, 1,1-Cl₂-cyclopropyl.

According to still another embodiment of formula I, R² is phenyl-C₁-C₆-alkyl, such as phenyl-CH₂, wherein the phenyl moiety in each case is unsubstituted or substituted by one, two or three identical or different groups R^{2b} which independently of one another are selected from halogen, C₁-C₂-alkyl, C₁-C₂-alkoxy, C₁-C₂-halogenalkyl and C₁-C₂-halogenalkoxy, in particular F, Cl, Br, CH₃, OCH₃, CF₃ and OCF₃.

According to still another embodiment of formula I, R² is aryl, in particular phenyl, wherein the aryl or phenyl moiety in each case is unsubstituted or substituted with identical or different groups R^{2b} which independently of one another are selected from CN, halogen, C₁-C₂-alkyl, C₁-C₂-alkoxy, C₁-C₂-halogenalkyl and C₁-C₂-halogenalkoxy, in particular CN, F, Cl, Br, CH₃, OCH₃, CHF₂, OCHF₂, CF₃ and OCF₃. According to one embodiment, R² is unsubstituted phenyl. According to another embodiment, R² is phenyl, that is substituted with one, two or three, in particular one, halogen, in particular selected from F, Cl and Br, more specifically selected from F and Cl.

According to still another embodiment of formula I, R² is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R² is a 6-membered heteroaryl such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to still another embodiment of formula I, R² is in each case independently selected from halogen, CN, C₁-C₆-alkyl, C₁-C₆-halogenalkyl, C₂-C₆-alkenyl, C₂-C₆-alkynyl, C₁-C₆-alkoxy, C₁-C₆-halogenalkoxy, C₃-C₆-alkenyloxy, C₃-C₆-alkynyloxy, C₃-C₆-cycloalkyl and C₃-C₆-halogencycloalkyl, wherein the acyclic moieties of R² are unsubstituted or substituted with identical or different groups R^{2a} as defined and preferably defined herein, and wherein the carbocyclic, phenyl and heteroaryl moieties of R² are unsubstituted or substituted with identical or different groups R^{2b} as defined and preferably defined herein.

According to still another embodiment of formula I, R² is in each case independently selected from halogen, CN, C₁-C₆-alkyl, C₁-C₆-halogenalkyl, C₂-C₆-alkenyl, C₂-C₆-alkynyl, C₁-C₆-alkoxy, C₁-C₆-halogenalkoxy; wherein the acyclic moieties of R² are unsubstituted or substituted with identical or different groups R^{2a} as defined and preferably defined herein.

According to still another embodiment of formula I, R² is in each case independently selected from CN, halogen, C₁-C₆-alkyl, C₁-C₆-halogenalkyl, C₂-C₆-alkynyl, OR^{Y}, C₃-C₆-cycloalkyl; wherein R^{Y} is C₁-C₆-alkyl, C₁-C₆-halogenalkyl, C₂-C₆-alkenyl or C₂-C₆-alkynyl.

R^{2a} are the possible substituents for the acyclic moieties of R².

According to one embodiment R^{2a} is independently selected from halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl and phenyl group is unsubstituted or substituted with substituents R^{21a} selected from the group consisting of halogen, OH, C₁-C₄-alkyl, C₁-C₄-halogenalkyl, C₁-C₄-alkoxy and C₁-C₄-halogenalkoxy.

According to one embodiment R^{2a} is independently selected from halogen, C₁-C₆-alkoxy, C₃-C₆-cycloalkyl, C₃-C₆-halogencycloalkyl and C₁-C₄-halogenalkoxy. Specifically, R^{2a} is independently selected from F, Cl, Br, I, C₁-C₂-alkoxy, cyclopropyl, 1-F-cyclopropyl, 1-Cl-cyclopropyl, 1,1-F₂-cyclopropyl, 1,1-Cl₂-cyclopropyl and C₁-C₂-halogenalkoxy.

According to still another embodiment of formula I, R^{2a} is independently halogen, in particular selected from F, Cl, Br and I, more specifically F, Cl and Br.

R^{2b} are the possible substituents for the carbocyclic, heteroaryl and phenyl moieties of R². R^{2b} according to the invention is independently selected from halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

According to one embodiment thereof R^{2b} is independently selected from halogen, CN, C₁-C₄-alkyl, C₁-C₄-alkoxy, C₁-C₄-halogenalkyl and C₁-C₄-halogenalkoxy, in particular halogen, C₁-C₄-alkyl and C₁-C₄-alkoxy. Specifically, R^{2b} is independently selected from F, Cl, CN, CH₃, OCH₃ and halogenmethoxy.

Particularly preferred embodiments of R² according to the invention are in Table P2 below, wherein each line of lines P2-1 to P2-41 corresponds to one particular embodiment of the invention, wherein P2-1 to P2-41 are also in any combination with one another a preferred embodiment of the present invention. The connection point to the carbon atom, to which R² is bound is marked with “#” in the drawings.

TABLE P2

| No. | R² |
|---|---|
| P2-1 | CH₃ |
| P2-2 | CH₂F |
| P2-3 | CHF₂ |
| P2-4 | CF₃ |
| P2-5 | C₂H₅ |
| P2-6 | CH(CH₃)₂ |
| P2-7 | CH₂CH₂CH₃ |
| P2-8 | CH₂CH₂CH₂CH₃ |
| P2-9 | CH₂CH(CH₃)₂ |
| P2-10 | C(CH₃)₃ |
| P2-11 | CH₂CH₂CH₂CH₂CH₃ |
| P2-12 | CH═CH₂ |
| P2-13 | CH₂CH═CH₂ |
| P2-14 | C≡CH |
| P2-15 | CH₂C≡CH |
| P2-16 | CH₂CH₂CH(CH₃)₂ |
| P2-17 | OH |
| P2-18 | OCH₃ |
| P2-19 | OCHF₂ |
| P2-20 | OC₂H₅ |
| P2-21 | CN |
| P2-22 | F |
| P2-23 | Cl |
| P2-24 | Br |
| P2-25 | NO₂ |
| P2-26 | NH₂ |
| P2-27 | CO—NH₂ |
| P2-28 | CO—NH(CH₃) |
| P2-29 | HNCH₃ |
| P2-30 | HNC₂H₅ |
| P2-31 | (CH₃)₂N |
| P2-32 | SO₂H |
| P2-33 | SO₂—CH₃ |
| P2-34 | SO—CH₃ |
| P2-35 | S—CH₃ |
| P2-36 | 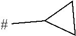 |
| P2-37 | 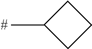 |
| P2-38 | 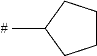 |
| P2-39 | 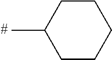 |
| P2-40 | 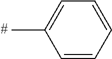 |
| P2-41 | 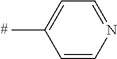 |

R³ is in each case independently selected from halogen, OH, COOH, CONH₂, CN, NO₂,SH, NH₂, NH(C₁-C₄-alkyl), N(C₁-C₄-alkyl)₂, NH(C₂-C₄-alkenyl), N(C₂-C₄-alkenyl)₂, NH(C₂-C₄-alkynyl), N(C₂-C₄-alkynyl)₂, NH(C₃-C₆-cycloalkyl), N(C₃-C₆-cycloalkyl)₂, N(C₂-C₄-alkyl)(C₂-C₄-alkenyl), N(C₂-C₄-alkyl)(C₂-C₄-alkynyl), N(C₂-C₄-alkyl)(C₃-C₆-cycloalkyl), N(C₂-C₄-alkenyl)(C₂-C₄-alkynyl), N(C₂-C₄-alkenyl)(C₃-C₆-cycloalkyl), N(C₂-C₄-alkynyl)(C₃-C₆-cycloalkyl), NH((═O)C₁-C₄-alkyl), N(C(═O)C₁-C₄-alkyl)₂, NH—SO₂—R^{x}, S(O)ₙ—C₁-C₆-alkyl, S(O)ₙ-aryl, C₁-C₆-cycloalkylthio, S(O)ₙ—C₂-C₆-alkenyl, S(O)ₙ—C₂-C₆-alkynyl, CH(═O), C(═O)C₁-C₆-alkyl, C(═O)C₂-C₆-alkenyl, C(═O)C₂-C₆-alkynyl, C(═O)C₃-C₆-cycloalkyl, C(═O)NH(C₁-C₆-alkyl), CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₂-C₆-alkenyl), C(═S)O(C₂-C₆-alkynyl), C(═S)O(C₃-C₇-cycloalkyl), C(═S)NH(C₁-C₆-alkyl), C(═S)NH(C₂-C₆-alkenyl), C(═S)NH(C₂-C₆-alkynyl), C(═S)NH(C₃-C₇-cycloalkyl),C(═S)N(C₁-C₆-alkyl)₂, C(═S)N(C₂-C₆-alkenyl)₂, C(═S)N(C₂-C₆-alkynyl)₂, C(═S)N(C₃-C₇-cycloalkyl)₂, C₁-C₆-alkyl, C₂-C₆-alkenyl, C₂-C₆-alkynyl, OR^{Y}, C₃-C₆-cycloalkyl, five- or six-membered heteroaryl and aryl; wherein the heteroaryl contains one, two or three heteroatoms selected from N, O and S; wherein
R^{x} is as defined above;
R^{Y} is as defined above;
wherein the acyclic moieties of R³ are unsubstituted or substituted with groups R^{3a} which independently of one another are selected from:
R^{3a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl and phenyl group is unsubstituted or substituted with substituents R^{31a} selected from the group consisting of halogen, OH, C₁-C₄-alkyl, C₁-C₄-halogenalkyl, C₁-C₄-alkoxy and C₁-C₄-halogenalkoxy;
wherein the carbocyclic, heteroaryl and aryl moieties of R³ are unsubstituted or substituted with groups R^{3b} which independently of one another are selected from:
R^{3b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

According to one embodiment of formula I, R³ is selected from the group consisting of halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy and OR^{Y}.

R³ is selected from the group consisting of halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy and OR^{Y}.

According to still another embodiment of formula I, R³ is halogen, in particular F, Cl, Br or I, more specifically F, Cl or Br, in particular F or Cl.

According to still another embodiment of formula I, R³ is F.

According to still another embodiment of formula I, R³ is Cl.

According to still another embodiment of formula I, R³ is Br.

According to still another embodiment of formula I, R³ is OH.

According to still another embodiment of formula I, R² is COOH.

According to still another embodiment of formula I, R² is CONH₂.

According to still another embodiment of formula I, R³ is CN.

According to still another embodiment of formula I, R³ is NO₂.

According to still another embodiment of formula I, R³ is SH.

According to still another embodiment of formula I, R³ is NH₂.

According to still another embodiment of formula I, R³ is, NH(C₁-C₄-alkyl), in particular NH(CH₃), NH(C₂H₅).

According to still another embodiment of formula I, R³ is, N(C₁-C₄-alkyl)₂, in particular NH(CH₃)₂, NH(C₂H₅)₂.

According to still another embodiment of formula I, R³ is NH(C₂-C₄-alkenyl), in particular NH(CH═CH₂), NH(CH₂CH═CH₂).

According to still another embodiment of formula I, R³ is, N(C₂-C₄-alkenyl)₂, in particular N(CH═CH₂)₂, N(CH₂CH═CH₂)₂.

According to still another embodiment of formula I, R³ is, NH(C₂-C₄-alkynyl), in particular NH(C≡CH), NH(CH₂C≡CH).

According to still another embodiment of formula I, R³ is, N(C₂-C₄-alkynyl)₂, in particular N(C≡CH)₂, N(CH₂C≡CH)₂.

According to still another embodiment of formula I, R³ is NH(C₃-C₆-cycloalkyl), in particular NH(C₃H₇), NH(C₄H₉).

According to still another embodiment of formula I, R³ is, N(C₃-C₆-cycloalkyl)₂, in particular N(C₃H₇)₂, N(C₄H₉)₂.

According to still another embodiment of formula I, R³ is N(C₁-C₄-alkyl)(C₂-C₄-alkenyl), in particular N(CH₃)(CH═CH₂), N(CH₃)(CH₂CH═CH₂), N(C₂H₅)(CH═CH₂), N(C₂H₅)(CH₂CH═CH₂).

According to still another embodiment of formula I, R³ is N(C₁-C₄-alkyl)(C₂-C₄-alkynyl), in particular N(CH₃)(C≡CH), N(CH₃)(CH₂C≡CH), N(C₂H₅)(C≡CH), N(C₂H₅)(CH₂C≡CH).

According to still another embodiment of formula I, R³ is N(C₁-C₄-alkyl)(C₃-C₆-cycloalkyl), in particular N(CH₃)(C₃H₇), N(CH₃)(C₄H₉), N(C₂H₅)(C₃H₇), N(CH₃)(C₄H₉).

According to still another embodiment of formula I, R³ is N(C₂-C₄-alkenyl)(C₂-C₄-alkynyl), in particular N(CH═CH₂)(C≡CH), N(CH₂CH═CH₂)(CH₂C≡CH), N(CH═CH₂)(C≡CH), N(CH₂CH═CH₂)(CH₂C≡CH).

According to still another embodiment of formula I, R³ is N(C₂-C₄-alkenyl)(C₃-C₆-cycloalkyl), in particular N(CH═CH₂)(C₃H₇), N(CH₂CH═CH₂)(C₄H₉), N(CH═CH₂)(C₃H₇), N(CH₂CH═CH₂)(C₄H₉).

According to still another embodiment of formula I, R³ is N(C₂-C₄-alkynyl)(C₃-C₆-cycloalkyl), in particular N(C≡CH)(C₃H₇), N(CH₂C≡CH)(C₄H₉), N(C≡CH)(C₃H₇), N(CH₂C≡CH)(C₄H₉).

According to still another embodiment of formula I, R³ is, NH(C(═O)(C₁-C₄-alkyl), in particular NH(C(═O)(CH₃), NH(C(═O)(C₂H₅).

According to still another embodiment of formula I, R³ is N(C(═O)(C₁-C₄-alkyl)₂, in particular N(C(═O)(CH₃)₂, N(C(═O)(C₂H₅)₂.

According to a further specific embodiment of formula I, R³ is NH—SO₂—R^{x} such as NH—SO₂—CH₃, NH—SO₂—CH₂—CH₃, NH—SO₂—CF₃, NH—SO₂-Ts.

According to still another embodiment of formula I, R³ is S(O)ₙ—C₁-C₆-alkyl such as SCH₃, S(═O) CH₃, S(O)₂CH₃.

According to still another embodiment of formula I, R³ is S(O)ₙ-aryl such as S-phenyl, S(═O) phenyl, S(O)₂phenyl.

According to still another embodiment of formula I, R³ is S(O)ₙ—C₂-C₆-alkenyl such as SCH═CH₂, S(═O)CH═CH₂, S(O)₂CH═CH₂, SCH₂CH═CH₂, S(═O)CH₂CH═CH₂, S(O)₂CH₂CH═CH₂.

According to still another embodiment of formula I, R³ is S(O)ₙ—C₂-C₆-alkynyl such as SC≡CH, S(═O)C≡CH, S(O)₂C≡CH, SCH₂C≡CH, S(═O)CH₂C≡CH, S(O)₂CH₂C≡CH.

According to a further specific embodiment of formula I, R³ is CH(═O).

According to a further specific embodiment of formula I, R³ is C(═O)C₁-C₆-alkyl, C(═O)O(C₁-C₆-alkyl) or C(═O)NH(C₁-C₆-alkyl), wherein alkyl is CH₃, C₂H₅, n-propyl, i-propyl, n-butyl, i-butyl, tert-butyl, n-pentyl or i-pentyl.

According to a further specific embodiment of formula I, R³ is C(═O)C₂-C₆-alkenyl, C(═O)O(C₂-C₆-alkenyl) or C(═O)NH(C₂-C₆-alkenyl), wherein alkenyl is CH═CH₂, C(CH₃)═CH₂, CH₂CH═CH₂.

According to a further specific embodiment of formula I, R³ is C(═O)C₂-C₆-alkynyl, C(═O)O(C₂-C₆-alkynyl) or C(═O)NH(C₂-C₆-alkynyl), wherein alkynyl is C≡CH, CH₂C≡CH, According to a further specific embodiment of formula I, R³ is C(═O)C₃-C₆-cycloalkyl, C(═O)O(C₃-C₆-cycloalkyl) or C(═O)NH(C₃-C₆-cycloalkyl), wherein cycloalkyl is cyclopropyl (C₃H₇) or cyclobutyl (C₄H₉).

According to a further specific embodiment of formula I, R³ is CH(═S).

According to a further specific embodiment of formula I, R³ is C(═S)C₁-C₆-alkyl, C(═S)OC₁-C₆-alkyl, C(═S)NH(C₁-C₆-alkyl) or C(═S)NH(C₁-C₆-alkyl), wherein alkyl is CH₃, C₂H₅, n-propyl, i-propyl, n-butyl, i-butyl, tert-butyl, n-pentyl or i-pentyl.

According to a further specific embodiment of formula I, R³ is C(═S)C₂-C₆-alkenyl, C(═S)OC₂-C₆-alkenyl, C(═S)NH(C₂-C₆-alkenyl) or C(═S)N(C₂-C₆-alkenyl)₂, wherein alkenyl is CH═CH₂, CH₂CH═CH₂.

According to a further specific embodiment of formula I, R³ is C(═S)C₂-C₆-alkynyl, C(═S)O(C₂-C₆-alkynyl), C(═S)NH(C₂-C₆-alkynyl) or C(═S)N(C₂-C₆-alkynyl), wherein alkynyl is C≡CH, CH₂C≡CH.

According to a further specific embodiment of formula I, R³ is C(═S)C₃-C₆-cycloalkyl, C(═S)O(C₃-C₇-cycloalkyl), C(═S)NH(C₃-C₇-cycloalkyl) or, C(═S)N(C₃-C₇-cycloalkyl)₂, wherein cycloalkyl is cyclopropyl (C₃H₇) or cyclobutyl (C₄H₉).

According to still another embodiment of formula I, R³ is C₁-C₆-alkyl, in particular C₁-C₄-alkyl, such as CH₃. or C₂H₅, in particular CH₃ or CH₂CH₃.

According to still another embodiment of formula I, R³ is C₁-C₆-halogenalkyl, in particular C₁-C₄-halogenalkyl, such as CF₃, CCl₃, FCH₂, ClCH₂, F₂CH, Cl₂CH, CF₃CH₂, CCl₃CH₂ or CF₂CHF₂.

According to still a further embodiment of formula I, R³ is C₂-C₆-alkenyl, in particular C₂-C₄-alkenyl, such as CH═CH₂.

According to a further specific embodiment of formula I, R³ is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CH₂CH═CHF, CH₂CH═CHCl, CH₂CH═CF₂, CH₂CH═CCl₂, CF₂CH═CF₂, CCl₂CH═CCl₂, CF₂CF═CF₂, CCl₂CCl═CCl₂.

According to still a further embodiment of formula I, R³ is C₂-C₆-alkynyl or C₂-C₆-halogenalkynyl, in particular C₂-C₄-alkynyl or C₂-C₄-halogenalkynyl, such as C≡CH, CH₂ C≡CH, C≡CCl, CH₂C≡CCl, or CCl₂C≡CCl.

According to a further specific embodiment of formula I, R³ is OR^{Y}, wherein R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl.

According to a further specific embodiment of formula I, R³ is OR^{Y}, wherein R^{Y} is C₁-C₆-alkyl, in particular C₁-C₄-alkyl, more specifically C₁-C₂-alkoxy. R³ is such as OCH₃ or OCH₂CH₃.

According to a further specific embodiment of formula I, R³ is OR^{Y}, wherein R^{Y} is C₁-C₆-halogenalkyl, in particular C₁-C₄-halogenalkyl, more specifically C₁-C₂-halogenalkyl. R³ is such as OCF₃, OCHF₂, OCH₂F, OCCl₃, OCHCl₂ or OCH₂Cl, in particular OCF₃, OCHF₂, OCCl₃ or OCHCl₂.

According to a further specific embodiment of formula I, R³ is OR^{Y}, wherein R^{Y} C₂-C₆-alkenyl, in particular C₂-C₄-alkenyl, more specifically C₁-C₂-alkenyl. R³ is such as OCH═CH₂, OCH₂CH═CH₂.

According to a further specific embodiment of formula I, R³ is OR^{Y}, wherein R^{Y} C₂-C₆-alkynyl, in particular C₂-C₆-alkynyl, in particular C₂-C₄-alkynyl, more specifically C₁-C₂-alkynyl. R³ is such as OC≡CH, OC≡CCl, OCH₂C≡CCl, or OCCl₂C≡CCl

According to still another embodiment of formula I R³ is OR^{Y}, wherein R^{Y} is C₃-C₆-cycloalkyl, in particular cyclopropyl.

According to still another embodiment of formula I, R³ is OR^{Y}, wherein R^{Y} is C₃-C₆-halogencycloalkyl. In a special embodiment R¹ is fully or partially halogenated cyclopropyl.

According to still another embodiment of formula I, R³ is is OR^{Y}, wherein R^{Y} C₃-C₆-cycloalkenyl, in particular cyclopropenyl.

According to still another embodiment of formula I, R³ is C₃-C₆-cycloalkyl, in particular cyclopropyl.

According to still another embodiment of formula I, R³ is C₃-C₆-halogencycloalkyl. In a special embodiment R^{3b} is fully or partially halogenated cyclopropyl, such as 1-F-cyclopropyl, 1-Cl-cyclopropyl, 1,1-F₂-cyclopropyl, 1,1-Cl₂-cyclopropyl

According to still another embodiment of formula I, R³ is phenyl-C₁-C₆-alkyl, such as phenyl-CH₂, wherein the phenyl moiety in each case is unsubstituted or substituted by one, two or three identical or different groups R^{3b} which independently of one another are selected from halogen, C₁-C₂-alkyl, C₁-C₂-alkoxy, C₁-C₂-halogenalkyl and C₁-C₂-halogenalkoxy, in particular F, Cl, Br, CH₃, OCH₃, CF₃ and OCF₃.

According to still another embodiment of formula I, R³ is aryl, in particular phenyl, wherein the aryl or phenyl moiety in each case is unsubstituted or substituted with identical or different groups R^{3b} which independently of one another are selected from CN, halogen, C₁-C₂-alkyl, C₁-C₂-alkoxy, C₁-C₂-halogenalkyl and C₁-C₂-halogenalkoxy, in particular CN, F, Cl, Br, CH₃, OCH₃, CHF₂, OCHF₂, CF₃ and OCF₃. According to one embodiment, R³ is unsubstituted phenyl. According to another embodiment, R³ is phenyl, that is substituted with one, two or three, in particular one, halogen, in particular selected from F, Cl and Br, more specifically selected from F and Cl.

According to still another embodiment of formula I, R³ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R³ is a 6-membered heteroaryl such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to still another embodiment of formula I, R³ is in each case independently selected from halogen, CN, C₁-C₆-alkyl, C₁-C₆-halogenalkyl, C₂-C₆-alkenyl, C₂-C₆-alkynyl, C₁-C₆-alkoxy, C₁-C₆-halogenalkoxy, C₃-C₆-alkenyloxy, C₃-C₆-alkynyloxy, C₃-C₆-cycloalkyl and C₃-C₆-halogencycloalkyl, wherein the acyclic moieties of R³ are unsubstituted or substituted with identical or different groups R^{3a} as defined and preferably defined herein, and wherein the carbocyclic, phenyl and heteroaryl moieties of R³ are unsubstituted or substituted with identical or different groups R^{3b} as defined and preferably defined herein.

According to still another embodiment of formula I, R³ is in each case independently selected from halogen, CN, C₁-C₆-alkyl, C₁-C₆-halogenalkyl, C₂-C₆-alkenyl, C₂-C₆-alkynyl, C₁-C₆-alkoxy, C₁-C₆-halogenalkoxy; wherein the acyclic moieties of R³ are unsubstituted or substituted with identical or different groups R^{3a} as defined and preferably defined herein.

According to still another embodiment of formula I, R³ is in each case independently selected from CN, halogen, C₁-C₆-alkyl, C₁-C₆-halogenalkyl, C₂-C₆-alkynyl, OR^{Y}, C₃-C₆-cycloalkyl; wherein R^{Y} is C₁-C₆-alkyl, C₁-C₆-halogenalkyl, C₂-C₆-alkenyl or C₂-C₆-alkynyl.

R^{3a} are the possible substituents for the acyclic moieties of R³.

According to one embodiment R^{3a} is independently selected from halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl and phenyl group is unsubstituted or substituted with substituents R^{31a} selected from the group consisting of halogen, OH, C₁-C₄-alkyl, C₁-C₄-halogenalkyl, C₁-C₄-alkoxy and C₁-C₄-halogenalkoxy.

According to one embodiment R^{3a} is independently selected from halogen, C₁-C₆-alkoxy, C₃-C₆-cycloalkyl, C₃-C₆-halogencycloalkyl and C₁-C₄-halogenalkoxy. Specifically, R^{3a} is independently selected from F, Cl, Br, I, C₁-C₂-alkoxy, cyclopropyl, 1-F-cyclopropyl, 1-Cl-cyclopropyl, 1,1-F₂-cyclopropyl, 1,1-Cl₂-cyclopropyl and C₁-C₂-halogenalkoxy.

According to still another embodiment of formula I, R^{3a} is independently halogen, in particular selected from F, Cl, Br and I, more specifically F, Cl and Br.

R^{3b} are the possible substituents for the carbocyclic, heteroaryl and phenyl moieties of R³. R^{3b} according to the invention is independently selected from halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

According to one embodiment thereof R^{3b} is independently selected from halogen, CN, C₁-C₄-alkyl, C₁-C₄-alkoxy, C₁-C₄-halogenalkyl and C₁-C₄-halogenalkoxy, in particular halogen, C₁-C₄-alkyl and C₁-C₄-alkoxy. Specifically, R^{3b} is independently selected from F, Cl, CN, CH₃, OCH₃ and halogenmethoxy.

Particularly preferred embodiments of R³ according to the invention are in Table P3 below, wherein each line of lines P3-1 to P3-41 corresponds to one particular embodiment of the invention, wherein P3-1 to P3-41 are also in any combination with one another a preferred embodiment of the present invention. The connection point to the carbon atom, to which R³ is bound is marked with “#” in the drawings.

TABLE P3

| No. | R³ |
|---|---|
| P3-1 | CH₃ |
| P3-2 | CH₂F |
| P3-3 | CHF₂ |
| P3-4 | CF₃ |
| P3-5 | C₂H₅ |
| P3-6 | CH(CH₃)₂ |
| P3-7 | CH₂CH₂CH₃ |
| P3-8 | CH₂CH₂CH₂CH₃ |
| P3-9 | CH₂CH(CH₃)₂ |
| P3-10 | C(CH₃)₃ |
| P3-11 | CH₂CH₂CH₂CH₂CH₃ |
| P3-12 | CH═CH₂ |
| P3-13 | CH₂CH═CH₂ |
| P3-14 | C≡CH |
| P3-15 | CH₂C≡CH |
| P3-16 | CH₂CH₂CH(CH₃)₂ |
| P3-17 | OH |
| P3-18 | OCH₃ |
| P3-19 | OCHF₂ |
| P3-20 | OC₂H₅ |
| P3-21 | CN |
| P3-22 | F |
| P3-23 | Cl |
| P3-24 | Br |
| P3-25 | NO₂ |
| P3-26 | NH₂ |
| P3-27 | CO—NH₂ |
| P3-28 | CO—NH(CH₃) |
| P3-29 | HNCH₃ |
| P3-30 | HNC₂H₅ |
| P3-31 | (CH₃)₂N |
| P3-32 | SO₂H |
| P3-33 | SO₂—CH₃ |
| P3-34 | SO—CH₃ |
| P3-35 | S—CH₃ |
| P3-36 | 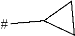 |
| P3-37 | 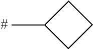 |
| P3-38 | 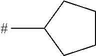 |
| P3-39 | 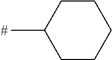 |
| P3-40 | 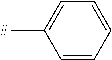 |
| P3-41 | 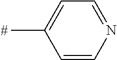 |

According to still another embodiment of formula I, R², R³ together with the carbon atoms to which they are bound form a five-, six-, or seven-membered carbo-, heterocyclic or heteroaromatic ring; wherein the heterocyclic or heteroaromatic ring contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted or substituted by substituents selected from C₁-C₄-alkyl, halogen, C₁-C₄-halogenalkyl, C₁-C₄-alkoxy, C₁-C₄-halogenalkoxy, and CN; and wherein S may be in the form of its oxide SO or SO₂; and wherein in each case one or two CH₂ groups of the carbo- or heterocycle may be replaced by a group independently selected from C(═O) and C(═S); and wherein the carbo-, heterocyclic or heteroaromatic ring is substituent by (R²³)ₘ, wherein m is 0, 1, 2, 3 or 4;
R²³ is in each case independently selected from halogen, OH, CN, COOH, CONH₂, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, saturated or partially unsaturated three-, four-, five-, six-, seven-, eight-, nine-, or ten-membered carbo- and heterocycle, five- or six-membered heteroaryl and aryl; wherein the heterocycle and heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
R^{x} is as defined above;

wherein the acyclic moieties of R²³ are unsubstituted or carry 1, 2, 3 or up to the maximum possible number of identical or different groups R^{23a} which independently of one another are selected from:
R^{23a} halogen, OH, CN, COOH, CONH₂, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or unsubstituted or substituted with R^{91a} selected from the group consisting of halogen, OH, C₁-C₄-alkyl, C₁-C₄-halogenalkyl, C₁-C₄-alkoxy, C₁-C₄-halogenalkoxy, CN, C₃-C₆-cycloalkyl, C₃-C₆-halogencycloalkyl, C₁-C₄-alkylthio;

wherein the carbocyclic, heterocyclic, heteroaryl and aryl moieties of R²³ are unsubstituted or substituted with identical or different groups R^{23b} which independently of one another are selected from:
R^{23b} halogen, OH, CN, COOH, CONH₂, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio.

According to still another embodiment of formula I, R², R³ together with the carbon atoms to which they are bound form a five-, six-, or seven-membered carbo-, heterocyclic or heteroaromatic ring; wherein the heterocyclic or heteroaromatic ring contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted or substituted with substituents selected from C₁-C₄-alkyl, halogen, C₁-C₄-halogenalkyl, C₁-C₄-alkoxy and C₁-C₄-halogenalkoxy, and CN; and wherein S may be in the form of its oxide SO or SO₂; and wherein in each case one or two CH₂ groups of the carbo- or heterocycle may be replaced by a group independently selected from C(═O) and C(═S); and wherein the carbo-, heterocyclic or heteroaromatic ring is substituent by (R²³)ₘ wherein m is 0, 1, 2, 3 or 4;

R^{N} is the substituent of the heteroatom N that is contained in the heterocycle formed by R² and R³ in some of the inventive compounds. R^{N} is selected from C₁-C₄-alkyl, C₁-C₄-halogenalkyl and SO₂Ph, wherein Ph is unsubstituted phenyl or phenyl that is substituted with one, two or three substituents selected from C₁-C₄-alkyl. In one preferred embodiment, R^{N} is in each case independently selected from C₁-C₂-alkyl, C₁-C₂-halogenalkyl and SO₂Ph, wherein Ph is unsubstituted phenyl or phenyl that is substituted with one methyl substituents. In one particular embodiment, R^{N} is in each case independently selected from C₁-C₂-alkyl, more particularly methyl. In one particular embodiment, R^{N} is in each case independently selected from SO₂Ph, wherein Ph is unsubstituted phenyl or phenyl that is substituted with one methyl.

According to still another embodiment of formula I, R² and R³ together with the carbon atoms to which they are bound form a saturated or partially unsaturated five-, six-or seven-membered carbo- and heterocycle that is unsubstituted or substituted.

According to one embodiment, R² and R³ form a 3-membered saturated carbocycle. According to one embodiment thereof, the carbocycle is unsubstituted, i.e. it does not carry any substituent R²³. According to still another embodiment of formula I, it is substituted with R²³.

According to one embodiment, R² and R³ form a 4-membered saturated carbocycle. According to one embodiment thereof, the carbocycle is unsubstituted, i.e. it does not carry any substituent R²³. According to still another embodiment of formula I, it is substituted with R²³.

According to one embodiment, R² and R³ form a 5-membered saturated carbocycle. According to one embodiment thereof, the carbocycle is unsubstituted, i.e. it does not carry any substituent R²³. According to still another embodiment of formula I, it is substituted with R²³.

According to one embodiment, R² and R³ form a 6-membered saturated carbocycle. According to one embodiment thereof, the carbocycle is unsubstituted, i.e. it does not carry any substituent R²³. According to still another embodiment of formula I, it is substituted with R²³.

According to one embodiment, R² and R³ form a 7-membered saturated carbocycle. According to one embodiment thereof, the carbocycle is unsubstituted, i.e. it does not carry any substituent R²³. According to still another embodiment of formula I, it is substituted with R²³.

According to one embodiment, R² and R³ form a 3-membered saturated heterocycle. According to one embodiment thereof, the carbocycle is unsubstituted, i.e. it does not carry any substituent R²³. According to still another embodiment of formula I, it is substituted with R²³.

According to one embodiment, R² and R³ form a 4-membered saturated heterocycle. According to one embodiment thereof, the carbocycle is unsubstituted, i.e. it does not carry any substituent R²³. According to still another embodiment of formula I, it is substituted with R²³.

According to one embodiment, R² and R³ form a 5-membered saturated heterocycle. According to one embodiment thereof, the carbocycle is unsubstituted, i.e. it does not carry any substituent R²³. According to still another embodiment of formula I, it is substituted with R²³.

According to one embodiment, R² and R³ form a 6-membered saturated heterocycle. According to one embodiment thereof, the carbocycle is unsubstituted, i.e. it does not carry any substituent R²³. According to still another embodiment of formula I, it is substituted with R²³.

According to one embodiment, R² and R³ form a 7-membered saturated heterocycle. According to one embodiment thereof, the carbocycle is unsubstituted, i.e. it does not carry any substituent R²³. According to still another embodiment of formula I, it is substituted with R²³.

According to one embodiment, R² and R³ form a 5-membered saturated heteroaryl. According to one embodiment thereof, the heteroaryl is unsubstituted, i.e. it does not carry any substituent R²³. According to still another embodiment of formula I, it is substituted with R²³.

According to one embodiment, R² and R³ form a 6-membered heteroaryl. According to one embodiment thereof, the heteroaryl is unsubstituted, i.e. it does not carry any substituent R²³.

According to still another embodiment of formula I, it is substituted with R²³.

According to one embodiment, R² and R³ do not form a cyclic substituent.

R²³ according to the invention is in each case independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, saturated or partially unsaturated three-, four-, five-, six-, seven-, eight-, nine-, or ten-membered carbo- and heterocycle, five- or six-membered heteroaryl and aryl;

wherein the heterocycle and heteroaryl contains one, two or three heteroatoms selected from N, O and S; and wherein in each case one or two CH₂ groups of the carbo- or heterocycle may be replaced by a group independently selected from C(═O) and C(═S); and wherein

R^{x} is as defined above;

wherein the acyclic moieties of R²³ are unsubstituted or substituted with identical or different groups R^{23a} which independently of one another are selected from:

R^{23a} halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or unsubstituted or substituted with R^{23a} selected from the group consisting of halogen, OH, C₁-C₄-alkyl, C₁-C₄-halogenalkyl, C₁-C₄-alkoxy, C₁-C₄-halogenalkoxy, CN, C₃-C₆-cycloalkyl, C₃-C₆-halogencycloalkyl, C₁-C₄-alkylthio;

wherein the carbocyclic, heterocyclic, heteroaryl and aryl of R²³ are unsubstituted or substituted with identical or different groups R^{23b} which independently of one another are selected from:

R^{23b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

For every R²³ that is present in the inventive compounds, the following embodiments and preferences apply independently of the meaning of any other R²³ that may be present in the ring.

According to one embodiment of formula I, wherein m is 0, 1, 2, 3 or 4.

According to still another embodiment of formula I, m is 0.

According to still another embodiment of formula I, m is 1.

According to still another embodiment of formula I, m is 2 or 3. According to one specific embodiment thereof, m is 2. According to still another embodiment of formula I, m is 3.

According to one embodiment of formula I, R²³ is halogen, C₁-C₆-alkyl, C₁-C₆-halogenalkyl, C₁-C₆-alkoxy or, C₁-C₆-halogenalkoxy, in particular CH₃, Et, CHF₂, OCH₃, OCHF₂, OCF₃, F, Cl, more specifically H, CH₃, F or Cl most preferred F or Cl.

According to still another embodiment of formula I, R²³ is halogen, in particular Br, F or Cl, more specifically F or Cl.

According to still another embodiment of formula I, R²³ is OH.

According to still another embodiment of formula I, R²³ is CN.

According to still another embodiment of formula I R²³ is NH₂, NH(C₁-C₄-alkyl), N(C₁-C₄-alkyl)₂ or NH—SO₂—R^{x}, wherein R^{x} is C₁-C₄-alkyl, C₁-C₄-halogenalkyl, unsubstituted aryl or aryl that is substituted with one, two, three, four or five substituents R^{x23} independently selected from C₁-C₄-alkyl.

According to still another embodiment of formula I, R²³ is C₁-C₆-alkyl, in particular C₁-C₄-alkyl, such as CH₃.

According to still another embodiment of formula I, R²³ is C₁-C₆-halogenalkyl, in particular C₁-C₄-halogenalkyl, such as CF₃, CHF₂, CH₂F, CCl₃, CHCl₂ or CH₂Cl.

According to still another embodiment of formula I, R²³ is C₂-C₆-alkenyl or C₂-C₆-halogenalkenyl, in particular C₂-C₄-alkenyl or C₂-C₄-halogenalkenyl, such as CH═CH₂, C(CH₃)═CH₂, CH₂CH═CH₂, CH═CHF, CH═CHCl, CH═CF₂, CH═CCl₂, CF═CF₂, CCl═CCl₂, CH₂CH═CHF, CH₂CH═CHCl, CH₂CH═CF₂, CH₂CH═CCl₂, CH₂CF═CF₂, CH₂CCl═CCl₂, CF₂CF═CF₂ or CCl₂CCl═CCl₂.

According to still another embodiment of formula I, R²³ is C₂-C₆-alkynyl or C₂-C₆-halogenalkynyl, in particular C₂-C₄-alkynyl or C₂-C₄-halogenalkynyl, such as C≡CH, CH₂C≡CH, C≡C—Cl, C≡C—CH₃, CH₂C≡CH, CH₂C≡CCl or CH₂C≡C—CH₃.

According to still another embodiment of formula I, R²³ is C₁-C₆-alkoxy, in particular C₁-C₄-alkoxy, more specifically C₁-C₂-alkoxy such as OCH₃ or OCH₂CH₃.

According to still another embodiment of formula I, R²³ is C₁-C₆-halogenalkoxy, in particular C₁-C₄-halogenalkoxy, more specifically C₁-C₂-halogenalkoxy such as OCF₃, OCHF₂, OCH₂F, OCCl₃, OCHCl₂ or OCH₂Cl, in particular OCF₃, OCHF₂, OCCl₃ or OCHCl₂.

According to still another embodiment of formula I R²³ is C₃-C₆-cycloalkyl, in particular cyclopropyl.

According to still another embodiment of formula I, R²³ is C₃-C₆-cycloalkyl, for example cyclopropyl, substituted with one, two, three or up to the maximum possible number of identical or different groups R^{23b} as defined and preferably herein.

According to still another embodiment of formula I, R²³ is C₃-C₆-halogencycloalkyl. In a special embodiment R²³ is fully or partially halogenated cyclopropyl.

According to still another embodiment of formula I, R²³ is unsubstituted aryl or aryl that is substituted with one, two, three or four R^{23b}, as defined herein. In particular, R²³ is unsubstituted phenyl or phenyl that is substituted with one, two, three or four R^{23b}, as defined herein.

According to still another embodiment of formula I, R²³ is unsubstituted 5- or 6-membered heteroaryl. According to still a further embodiment, R²³ is 5- or 6-membered heteroaryl that is substituted with one, two or three R^{23b}, as defined herein.

According to still another embodiment of formula I, R²³ is in each case independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy and C₃-C₆-cycloalkyl; wherein the acyclic moieties of R²³ are not further substituted or carry one, two, three, four or five identical or different groups R^{23a} as defined below and wherein the carbocyclic, heterocyclic and heteroaryl moieties of R²³ are not further substituted or carry one, two, three, four or five identical or different groups R^{23b} as defined below.

According to still another embodiment of formula I, R²³ is independently selected from halogen, OH, C₁-C₆-alkyl, C₁-C₆-halogenalkyl, C₁-C₆-alkoxy and C₁-C₆-halogenalkoxy, in particular independently selected from F, Cl, Br, CN, OH, C₁-C₄-alkyl, C₁-C₄-halogenalkyl, C₁-C₄-alkoxy and C₁-C₄-halogenalkoxy.

R^{23a} are the possible substituents for the acyclic moieties of R²³.

R^{23a} according to the invention is independently selected from halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl and phenyl group is unsubstituted or unsubstituted or substituted with R^{23a} selected from the group consisting of halogen, OH, C₁-C₄-alkyl, C₁-C₄-halogenalkyl, C₁-C₄-alkoxy, C₁-C₄-halogenalkoxy, CN, C₃-C₆-cycloalkyl, C₃-C₆-halogencycloalkyl, C₁-C₄-alkylthio.

R^{23a} according to the invention is independently selected from halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl and phenyl group is unsubstituted or unsubstituted or substituted with R^{23a} selected from the group consisting of halogen, OH, C₁-C₄-alkyl, C₁-C₄-halogenalkyl, C₁-C₄-alkoxy and C₁-C₄-halogenalkoxy, in particular selected from halogen, C₁-C₂-alkyl, C₁-C₂-halogenalkyl, C₁-C₂-alkoxy, C₁-C₂-halogenalkoxy, more specifically selected from halogen, such as F, Cl and Br.

In to one embodiment R^{23a} is independently selected from halogen, OH, CN, C₁-C₂-alkoxy, C₃-C₆-cycloalkyl, C₃-C₆-halogencycloalkyl and C₁-C₂-halogenalkoxy. Specifically, R^{23a} is independently selected from F, Cl, OH, CN, C₁-C₂-alkoxy, cyclopropyl, 1-F-cyclopropyl, 1-Cl-cyclopropyl, 1,1-F₂-cyclopropyl, 1,1-Cl₂-cyclopropyl and C₁-C₂-halogenalkoxy.

According to one embodiment R^{23a} is independently selected from halogen, such as F, Cl, Br and I, more specifically F, Cl and Br.

According to still another embodiment of formula I, R^{23a} is independently selected from OH, C₃-C₆-cycloalkyl, C₃-C₆-halogencycloalkyl and C₁-C₂-halogenalkoxy. Specifically, R^{23a} is independently selected from OH, cyclopropyl and C₁-C₂-halogenalkoxy.

R^{23b} are the possible substituents for the carbocyclic, heterocyclic and heteroaryl moieties of R²³.

R^{23b} according to the invention is independently selected from halogen, OH, CN, C₁-C₄-alkyl, C₁-C₄-alkoxy, C₁-C₄-halogenalkyl, C₃-C₆-cycloalkyl, C₃-C₆-halogencycloalkyl and C₁-C₄-halogenalkoxy.

According to one embodiment thereof R^{23b} is independently selected from halogen, CN, C₁-C₂-alkyl, C₁-C₂-alkoxy, C₁-C₂-halogenalkyl, C₃-C₆-cycloalkyl, C₃-C₆-halogencycloalky and C₁-C₂-halogenalkoxy. Specifically, R^{23b} is independently selected from F, Cl, OH, CN, CH₃, OCH₃, cyclopropyl, 1-F-cyclopropyl, 1-Cl-cyclopropyl, 1,1-F₂-cyclopropyl, 1,1-Cl₂-cyclopropyl and halogenmethoxy.

According to still another embodiment thereof R^{23b} is independently selected from C₁-C₂-alkyl, C₁-C₂-alkoxy, C₁-C₂-halogenalkyl, C₃-C₆-cycloalkyl, C₃-C₆-halogencycloalkyl and C₁-C₂-halogenalkoxy. Specifically, R^{23b} is independently selected from OH, CH₃, OCH₃, cyclopropyl, 1-F-cyclopropyl, 1-Cl-cyclopropyl, 1,1-F₂-cyclopropyl, 1,1-Cl₂-cyclopropyl and halogenmethoxy, more specifically independently selected from OH, CH₃, OCH₃, cyclopropyl, 1-F-cyclopropyl, 1-Cl-cyclopropyl, 1,1-F₂-cyclopropyl, 1,1-Cl₂-cyclopropyl cyclopropyl and OCHF₂.

Particularly preferred embodiments of combinations of R² and R³ according to the invention are in Table P35 below, wherein each line of lines P35-1 to P35-305 corresponds to one particular embodiment of the invention, wherein P35-1 to P35-305 are also in any combination with one another a preferred embodiment of the present invention. The carbon atom, to which R² bound is marked with * in the drawings and the carbon atom, to which R³ is bound is marked with # in the drawings. cPr stands for cyclopropyl.

TABLE P23

| | | |
|---|---|---|
| line | R² | R³ |

| | |
|---|---|
| P23-1 | 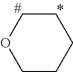 |
| P23-2 | 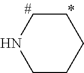 |
| P23-3 | 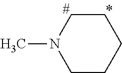 |
| P23-4 | 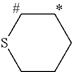 |
| P23-5 | 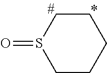 |
| P23-6 | 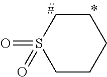 |
| P23-7 | 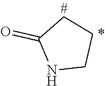 |
| P23-8 | 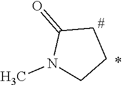 |
| P23-9 | 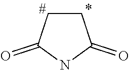 |
| P23-10 | 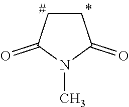 |
| P23-11 | 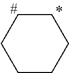 |
| P23-12 | 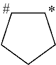 |
| P23-13 | 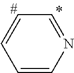 |
| P23-14 | 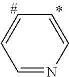 |
| P23-15 | 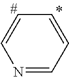 |
| P23-16 | 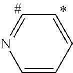 |

R⁴ according to the invention is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl;

wherein the heteroaryl contains one, two or three heteroatoms selected from N, O and S; and

wherein

R^{x} is C₁-C₄-alkyl, C₁-C₄-halogenalkyl, unsubstituted aryl or aryl that is substituted with one, two, three, four or five substituents R^{x4} independently selected from C₁-C₄-alkyl, halogen, OH, ON, C₁-C₄-halogenalkyl, C₁-C₄-alkoxy and C₁-C₄-halogenalkoxy;

wherein the acyclic moieties of R⁴ are unsubstituted or substituted with identical or different groups R^{4a} which independently of one another are selected from:

R^{4a} halogen, OH, CN, C₁-C₆-alkoxy, C₃-C₆-cycloalkyl, C₃-C₆-halogencycloalky, C₁-C₄-halogenalkoxy, C₁-C₆-alkylthio, aryl and phenoxy, wherein the aryl and phenoxy group is unsubstituted or unsubstituted or substituted with R^{44a} selected from the group consisting of halogen, OH, C₁-C₄-alkyl, C₁-C₄-halogenalkyl, C₁-C₄-alkoxy and C₁-C₄-halogenalkoxy;

wherein the carbocyclic, heteroaryl and aryl moieties of R⁴ are unsubstituted or substituted with identical or different groups R^{4b} which independently of one another are selected from:

R^{4b} halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

For every R⁴ that is present in the inventive compounds, the following embodiments and preferences apply independently of the meaning of any other R⁴ that may be present in the ring.

According to one embodiment of formula I, R⁴ is H, halogen or C₁-C₆-alkyl, in particular H, CH₃, Et, F, Cl, more specifically H, CH₃, F or Cl most preferred H, F or Cl.

According to another embodiment of formula I, R⁴ is hydrogen.

According to still another embodiment of formula I, R⁴ is halogen, in particular Br, F or Cl, more specifically F or Cl.

According to another embodiment of formula I, R⁴ is F

According to another embodiment of formula I, R⁴ is Cl

According to another embodiment of formula I, R⁴ is Br.

According to still another embodiment of formula I, R⁴ is OH.

According to still another embodiment of formula I, R⁴ is COOH.

According to still another embodiment of formula I, R⁴ is CONH₂.

According to still another embodiment of formula I, R⁴ is CN.

According to still another embodiment of formula I, R⁴ is NO₂.

According to still another embodiment of formula I, R⁴ is SH.

According to still another embodiment of formula I R⁴ is NH₂, NH(C₁-C₄-alkyl), N(C₁-C₄-alkyl)₂ or NH—SO₂—R^{x}, wherein R^{x} is C₁-C₄-alkyl, C₁-C₄-halogenalkyl, unsubstituted aryl or aryl that is substituted with one, two, three, four or five substituents R^{x4} independently selected from C₁-C₄-alkyl, halogen, OH, CN, C₁-C₄-halogenalkyl, C₁-C₄-alkoxy, or C₁-C₄-halogenalkoxy. In particular C₁-C₄-alkyl, such as NHCH₃ and N(CH₃)₂. In particular R^{x} is C₁-C₄-alkyl, and phenyl that is substituted with one CH₃, more specifically SO₂—R^{x} is CH₃ and tosyl group (“Ts”).

According to still another embodiment of formula I, R⁴ is C₁-C₆-alkyl, in particular C₁-C₄-alkyl, such as CH₃ or CH₂CH₃.

According to still another embodiment of formula I, R⁴ is C₁-C₆-halogenalkyl, in particular C₁-C₄-halogenalkyl, such as CF₃, CHF₂, CH₂F, CCl₃, CHCl₂, CH₂Cl, CF₃CH₂, CCl₃CH₂ or CF₂CHF₂.

According to still another embodiment of formula I, R⁴ is C₂-C₆-alkenyl or C₂-C₆-halogenalkenyl, in particular C₂-C₄-alkenyl or C₂-C₄-halogenalkenyl, such as CH═CH₂, C(CH₃)═CH₂, CH═CCl₂, CH═CF₂, CCl═CCl₂, CF═CF₂, CH═CH₂, CH₂CH═CCl₂, CH₂CH═CF₂, CH₂CCl═CCl₂, CH₂CF═CF₂, CCl₂CH═CCl₂, CF₂CH═CF₂, CCl₂CCl═CCl₂, or CF₂CF═CF₂.

According to still another embodiment of formula I, R⁴ is C₂-C₆-alkynyl or C₂-C₆-halogenalkynyl, in particular C₂-C₄-alkynyl or C₂-C₄-halogenalkynyl, such as C≡CH, C≡CCl, C≡CF. CH₂C≡CH, CH₂C≡CCl, or CH₂C≡CF.

According to still another embodiment of formula I, R⁴ is C₁-C₆-alkoxy, in particular C₁-C₄-alkoxy, more specifically C₁-C₂-alkoxy such as OCH₃ or OCH₂CH₃.

According to still another embodiment of formula I, R⁴ is C₁-C₆-halogenalkoxy, in particular C₁-C₄-halogenalkoxy, more specifically C₁-C₂-halogenalkoxy such as OCF₃, OCHF₂, OCH₂F, OCCl₃, OCHCl₂ or OCH₂Cl, in particular OCF₃, OCHF₂, OCCl₃ or OCHCl₂.

According to still another embodiment of formula I R⁴ is C₃-C₆-cycloalkyl, in particular cyclopropyl.

According to still another embodiment of formula I, R⁴ is C₃-C₆-cycloalkyl, for example cyclopropyl, substituted with one, two, three or up to the maximum possible number of identical or different groups R^{4b} as defined and preferably herein.

According to still another embodiment of formula I, R⁴ is C₃-C₆-halogencycloalkyl. In a special embodiment R⁴ is fully or partially halogenated cyclopropyl.

According to still another embodiment of formula I, R⁴ is unsubstituted aryl or aryl that is substituted with one, two, three or four R^{4b}, as defined herein. In particular, R⁴ is unsubstituted phenyl or phenyl that is substituted with one, two, three or four R^{4b}, as defined herein.

According to still another embodiment of formula I, R⁴ is unsubstituted 5- or 6-membered heteroaryl. According to still a further embodiment, R⁴ is 5- or 6-membered heteroaryl that is substituted with one, two or three R^{4b}, as defined herein.

According to still another embodiment of formula I, R⁴ is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy and C₃-C₆-cycloalkyl; wherein the acyclic moieties of R⁴ are not further substituted or carry one, two, three, four or five identical or different groups R^{4a} as defined below and wherein the carbocyclic, heteroaryl and aryl moieties of R⁴ are not further substituted or carry one, two, three, four or five identical or different groups R^{4b} as defined below.

According to still another embodiment of formula I, R⁴ is independently selected from hydrogen, halogen, CN, OH, C₁-C₆-alkyl, C₁-C₆-alkoxy, C₆-alkenyl, C₂-C₆-alkynyl, C₃-C₆-cycloalkyl, wherein the acyclic and cyclic moieties of R⁴ are unsubstituted or substituted by halogen.

According to still another embodiment of formula I, R⁴ is independently selected from hydrogen, halogen, CN, OH, C₁-C₆-alkyl, C₁-C₆-halogenalkyl, C₁-C₆-alkoxy and C₁-C₆-halogenalkoxy, in particular independently selected from H, F, Cl, Br, CN, OH, C₁-C₄-alkyl, C₁-C₄-halogenalkyl, C₁-C₄-alkoxy and C₁-C₄-halogenalkoxy.

According to still another embodiment of formula I, R⁴ is independently selected from H, CN, halogen or C₁-C₆-alkyl, in particular H, CN, CH₃, Et, F, Cl, more specifically H, CN, CH₃, F or Cl most preferred H, CH₃, F or Cl.

R^{4a} are the possible substituents for the acyclic moieties of R⁴.

R^{4a} according to the invention is independently selected from halogen, OH, CN, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or unsubstituted or substituted with R^{44a} selected from the group consisting of halogen, OH, C₁-C₄-alkyl, C₁-C₄-halogenalkyl, C₁-C₄-alkoxy and C₁-C₄-halogenalkoxy, in particular selected from halogen, C₁-C₂-alkyl, C₁-C₂-halogenalkyl, C₁-C₂-alkoxy and C₁-C₂-halogenalkoxy, more specifically selected from halogen, such as F, Cl and Br.

In to one embodiment R^{4a} is independently selected from halogen, OH, CN, C₁-C₂-alkoxy, C₃-C₆-cycloalkyl, C₃-C₆-halogencycloalkyl and C₁-C₂-halogenalkoxy. Specifically, R^{4a} is independently selected from F, Cl, OH, CN, C₁-C₂-alkoxy, cyclopropyl, 1-F-cyclopropyl, 1-Cl-cyclopropyl, 1,1-F₂-cyclopropyl, 1,1-Cl₂-cyclopropyl and C₁-C₂-halogenalkoxy.

According to one embodiment R^{4a} is independently selected from halogen, such as F, Cl, Br and I, more specifically F, Cl and Br.

According to still another embodiment of formula I, R^{4a} is independently selected from OH, C₃-C₆-cycloalkyl, C₃-C₆-halogencycloalkyl and C₁-C₂-halogenalkoxy. Specifically, R^{4a} is independently selected from OH, cyclopropyl and C₁-C₂-halogenalkoxy.

According to still another embodiment of formula I, R^{4a} is independently selected from aryl and phenoxy, wherein the aryl group is unsubstituted or substituted with R^{44a} selected from the group consisting of halogen, OH, C₁-C₄-alkyl, C₁-C₄-halogenalkyl, C₁-C₄-alkoxy and C₁-C₄-halogenalkoxy, in particular selected from halogen, C₁-C₂-alkyl, C₁-C₂-halogenalkyl, C₁-C₂-alkoxy and C₁-C₂-halogenalkoxy, more specifically selected from halogen, such as F, Cl and Br.

R^{4b} are the possible substituents for the carbocyclic, heteroaryl and aryl moieties of R⁴.

R^{4b} according to the invention is independently selected from halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;

According to one embodiment thereof R^{4b} is independently selected from halogen, CN, C₁-C₂-alkyl, C₁-C₂-alkoxy, C₁-C₂-halogenalkyl, C₃-C₆-cycloalkyl, C₃-C₆-halogencycloalkyl and C₁-C₂-halogenalkoxy. Specifically, R^{4b} is independently selected from F, Cl, Br, OH, CN, CH₃, OCH₃, CHF₂, OCHF₂, cyclopropyl, 1-F-cyclopropyl, 1-Cl-cyclopropyl, 1,1-F₂-cyclopropyl, 1,1-Cl₂-cyclopropyl, OCF₃, and OCHF₂.

According to still another embodiment thereof R^{4b} is independently selected from halogen, C₁-C₂-alkyl, C₁-C₂-alkoxy, C₁-C₂-halogenalkyl, C₃-C₆-cycloalkyl, C₃-C₆-halogencycloalkyl and C₁-C₂-halogenalkoxy. Specifically, R^{4b} is independently selected from halogen, CN, OH, CH₃, CHF₂, OCHF₂, OCF₃, OCH₃, cyclopropyl, 1-F-cyclopropyl, 1-Cl-cyclopropyl, 1,1-F₂-cyclopropyl, 1,1-Cl₂-cyclopropyl and halogenmethoxy, more specifically independently selected from F, Cl, OH, CH₃, OCH₃, CHF₂, OCH₃, cyclopropyl, 1-F-cyclopropyl, 1-Cl-cyclopropyl, 1,1-F₂-cyclopropyl, 1,1-Cl₂-cyclopropyl, OCHF₂ and OCF₃.

R^{x} in the substituent NH—SO₂—R^{x} is in each case independently selected from C₁-C₄-alkyl, C₁-C₄-halogenalkyl, unsubstituted aryl and aryl that is substituted with one, two, three, four or five substituents R^{x4} independently selected from C₁-C₄-alkyl, halogen, OH, CN, C₁-C₄-halogenalkyl, C₁-C₄-alkoxy and C₁-C₄-halogenalkoxy. In particular, R^{x} is in each case independently selected from C₁-C₄-alkyl, halogen, OH, CN and phenyl that is substituted with one, two or three R^{x4} independently selected from C₁-C₂-alkyl, more specifically R^{x} is in each case independently selected from C₁-C₄-alkyl and phenyl that is substituted with one CH₃, more specifically SO₂—R^{x} is the tosyl group (“Ts”).

Particularly preferred embodiments of R⁴ according to the invention are in Table P4 below, wherein each line of lines P4-1 to P4-16 corresponds to one particular embodiment of the invention. Thereby, for every R⁴ that is present in the inventive compounds, these specific embodiments and preferences apply independently of the meaning of any other R⁴ that may be present in the ring:

TABLE P4

| No. | R⁴ |
|---|---|
| P4-1 | H |
| P4-2 | Cl |
| P4-3 | F |
| P4-4 | Br |
| P4-5 | OH |
| P4-6 | ON |
| P4-7 | NO₂ |
| P4-8 | CH₃ |
| P4-9 | CH₂CH₃ |
| P4-10 | CF₃ |
| P4-11 | CHF₂ |
| P4-12 | OCH₃ |
| P4-13 | OCH₂CH₃ |
| P4-14 | OCF₃ |
| P4-15 | OCHF₂ |
| P4-16 | NH-Ts |

“Ts” in the table stands for the tosylgroup SO₂-(p-CH₃)phenyl.

R⁵ is in each case independently selected from hydrogen, OH, CH(═O), C(═O)C₁-C₆-alkyl, C(═O)C₂-C₆-alkenyl, C(═O)C₃-C₆-cycloalkyl, C₁-C₆-alkyl, C₁-C₄-halogenalkyl, C₃-C₆-cycloalkyl, C₃-C₆-halogencycloalkyl, C₁-C₄-alkoxy, C₁-C₄-halogenalkoxy, OR^{Y}, C₂-C₆-alkenyl, C₂-C₆-halogenalkenyl C₂-C₆-alkynyl, C₂-C₆-halogenalkynyl, S(O)ₙ—C₁-C₆-alkyl, S(O)ₙ—C₁-C₆-halogenalkyl, five- or six-membered heteroaryl and aryl; wherein the heteroaryl contains one, two or three heteroatoms selected from N, O and S; wherein the aryl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy; wherein n and R^{Y} are as defined above.

R^{5a} is the substituent of the acyclic moieties of R⁵. The acyclic moieties of R⁵ are not further substituted or carry one, two, three or up to the maximum possible number of identical or different groups R^{5a} which independently of one another are selected from halogen, OH, CN, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, aryl and phenoxy, wherein the heteroaryl, aryl and phenoxy group is unsubstituted or carries one, two, three, four or five substituents R^{78a′} selected from the group consisting of halogen, OH, C₁-C₄-alkyl, C₁-C₄-halogenalkyl, C₁-C₄-alkoxy and C₁-C₄-halogenalkoxy;

R^{5b} is the substituent of carbocyclic, phenyl, heterocyclic and heteroaryl moieties of R⁵. The carbocyclic, phenyl, heterocyclic and heteroaryl moieties of R⁵ are not further substituted or carry one, two, three, four, five or up to the maximum number of identical or different groups R^{5b} which independently of one another are selected from halogen, OH, CN, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

According to one embodiment of formula I, R⁵ is H.

According to still another embodiment of formula I, R⁵ is OH.

According to a further specific embodiment of formula I, R⁵ is CH(═O).

According to a further specific embodiment of formula I, R⁵ is C(═O)C₁-C₆-alkyl, wherein alkyl is CH₃, C₂H₅, n-propyl, i-propyl, n-butyl, i-butyl, tert-butyl, n-pentyl or i-pentyl.

According to a further specific embodiment of formula I, R⁵ is C(═O)C₂-C₆-alkenyl, wherein alkenyl is CH═CH₂, CH₂CH═CH₂.

According to a further specific embodiment of formula I, R⁵ is C(═O)C₂-C₆-alkynyl wherein alkynyl is C≡CH, CH₂C≡CH.

According to a further specific embodiment of formula I, R⁵ is C(═O)C₃-C₆-cycloalkyl, wherein cycloalkyl is cyclopropyl (C₃H₇) or cyclobutyl (C₄H₉).

According to still another embodiment of formula I, R⁵ is C₁-C₆-alkyl, such as CH₃, C₂H₅, n-propyl, i-propyl, n-butyl, i-butyl, tert-butyl, n-pentyl or i-pentyl.

According to still another embodiment of formula I, R⁵ is C₁-C₆-alkyl, in particular C₁-C₄-alkyl, such as CH₃, C₂H₅, n-propyl, i-propyl.

According to still another embodiment of formula I, R⁵ is C₁-C₆-halogenalkyl, in particular C₁-C₄-halogenalkyl, such as CF₃, CCl₃, FCH₂, ClCH₂, F₂CH, Cl₂CH, CF₃CH₂, CCl₃CH₂ or CF₂CHF₂.

According to still another embodiment of formula I R⁵ is C₃-C₆-cycloalkyl, in particular cyclopropyl.

According to still another embodiment of formula I, R⁵ is C₃-C₆-halogencycloalkyl. In a special embodiment R^{5b} is fully or partially halogenated cyclopropyl, such as 1-F-cyclopropyl, 1-Cl-cyclopropyl, 1,1-F₂-cyclopropyl, 1,1-Cl₂-cyclopropyl.

According to still another embodiment of formula I, R⁵ is C₁-C₄-alkoxy and C₁-C₄-halogenalkoxy, in particular C₁-C₃-alkoxy, C₁-C₃-halogenalkoxy, such as CH₂OCH₃, CH₂OCF₃ or CH₂OCHF₂.

According to a further specific embodiment of formula I, R⁵ is OR^{Y}, wherein R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, phenyl and phenyl-C₁-C₆-alkyl; wherein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;

According to a further specific embodiment of formula I, R⁵ is OR^{Y}, wherein R^{Y} is C₁-C₆-alkyl, in particular C₁-C₄-alkyl, more specifically C₁-C₂-alkyl. R⁵ is such as OCH₃ or OCH₂CH₃.

According to a further specific embodiment of formula I, R⁵ is OR^{Y}, wherein R^{Y} is C₁-C₆-halogenalkyl, in particular C₁-C₄-halogenalkyl, more specifically C₁-C₂-halogenalkyl. R⁵ is such as OCF₃, OCHF₂, OCH₂F, OCCl₃, OCHCl₂ or OCH₂Cl, in particular OCF₃, OCHF₂, OCCl₃ or OCHCl₂.

According to a further specific embodiment of formula I, R⁵ is OR^{Y}, wherein R^{Y} C₂-C₆-alkenyl, in particular C₂-C₄-alkenyl, more specifically C₁-C₂-alkenyl. R⁵ is such as OCH═CH₂, OCH₂CH═CH₂.

According to a further specific embodiment of formula I, R⁵ is OR^{Y}, wherein R^{Y} C₂-C₆-alkynyl, in particular C₂-C₆-alkynyl, in particular C₂-C₄-alkynyl, more specifically C₁-C₂-alkynyl. R⁵ is such as OC≡CH

According to still another embodiment of formula I, R⁵ is OR^{Y}, wherein R^{Y} is C₃-C₆-halogencycloalkyl. In a special embodiment R¹ is fully or partially halogenated cyclopropyl.

According to still another embodiment of formula I, R⁵ is is OR^{Y}, wherein R^{Y} and phenyl; wherein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy.

According to still another embodiment of formula I, R⁵ is is OR^{Y}, wherein R^{Y} phenyl-C₁-C₆-alkyl, such as phenyl-CH₂, herein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy. R⁵ is such as OCH₂Ph.

According to still a further embodiment of formula I, R⁵ is C₂-C₆-alkenyl, in particular C₂-C₄-alkenyl, such as CH═CH₂, C(CH₃)═CH₂, CH₂CH═CH₂.

According to a further specific embodiment of formula I, R⁵ is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CH₂CH═CHF, CH₂CH═CHCl, CH₂CH═CF₂, CH₂CH═CCl₂, CF₂CH═CF₂, CCl₂CH═CCl₂, CF₂CF═CF₂, CCl₂CCl═CCl₂.

According to still a further embodiment of formula I, R⁵ is C₂-C₆-alkynyl or C₂-C₆-halogenalkynyl, in particular C₂-C₄-alkynyl or C₂-C₄-halogenalkynyl, such as C≡CH, CH₂C≡CH.

According to still another embodiment of formula I, R⁵ is phenyl-C₁-C₆-alkyl, such as phenyl-CH₂, wherein the phenyl moiety in each case is unsubstituted or substituted by one, two or three identical or different groups R^{5b} which independently of one another are selected from halogen, C₁-C₂-alkyl, C₁-C₂-alkoxy, C₁-C₂-halogenalkyl and C₁-C₂-halogenalkoxy, in particular F, Cl, Br, CH₃, OCH₃, CF₃ and OCF₃.

According to still another embodiment of formula I, R⁵ is aryl, in particular phenyl, wherein the aryl or phenyl moiety in each case is unsubstituted or substituted by identical or different groups R^{5b} which independently of one another are selected from halogen, C₁-C₂-alkyl, C₁-C₂-alkoxy, C₁-C₂-halogenalkyl and C₁-C₂-halogenalkoxy, in particular F, Cl, Br, CH₃, OCH₃, CF₃ and OCF₃. According to one embodiment, R⁵ is unsubstituted phenyl. According to another embodiment, R⁵ is phenyl, that is substituted by one, two or three, in particular one, halogen, in particular selected from F, Cl and Br, more specifically selected from F and Cl.

According to still another embodiment of formula I, R⁵ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R⁵ is a 6-membered heteroaryl such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to still another embodiment of formula I, R⁵ is in each case independently selected from H, halogen, OH, CN, C₁-C₆-alkyl, C₁-C₆-halogenalkyl, C₂-C₆-alkenyl, C₂-C₆-alkynyl, C₁-C₆-alkoxy, C₁-C₆-halogenalkoxy, C₃-C₆-alkenyloxy, C₃-C₆-alkynyloxy and C₃-C₆-cycloalkyl wherein the acyclic moieties of R⁵ are unsubstituted or substituted with identical or different groups R^{5a} as defined and preferably defined herein, and wherein the carbocyclic, phenyl and heteroaryl moieties of R⁵ are unsubstituted or substituted with identical or different groups R^{5b} as defined and preferably defined herein.

According to still another embodiment of formula I, R⁵ is in each case independently selected from H, halogen, OH, CN, C₁-C₆-alkyl, C₂-C₆-alkenyl, C₂-C₆-alkynyl, C₁-C₆-alkoxy, C₃-C₆-alkenyloxy, C₃-C₆-alkynyloxy and C₃-C₆-cycloalkyl, wherein the acyclic moieties of R⁵ are unsubstituted or substituted with identical or different groups R^{5a} as defined and preferably defined herein, and wherein the cycloalkyl moieties of R⁵ are unsubstituted or substituted with identical or different groups R^{5b} as defined and preferably defined herein.

According to still another embodiment of formula I, R⁵ is in each case independently selected from H and OR^{Y}, wherein R^{Y} is most preferably C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, phenyl and phenyl-C₁-C₆-alkyl; wherein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy.

According to still another embodiment of formula I, R⁵ is in each case independently selected from H and OR^{Y}, wherein R^{Y} is most preferably C₂-C₆-alkenyl, C₂-C₆-alkynyl, phenyl and phenyl-C₁-C₆-alkyl; wherein the phenyl groups are unsubstituted or carry one, two, three, four or five substituents selected from the group consisting of CN, halogen, C₁-C₄-alkyl, C₁-C₄-halogenalkyl, C₁-C₄-alkoxy and C₁-C₄-halogenalkoxy.

According to still another embodiment of formula I, R⁵ is in each case independently selected from H, CH(═O), C(═O)C₁-C₆-alkyl, wherein the acyclic moieties of R⁵ are unsubstituted or substituted with identical or different groups R^{5a} as defined and preferably defined herein, and wherein the cycloalkyl moieties of R⁵ are unsubstituted or substituted with identical or different groups R^{5b} as defined and preferably defined herein.

According to one embodiment R^{5a} is independently selected from halogen, C₁-C₆-alkoxy, C₃-C₆-cycloalkyl, C₃-C₆-halogencycloalkyl and C₁-C₄-halogenalkoxy. Specifically, R^{5a} is independently selected from F, Cl, Br, I, C₁-C₂-alkoxy, cyclopropyl, 1-F-cyclopropyl, 1-Cl-cyclopropyl, 1,1-F₂-cyclopropyl, 1,1-Cl₂-cyclopropyl and C₁-C₂-halogenalkoxy.

According to still another embodiment of formula I, R^{5a} is independently halogen, in particular selected from F, Cl, Br and I, more specifically F, Cl and Br.

R^{5b} are the possible substituents for the cycloalkyl, heteroaryl and phenyl moieties of R⁵. R^{5b} according to the invention is independently selected from halogen, OH, ON, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio.

According to one embodiment thereof R^{5b} is independently selected from halogen, CN, C₁-C₄-alkyl, C₁-C₄-alkoxy, C₁-C₄-halogenalkyl and C₁-C₄-halogenalkoxy, in particular halogen, C₁-C₄-alkyl and C₁-C₄-alkoxy. Specifically, R^{5b} is independently selected from F, Cl, N, CH₃, CHF₂, CF₃OCH₃ and halogenmethoxy.

Particularly preferred embodiments of R⁵ according to the invention are in Table P5 below, wherein each line of lines P5-1 to P5-32 corresponds to one particular embodiment of the invention, wherein P5-1 to P5-32 are also in any combination with one another a preferred embodiment of the present invention. The connection point to the carbon atom, to which R⁵ is bound is marked with “#” in the drawings.

TABLE P5

| No. | R⁵ |
|---|---|
| P5-1 | H |
| P5-2 | CH₃ |
| P5-3 | CH₂F |
| P5-4 | CHF₂ |
| P5-5 | CF₃ |
| P5-6 | C₂H₅ |
| P5-7 | C₃H₇ |
| P5-8 | CH(CH₃)₂ |
| P5-9 | CH₂CH₂CH₃ |
| P5-10 | CH₂CH₂CH₂CH₃ |
| P5-11 | CH₂CH(CH₃)₂ |
| P5-12 | C(CH₃)₃ |
| P5-13 | CH₂CH₂CH₂CH₂CH₃ |
| P5-14 | CH═CH₂ |
| P5-15 | CH₂CH═CH₂ |
| P5-16 | C≡CH |
| P5-17 | CH₂C≡CH |
| P5-18 | CH₂CH₂CH(CH₃)₂ |
| P5-19 | OH |
| P5-20 | OCH₃ |
| P5-21 | OCHF₂ |
| P5-22 | OC₂H₅ |
| P5-23 | OCH₂OCH₃ |
| P5-24 | OCH₂Ph |
| P5-25 | OCH₂CH═CH₂ |
| P5-26 | C(O)CH₃ |
| P5-27 | 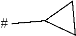 |
| P5-28 | 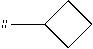 |
| P5-29 | 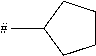 |
| P5-30 | 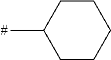 |
| P5-31 | 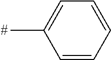 |
| P5-32 | 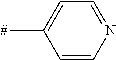 |

R⁶ is independently selected from H, halogen, OH, CN, NO₂, SH, C₁-C₆-alkylthio, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, aryloxy, heteroaryloxy, arylamino, heteroarylamino, arylthio, heteroarylthio, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight, nine-, or ten-membered carbocycle or heterocycle, a five- or six-membered heteroaryl or aryl;

wherein in each case one or two CH₂ groups of the carbo- and heterocycle may be replaced by a group independently selected from C(═O) and C(═S), and wherein the heterocycle and heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted with R′″ which is independently selected from halogen, OH, CN, NO₂, SH, NH₂, NH(C₁-C₄-alkyl), N(C₁-C₄-alkyl)₂, NHSO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl;

wherein R^{x} is as defined above;

wherein the acyclic moieties of R⁶ are independently not further substituted or carry one, two, three or up to the maximum possible number of identical or different groups R^{6a}, which independently of one another are selected from:

R^{6a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₃-C₆-cycloalkyl, C₃-C₆-halogencycloalkyl, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbo- or heterocycle, a five-, six- or ten-membered heteroaryl, aryl or phenoxy, wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbo-, heterocyclic, heteroaryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′ and R″ are as defined above; n is 0, 1, 2; and

wherein the carbo-, heterocyclic, heteroaryl and aryl moieties of R⁶ are independently unsubstituted or substituted with identical or different groups R^{6b}, which independently of one another are selected from:

R^{6b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or carry one, two, three, four or five substituents selected from the group consisting of halogen, OH, C₁-C₄-alkyl, C₁-C₄-halogenalkyl, C₁-C₄-alkoxy and C₁-C₄-halogenalkoxy; and wherein R^{x} and n are as defined above.

According to one embodiment of formula I, R⁶ is independently selected from H, halogen, OH, CN, NO₂, SH, C₁-C₆-alkylthio, NH₂, NH(C₁-C₄-alkyl), N(C₁-C₄-alkyl)₂, NH—SO₂—R^{x}, substituted C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or

wherein the aliphatic moieties of R⁶ are independently not further substituted or carry 1, 2, 3 or up to the maximum possible number of identical or different groups R^{6a}, respectively, which independently of one another are selected from:
R^{6a} halogen, OH, CN, NO₂, SH, NH₂, NH(C₁-C₄-alkyl), N(C₁-C₄-alkyl)₂, NH-aryl, N(aryl)₂, NH(C(═O)C₁-C₄-alkyl), N(C(═O)C₁-C₄-alkyl)₂, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S), five- or six-membered heteroaryl and aryl; wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′, R″ and R″ are as defined above
   wherein the carbocyclic, heterocyclic, heteroaryl and aryl moieties of R⁶ are independently not further substituted or carry 1, 2, 3, 4, 5 or up to the maximum number of identical or different groups R^{6b}, respectively, which independently of one another are selected from:
R^{6b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy.

According to one embodiment of formula I, R⁶ is selected from substituted H, C₁-C₆-alkyl, C₁-C₆-halogenalkyl, C₂-C₆-halogenalkyl, C₂-C₆-alkenyl, C₂-C₆-halogenalkenyl, C₃-C₆-cycloalkenyl, C₂-C₆-alkynyl, C₂-C₆-halogenalkynyl, C₃-C₆-cycloalkynyl, C₁-C₆-alkoxy, aryloxy, heteroaryloxy, arylamino, heteroarylamino, arylthio, heteroarylthio, CN, CH(═O), C(═O)C₂-C₆-alkyl, C(═O)O(C₂-C₆-alkyl), CR′═NOR″, C₃-C₆-cycloalkyl, C₃-C₆-halogencycloalkyl, C₁-C₆-alkyl-five- and six-membered heteroaryl, a five- or six-membered heteroaryl, benzyl, aryl; wherein R′ and R″ are defined below; and wherein the acyclic moieties of R⁶ are unsubstituted or substituted with identical or different groups R^{6a} as defined below and wherein wherein the carbocycle, heterocycle and heteroaryl and aryl moieties are unsubstituted or substituted with substituents R^{6b} as defined below.

According to one embodiment of formula I, R⁶ is selected from H, C₁-C₆-alkyl substituted with halogen, CN, C₁-C₆-alkoxy, aryloxy, arylamino, arylthio, C₁-C₄-halogenalkoxy, C₁-C₆-alkylthio, S(O)ₙ—C₁-C₆-alkyl, NH—SO₂—R^{x}, N(C₁-C₆-alkyl)₂, NH—SO₂—R^{x}, NH(C₁-C₆-alkyl), N(C₁-C₆-alkyl)₂, CH(═O), C(═O)C₁-C₆-alkyl, C(═O)O(C₁-C₆-alkyl), a saturated three-, four-, five-, six-, membered carbo- or heterocycle, aryl, a five- or six-membered heteroaryl; wherein R^{x} is defined below; and wherein the acyclic moieties of R⁶ are unsubstituted or substituted with identical or different groups R^{6a} as defined below and wherein wherein the carbocycle, heterocycle and heteroaryl and aryl moieties are unsubstituted or substituted with substituents R^{6b} as defined below.

According to another embodiment of formula I, R⁶ is H.

According to another embodiment of formula I, R⁶ is F.

According to another embodiment of formula I, R⁶ is Cl.

According to another embodiment of formula I, R⁶ is Br.

According to still another embodiment of formula I, R⁶ is OH.

According to still another embodiment of formula I, R⁶ is CN.

According to still another embodiment of formula I, R⁶ is NO₂.

According to still another embodiment of formula I, R⁶ is SH.

According to still another embodiment of formula I, R⁶ is C₁-C₆-alkylthio, such as SCH₃, SC₂H₅, S-n-propyl, S-i-propyl, S-n-butyl, S-i-butyl, S-tert-butyl, S-n-pentyl, S-i-pentyl, CH₂SCH₃ or CH₂SCH₂CH₃.

According to still another embodiment of formula I, R⁶ is C₁-C₆-halogenalkylthio, such as SCF₃, SCCl₃, CH₂SCF₃ or CH₂SCF₃.

According to still another embodiment of formula I, R⁶ is selected from CN, substituted C₁-C₆-alkyl, C₁-C₆-halogenalkyl or C₁-C₆-alkyl which is substituted, C₁-C₆-halogenalkyl, phenyl, halogenphenyl and three-, four-, five- or six-membered carbo- and heterocycle, wherein the carbo- and heterocycle is unsubstituted or is substituted with substituents R^{6b} as defined below. According to one embodiment thereof, the carbocycle is unsubstituted. In a particular embodiment, R⁶ is selected from C₁-C₆-halogenalkyl, phenyl-CH₂, halogenphenyl-CH₂, phenyl, halogenphenyl and three-, four-, five- or six-membered carbo- and heterocycle, wherein the carbo- and heterocycle is unsubstituted or is substituted with substituents R^{6b} as defined below.

According to still another embodiment of formula I, R⁶ is selected from H, CN, substituted C₁-C₆-alkyl, C₁-C₆-halogenalkyl or C₁-C₆-alkyl which is substituted, C₁-C₆-halogenalkyl, phenyl, halogenphenyl, aryloxy, heteroaryloxy, arylamino, heteroarylamino, arylthio, heteroarylthio and three-, four-, five- or six-membered carbo- and heterocycle, wherein the carbo- and heterocycle is unsubstituted or substituted by substituents R^{6b} as defined below. According to one embodiment thereof, the carbo- and heterocycle is unsubstituted. In a particular embodiment, R⁶ is selected from substituted C₁-C₆-halogenalkyl, phenyl, halogenphenyl and three-, four-, five- or six-membered carbo- and heterocycle, wherein the carbo- and heterocycle is unsubstituted or substituted by substituents R^{6b} as defined below.

According to another embodiment of formula I, R⁶ is selected from H, CN, substituted C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, C₁-C₆-alkylaryl, aryloxy, arylamino, arylthio, five- or six-membered heteroaryl or aryl which is unsubstituted or substituted with halogen or C₁-C₆-halogenalkyl, and wherein the acyclic moieties of R⁶ are unsubstituted or substituted with identical or different groups R^{6a} as defined below and wherein wherein the carbocycle, heterocycle and heteroaryl and aryl moieties are unsubstituted or substituted with substituents R^{6b} as defined below.

According to still another embodiment of formula I, R⁶ is selected from H, CN, substituted C₁-C₆-alkyl, C₁-C₆-halogenalkyl, CN, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, C₁-C₆-alkylaryl, phenyl, pyridine, pyrimidine, thiophene, imidazole, triazol, oxadiazol wherein the acyclic moieties of R⁶ are unsubstituted or substituted with identical or different groups R^{6a} as defined below and wherein wherein the carbocycle, heterocycle and heteroaryl and aryl moieties are unsubstituted or substituted with substituents R^{6b} as defined below.

According to still another embodiment of formula I, R⁶ is selected from CN, substituted C₁-C₆-alkyl, C₁-C₆-halogenalkyl, CN, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, C₁-C₆-alkylaryl, phenyl, pyridine, pyrimidine, thiophene, imidazole, triazol, oxadiazol wherein the acyclic moieties of R⁶ are unsubstituted or substituted with identical or different groups R^{6a} as defined below and wherein wherein the carbocycle, heterocycle and heteroaryl and aryl moieties are unsubstituted or substituted with substituents R^{6b} as defined below.

According to still another embodiment of formula I, R⁶ is C₁-C₆-alkyl such as CH₃, C₂H₅, n-propyl, i-propyl, n-butyl, i-butyl, tert-butyl, n-pentyl or i-pentyl.

According to still another embodiment of formula I, R⁶ is C₁-C₆-alkyl such as CH₃.

According to still another embodiment of formula I, R⁶ is C₁-C₆-alkyl such as C₂H₅.

According to still another embodiment of formula I, R⁶ is C₁-C₆-alkyl such as CH₃, C₂H₅, n-propyl, i-propyl, n-butyl, i-butyl, tert-butyl, n-pentyl or i-pentyl which is substituted with at least one group R^{6a}, which independently of one another are selected from:

R^{6a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″ a saturated or partially unsaturated three-, four-, five-, six-, seven-, eight-, nine-, or ten-membered carbocycle or heterocycle, five-, six- or ten-membered heteroaryl, aryl or phenoxy, wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbocyclic, heterocyclic, heteroaryl, aryl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl.

According to still another embodiment of formula I, R⁶ is CH₃ is substituted with at least one group R^{6a}, which independently of one another are selected from:

R^{6a} halogen, OH, CN, NO₂, SH, NH₂, NH(C₁-C₄-alkyl), N(C₁-C₄-alkyl)₂, NH-aryl, N(aryl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, five-, six- or ten-membered heteroaryl, an aryl or phenoxy, wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbocyclic, heterocyclic, heteroaryl, heteroaryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl.

According to still another embodiment of formula I, R⁶ is C₂H₅ is substituted with at least one group R^{6a}, which independently of one another are selected from:

R^{6a} halogen, OH, CN, NO₂, SH, NH₂, NH(C₁-C₄-alkyl), N(C₁-C₄-alkyl)₂, NH-aryl, N(aryl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″ a saturated or partially unsaturated three-, four-, five-, six-, seven-, eight-, nine-, or ten-membered carbocycle or heterocycle, five-, six- or ten-membered heteroaryl, phenyl or phenoxy; wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbocyclic, heterocyclic, heteroaryl, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl.

According to still another embodiment of formula I, R⁶ is CH₂CN.

According to still another embodiment of formula I, R⁶ is CH₂OH.

According to still another embodiment of formula I, R⁶ is C₁-C₆-halogenalkyl, in particular C₁-C₄-halogenalkyl, more specifically C₁-C₂-halogenalkyl, such as CF₃, CCl₃, FCH₂, ClCH₂, F₂CH, Cl₂CH, CF₃CH₂, CCl₃CH₂ or CF₂CHF₂.

According to still another embodiment of formula I, R⁶ is CH₂F.

According to still another embodiment of formula I, R⁶ is CHF₂.

According to still another embodiment of formula I, R⁶ is CF₃.

According to still a further embodiment of formula I, R⁶ is C₂-C₆-alkenyl, in particular C₂-C₄-alkenyl, such as CH═CH₂, CH₂CH═CH₂ or C(CH₃)C═CH₂.

According to a further specific embodiment of formula I, R⁶ is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CF═CF₂, CCl═CCl₂, CH₂CH═CHF, CH₂CH═CHCl, CH₂CH═CF₂, CH₂CH═CCl₂, CH₂CF═CF₂, CH₂CCl═CCl₂, CF₂CF═CF₂ or CCl₂CCl═CCl₂.

According to still a further embodiment of formula I, R⁶ is C₂-C₆-cycloalkenyl, in particular C₂-C₄-cycloalkenyl, such as CH═CH₂-cPr.

According to still a further embodiment of formula I, R⁶ is C₂-C₆-alkynyl or C₂-C₆-halogenalkynyl, in particular C₂-C₄-alkynyl or C₂-C₄-halogenalkynyl, such as C≡CH, C≡C—Cl, C≡C—CH₃, CH₂—C≡CH, CH₂—C≡CCl or CH₂—C≡C—CH₃.

According to still a further embodiment of formula I, R⁶ is C₂-C₆-cycloalkynyl in particular C₂-C₄-cycloalkynyl, such as C≡C-cPr.

According to a further specific embodiment of formula I, R⁶ is C₁-C₆-alkoxy, in particular C₁-C₄-alkoxy, more specifically C₁-C₂-alkoxy such as OCH₃, CH₂CH₃ or CH₂OCH₃.

According to a further specific embodiment of formula I, R⁶ is C₁-C₆-alkyl-C₁-C₆-alkoxy, in particular C₁-C₄-alkyl-C₁-C₄-alkoxy, more specifically C₁-C₂-alkyl-C₁-C₂-alkoxy, such as CH₂OCH₃ or CH₂OCH₂CH₃.

According to a further specific embodiment of formula I, R⁶ is C₂-C₆-alkenyloxy, in particular C₂-C₄-alkenyloxy, more specifically C₁-C₂-alkenyloxy such as OCH═CH₂, OCH₂CH═CH₂ OC(CH₃)CH═CH₂, CH₂OCH═CH₂, or CH₂OCH₂CH═CH₂.

According to a further specific embodiment of formula I, R⁶ is C₂-C₆-alkynyloxy, in particular C₂-C₄-alkynyloxy, more specifically C₁-C₂-alkynyloxy such as OC≡CH, OCH₂C≡CH or CH₂OC≡CH

According to a further specific embodiment of formula I, R⁶ is C₁-C₆-halogenalkoxy, in particular C₁-C₄-halogenalkoxy, more specifically C₁-C₂-halogenalkoxy such as OCF₃, OCHF₂, OCH₂F, OCCl₃, OCHCl₂ or OCH₂Cl, in particular OCF₃, OCHF₂, OCCl₃ or OCHCl₂.

According to a further specific embodiment of formula I, R⁶ is C₁-C₆-alkyl-C₁-C₆-halogenalkoxy, in particular C₁-C₄-alkyl-C₁-C₄-halogenalkoxy, more specifically C₁-C₂-alkyl-C₁-C₂-halogenalkoxy such as CH₂OCF₃, CH₂OCHF₂, CH₂OCH₂F, CH₂OCCl₃, CH₂OCHCl₂ or CH₂OCH₂Cl, in particular CH₂OCF₃, CH₂OCHF₂, CH₂OCCl₃ or CH₂OCHCl₂.

According to a further specific embodiment of formula I, R⁶ is CH(═O), C(═O)C₁-C₆-alkyl, C(═O)O(C₁-C₆-alkyl), C(═O)NH(C₁-C₆-alkyl) or C(═O)N(C₁-C₆-alkyl)₂, wherein alkyl is CH₃, C₂H₅, n-propyl, i-propyl, n-butyl, i-butyl, tert-butyl, n-pentyl or i-pentyl.

According to a further specific embodiment of formula I, R⁶ is C₁-C₄-alkyl-CH(═O), C₁-C₄-alkyl-C(═O)C₁-C₆-alkyl, C₁-C₄-alkyl-C(═O)O(C₁-C₆-alkyl), C₁-C₄-alkyl-C(═O)NH(C₁-C₆-alkyl) or C₁-C₄-alkyl-C(═O)N(C₁-C₆-alkyl)₂, especially CH₂CH(═O), CH₂C(═O)C₁-C₆-alkyl, CH₂C(═O)O(C₁-C₆-alkyl), CH₂C(═O)NH(C₁-C₆-alkyl) or CH₂C(═O)N(C₁-C₆-alkyl)₂ wherein alkyl is CH₃, C₂H₅, n-propyl, i-propyl, n-butyl, i-butyl, tert-butyl, n-pentyl or i-pentyl.

According to a further specific embodiment of formula I, R⁶ is CR′═NOR″ such as C(CH₃)═NOCH₃, C(CH₃)═NOCH₂CH₃ or C(CH₃)═NOCF₃.

According to a further specific embodiment of formula I, R⁶ is C₁-C₆-alkyl-NH(C₁-C₄-alkyl) or C₁-C₆-alkyl-N(C₁-C₄-alkyl)₂, wherein alkyl is CH₃, C₂H₅, n-propyl, i-propyl, n-butyl, i-butyl, tert-butyl, n-pentyl or i-pentyl.

According to a further specific embodiment of formula I, R⁶ is C₁-C₆-alkylthio, in particular C₁-C₄-alkoxy, more specifically C₁-C₃-alkylthio such as CH₂SCH₃ or CH₂SCH₂CH₃.

According to a further specific embodiment of formula I, R⁶ is C₁-C₆-alkyl-S(O)ₙ—C₁-C₆-alkyl, wherein alkyl is CH₃, C₂H₅, n-propyl, i-propyl, n-butyl, i-butyl, tert-butyl, n-pentyl or i-pentyl and n is 1, 2 or 3.

According to a further specific embodiment of formula I, R⁶ is C₁-C₆-alkyl-S(O)ₙ—C₁-C₆-halogenalkyl, wherein halogenalkyl is CF₃ or CHF₂ and n is 1, 2 or 3.

According to a further specific embodiment of formula I, R⁶ is C₁-C₆-alkyl-S(O)ₙ-aryl, wherein the aryl or phenyl moiety in each case is unsubstituted or substituted with identical or different groups R^{6b} which independently of one another are selected from halogen, C₁-C₂-alkyl, C₁-C₂-alkoxy, C₁-C₂-halogenalkyl, C₁-C₂-halogenalkoxy and S(O)ₙ—C₁-C₆-alkyl, in particular F, Cl, Br, CH₃, OCH₃, CF₃, CHF₂, OCHF₂, OCF₃. According to one embodiment, R⁶ is unsubstituted phenyl. According to another embodiment, R⁶ is phenyl, that is substituted with one, two or three, in particular one, halogen, in particular selected from F, Cl and Br, more specifically selected from F and Cl.

According to a further specific embodiment of formula I, R¹ is C₁-C₆-alkyl-NH—SO₂—R^{x} wherein R^{x} is C₁-C₄-alkyl, C₁-C₄-halogenalkyl, unsubstituted aryl or aryl that is substituted with one, two, three, four or five substituents R^{x2} independently selected from C₁-C₄-alkyl, halogen, OH, CN, C₁-C₄-halogenalkyl, C₁-C₄-alkoxy, or C₁-C₄-halogenalkoxy, such as CH₂NHSO₂CF₃ or CH₂NHSO₂OCH₃.

According to still another embodiment of formula I, R⁶ is selected from C₁-C₆-alkyl which is substituted, a saturated three-, four-, five-, six-, seven-, eight-, nine-, or ten-membered carbocycle, in particular three-, four-, five- or six-membered, wherein the carbocycle is unsubstituted or substituted with substituents R^{6b} as defined below. According to one embodiment thereof, the carbocycle is unsubstituted.

According to one embodiment, R⁶ is selected from C₁-C₆-alkyl, especially CH₂ which is substituted with a 3-membered saturated carbocycle. According to one embodiment thereof, the carbocycle is unsubstituted, i.e. it does not carry any substituent R^{6b}. According to still another embodiment of formula I, it is substituted with R^{6b}.

According to one embodiment, R⁶ is selected from C₁-C₆-alkyl, especially CH₂ which is substituted with a 4-membered saturated carbocycle. According to one embodiment thereof, the carbocycle is unsubstituted, i.e. it does not carry any substituent R^{6b}. According to still another embodiment of formula I, it is substituted with R^{6b}.

According to one embodiment, R⁶ is selected from C₁-C₆-alkyl, especially CH₂ which is substituted with a 5-membered saturated carbocycle. According to one embodiment thereof, the carbocycle is unsubstituted, i.e. it does not carry any substituent R^{6b}. According to still another embodiment of formula I, it is substituted with R^{6b}.

According to one embodiment, R⁶ is selected from C₁-C₆-alkyl, especially CH₂ which is substituted with a 6-membered saturated carbocycle. According to one embodiment thereof, the carbocycle is unsubstituted, i.e. it does not carry any substituent R^{6b}. According to still another embodiment of formula I, it is substituted with R^{6b}.

According to a further specific embodiment of formula I, R⁶ is C₁-C₆-alkylheterocycle, especially CH₂ substituted with a 4-membered saturated heterocycle which contains 1 or 2 heteroatoms, in particular 1 heteroatom, from the group consisting of N, O and S, as ring members. According to one embodiment, the heterocycle contains one O as heteroatom. For example, the formed heterocycle is oxetane. According to one embodiment thereof, the heterocycle is unsubstituted, i.e. it does not carry any substituent R^{6b}. According to still another embodiment of formula I, it is substituted with R^{6b}.

According to a further specific embodiment of formula I, R⁶ is C₁-C₆-alkylheterocycle, especially CH₂ substituted with a 5-membered saturated heterocycle which contains 1, 2 or 3, in particular 1 or 2, heteroatoms from the group consisting of N, O and S, as ring members. According to one embodiment, the heterocycle contains one O as heteroatom. According to one embodiment thereof, the heterocycle is unsubstituted, i.e. it does not carry any substituent R^{6b}. According to still another embodiment of formula I, it is substituted with R^{6b}.

According to a further specific embodiment of formula I, R⁶ is C₁-C₆-alkylheterocycle, especially CH₂ substituted by a 6-membered saturated heterocycle which contains 1, 2 or 3, in particular 1 or 2, heteroatoms from the group consisting of N, O and S as ring members. According to one embodiment thereof, the heterocycle is unsubstituted, i.e. it does not carry any substituent R^{6b}.

According to still another embodiment of formula I, it is substituted with R^{6b}. According to one specific embodiment thereof, said 6-membered saturated heterocycle contains 1 or 2, in particular 1, heteroatom(s) O. According to one embodiment thereof, the respective 6-membered heterocycle is unsubstituted, i.e. it does not carry any substituent R^{6b}. According to still another embodiment of formula I, it is substituted with R^{6b}.

According to a further specific embodiment of formula I, R⁶ is C₁-C₆-alkylheterocycle, especially CH₂ substituted with a 5-membered saturated heterocycle which contains one N as ring member and optionally one or two groups CH₂ are replaced by C(═O).

According to still another embodiment of formula I, R⁶ is a partially unsaturated three-, four-, five-, six-, seven-, eight-, nine-, or ten-membered carbocycle, in particular three-, four-, five- or six-membered, wherein the carbocycle is unsubstituted or substituted with substituents R^{6b} as defined below. According to one embodiment thereof, the carbocycle is unsubstituted.

According to still another embodiment of formula I, R⁶ is a partially unsaturated three-, four-, five-, six-, seven-, eight-, nine-, or ten-membered carbocycle or heterocycle, in particular three-, four-, five- or six-membered, wherein the heterocycle contains one, two, three or four heteroatoms selected from N, O and S, and wherein the carbocycle and heterocycle are unsubstituted or substituted with substituents R^{6b} as defined below. According to one embodiment thereof, the carbocycle or heterocycle is unsubstituted.

According to still a further embodiment, R⁶ is a saturated three-, four-, five-, six-, seven-, eight-, nine-, or ten-membered carbocycle or heterocycle, in particular three-, four-, five- or six-membered, wherein the heterocycle contains one, two, three or four heteroatoms selected from N, O and S, and wherein the carbocycle and heterocycle are unsubstituted or substituted with substituents R^{6b} as defined below. According to one embodiment thereof, the carbocycle or heterocycle is unsubstituted.

According to still another embodiment of formula I, R⁶ is a saturated three-, four-, five-, six-, seven-, eight-, nine-, or ten-membered carbocycle, in particular three-, four-, five- or six-membered, wherein the carbocycle is unsubstituted or substituted with substituents R^{6b} as defined below. According to one embodiment thereof, the carbocycle is unsubstituted.

According to one embodiment, R⁶ is a 3-membered saturated carbocycle. According to one embodiment thereof, the carbocycle is unsubstituted, i.e. it does not carry any substituent R^{6b}. According to still another embodiment of formula I, it is substituted with R^{6b}.

According to one embodiment, R⁶ is a 3-membered saturated carbocycle, which is unsubstituted such as cyclopropyl.

According to one embodiment, R⁶ is a 3-membered saturated carbocycle, which is substituted with halogen, more specifically by F, such as C₃H₃F₂.

According to one embodiment, R⁶ is a 3-membered saturated carbocycle, which is substituted with halogen. More specifically by Cl, such as C₃H₃Cl₂.

According to one embodiment, R⁶ is a 4-membered saturated carbocycle. According to one embodiment thereof, the carbocycle is unsubstituted, i.e. it does not carry any substituent R^{6b}. According to still another embodiment of formula I, it is substituted with R^{6b}.

According to one embodiment, R⁶ is a 5-membered saturated carbocycle. According to one embodiment thereof, the carbocycle is unsubstituted, i.e. it does not carry any substituent R^{6b}. According to still another embodiment of formula I, it is substituted with R^{6b}.

According to one embodiment, R⁶ is a 6-membered saturated carbocycle. According to one embodiment thereof, the carbocycle is unsubstituted, i.e. it does not carry any substituent R^{6b}. According to still another embodiment of formula I, it is substituted with R^{6b}.

According to still another embodiment of formula I, R⁶ is a partially unsaturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted with substituents R^{6b} as defined below. According to one embodiment thereof, the heterocycle is unsubstituted.

According to still another embodiment of formula I, R⁶ is a saturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted with substituents R^{6b} as defined below. According to one embodiment thereof, the heterocycle is unsubstituted.

According to still another embodiment of formula I, in the embodiments of R⁶ described above, the heterocycle contains preferably one, two or three, more specifically one or two heteroatoms selected from N, O and S. More specifically, the heterocycle contains one heteroatom selected from N, O and S. In particular, the heterocycle contains one or two, in particular one O.

According to one embodiment, R⁶ is a 4-membered saturated heterocycle which contains 1 or 2 heteroatoms, in particular 1 heteroatom, from the group consisting of N, O and S, as ring members. According to one embodiment, the heterocycle contains one O as heteroatom. For example, the formed heterocycle is oxetane. According to one embodiment thereof, the heterocycle is unsubstituted, i.e. it does not carry any substituent R^{6b}. According to still another embodiment of formula I, it is substituted with R^{6b}.

According to still another embodiment of formula I, R⁶ is a 5-membered saturated heterocycle which contains 1, 2 or 3, in particular 1 or 2, heteroatoms from the group consisting of N, O and S, as ring members. According to one embodiment, the heterocycle contains one O as heteroatom. According to one embodiment thereof, the heterocycle is unsubstituted, i.e. it does not carry any substituent R^{6b}. According to still another embodiment of formula I, it is substituted with R^{6b}.

According to still another embodiment of formula I, R⁶ is a 6-membered saturated heterocycle which contains 1, 2 or 3, in particular 1 or 2, heteroatoms from the group consisting of N, O and S as ring members. According to one embodiment thereof, the heterocycle is unsubstituted, i.e. it does not carry any substituent R^{6b}. According to still another embodiment of formula I, it is substituted with R^{6b}. According to one specific embodiment thereof, said 6-membered saturated heterocycle contains 1 or 2, in particular 1, heteroatom(s) O. According to one embodiment thereof, the respective 6-membered heterocycle is unsubstituted, i.e. it does not carry any substituent R^{6b}. According to still another embodiment of formula I, it is substituted with R^{6b}.

According to still another embodiment of formula I, R⁶ is phenyl-C₁-C₆-alkyl, such as phenyl-CH₂, wherein the phenyl moiety in each case is unsubstituted or substituted with one, two or three identical or different groups R^{6b} which independently of one another are selected from CN, halogen, C₁-C₂-alkyl, C₁-C₂-alkoxy, C₁-C₂-halogenalkyl, C₁-C₂-halogenalkoxy and S(O)ₙ—C₁-C₆-alkyl, in particular from CN, F, Cl, Br, CH₃, OCH₃O CF₃. CHF₂, OCHF₂, OCF₃ and S(O)₂CH₃.

According to still another embodiment of formula I, R⁶ is aryl, in particular phenyl, wherein the aryl or phenyl moiety in each case is unsubstituted or substituted with identical or different groups R^{6b} which independently of one another are selected from CN, halogen, C₁-C₂-alkyl, C₁-C₂-alkoxy, C₁-C₂-halogenalkyl, C₁-C₂-halogenalkoxy and S(O)ₙ—C₁-C₆-alkyl, in particular from CN, F, Cl, Br, CH₃, OCH₃, CF₃, CHF₂, OCHF₂, OCF₃. According to one embodiment, R⁶ is unsubstituted phenyl. According to another embodiment, R⁶ is phenyl, that is substituted with one, two or three, in particular one, halogen, in particular selected from F, Cl and Br, more specifically selected from F and Cl.

According to still another embodiment of formula I, R⁶ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R⁶ is a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to a further specific embodiment of formula I, R⁶ is C₁-C₆-alkyl, especially CH₂ substituted by a 5-membered saturated heteroaryl which contains 1, 2 or 3, in particular 1 or 2, heteroatoms from the group consisting of N, O and S as ring members. According to one embodiment thereof, the heteroaryl is unsubstituted, i.e. it does not carry any substituent R^{6b}. According to still another embodiment of formula I, it is substituted by R^{6b}.

According to a further specific embodiment of formula I, R⁶ is C₁-C₆-alkyl, especially CH₂ substituted by a 5-membered saturated heteroaryl which contains one N as ring member. According to one embodiment thereof, the heteroaryl is unsubstituted, i.e. it does not carry any substituent R^{6b}. According to still another embodiment of formula I, it is substituted by R^{6b}.

According to a further specific embodiment of formula I, R⁶ is C₁-C₆-alkyl, especially CH₂ substituted by a 5-membered saturated heteroaryl which contains two N as ring members. According to one embodiment thereof, the heteroaryl is unsubstituted, i.e. it does not carry any substituent R^{6b}. According to still another embodiment of formula I, it is substituted by R^{6b}.

According to a further specific embodiment of formula I, R⁶ is C₁-C₆-alkyl, especially CH₂ substituted by a 5-membered saturated heteroaryl which contains three N as ring members. According to one embodiment thereof, the heteroaryl is unsubstituted, i.e. it does not carry any substituent R^{6b}. According to still another embodiment of formula I, it is substituted by R^{6b}. According to one specific embodiment thereof, said 5-membered saturated heterocycle contains 1 or 2, in particular 1, heteroatom(s) O.

According to a further specific embodiment of formula I, R⁶ is C₁-C₆-alkyl, especially CH₂ substituted by a 5-membered saturated heteroaryl which contains one S as ring member. According to one embodiment thereof, the heteroaryl is unsubstituted, i.e. it does not carry any substituent R^{6b}. According to still another embodiment of formula I, it is substituted by R^{6b}.

According to a further specific embodiment of formula I, R⁶ is C₁-C₆-alkyl, especially CH₂ substituted by a 5-membered saturated heteroaryl which contains one S and one N as ring members. According to one embodiment thereof, the heteroaryl is unsubstituted, i.e. it does not carry any substituent R^{6b}. According to still another embodiment of formula I, it is substituted by R^{6b}.

According to a further specific embodiment of formula I, R⁶ is C₁-C₆-alkyl, especially CH₂ substituted by a 5-membered saturated heteroaryl which contains one S and two N as ring members. According to one embodiment thereof, the heteroaryl is unsubstituted, i.e. it does not carry any substituent R^{6b}. According to still another embodiment of formula I, it is substituted by R^{6b}.

According to a further specific embodiment of formula I, R⁶ is C₁-C₆-alkyl, especially CH₂ substituted by a 5-membered saturated heteroaryl which contains one oxygen and one N as ring members. According to one embodiment thereof, the heteroaryl is unsubstituted, i.e. it does not carry any substituent R^{6b}. According to still another embodiment of formula I, it is substituted by R^{6b}.

According to a further specific embodiment of formula I, R⁶ is C₁-C₆-alkyl, especially CH₂ substituted by a 5-membered saturated heteroaryl which contains one oxygen and two N as ring members. According to one embodiment thereof, the heteroaryl is unsubstituted, i.e. it does not carry any substituent R^{6b}. According to still another embodiment of formula I, it is substituted by R^{6b}.

According to a further specific embodiment of formula I, R⁶ is C₁-C₆-alkyl, especially CH₂ substituted by a 6-membered saturated heteroaryl which contains 1, 2 or 3, in particular 1 or 2, heteroatoms from the group consisting of N, O and S as ring members. According to one embodiment thereof, the heteroaryl is unsubstituted, i.e. it does not carry any substituent R^{6b}. According to still another embodiment of formula I, it is substituted by R^{6b}.

According to a further specific embodiment of formula I, R⁶ is C₁-C₆-alkyl, especially CH₂ substituted by a 6-membered saturated heteroaryl which one N as ring member. According to one embodiment thereof, the heteroaryl is unsubstituted, i.e. it does not carry any substituent R^{6b}. According to still another embodiment of formula I, it is substituted by R^{6b}.

According to a further specific embodiment of formula I, R⁶ is C₁-C₆-alkyl, especially CH₂ substituted by a 6-membered saturated heteroaryl which two N as ring members. According to one embodiment thereof, the heteroaryl is unsubstituted, i.e. it does not carry any substituent R^{6b}. According to still another embodiment of formula I, it is substituted by R^{6b}.

According to a further specific embodiment of formula I, R⁶ is C₁-C₆-alkyl, especially CH₂ substituted by a 10-membered saturated heteroaryl which contains 1, 2 or 3, in particular 1 or 2, heteroatoms from the group consisting of N, O and S as ring members. According to one embodiment thereof, the heteroaryl is unsubstituted, i.e. it does not carry any substituent R^{6b}. According to still another embodiment of formula I, it is substituted by R^{6b}. According to one specific embodiment thereof, said 10-membered saturated heterocycle contains 1 or 2, in particular 1, heteroatom(s) N.

According to a further specific embodiment of formula I, R⁶ is C₁-C₆-alkyl, especially CH₂ substituted by a 10-membered saturated heteroaryl which one N as ring members. According to one embodiment thereof, the heteroaryl is unsubstituted, i.e. it does not carry any substituent R^{6b}. According to still another embodiment of formula I, it is substituted by R^{6b}.

According to still another embodiment of formula I, R⁶ is CH₂ substituted by a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R⁶ is CH₂ substituted by a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to a further particular embodiment, R⁶ is selected from C₁-C₆-alkyl, C₁-C₆-halogenalkyl, CN, C₂-C₆-alkenyl, C₂-C₆-halogenalkenyl, C₃-C₆-cycloalkenyl, C₂-C₆-alkynyl, C₂-C₆-halogenalkynyl, C₁-C₆-alkoxy, C₃-C₆-cycloalkynyl, C(═O)C₁-C₆-alkyl, C(═O)O(C₁-C₆-alkyl), CR′═NOR″, C₃-C₆-halogencycloalkyl a saturated three-, four-, five-, six-, membered carbocycle or heterocycle, a five- or six-membered heteroaryl or aryl; and C₁-C₆-alkyl substituted by CN, C₁-C₆-alkoxy, C₁-C₄-halogenalkoxy, C₁-C₆-alkylthio, S(O)ₙ—C₁-C₆-alkyl, NH—SO₂—R^{x}, NH(C₁-C₆-alkyl), N(C₁-C₆-alkyl)₂, C(═O)C₁-C₆-alkyl, C(═O)O(C₁-C₆-alkyl), a saturated three-, four-, five-, six-, membered carbocycle, heterocycle, aryl or heteroaryl; wherein the carbocycle, heterocycle, aryl and heteroaryl is unsubstituted or carries one, two, three or four substituents R^{6b} as defined below. According to one embodiment thereof, the carbocycle, heterocycle, heteroaryl and aryl are unsubstituted. In a particular embodiment, R⁶ is selected from CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, a saturated three-, four-, five-, six-, membered carbocycle or heterocycle, a five- or six-membered heteroaryl or aryl; and C₁-C₆-alkyl substituted by a saturated three-, four-, five-, six-, membered carbocycle, heterocycle, aryl or heteroaryl; wherein the carbocycle, heterocycle, aryl and heteroaryl are unsubstituted or carries one, two, three or four substituents R^{6b} as defined below.

According to a further particular embodiment, R⁶ is selected from C₁-C₆-halogenalkyl, CN, C₂-C₆-alkenyl, C₂-C₆-halogenalkenyl, C₃-C₆-cycloalkenyl, C₂-C₆-alkynyl, C₂-C₆-halogenalkynyl, C₁-C₆-alkoxy, C₃-C₆-cycloalkynyl, C(═O)C₁-C₆-alkyl, C(═O)O(C₁-C₆-alkyl), CR′═NOR″, C₃-C₆-halogencycloalkyl a saturated three-, four-, five-, six-, membered carbocycle or heterocycle, a five- or six-membered heteroaryl or aryl; and C₁-C₆-alkyl substituted by CN, C₁-C₆-alkoxy, C₁-C₄-halogenalkoxy, C₁-C₆-alkylthio, S(O)ₙ—C₁-C₆-alkyl, NH—SO₂—R^{x}, NH(C₁-C₆-alkyl), N(C₁-C₆-alkyl)₂, C(═O)C₁-C₆-alkyl, C(═O)O(C₁-C₆-alkyl), a saturated three-, four-, five-, six-, membered carbocycle, heterocycle, aryl or heteroaryl; wherein the carbocycle, heterocycle, aryl and heteroaryl is unsubstituted or carries one, two, three or four substituents R^{6b} as defined below. According to one embodiment thereof, the carbocycle, heterocycle, heteroaryl and aryl are unsubstituted. In a particular embodiment, R⁶ is selected from CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, a saturated three-, four-, five-, six-, membered carbocycle or heterocycle, a five- or six-membered heteroaryl or aryl; and C₁-C₆-alkyl substituted by a saturated three-, four-, five-, six-, membered carbocycle, heterocycle, aryl or heteroaryl; wherein the carbocycle, heterocycle, aryl and heteroaryl are unsubstituted or carries one, two, three or four substituents R^{6b} as defined below.

Particularly preferred embodiments of R⁶ according to the invention are in Table P6 below, wherein each line of lines P6-1 to P6-209 corresponds to one particular embodiment of the invention, wherein P6-1 to P6-209 are also in any combination with one another a preferred embodiment of the present invention. The connection point to the carbon atom, to which R⁶ is bound is marked with “#” in the drawings.

TABLE P6
(py = pyridyl)

| No. | R⁶ |
|---|---|
| P6-1 | CH₃ |
| P6-2 | CH₂CH₃ |
| P6-3 | CF₃ |
| P6-4 | CH₂F |
| P6-5 | CH₂Cl |
| P6-6 | CHF₂ |
| P6-7 | CHCl₂ |
| P6-8 | CH₂CF₃ |
| P6-9 | CH₂CCl₃ |
| P6-10 | CF₂CHF₂ |
| P6-11 | CH₂OCH₃ |
| P6-12 | CH₂OCH₂F |
| P6-13 | CH₂OCHF₂ |
| P6-14 | CH₂OCF₃ |
| P6-15 | CH₂OCF₂CHF₂ |
| P6-16 | CH₂NHMe |
| P6-17 | CH₂SMe |
| P6-18 | CH₂SOMe |
| P6-19 | CH₂SO₂Me |
| P6-20 | CH₂NMe₂ |
| P6-21 | CH₂NSO₂CF₃ |
| P6-22 | CH₂NSO₂CH₃ |
| P6-23 | CN |
| P6-24 | CH₂CN |
| P6-25 | CHO |
| P6-26 | COMe |
| P6-27 | CO₂Me |
| P6-28 | CH₂CHO |
| P6-29 | CH₂COMe |
| P6-30 | CH₂CO₂Me |
| P6-31 | 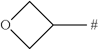 |
| P6-32 | 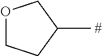 |
| P6-33 | 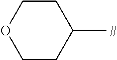 |
| P6-34 | 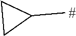 |
| P6-35 | 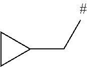 |
| P6-36 | 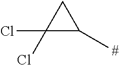 |
| P6-37 | 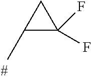 |
| P6-38 | 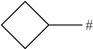 |
| P6-39 | 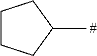 |
| P6-40 | 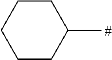 |
| P6-41 | 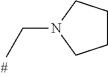 |
| P6-42 | 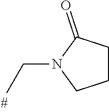 |
| P6-43 | 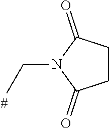 |
| P6-44 | 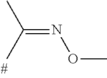 |
| P6-45 | 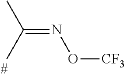 |
| P6-46 | 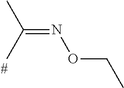 |
| P6-47 | 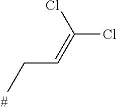 |
| P6-48 | 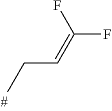 |
| P6-49 | 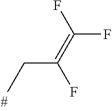 |
| P6-50 | 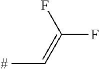 |
| P6-51 | 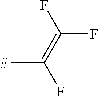 |
| P6-52 | 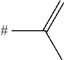 |
| P6-53 | 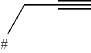 |
| P6-54 | 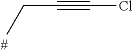 |
| P6-55 | 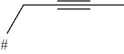 |
| P6-56 | 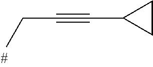 |
| P6-57 | 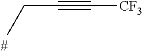 |
| P6-58 | 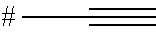 |
| P6-59 | 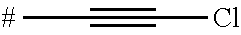 |
| P6-60 | 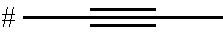 |
| P6-61 | 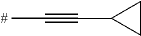 |
| P6-62 | 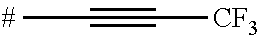 |
| P6-63 | C₆H₅ |
| P6-64 | 4-Cl—C₆H₄ |
| P6-65 | 3-Cl—C₆H₄ |
| P6-66 | 2-Cl—C₆H₄ |
| P6-67 | 2,4-Cl₂—C₆H₃ |
| P6-68 | 4-F—C₆H₄ |
| P6-69 | 3-F—C₆H₄ |
| P6-70 | 2-F—C₆H₄ |
| P6-71 | 2,4-F₂—C₆H₃ |
| P6-72 | 4-MeO—C₆H₄ |
| P6-73 | 3-MeO—C₆H₄ |
| P6-74 | 2-MeO—C₆H₄ |
| P6-75 | 4-MeO₂S—C₆H₄ |
| P6-76 | 3-MeO₂S—C₆H₄ |
| P6-77 | 2-MeO₂S—C₆H₄ |
| P6-78 | —CH₂—C₆H₅ |
| P6-79 | —CH₂—C₆H₄—4-F |
| P6-80 | —CH₂—C₆H₄—4-Cl |
| P6-81 | —CH₂—C₆H₃—2,4-Cl₂ |
| P6-82 | —CH₂—C₆H₄—4-SO₂Me |
| P6-83 | 3-py |
| P6-84 | 2-py |
| P6-85 | 4-py |
| P6-86 | 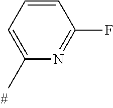 |
| P6-87 | 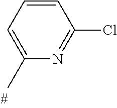 |
| P6-88 | 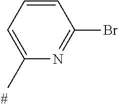 |
| P6-89 | 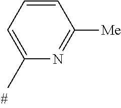 |
| P6-90 | 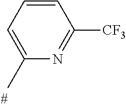 |
| P6-91 | 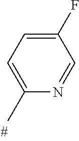 |
| P6-92 | 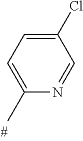 |
| P6-93 | 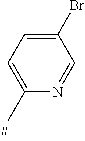 |
| P6-94 | 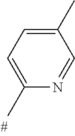 |
| P6-95 | 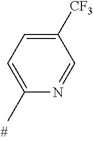 |
| P6-96 | 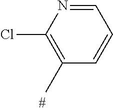 |
| P6-97 | 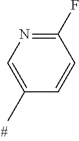 |
| P6-98 | 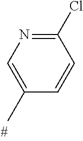 |
| P6-99 | 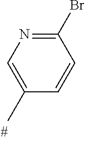 |
| P6-100 | 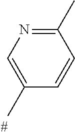 |
| P6-101 | 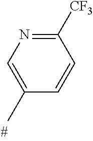 |
| P6-102 | 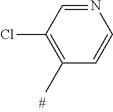 |
| P6-103 | 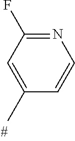 |
| P6-104 | 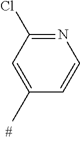 |
| P6-105 | 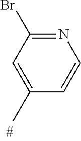 |
| P6-106 | 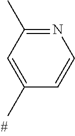 |
| P6-107 | 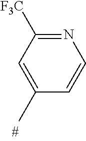 |
| P6-108 | 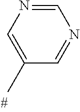 |
| P6-109 | 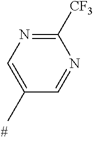 |
| P6-110 | 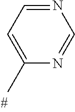 |
| P6-111 | 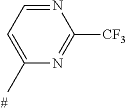 |
| P6-112 | 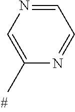 |
| P6-113 | 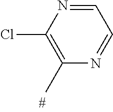 |
| P6-114 | 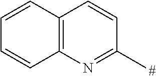 |
| P6-115 | 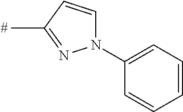 |
| P6-116 | 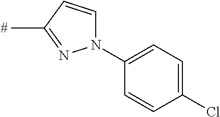 |
| P6-117 | 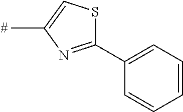 |
| P6-118 | 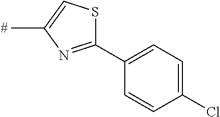 |
| P6-119 | 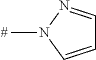 |
| P6-120 | 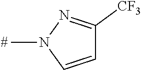 |
| P6-121 | 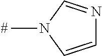 |
| P6-122 | 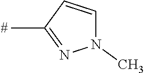 |
| P6-123 | 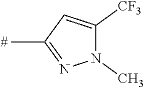 |
| P6-124 | 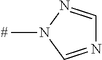 |
| P6-125 | 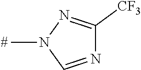 |
| P6-126 | 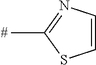 |
| P6-127 | 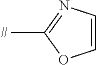 |
| P6-128 | 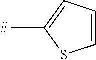 |
| P6-129 | 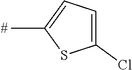 |
| P6-130 | 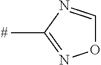 |
| P6-131 | 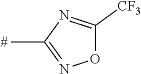 |
| P6-132 | 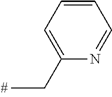 |
| P6-133 | 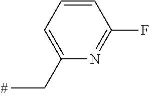 |
| P6-134 | 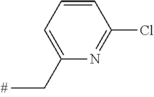 |
| P6-135 | 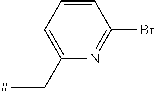 |
| P6-136 | 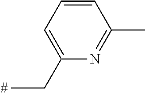 |
| P6-137 | 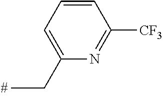 |
| P6-138 | 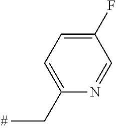 |
| P6-139 | 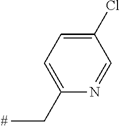 |
| P6-140 | 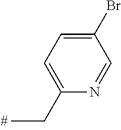 |
| P6-141 | 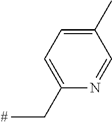 |
| P6-142 | 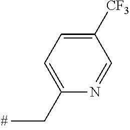 |
| P6-143 | 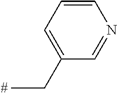 |
| P6-144 | 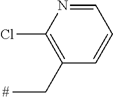 |
| P6-145 | 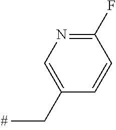 |
| P6-146 | 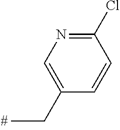 |
| P6-147 | 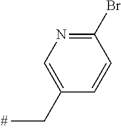 |
| P6-148 | 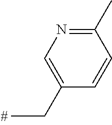 |
| P6-149 | 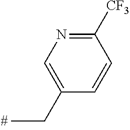 |
| P6-150 | 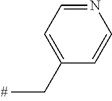 |
| P6-151 | 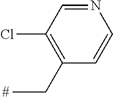 |
| P6-152 | 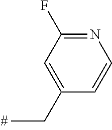 |
| P6-153 | 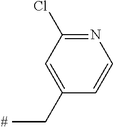 |
| P6-154 | 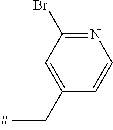 |
| P6-155 | 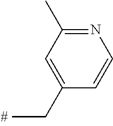 |
| P6-156 | 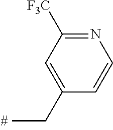 |
| P6-157 | 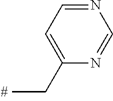 |
| P6-158 | 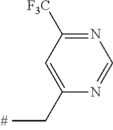 |
| P6-159 | 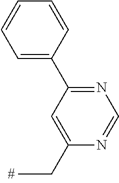 |
| P6-160 | 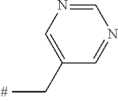 |
| P6-161 | 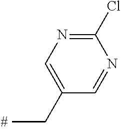 |
| P6-162 | 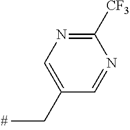 |
| P6-163 | 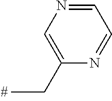 |
| P6-164 | 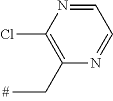 |
| P6-165 | 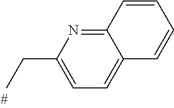 |
| P6-166 | 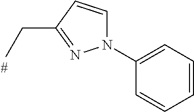 |
| P6-167 | 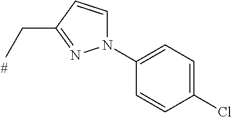 |
| P6-168 | 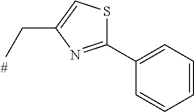 |
| P6-169 | 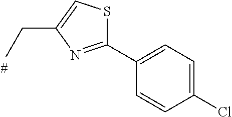 |
| P6-170 | 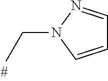 |
| P6-171 | 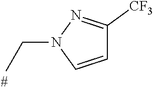 |
| P6-172 | 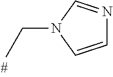 |
| P6-173 | 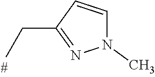 |
| P6-174 | 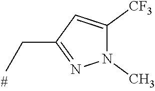 |
| P6-175 | 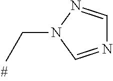 |
| P6-176 | 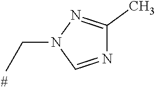 |
| P6-177 | 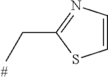 |
| P6-178 | 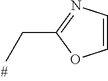 |
| P6-179 | 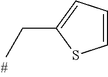 |
| P6-180 | 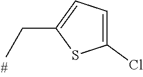 |
| P6-181 | 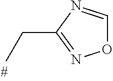 |
| P6-182 | 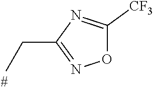 |
| P6-183 | H |
| P6-184 | CH₂CH₂CH₃ |
| P6-185 | CH(CH₃)₂ |
| P6-186 | CH₂CH₂CH₂CH₃ |
| P6-187 | CH(CH₃)CH₂CH₃ |
| P6-188 | CH₂CH(CH₃)CH₃ |
| P6-189 | C(CH₃)₃ |
| P6-190 | —O—C₆H₅ |
| P6-191 | —O—C₆H₄—2-F |
| P6-192 | —O—C₆H₄—3-F |
| P6-193 | —O—C₆H₄—4-F |
| P6-194 | —O—C₆H₄—2-Cl |
| P6-195 | —O—C₆H₄—3-Cl |
| P6-196 | —O—C₆H₄—4-Cl |
| P6-197 | —O—C₆H₄—2-OCH₃ |
| P6-198 | —O—C₆H₄—3-OCH₃ |
| P6-199 | —O—C₆H₄—4-OCH₃ |
| P6-200 | —NH—C₆H₅ |
| P6-201 | —NH—C₆H₄—2-F |
| P6-202 | —NH—C₆H₄—3-F |
| P6-203 | —NH—C₆H₄—4-F |
| P6-204 | —NH—C₆H₄—2-Cl |
| P6-205 | —NH—C₆H₄—3-Cl |
| P6-206 | —NH—C₆H₄—4-Cl |
| P6-207 | —NH—C₆H₄—2-OCH₃ |
| P6-208 | —NH—C₆H₄—3-OCH₃ |
| P6-209 | —NH—C₆H₄—4-OCH₃ |

R⁷ is independently selected from halogen, OH, CN, NO₂, SH, C₁-C₆-alkylthio, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, aryloxy, heteroaryloxy, arylamino, heteroarylamino, arylthio, heteroarylthio, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted with R′″ which is independently selected from halogen, OH, CN, NO₂, SH, NH₂, NH(C₁-C₄-alkyl), N(C₁-C₄-alkyl)₂, NHSO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl;

wherein R^{x} is as defined above;

wherein the acyclic moieties of R⁷ are independently not further substituted or carry one, two, three or up to the maximum possible number of identical or different groups R^{7a}, which independently of one another are selected from:

R^{7a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₃-C₆-cycloalkyl, C₃-C₆-halogencycloalkyl, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbo- or heterocycle, a five-, six- or ten-membered heteroaryl, aryl or phenoxy, wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbo-, heterocyclic, heteroaryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′ and R″ are as defined above; n is 0, 1, 2; and

wherein the carbo-, heterocyclic, heteroaryl and aryl moieties of R⁷ are independently unsubstituted or substituted with identical or different groups R^{7b}, which independently of one another are selected from:

R^{7b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or carry one, two, three, four or five substituents selected from the group consisting of halogen, OH, C₁-C₄-alkyl, C₁-C₄-halogenalkyl, C₁-C₄-alkoxy and C₁-C₄-halogenalkoxy; and wherein R^{x} and n are as defined above.

According to one embodiment of formula I, R⁷ is independently selected from halogen, OH, CN, NO₂, SH, C₁-C₆-alkylthio, NH₂, NH(C₁-C₄-alkyl), N(C₁-C₄-alkyl)₂, NH—SO₂—R^{x}, substituted C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or

wherein the aliphatic moieties of R⁷ are independently not further substituted or carry 1, 2, 3 or up to the maximum possible number of identical or different groups R^{7a}, respectively, which independently of one another are selected from:
R^{7a} halogen, OH, CN, NO₂, SH, NH₂, NH(C₁-C₄-alkyl), N(C₁-C₄-alkyl)₂, NH-aryl, N(aryl)₂, NH(C(═O)C₁-C₄-alkyl), N(C(═O)C₁-C₄-alkyl)₂, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S), five- or six-membered heteroaryl and aryl; wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′, R″ and R″ are as defined above
   wherein the carbocyclic, heterocyclic, heteroaryl and aryl moieties of R⁷ are independently not further substituted or carry 1, 2, 3, 4, 5 or up to the maximum number of identical or different groups R^{7b}, respectively, which independently of one another are selected from:
R^{7b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy.

According to one embodiment of formula I, R⁷ is selected from substituted C₁-C₆-alkyl, C₁-C₆-halogenalkyl, C₂-C₆-halogenalkyl, C₂-C₆-alkenyl, C₂-C₆-halogenalkenyl, C₃-C₆-cycloalkenyl, C₂-C₆-alkynyl, C₂-C₆-halogenalkynyl, C₃-C₆-cycloalkynyl, C₁-C₆-alkoxy, aryloxy, heteroaryloxy, arylamino, heteroarylamino, arylthio, heteroarylthio, CN, CH(═O), C(═O)C₂-C₆-alkyl, C(═O)O(C₂-C₆-alkyl), CR′═NOR″, C₃-C₆-cycloalkyl, C₃-C₆-halogencycloalkyl, C₁-C₆-alkyl-five- and six-membered heteroaryl, a five- or six-membered heteroaryl, benzyl, aryl; wherein R′ and R″ are defined below; and wherein the acyclic moieties of R⁷ are unsubstituted or substituted with identical or different groups R^{7a} as defined below and wherein wherein the carbocycle, heterocycle and heteroaryl and aryl moieties are unsubstituted or substituted with substituents R^{7b} as defined below.

According to one embodiment of formula I, R⁷ is selected from C₁-C₆-alkyl substituted with halogen, CN, C₁-C₆-alkoxy, aryloxy, arylamino, arylthio, C₁-C₄-halogenalkoxy, C₁-C₆-alkylthio, S(O)ₙ—C₁-C₆-alkyl, NH—SO₂—R^{x}, N(C₁-C₆-alkyl)₂, NH—SO₂—R^{x}, NH(C₁-C₆-alkyl), N(C₁-C₆-alkyl)₂, CH(═O), C(═O)C₁-C₆-alkyl, C(═O)O(C₁-C₆-alkyl), a saturated three-, four-, five-, six-, membered carbo- or heterocycle, aryl, a five- or six-membered heteroaryl; wherein R^{x} is defined below; and wherein the acyclic moieties of R⁷ are unsubstituted or substituted with identical or different groups R^{7a} as defined below and wherein wherein the carbocycle, heterocycle and heteroaryl and aryl moieties are unsubstituted or substituted with substituents R^{7b} as defined below.

According to another embodiment of formula I, R⁷ is F.

According to another embodiment of formula I, R⁷ is Cl.

According to another embodiment of formula I, R⁷ is Br.

According to still another embodiment of formula I, R⁷ is OH.

According to still another embodiment of formula I, R⁷ is CN.

According to still another embodiment of formula I, R⁷ is NO₂.

According to still another embodiment of formula I, R⁷ is SH.

According to still another embodiment of formula I, R⁷ is C₁-C₆-alkylthio, such as SCH₃, SC₂H₅, S-n-propyl, S-i-propyl, S-n-butyl, S-i-butyl, S-tert-butyl, S-n-pentyl, S-i-pentyl, CH₂SCH₃ or CH₂SCH₂CH₃.

According to still another embodiment of formula I, R⁷ is C₁-C₆-halogenalkylthio, such as SCF₃, SCCl₃, CH₂SCF₃ or CH₂SCF₃.

According to still another embodiment of formula I, R⁷ is selected from CN, substituted C₁-C₆-alkyl, C₁-C₆-halogenalkyl or C₁-C₆-alkyl which is substituted, C₁-C₆-halogenalkyl, phenyl, halogenphenyl and three-, four-, five- or six-membered carbo- and heterocycle, wherein the carbo- and heterocycle is unsubstituted or is substituted with substituents R^{7b} as defined below. According to one embodiment thereof, the carbocycle is unsubstituted. In a particular embodiment, R⁷ is selected from C₁-C₆-halogenalkyl, phenyl-CH₂, halogenphenyl-CH₂, phenyl, halogenphenyl and three-, four-, five- or six-membered carbo- and heterocycle, wherein the carbo- and heterocycle is unsubstituted or is substituted with substituents R^{7b} as defined below.

According to still another embodiment of formula I, R⁷ is selected from CN, substituted C₁-C₆-alkyl, C₁-C₆-halogenalkyl or C₁-C₆-alkyl which is substituted, C₁-C₆-halogenalkyl, phenyl, halogenphenyl, aryloxy, heteroaryloxy, arylamino, heteroarylamino, arylthio, heteroarylthio and three-, four-, five- or six-membered carbo- and heterocycle, wherein the carbo- and heterocycle is unsubstituted or substituted by substituents R^{7b} as defined below. According to one embodiment thereof, the carbo- and heterocycle is unsubstituted. In a particular embodiment, R⁷ is selected from substituted C₁-C₆-halogenalkyl, phenyl, halogenphenyl and three-, four-, five- or six-membered carbo- and heterocycle, wherein the carbo- and heterocycle is unsubstituted or substituted by substituents R^{7b} as defined below.

According to another embodiment of formula I, R⁷ is selected from CN, substituted C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, C₁-C₆-alkylaryl, aryloxy, arylamino, arylthio, five- or six-membered heteroaryl or aryl which is unsubstituted or substituted with halogen or C₁-C₆-halogenalkyl, and wherein the acyclic moieties of R⁷ are unsubstituted or substituted with identical or different groups R^{7a} as defined below and wherein wherein the carbocycle, heterocycle and heteroaryl and aryl moieties are unsubstituted or substituted with substituents R^{7b} as defined below.

According to still another embodiment of formula I, R⁷ is selected from CN, substituted C₁-C₆-alkyl, C₁-C₆-halogenalkyl, CN, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, C₁-C₆-alkylaryl, phenyl, pyridine, pyrimidine, thiophene, imidazole, triazol, oxadiazol wherein the acyclic moieties of R⁷ are unsubstituted or substituted with identical or different groups R^{7a} as defined below and wherein wherein the carbocycle, heterocycle and heteroaryl and aryl moieties are unsubstituted or substituted with substituents R^{7b} as defined below.

According to still another embodiment of formula I, R⁷ is C₁-C₆-alkyl such as CH₃, C₂H₅, n-propyl, i-propyl, n-butyl, i-butyl, tert-butyl, n-pentyl or i-pentyl.

According to still another embodiment of formula I, R⁷ is C₁-C₆-alkyl such as CH₃.

According to still another embodiment of formula I, R⁷ is C₁-C₆-alkyl such as C₂H₅.

According to still another embodiment of formula I, R⁷ is C₁-C₆-alkyl such as CH₃, C₂H₅, n-propyl, i-propyl, n-butyl, i-butyl, tert-butyl, n-pentyl or i-pentyl which is substituted with at least one group R^{7a}, which independently of one another are selected from:

R^{7a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″ a saturated or partially unsaturated three-, four-, five-, six-, seven-, eight-, nine-, or ten-membered carbocycle or heterocycle, five-, six- or ten-membered heteroaryl, aryl or phenoxy, wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbocyclic, heterocyclic, heteroaryl, aryl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl.

According to still another embodiment of formula I, R⁷ is CH₃ is substituted with at least one group R^{7a}, which independently of one another are selected from:

R^{7a} halogen, OH, CN, NO₂, SH, NH₂, NH(C₁-C₄-alkyl), N(C₁-C₄-alkyl)₂, NH-aryl, N(aryl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, five-, six- or ten-membered heteroaryl, an aryl or phenoxy, wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbocyclic, heterocyclic, heteroaryl, heteroaryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl.

According to still another embodiment of formula I, R⁷ is C₂H₅ is substituted with at least one group R^{7a}, which independently of one another are selected from:

R^{7a} halogen, OH, CN, NO₂, SH, NH₂, NH(C₁-C₄-alkyl), N(C₁-C₄-alkyl)₂, NH-aryl, N(aryl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″ a saturated or partially unsaturated three-, four-, five-, six-, seven-, eight-, nine-, or ten-membered carbocycle or heterocycle, five-, six- or ten-membered heteroaryl, phenyl or phenoxy; wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbocyclic, heterocyclic, heteroaryl, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl.

According to still another embodiment of formula I, R⁷ is CH₂CN.

According to still another embodiment of formula I, R⁷ is CH₂OH.

According to still another embodiment of formula I, R⁷ is C₁-C₆-halogenalkyl, in particular C₁-C₄-halogenalkyl, more specifically C₁-C₂-halogenalkyl, such as CF₃, CCl₃, FCH₂, ClCH₂, F₂CH, Cl₂CH, CF₃CH₂, CCl₃CH₂ or CF₂CHF₂.

According to still another embodiment of formula I, R⁷ is CH₂F.

According to still another embodiment of formula I, R⁷ is CHF₂.

According to still another embodiment of formula I, R⁷ is CF₃.

According to still a further embodiment of formula I, R⁷ is C₂-C₆-alkenyl, in particular C₂-C₄-alkenyl, such as CH═CH₂, CH₂CH═CH₂ or C(CH₃)C═CH₂.

According to a further specific embodiment of formula I, R⁷ is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CF═CF₂, CCl═CCl₂, CH₂CH═CHF, CH₂CH═CHCl, CH₂CH═CF₂, CH₂CH═CCl₂, CH₂CF═CF₂, CH₂CCl═CCl₂, CF₂CF═CF₂ or CCl₂CCl═CCl₂.

According to still a further embodiment of formula I, R⁷ is C₂-C₆-cycloalkenyl, in particular C₂-C₄-cycloalkenyl, such as CH═CH₂-cPr.

According to still a further embodiment of formula I, R⁷ is C₂-C₆-alkynyl or C₂-C₆-halogenalkynyl, in particular C₂-C₄-alkynyl or C₂-C₄-halogenalkynyl, such as C≡CH, C≡C—Cl, C≡C—CH₃, CH₂—C≡CH, CH₂—C≡CCl or CH₂—C≡C—CH₃.

According to still a further embodiment of formula I, R⁷ is C₂-C₆-cycloalkynyl in particular C₂-C₄-cycloalkynyl, such as C≡C-cPr.

According to a further specific embodiment of formula I, R⁷ is C₁-C₆-alkoxy, in particular C₁-C₄-alkoxy, more specifically C₁-C₂-alkoxy such as OCH₃, CH₂CH₃ or CH₂OCH₃.

According to a further specific embodiment of formula I, R⁷ is C₁-C₆-alkyl-C₁-C₆-alkoxy, in particular C₁-C₄-alkyl-C₁-C₄-alkoxy, more specifically C₁-C₂-alkyl-C₁-C₂-alkoxy, such as CH₂OCH₃ or CH₂OCH₂CH₃.

According to a further specific embodiment of formula I, R⁷ is C₂-C₆-alkenyloxy, in particular C₂-C₄-alkenyloxy, more specifically C₁-C₂-alkenyloxy such as OCH═CH₂, OCH₂CH═CH₂ OC(CH₃)CH═CH₂, CH₂OCH═CH₂, or CH₂OCH₂CH═CH₂.

According to a further specific embodiment of formula I, R⁷ is C₂-C₆-alkynyloxy, in particular C₂-C₄-alkynyloxy, more specifically C₁-C₂-alkynyloxy such as OC≡CH, OCH₂C≡CH or CH₂OC≡CH

According to a further specific embodiment of formula I, R⁷ is C₁-C₆-halogenalkoxy, in particular C₁-C₄-halogenalkoxy, more specifically C₁-C₂-halogenalkoxy such as OCF₃, OCHF₂, OCH₂F, OCCl₃, OCHCl₂ or OCH₂Cl, in particular OCF₃, OCHF₂, OCCl₃ or OCHCl₂.

According to a further specific embodiment of formula I, R⁷ is C₁-C₆-alkyl-C₁-C₆-halogenalkoxy, in particular C₁-C₄-alkyl-C₁-C₄-halogenalkoxy, more specifically C₁-C₂-alkyl-C₁-C₂-halogenalkoxy such as CH₂OCF₃, CH₂OCHF₂, CH₂OCH₂F, CH₂OCCl₃, CH₂OCHCl₂ or CH₂OCH₂Cl, in particular CH₂OCF₃, CH₂OCHF₂, CH₂OCCl₃ or CH₂OCHCl₂.

According to a further specific embodiment of formula I, R⁷ is CH(═O), C(═O)C₁-C₆-alkyl, C(═O)O(C₁-C₆-alkyl), C(═O)NH(C₁-C₆-alkyl) or C(═O)N(C₁-C₆-alkyl)₂, wherein alkyl is CH₃, C₂H₅, n-propyl, i-propyl, n-butyl, i-butyl, tert-butyl, n-pentyl or i-pentyl.

According to a further specific embodiment of formula I, R⁷ is C₁-C₄-alkyl-CH(═O), C₁-C₄-alkyl-C(═O)C₁-C₆-alkyl, C₁-C₄-alkyl-C(═O)O(C₁-C₆-alkyl), C₁-C₄-alkyl-C(═O)NH(C₁-C₆-alkyl) or C₁-C₄-alkyl-C(═O)N(C₁-C₆-alkyl)₂, especially CH₂CH(═O), CH₂C(═O)C₁-C₆-alkyl, CH₂C(═O)O(C₁-C₆-alkyl), CH₂C(═O)NH(C₁-C₆-alkyl) or CH₂C(═O)N(C₁-C₆-alkyl)₂ wherein alkyl is CH₃, C₂H₅, n-propyl, i-propyl, n-butyl, i-butyl, tert-butyl, n-pentyl or i-pentyl.

According to a further specific embodiment of formula I, R⁷ is CR′═NOR″ such as C(CH₃)═NOCH₃, C(CH₃)═NOCH₂CH₃ or C(CH₃)═NOCF₃.

According to a further specific embodiment of formula I, R⁷ is C₁-C₆-alkyl-NH(C₁-C₄-alkyl) or C₁-C₆-alkyl-N(C₁-C₄-alkyl)₂, wherein alkyl is CH₃, C₂H₅, n-propyl, i-propyl, n-butyl, i-butyl, tert-butyl, n-pentyl or i-pentyl.

According to a further specific embodiment of formula I, R⁷ is C₁-C₆-alkylthio, in particular C₁-C₄-alkoxy, more specifically C₁-C₃-alkylthio such as CH₂SCH₃ or CH₂SCH₂CH₃.

According to a further specific embodiment of formula I, R⁷ is C₁-C₆-alkyl-S(O)ₙ—C₁-C₆-alkyl, wherein alkyl is CH₃, C₂H₅, n-propyl, i-propyl, n-butyl, i-butyl, tert-butyl, n-pentyl or i-pentyl and n is 1, 2 or 3.

According to a further specific embodiment of formula I, R⁷ is C₁-C₆-alkyl-S(O)ₙ—C₁-C₆-halogenalkyl, wherein halogenalkyl is CF₃ or CHF₂ and n is 1, 2 or 3.

According to a further specific embodiment of formula I, R⁷ is C₁-C₆-alkyl-S(O)ₙ-aryl, wherein the aryl or phenyl moiety in each case is unsubstituted or substituted with identical or different groups R^{7b} which independently of one another are selected from halogen, C₁-C₂-alkyl, C₁-C₂-alkoxy, C₁-C₂-halogenalkyl, C₁-C₂-halogenalkoxy and S(O)ₙ—C₁-C₆-alkyl, in particular F, Cl, Br, CH₃, OCH₃, CF₃, CHF₂, OCHF₂, OCF₃. According to one embodiment, R⁷ is unsubstituted phenyl. According to another embodiment, R⁷ is phenyl, that is substituted with one, two or three, in particular one, halogen, in particular selected from F, Cl and Br, more specifically selected from F and Cl.

According to a further specific embodiment of formula I, R⁷ is C₁-C₆-alkyl-NH—SO₂—R^{x} wherein R^{x} is C₁-C₄-alkyl, C₁-C₄-halogenalkyl, unsubstituted aryl or aryl that is substituted with one, two, three, four or five substituents R^{x2} independently selected from C₁-C₄-alkyl, halogen, OH, CN, C₁-C₄-halogenalkyl, C₁-C₄-alkoxy, or C₁-C₄-halogenalkoxy, such as CH₂NHSO₂CF₃ or CH₂NHSO₂CH₃.

According to still another embodiment of formula I, R⁷ is selected from C₁-C₆-alkyl which is substituted, a saturated three-, four-, five-, six-, seven-, eight-, nine-, or ten-membered carbocycle, in particular three-, four-, five- or six-membered, wherein the carbocycle is unsubstituted or substituted with substituents R^{7b} as defined below. According to one embodiment thereof, the carbocycle is unsubstituted.

According to one embodiment, R⁷ is selected from C₁-C₆-alkyl, especially CH₂ which is substituted with a 3-membered saturated carbocycle. According to one embodiment thereof, the carbocycle is unsubstituted, i.e. it does not carry any substituent R^{7b}. According to still another embodiment of formula I, it is substituted with R^{7b}.

According to one embodiment, R⁷ is selected from C₁-C₆-alkyl, especially CH₂ which is substituted with a 4-membered saturated carbocycle. According to one embodiment thereof, the carbocycle is unsubstituted, i.e. it does not carry any substituent R^{7b}. According to still another embodiment of formula I, it is substituted with R^{7b}.

According to one embodiment, R⁷ is selected from C₁-C₆-alkyl, especially CH₂ which is substituted with a 5-membered saturated carbocycle. According to one embodiment thereof, the carbocycle is unsubstituted, i.e. it does not carry any substituent R^{7b}. According to still another embodiment of formula I, it is substituted with R^{7b}.

According to one embodiment, R⁷ is selected from C₁-C₆-alkyl, especially CH₂ which is substituted with a 6-membered saturated carbocycle. According to one embodiment thereof, the carbocycle is unsubstituted, i.e. it does not carry any substituent R^{7b}. According to still another embodiment of formula I, it is substituted with R^{7b}.

According to a further specific embodiment of formula I, R⁷ is C₁-C₆-alkylheterocycle, especially CH₂ substituted with a 4-membered saturated heterocycle which contains 1 or 2 heteroatoms, in particular 1 heteroatom, from the group consisting of N, O and S, as ring members. According to one embodiment, the heterocycle contains one O as heteroatom. For example, the formed heterocycle is oxetane. According to one embodiment thereof, the heterocycle is unsubstituted, i.e. it does not carry any substituent R^{7b}. According to still another embodiment of formula I, it is substituted with R^{7b}.

According to a further specific embodiment of formula I, R⁷ is C₁-C₆-alkylheterocycle, especially CH₂ substituted with a 5-membered saturated heterocycle which contains 1, 2 or 3, in particular 1 or 2, heteroatoms from the group consisting of N, O and S, as ring members. According to one embodiment, the heterocycle contains one O as heteroatom. According to one embodiment thereof, the heterocycle is unsubstituted, i.e. it does not carry any substituent R^{7b}. According to still another embodiment of formula I, it is substituted with R^{7b}.

According to a further specific embodiment of formula I, R⁷ is C₁-C₆-alkylheterocycle, especially CH₂ substituted by a 6-membered saturated heterocycle which contains 1, 2 or 3, in particular 1 or 2, heteroatoms from the group consisting of N, O and S as ring members. According to one embodiment thereof, the heterocycle is unsubstituted, i.e. it does not carry any substituent R^{7b}. According to still another embodiment of formula I, it is substituted with R^{7b}. According to one specific embodiment thereof, said 6-membered saturated heterocycle contains 1 or 2, in particular 1, heteroatom(s) O. According to one embodiment thereof, the respective 6-membered heterocycle is unsubstituted, i.e. it does not carry any substituent R^{7b}. According to still another embodiment of formula I, it is substituted with R^{7b}.

According to a further specific embodiment of formula I, R⁷ is C₁-C₆-alkylheterocycle, especially CH₂ substituted with a 5-membered saturated heterocycle which contains one N as ring member and optionally one or two groups CH₂ are replaced by C(═O).

According to still another embodiment of formula I, R⁷ is a partially unsaturated three-, four-, five-, six-, seven-, eight-, nine-, or ten-membered carbocycle, in particular three-, four-, five- or six-membered, wherein the carbocycle is unsubstituted or substituted with substituents R^{7b} as defined below. According to one embodiment thereof, the carbocycle is unsubstituted.

According to still another embodiment of formula I, R⁷ is a partially unsaturated three-, four-, five-, six-, seven-, eight-, nine-, or ten-membered carbocycle or heterocycle, in particular three-, four-, five- or six-membered, wherein the heterocycle contains one, two, three or four heteroatoms selected from N, O and S, and wherein the carbocycle and heterocycle are unsubstituted or substituted with substituents R^{7b} as defined below. According to one embodiment thereof, the carbocycle or heterocycle is unsubstituted.

According to still a further embodiment, R⁷ is a saturated three-, four-, five-, six-, seven-, eight-, nine-, or ten-membered carbocycle or heterocycle, in particular three-, four-, five- or six-membered, wherein the heterocycle contains one, two, three or four heteroatoms selected from N, O and S, and wherein the carbocycle and heterocycle are unsubstituted or substituted with substituents R^{7b} as defined below. According to one embodiment thereof, the carbocycle or heterocycle is unsubstituted.

According to still another embodiment of formula I, R⁷ is a saturated three-, four-, five-, six-, seven-, eight-, nine-, or ten-membered carbocycle, in particular three-, four-, five- or six-membered, wherein the carbocycle is unsubstituted or substituted with substituents R^{7b} as defined below. According to one embodiment thereof, the carbocycle is unsubstituted.

According to one embodiment, R⁷ is a 3-membered saturated carbocycle. According to one embodiment thereof, the carbocycle is unsubstituted, i.e. it does not carry any substituent R^{7b}. According to still another embodiment of formula I, it is substituted with R^{7b}.

According to one embodiment, R⁷ is a 3-membered saturated carbocycle, which is unsubstituted such as cyclopropyl.

According to one embodiment, R⁷ is a 3-membered saturated carbocycle, which is substituted with halogen, more specifically by F, such as C₃H₃F₂.

According to one embodiment, R⁷ is a 3-membered saturated carbocycle, which is substituted with halogen. More specifically by Cl, such as C₃H₃Cl₂.

According to one embodiment, R⁷ is a 4-membered saturated carbocycle. According to one embodiment thereof, the carbocycle is unsubstituted, i.e. it does not carry any substituent R^{7b}. According to still another embodiment of formula I, it is substituted with R^{7b}.

According to one embodiment, R⁷ is a 5-membered saturated carbocycle. According to one embodiment thereof, the carbocycle is unsubstituted, i.e. it does not carry any substituent R^{7b}. According to still another embodiment of formula I, it is substituted with R^{7b}.

According to one embodiment, R⁷ is a 6-membered saturated carbocycle. According to one embodiment thereof, the carbocycle is unsubstituted, i.e. it does not carry any substituent R^{7b}. According to still another embodiment of formula I, it is substituted with R^{7b}.

According to still another embodiment of formula I, R⁷ is a partially unsaturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted with substituents R^{7b} as defined below. According to one embodiment thereof, the heterocycle is unsubstituted.

According to still another embodiment of formula I, R⁷ is a saturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted with substituents R^{7b} as defined below. According to one embodiment thereof, the heterocycle is unsubstituted.

According to still another embodiment of formula I, in the embodiments of R⁷ described above, the heterocycle contains preferably one, two or three, more specifically one or two heteroatoms selected from N, O and S. More specifically, the heterocycle contains one heteroatom selected from N, O and S. In particular, the heterocycle contains one or two, in particular one O.

According to one embodiment, R⁷ is a 4-membered saturated heterocycle which contains 1 or 2 heteroatoms, in particular 1 heteroatom, from the group consisting of N, O and S, as ring members. According to one embodiment, the heterocycle contains one O as heteroatom. For example, the formed heterocycle is oxetane. According to one embodiment thereof, the heterocycle is unsubstituted, i.e. it does not carry any substituent R^{7b}. According to still another embodiment of formula I, it is substituted with R^{7b}.

According to still another embodiment of formula I, R⁷ is a 5-membered saturated heterocycle which contains 1, 2 or 3, in particular 1 or 2, heteroatoms from the group consisting of N, O and S, as ring members. According to one embodiment, the heterocycle contains one O as heteroatom. According to one embodiment thereof, the heterocycle is unsubstituted, i.e. it does not carry any substituent R^{7b}. According to still another embodiment of formula I, it is substituted with R^{7b}.

According to still another embodiment of formula I, R⁷ is a 6-membered saturated heterocycle which contains 1, 2 or 3, in particular 1 or 2, heteroatoms from the group consisting of N, O and S as ring members. According to one embodiment thereof, the heterocycle is unsubstituted, i.e. it does not carry any substituent R^{7b}. According to still another embodiment of formula I, it is substituted with R^{7b}. According to one specific embodiment thereof, said 6-membered saturated heterocycle contains 1 or 2, in particular 1, heteroatom(s) O. According to one embodiment thereof, the respective 6-membered heterocycle is unsubstituted, i.e. it does not carry any substituent R^{7b}. According to still another embodiment of formula I, it is substituted with R^{7b}.

According to still another embodiment of formula I, R⁷ is phenyl-C₁-C₆-alkyl, such as phenyl-CH₂, wherein the phenyl moiety in each case is unsubstituted or substituted with one, two or three identical or different groups R^{7b} which independently of one another are selected from CN, halogen, C₁-C₂-alkyl, C₁-C₂-alkoxy, C₁-C₂-halogenalkyl, C₁-C₂-halogenalkoxy and S(O)ₙ—C₁-C₆-alkyl, in particular from CN, F, Cl, Br, CH₃, OCH₃, CF₃, CHF₂, OCHF₂, OCF₃ and S(O)₂CH₃.

According to still another embodiment of formula I, R⁷ is aryl, in particular phenyl, wherein the aryl or phenyl moiety in each case is unsubstituted or substituted with identical or different groups R^{7b} which independently of one another are selected from CN, halogen, C₁-C₂-alkyl, C₁-C₂-alkoxy, C₁-C₂-halogenalkyl, C₁-C₂-halogenalkoxy and S(O)ₙ—C₁-C₆-alkyl, in particular from CN, F, Cl, Br, CH₃, OCH₃, CF₃, CHF₂, OCHF₂, OCF₃. According to one embodiment, R⁷ is unsubstituted phenyl. According to another embodiment, R⁷ is phenyl, that is substituted with one, two or three, in particular one, halogen, in particular selected from F, Cl and Br, more specifically selected from F and Cl.

According to still another embodiment of formula I, R⁷ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R⁷ is a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to a further specific embodiment of formula I, R⁷ is C₁-C₆-alkyl, especially CH₂ substituted by a 5-membered saturated heteroaryl which contains 1, 2 or 3, in particular 1 or 2, heteroatoms from the group consisting of N, O and S as ring members. According to one embodiment thereof, the heteroaryl is unsubstituted, i.e. it does not carry any substituent R^{7b}. According to still another embodiment of formula I, it is substituted by R^{7b}.

According to a further specific embodiment of formula I, R⁷ is C₁-C₆-alkyl, especially CH₂ substituted by a 5-membered saturated heteroaryl which contains one N as ring member. According to one embodiment thereof, the heteroaryl is unsubstituted, i.e. it does not carry any substituent R^{7b}. According to still another embodiment of formula I, it is substituted by R^{7b}.

According to a further specific embodiment of formula I, R⁷ is C₁-C₆-alkyl, especially CH₂ substituted by a 5-membered saturated heteroaryl which contains two N as ring members. According to one embodiment thereof, the heteroaryl is unsubstituted, i.e. it does not carry any substituent R^{7b}. According to still another embodiment of formula I, it is substituted by R^{7b}.

According to a further specific embodiment of formula I, R⁷ is C₁-C₆-alkyl, especially CH₂ substituted by a 5-membered saturated heteroaryl which contains three N as ring members. According to one embodiment thereof, the heteroaryl is unsubstituted, i.e. it does not carry any substituent R^{7b}. According to still another embodiment of formula I, it is substituted by R^{7b}. According to one specific embodiment thereof, said 5-membered saturated heterocycle contains 1 or 2, in particular 1, heteroatom(s) O.

According to a further specific embodiment of formula I, R⁷ is C₁-C₆-alkyl, especially CH₂ substituted by a 5-membered saturated heteroaryl which contains one S as ring member. According to one embodiment thereof, the heteroaryl is unsubstituted, i.e. it does not carry any substituent R^{7b}. According to still another embodiment of formula I, it is substituted by R^{7b}.

According to a further specific embodiment of formula I, R⁷ is C₁-C₆-alkyl, especially CH₂ substituted by a 5-membered saturated heteroaryl which contains one S and one N as ring members. According to one embodiment thereof, the heteroaryl is unsubstituted, i.e. it does not carry any substituent R^{7b}. According to still another embodiment of formula I, it is substituted by R^{7b}.

According to a further specific embodiment of formula I, R⁷ is C₁-C₆-alkyl, especially CH₂ substituted by a 5-membered saturated heteroaryl which contains one S and two N as ring members. According to one embodiment thereof, the heteroaryl is unsubstituted, i.e. it does not carry any substituent R^{7b}. According to still another embodiment of formula I, it is substituted by R^{7b}.

According to a further specific embodiment of formula I, R⁷ is C₁-C₆-alkyl, especially CH₂ substituted by a 5-membered saturated heteroaryl which contains one oxygen and one N as ring members. According to one embodiment thereof, the heteroaryl is unsubstituted, i.e. it does not carry any substituent R^{7b}. According to still another embodiment of formula I, it is substituted by R^{7b}.

According to a further specific embodiment of formula I, R⁷ is C₁-C₆-alkyl, especially CH₂ substituted by a 5-membered saturated heteroaryl which contains one oxygen and two N as ring members. According to one embodiment thereof, the heteroaryl is unsubstituted, i.e. it does not carry any substituent R^{7b}. According to still another embodiment of formula I, it is substituted by R^{7b}.

According to a further specific embodiment of formula I, R⁷ is C₁-C₆-alkyl, especially CH₂ substituted by a 6-membered saturated heteroaryl which contains 1, 2 or 3, in particular 1 or 2, heteroatoms from the group consisting of N, O and S as ring members. According to one embodiment thereof, the heteroaryl is unsubstituted, i.e. it does not carry any substituent R^{7b}. According to still another embodiment of formula I, it is substituted by R^{7b}.

According to a further specific embodiment of formula I, R⁷ is C₁-C₆-alkyl, especially CH₂ substituted by a 6-membered saturated heteroaryl which one N as ring member. According to one embodiment thereof, the heteroaryl is unsubstituted, i.e. it does not carry any substituent R^{7b}. According to still another embodiment of formula I, it is substituted by R^{7b}.

According to a further specific embodiment of formula I, R⁷ is C₁-C₆-alkyl, especially CH₂ substituted by a 6-membered saturated heteroaryl which two N as ring members. According to one embodiment thereof, the heteroaryl is unsubstituted, i.e. it does not carry any substituent R^{7b}. According to still another embodiment of formula I, it is substituted by R^{7b}.

According to a further specific embodiment of formula I, R⁷ is C₁-C₆-alkyl, especially CH₂ substituted by a 10-membered saturated heteroaryl which contains 1, 2 or 3, in particular 1 or 2, heteroatoms from the group consisting of N, O and S as ring members. According to one embodiment thereof, the heteroaryl is unsubstituted, i.e. it does not carry any substituent R^{7b}. According to still another embodiment of formula I, it is substituted by R^{7b}. According to one specific embodiment thereof, said 10-membered saturated heterocycle contains 1 or 2, in particular 1, heteroatom(s) N.

According to a further specific embodiment of formula I, R⁷ is C₁-C₆-alkyl, especially CH₂ substituted by a 10-membered saturated heteroaryl which one N as ring members. According to one embodiment thereof, the heteroaryl is unsubstituted, i.e. it does not carry any substituent R^{7b}. According to still another embodiment of formula I, it is substituted by R^{7b}.

According to still another embodiment of formula I, R⁷ is CH₂ substituted by a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R⁷ is CH₂ substituted by a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to a further particular embodiment, R⁷ is selected from C₁-C₆-alkyl, C₁-C₆-halogenalkyl, CN, C₂-C₆-alkenyl, C₂-C₆-halogenalkenyl, C₃-C₆-cycloalkenyl, C₂-C₆-alkynyl, C₂-C₆-halogenalkynyl, C₁-C₆-alkoxy, C₃-C₆-cycloalkynyl, C(═O)C₁-C₆-alkyl, C(═O)O(C₁-C₆-alkyl), CR′═NOR″, C₃-C₆-halogencycloalkyl a saturated three-, four-, five-, six-, membered carbocycle or heterocycle, a five- or six-membered heteroaryl or aryl; and C₁-C₆-alkyl substituted by CN, C₁-C₆-alkoxy, C₁-C₄-halogenalkoxy, C₁-C₆-alkylthio, S(O)ₙ—C₁-C₆-alkyl, NH—SO₂—R^{x}, NH(C₁-C₆-alkyl), N(C₁-C₆-alkyl)₂, C(═O)C₁-C₆-alkyl, C(═O)O(C₁-C₆-alkyl), a saturated three-, four-, five-, six-, membered carbocycle, heterocycle, aryl or heteroaryl; wherein the carbocycle, heterocycle, aryl and heteroaryl is unsubstituted or carries one, two, three or four substituents R^{7b} as defined below. According to one embodiment thereof, the carbocycle, heterocycle, heteroaryl and aryl are unsubstituted. In a particular embodiment, R⁷ is selected from CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, a saturated three-, four-, five-, six-, membered carbocycle or heterocycle, a five- or six-membered heteroaryl or aryl; and C₁-C₆-alkyl substituted by a saturated three-, four-, five-, six-, membered carbocycle, heterocycle, aryl or heteroaryl; wherein the carbocycle, heterocycle, aryl and heteroaryl are unsubstituted or carries one, two, three or four substituents R^{7b} as defined below.

According to a further particular embodiment, R⁷ is selected from C₁-C₆-halogenalkyl, CN, C₂-C₆-alkenyl, C₂-C₆-halogenalkenyl, C₃-C₆-cycloalkenyl, C₂-C₆-alkynyl, C₂-C₆-halogenalkynyl, C₁-C₆-alkoxy, C₃-C₆-cycloalkynyl, C(═O)C₁-C₆-alkyl, C(═O)O(C₁-C₆-alkyl), CR′═NOR″, C₃-C₆-halogencycloalkyl a saturated three-, four-, five-, six-, membered carbocycle or heterocycle, a five- or six-membered heteroaryl or aryl; and C₁-C₆-alkyl substituted by CN, C₁-C₆-alkoxy, C₁-C₄-halogenalkoxy, C₁-C₆-alkylthio, S(O)ₙ—C₁-C₆-alkyl, NH—SO₂—R^{x}, NH(C₁-C₆-alkyl), N(C₁-C₆-alkyl)₂, C(═O)C₁-C₆-alkyl, C(═O)O(C₁-C₆-alkyl), a saturated three-, four-, five-, six-, membered carbocycle, heterocycle, aryl or heteroaryl; wherein the carbocycle, heterocycle, aryl and heteroaryl is unsubstituted or carries one, two, three or four substituents R^{7b} as defined below. According to one embodiment thereof, the carbocycle, heterocycle, heteroaryl and aryl are unsubstituted. In a particular embodiment, R⁷ is selected from CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, a saturated three-, four-, five-, six-, membered carbocycle or heterocycle, a five- or six-membered heteroaryl or aryl; and C₁-C₆-alkyl substituted by a saturated three-, four-, five-, six-, membered carbocycle, heterocycle, aryl or heteroaryl; wherein the carbocycle, heterocycle, aryl and heteroaryl are unsubstituted or carries one, two, three or four substituents R^{7b} as defined below.

Particularly preferred embodiments of R⁷ according to the invention are in Table P6 below, wherein each line of lines P6-1 to P6-208 corresponds to one particular embodiment of the invention, wherein P6-1 to P6-208 are also in any combination with one another a preferred embodiment of the present invention. The connection point to the carbon atom, to which R⁷ is bound is marked with “#” in the drawings.

TABLE P7:
(py = pyridyl):

| No. | R⁷ |
|---|---|
| P7-1 | CH₃ |
| P7-2 | CH₂CH₃ |
| P7-3 | CF₃ |
| P7-4 | CH₂F |
| P7-5 | CH₂Cl |
| P7-6 | CHF₂ |
| P7-7 | CHCl₂ |
| P7-8 | CH₂CF₃ |
| P7-9 | CH₂CCl₃ |
| P7-10 | CF₂CHF₂ |
| P7-11 | CH₂OCH₃ |
| P7-12 | CH₂OCH₂F |
| P7-13 | CH₂OCHF₂ |
| P7-14 | CH₂OCF₃ |
| P7-15 | CH₂OCF₂CHF₂ |
| P7-16 | CH₂NHMe |
| P7-17 | CH₂SMe |
| P7-18 | CH₂SOMe |
| P7-19 | CH₂SO₂Me |
| P7-20 | CH₂NMe₂ |
| P7-21 | CH₂NSO₂CF₃ |
| P7-22 | CH₂NSO₂CH₃ |
| P7-23 | CN |
| P7-24 | CH₂CN |
| P7-25 | CHO |
| P7-26 | COMe |
| P7-27 | CO₂Me |
| P7-28 | CH₂CHO |
| P7-29 | CH₂COMe |
| P7-30 | CH₂CO₂Me |
| P7-31 | 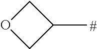 |
| P7-32 | 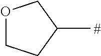 |
| P7-33 | 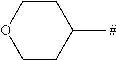 |
| P7-34 | 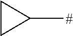 |
| P7-35 | 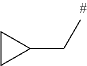 |
| P7-36 | 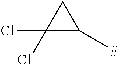 |
| P7-37 | 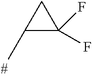 |
| P7-38 | 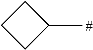 |
| P7-39 | 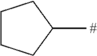 |
| P7-40 | 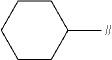 |
| P7-41 | 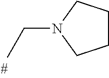 |
| P7-42 | 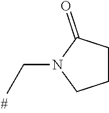 |
| P7-43 | 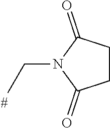 |
| P7-44 | 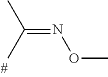 |
| P7-45 | 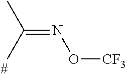 |
| P7-46 | 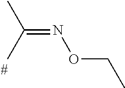 |
| P7-47 | 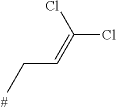 |
| P7-48 | 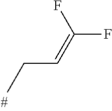 |
| P7-49 | 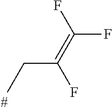 |
| P7-50 | 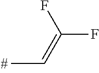 |
| P7-51 | 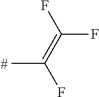 |
| P7-52 | 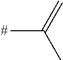 |
| P7-53 | 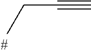 |
| P7-54 | 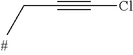 |
| P7-55 | 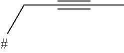 |
| P7-56 | 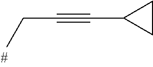 |
| P7-57 | 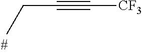 |
| P7-58 | 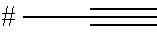 |
| P7-59 | 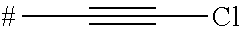 |
| P7-60 | 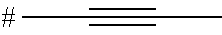 |
| P7-61 | 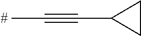 |
| P7-62 | 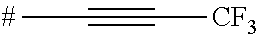 |
| P7-63 | C₆H₅ |
| P7-64 | 4-Cl—C₆H₄ |
| P7-65 | 3-Cl—C₆H₄ |
| P7-66 | 2-Cl—C₆H₄ |
| P7-67 | 2,4-Cl₂—C₆H₃ |
| P7-68 | 4-F—C₆H₄ |
| P7-69 | 3-F—C₆H₄ |
| P7-70 | 2-F—C₆H₄ |
| P7-71 | 2,4-F₂—C₆H₃ |
| P7-72 | 4-MeO—C₆H₄ |
| P7-73 | 3-MeO—C₆H₄ |
| P7-74 | 2-MeO—C₆H₄ |
| P7-75 | 4-MeO₂S—C₆H₄ |
| P7-76 | 3-MeO₂S—C₆H₄ |
| P7-77 | 2-MeO₂S—C₆H₄ |
| P7-78 | —CH₂—C₆H₅ |
| P7-79 | —CH₂—C₆H₄—4-F |
| P7-80 | —CH₂—C₆H₄—4-Cl |
| P7-81 | —CH₂—C₆H₃—2,4-Cl₂ |
| P7-82 | —CH₂—C₆H₄—4-SO₂Me |
| P7-83 | 3-py |
| P7-84 | 2-py |
| P7-85 | 4-py |
| P7-86 | 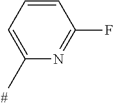 |
| P7-87 | 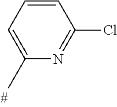 |
| P7-88 | 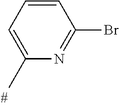 |
| P7-89 | 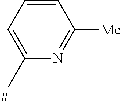 |
| P7-90 | 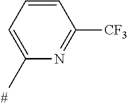 |
| P7-91 | 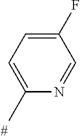 |
| P7-92 | 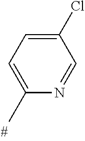 |
| P7-93 | 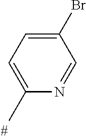 |
| P7-94 | 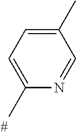 |
| P7-95 | 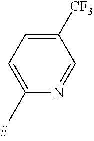 |
| P7-96 | 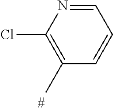 |
| P7-97 | 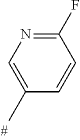 |
| P7-98 | 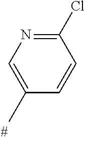 |
| P7-99 | 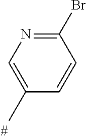 |
| P7-100 | 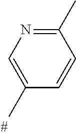 |
| P7-101 | 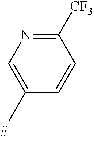 |
| P7-102 | 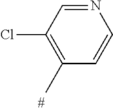 |
| P7-103 | 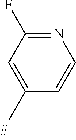 |
| P7-104 | 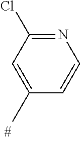 |
| P7-105 | 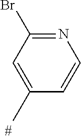 |
| P7-106 | 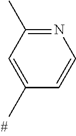 |
| P7-107 | 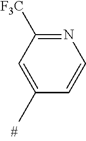 |
| P7-108 | 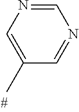 |
| P7-109 | 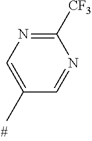 |
| P7-110 | 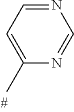 |
| P7-111 | 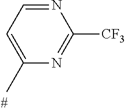 |
| P7-112 | 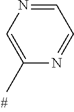 |
| P7-113 | 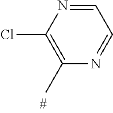 |
| P7-114 | 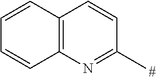 |
| P7-115 | 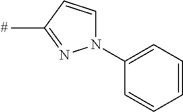 |
| P7-116 | 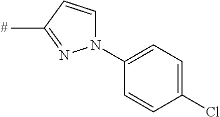 |
| P7-117 | 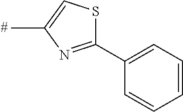 |
| P7-118 | 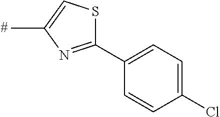 |
| P7-119 | 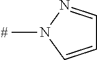 |
| P7-120 | 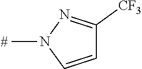 |
| P7-121 | 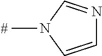 |
| P7-122 | 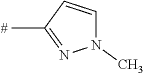 |
| P7-123 | 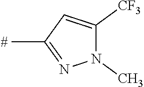 |
| P7-124 | 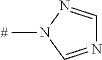 |
| P7-125 | 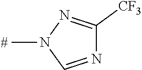 |
| P7-126 | 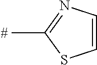 |
| P7-127 | 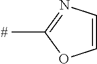 |
| P7-128 | 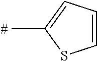 |
| P7-129 | 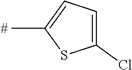 |
| P7-130 | 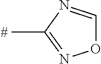 |
| P7-131 | 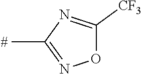 |
| P7-132 | 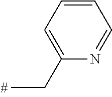 |
| P7-133 | 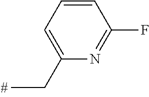 |
| P7-134 | 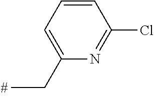 |
| P7-135 | 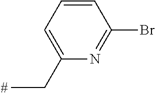 |
| P7-136 | 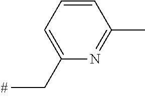 |
| P7-137 | 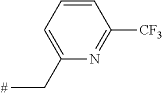 |
| P7-138 | 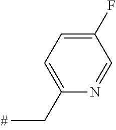 |
| P7-139 | 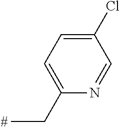 |
| P7-140 | 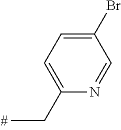 |
| P7-141 | 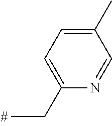 |
| P7-142 | 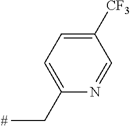 |
| P7-143 | 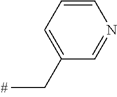 |
| P7-144 | 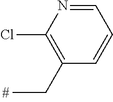 |
| P7-145 | 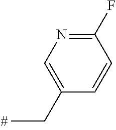 |
| P7-146 | 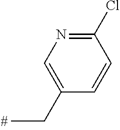 |
| P7-147 | 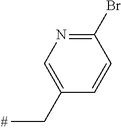 |
| P7-148 | 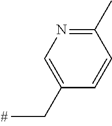 |
| P7-149 | 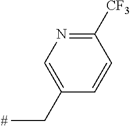 |
| P7-150 | 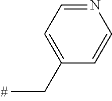 |
| P7-151 | 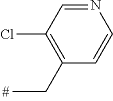 |
| P7-152 | 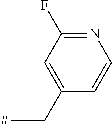 |
| P7-153 | 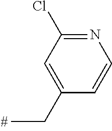 |
| P7-154 | 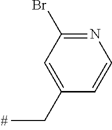 |
| P7-155 | 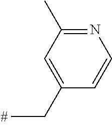 |
| P7-156 | 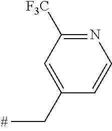 |
| P7-157 | 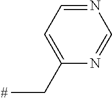 |
| P7-158 | 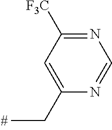 |
| P7-159 | 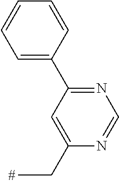 |
| P7-160 | 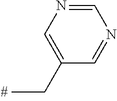 |
| P7-161 | 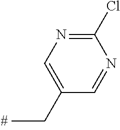 |
| P7-162 | 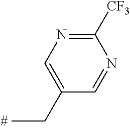 |
| P7-163 | 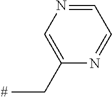 |
| P7-164 | 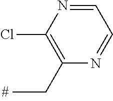 |
| P7-165 | 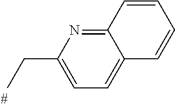 |
| P7-166 | 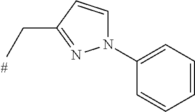 |
| P7-167 | 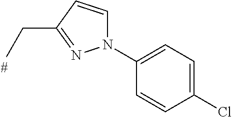 |
| P7-168 | 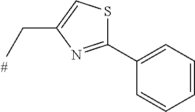 |
| P7-169 | 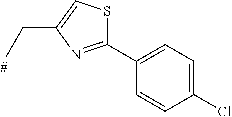 |
| P7-170 | 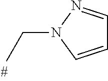 |
| P7-171 | 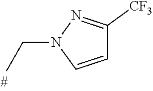 |
| P7-172 | 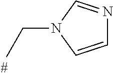 |
| P7-173 | 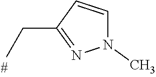 |
| P7-174 | 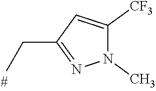 |
| P7-175 | 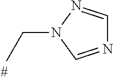 |
| P7-176 | 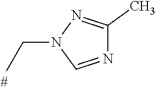 |
| P7-177 | 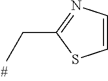 |
| P7-178 | 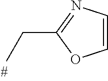 |
| P7-179 | 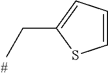 |
| P7-180 | 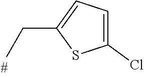 |
| P7-181 | 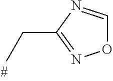 |
| P7-182 | 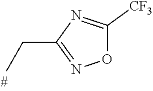 |
| P7-183 | CH₂CH₂CH₃ |
| P7-184 | CH(CH₃)₂ |
| P7-185 | CH₂CH₂CH₂CH₃ |
| P7-186 | CH(CH₃)CH₂CH₃ |
| P7-187 | CH₂CH(CH₃)CH₃ |
| P7-188 | C(CH₃)₃ |
| P7-189 | —O—C₆H₅ |
| P7-190 | —O—C₆H₄—2-F |
| P7-191 | —O—C₆H₄—3-F |
| P7-192 | —O—C₆H₄—4-F |
| P7-193 | —O—C₆H₄—2-Cl |
| P7-194 | —O—C₆H₄—3-Cl |
| P7-195 | —O—C₆H₄—4-Cl |
| P7-196 | —O—C₆H₄—2-OCH₃ |
| P7-197 | —O—C₆H₄—3-OCH₃ |
| P7-198 | —O—C₆H₄—4-OCH₃ |
| P7-199 | —NH—C₆H₅ |
| P7-200 | —NH—C₆H₄—2-F |
| P7-201 | —NH—C₆H₄—3-F |
| P7-202 | —NH—C₆H₄—4-F |
| P7-203 | —NH—C₆H₄—2-Cl |
| P7-204 | —NH—C₆H₄—3-Cl |
| P7-205 | —NH—C₆H₄—4-Cl |
| P7-206 | —NH—C₆H₄—2-OCH₃ |
| P7-207 | —NH—C₆H₄—3-OCH₃ |
| P7-208 | —NH—C₆H₄—4-OCH₃ |

R⁸ is independently selected from CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and heteroaryl contain independently one, two, three or four heteroatoms selected from N, O and S, and wherein heterocycle and heteroaryl are connected via C atom; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl;

wherein the heterocycle or heteroaryl contains one, two or three heteroatoms selected from N, O and S, and wherein R′ and R″ are independently unsubstituted or substituted with R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl;

wherein R^{x} is as defined above;

wherein the acyclic moieties of R⁸ are independently not further substituted or carry one, two, three or up to the maximum possible number of identical or different groups R^{8a}, which independently of one another are selected from:

R^{8a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₃-C₆-cycloalkyl, C₃-C₆-halogencycloalkyl, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbo- or heterocycle, a five-, six- or ten-membered heteroaryl, aryl or phenoxy, wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S, and wherein heterocycle and heteroaryl are connected via C atom; wherein the carbo-, heterocyclic, heteroaryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′ and R″ are as defined above; n is 0, 1, 2; and

wherein the carbo-, heterocyclic, heteroaryl and aryl moieties of R⁸ are independently unsubstituted or substituted with identical or different groups R^{8b}, which independently of one another are selected from:

R^{8b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or carry one, two, three, four or five substituents selected from the group consisting of halogen, OH, C₁-C₄-alkyl, C₁-C₄-halogenalkyl, C₁-C₄-alkoxy and C₁-C₄-halogenalkoxy; and wherein R^{x} and n are as defined above.

According to one embodiment of formula I, R⁸ is selected from substituted C₁-C₆-alkyl, C₁-C₆-halogenalkyl, C₂-C₆-halogenalkyl, C₂-C₆-alkenyl, C₂-C₆-halogenalkenyl, C₃-C₆-cycloalkenyl, C₂-C₆-alkynyl, C₂-C₆-halogenalkynyl, C₃-C₆-cycloalkynyl, CN, CH(═O), C(═O)C₂-C₆-alkyl, C(═O)O(C₂-C₆-alkyl), CR′═NOR″, C₃-C₆-cycloalkyl, C₃-C₆-halogencycloalkyl, C₁-C₆-alkyl-five- and six-membered heteroaryl, a five- or six-membered heteroaryl, benzyl, aryl; wherein R′ and R″ are defined below; and wherein the acyclic moieties of R⁸ are unsubstituted or substituted with identical or different groups R^{8a} as defined below and wherein wherein the carbocycle, heterocycle and heteroaryl and aryl moieties are unsubstituted or substituted with substituents R^{8b} as defined below.

According to still another embodiment of formula I, R⁸ is CN.

According to still another embodiment of formula I, R⁸ is selected from CN, substituted C₁-C₆-alkyl, C₁-C₆-halogenalkyl or C₁-C₆-alkyl which is substituted, C₁-C₆-halogenalkyl, phenyl, halogenphenyl and three-, four-, five- or six-membered carbo- and heterocycle, wherein the carbo- and heterocycle is unsubstituted or is substituted with substituents R^{8b} as defined below.

According to one embodiment thereof, the carbocycle is unsubstituted. In a particular embodiment, R⁸ is selected from C₁-C₆-halogenalkyl, phenyl-CH₂, halogenphenyl-CH₂, phenyl, halogenphenyl and three-, four-, five- or six-membered carbo- and heterocycle, wherein the carbo- and heterocycle is unsubstituted or is substituted with substituents R^{8b} as defined below.

According to still another embodiment of formula I, R⁸ is selected from CN, substituted C₁-C₆-alkyl, C₁-C₆-halogenalkyl or C₁-C₆-alkyl which is substituted, C₁-C₆-halogenalkyl, phenyl, halogenphenyl and three-, four-, five- or six-membered carbo- and heterocycle, wherein the carbo- and heterocycle is unsubstituted or substituted by substituents R^{8b} as defined below. According to one embodiment thereof, the carbo- and heterocycle is unsubstituted. In a particular embodiment, R⁸ is selected from substituted C₁-C₆-halogenalkyl, phenyl, halogenphenyl and three-, four-, five- or six-membered carbo- and heterocycle, wherein the carbo- and heterocycle is unsubstituted or substituted by substituents R^{8b} as defined below.

According to another embodiment of formula I, R⁸ is selected from CN, substituted C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, C₁-C₆-alkylaryl, five- or six-membered heteroaryl or aryl which is unsubstituted or substituted with halogen or C₁-C₆-halogenalkyl, and wherein the acyclic moieties of R⁸ are unsubstituted or substituted with identical or different groups R^{8a} as defined below and wherein wherein the carbocycle, heterocycle and heteroaryl and aryl moieties are unsubstituted or substituted with substituents R^{8b} as defined below.

According to still another embodiment of formula I, R⁸ is selected from CN, substituted C₁-C₆-alkyl, C₁-C₆-halogenalkyl, CN, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, C₁-C₆-alkylaryl, phenyl, pyridine, pyrimidine, thiophene, imidazole, triazol, oxadiazol wherein the acyclic moieties of R⁸ are unsubstituted or substituted with identical or different groups R^{8a} as defined below and wherein wherein the carbocycle, heterocycle and heteroaryl and aryl moieties are unsubstituted or substituted with substituents R^{8b} as defined below.

According to still another embodiment of formula I, R⁸ is C₁-C₆-alkyl such as CH₃, C₂H₅, n-propyl, i-propyl, n-butyl, i-butyl, tert-butyl, n-pentyl or i-pentyl.

According to still another embodiment of formula I, R⁸ is C₁-C₆-alkyl such as CH₃.

According to still another embodiment of formula I, R⁸ is C₁-C₆-alkyl such as C₂H₅.

According to still another embodiment of formula I, R⁸ is C₁-C₆-alkyl such as CH₃, C₂H₅, n-propyl, i-propyl, n-butyl, i-butyl, tert-butyl, n-pentyl or i-pentyl which is substituted with at least one group R^{8a}, which independently of one another are selected from:

R^{8a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″ a saturated or partially unsaturated three-, four-, five-, six-, seven-, eight-, nine-, or ten-membered carbocycle or heterocycle, five-, six- or ten-membered heteroaryl, aryl or phenoxy, wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbocyclic, heterocyclic, heteroaryl, aryl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl.

According to still another embodiment of formula I, R⁸ is CH₃ is substituted with at least one group R^{8a}, which independently of one another are selected from:

R^{8a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, five-, six- or ten-membered heteroaryl, an aryl or phenoxy, wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbocyclic, heterocyclic, heteroaryl, heteroaryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl.

According to still another embodiment of formula I, R⁸ is C₂H₅ is substituted with at least one group R^{8a}, which independently of one another are selected from:

R^{8a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″ a saturated or partially unsaturated three-, four-, five-, six-, seven-, eight-, nine-, or ten-membered carbocycle or heterocycle, five-, six- or ten-membered heteroaryl, phenyl or phenoxy; wherein in each case one or two CH₂ groups of the carbo- and heterocycle may be replaced by a group independently selected from C(═O) and C(═S), and wherein the heterocycle and heteroaryl contains independently one, two, three or four heteroatoms selected from N, O and S; wherein the carbocyclic, heterocyclic, heteroaryl, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl.

According to still another embodiment of formula I, R⁸ is CH₂CN.

According to still another embodiment of formula I, R⁸ is CH₂OH.

According to still another embodiment of formula I, R⁸ is C₁-C₆-halogenalkyl, in particular C₁-C₄-halogenalkyl, more specifically C₁-C₂-halogenalkyl, such as CF₃, CCl₃, FCH₂, ClCH₂, F₂CH, Cl₂CH, CF₃CH₂, CCl₃CH₂ or CF₂CHF₂.

According to still another embodiment of formula I, R⁸ is CH₂F.

According to still another embodiment of formula I, R⁸ is CHF₂.

According to still another embodiment of formula I, R⁸ is CF₃.

According to still a further embodiment of formula I, R⁸ is C₂-C₆-alkenyl, in particular C₂-C₄-alkenyl, such as CH═CH₂, CH₂CH═CH₂ or C(CH₃)C═CH₂.

According to a further specific embodiment of formula I, R⁸ is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CF═CF₂, CCl═CCl₂, CH₂CH═CHF, CH₂CH═CHCl, CH₂CH═CF₂, CH₂CH═CCl₂, CH₂CF═CF₂, CH₂CCl═CCl₂, CF₂CF═CF₂ or CCl₂CCl═CCl₂.

According to still a further embodiment of formula I, R⁸ is C₂-C₆-cycloalkenyl, in particular C₂-C₄-cycloalkenyl, such as CH═CH₂-cPr.

According to still a further embodiment of formula I, R⁸ is C₂-C₆-alkynyl or C₂-C₆-halogenalkynyl, in particular C₂-C₄-alkynyl or C₂-C₄-halogenalkynyl, such as C≡CH, C≡C—Cl, C≡C—CH₃, CH₂—C≡CH, CH₂—C≡CCl or CH₂—C≡C—CH₃.

According to still a further embodiment of formula I, R⁸ is C₂-C₆-cycloalkynyl in particular C₂-C₄-cycloalkynyl, such as C≡C-cPr.

According to a further specific embodiment of formula I, R⁸ is CH(═O), C(═O)C₁-C₆-alkyl, C(═O)O(C₁-C₆-alkyl), C(═O)NH(C₁-C₆-alkyl) or C(═O)N(C₁-C₆-alkyl)₂, wherein alkyl is CH₃, C₂H₅, n-propyl, i-propyl, n-butyl, i-butyl, tert-butyl, n-pentyl or i-pentyl.

According to a further specific embodiment of formula I, R⁸ is C₁-C₄-alkyl-CH(═O), C₁-C₄-alkyl-C(═O)C₁-C₆-alkyl, C₁-C₄-alkyl-C(═O)O(C₁-C₆-alkyl), C₁-C₄-alkyl-C(═O)NH(C₁-C₆-alkyl) or C₁-C₄-alkyl-C(═O)N(C₁-C₆-alkyl)₂, especially CH₂CH(═O), CH₂C(═O)C₁-C₆-alkyl, CH₂C(═O)O(C₁-C₆-alkyl), CH₂C(═O)NH(C₁-C₆-alkyl) or CH₂C(═O)N(C₁-C₆-alkyl)₂ wherein alkyl is CH₃, C₂H₅, n-propyl, i-propyl, n-butyl, i-butyl, tert-butyl, n-pentyl or i-pentyl.

According to a further specific embodiment of formula I, R⁸ is CR′═NOR″ such as C(CH₃)═NOCH₃, C(CH₃)═NOCH₂CH₃ or C(CH₃)═NOCF₃.

According to a further specific embodiment of formula I, R⁸ is C₁-C₆-alkyl-NH(C₁-C₄-alkyl) or C₁-C₆-alkyl-N(C₁-C₄-alkyl)₂, wherein alkyl is CH₃, C₂H₅, n-propyl, i-propyl, n-butyl, i-butyl, tert-butyl, n-pentyl or i-pentyl.

According to a further specific embodiment of formula I, R⁸ is C₁-C₆-alkylthio, in particular C₁-C₄-alkoxy, more specifically C₁-C₃-alkylthio such as CH₂SCH₃ or CH₂SCH₂CH₃.

According to a further specific embodiment of formula I, R⁸ is C₁-C₆-alkyl-S(O)ₙ—C₁-C₆-alkyl, wherein alkyl is CH₃, C₂H₅, n-propyl, i-propyl, n-butyl, i-butyl, tert-butyl, n-pentyl or i-pentyl and n is 1, 2 or 3.

According to a further specific embodiment of formula I, R⁸ is C₁-C₆-alkyl-S(O)ₙ—C₁-C₆-halogenalkyl, wherein halogenalkyl is CF₃ or CHF₂ and n is 1, 2 or 3.

According to a further specific embodiment of formula I, R⁸ is C₁-C₆-alkyl-S(O)ₙ-aryl, wherein the aryl or phenyl moiety in each case is unsubstituted or substituted with identical or different groups R^{8b} which independently of one another are selected from halogen, C₁-C₂-alkyl, C₁-C₂-alkoxy, C₁-C₂-halogenalkyl, C₁-C₂-halogenalkoxy and S(O)ₙ—C₁-C₆-alkyl, in particular F, Cl, Br, CH₃, OCH₃, CF₃, CHF₂, OCHF₂, OCF₃. According to one embodiment, R⁸ is unsubstituted phenyl. According to another embodiment, R⁸ is phenyl, that is substituted with one, two or three, in particular one, halogen, in particular selected from F, Cl and Br, more specifically selected from F and Cl.

According to a further specific embodiment of formula I, R⁸ is C₁-C₆-alkyl-NH—SO₂—R^{x} wherein R^{x} is C₁-C₄-alkyl, C₁-C₄-halogenalkyl, unsubstituted aryl or aryl that is substituted with one, two, three, four or five substituents R^{x2} independently selected from C₁-C₄-alkyl, halogen, OH, CN, C₁-C₄-halogenalkyl, C₁-C₄-alkoxy, or C₁-C₄-halogenalkoxy, such as CH₂NHSO₂CF₃ or CH₂NHSO₂CH₃.

According to still another embodiment of formula I, R⁸ is selected from C₁-C₆-alkyl which is substituted, a saturated three-, four-, five-, six-, seven-, eight-, nine-, or ten-membered carbocycle, in particular three-, four-, five- or six-membered, wherein the carbocycle is unsubstituted or substituted with substituents R^{8b} as defined below. According to one embodiment thereof, the carbocycle is unsubstituted.

According to one embodiment, R⁸ is selected from C₁-C₆-alkyl, especially CH₂ which is substituted with a 3-membered saturated carbocycle. According to one embodiment thereof, the carbocycle is unsubstituted, i.e. it does not carry any substituent R^{8b}. According to still another embodiment of formula I, it is substituted with R^{8b}.

According to one embodiment, R⁸ is selected from C₁-C₆-alkyl, especially CH₂ which is substituted with a 4-membered saturated carbocycle. According to one embodiment thereof, the carbocycle is unsubstituted, i.e. it does not carry any substituent R^{b}. According to still another embodiment of formula I, it is substituted with R^{8b}.

According to one embodiment, R⁸ is selected from C₁-C₆-alkyl, especially CH₂ which is substituted with a 5-membered saturated carbocycle. According to one embodiment thereof, the carbocycle is unsubstituted, i.e. it does not carry any substituent R^{8b}. According to still another embodiment of formula I, it is substituted with R^{8b}.

According to one embodiment, R⁸ is selected from C₁-C₆-alkyl, especially CH₂ which is substituted with a 6-membered saturated carbocycle. According to one embodiment thereof, the carbocycle is unsubstituted, i.e. it does not carry any substituent R^{b}. According to still another embodiment of formula I, it is substituted with R^{8b}.

According to a further specific embodiment of formula I, R⁸ is C₁-C₆-alkylheterocycle, especially CH₂ substituted with a 4-membered saturated heterocycle which contains 1 or 2 heteroatoms, in particular 1 heteroatom, from the group consisting of N, O and S, as ring members. According to one embodiment, the heterocycle contains one O as heteroatom. For example, the formed heterocycle is oxetane. According to one embodiment thereof, the heterocycle is unsubstituted, i.e. it does not carry any substituent R^{8b}. According to still another embodiment of formula I, it is substituted with R^{8b}.

According to a further specific embodiment of formula I, R⁸ is C₁-C₆-alkylheterocycle, especially CH₂ substituted with a 5-membered saturated heterocycle which contains 1, 2 or 3, in particular 1 or 2, heteroatoms from the group consisting of N, O and S, as ring members. According to one embodiment, the heterocycle contains one O as heteroatom. According to one embodiment thereof, the heterocycle is unsubstituted, i.e. it does not carry any substituent R^{8b}. According to still another embodiment of formula I, it is substituted with R^{8b}.

According to a further specific embodiment of formula I, R⁸ is C₁-C₆-alkylheterocycle, especially CH₂ substituted by a 6-membered saturated heterocycle which contains 1, 2 or 3, in particular 1 or 2, heteroatoms from the group consisting of N, O and S as ring members. According to one embodiment thereof, the heterocycle is unsubstituted, i.e. it does not carry any substituent R^{b}.

According to still another embodiment of formula I, it is substituted with R^{8b}. According to one specific embodiment thereof, said 6-membered saturated heterocycle contains 1 or 2, in particular 1, heteroatom(s) O. According to one embodiment thereof, the respective 6-membered heterocycle is unsubstituted, i.e. it does not carry any substituent R^{8b}. According to still another embodiment of formula I, it is substituted with R^{8b}.

According to a further specific embodiment of formula I, R⁸ is C₁-C₆-alkylheterocycle, especially CH₂ substituted with a 5-membered saturated heterocycle which contains one N as ring member and optionally one or two groups CH₂ are replaced by C(═O).

According to still another embodiment of formula I, R⁸ is a partially unsaturated three-, four-, five-, six-, seven-, eight-, nine-, or ten-membered carbocycle, in particular three-, four-, five- or six-membered, wherein the carbocycle is unsubstituted or substituted with substituents R^{8b} as defined below. According to one embodiment thereof, the carbocycle is unsubstituted.

According to still another embodiment of formula I, R⁸ is a partially unsaturated three-, four-, five-, six-, seven-, eight-, nine-, or ten-membered carbocycle or heterocycle, in particular three-, four-, five- or six-membered, wherein the heterocycle contains one, two, three or four heteroatoms selected from N, O and S, and wherein the carbocycle and heterocycle are unsubstituted or substituted with substituents R^{8b} as defined below. According to one embodiment thereof, the carbocycle or heterocycle is unsubstituted.

According to still a further embodiment, R⁸ is a saturated three-, four-, five-, six-, seven-, eight-, nine-, or ten-membered carbocycle or heterocycle, in particular three-, four-, five- or six-membered, wherein the heterocycle contains one, two, three or four heteroatoms selected from N, O and S, and wherein the carbocycle and heterocycle are unsubstituted or substituted with substituents R^{8b} as defined below. According to one embodiment thereof, the carbocycle or heterocycle is unsubstituted.

According to still another embodiment of formula I, R⁸ is a saturated three-, four-, five-, six-, seven-, eight-, nine-, or ten-membered carbocycle, in particular three-, four-, five- or six-membered, wherein the carbocycle is unsubstituted or substituted with substituents R^{8b} as defined below. According to one embodiment thereof, the carbocycle is unsubstituted.

According to one embodiment, R⁸ is a 3-membered saturated carbocycle. According to one embodiment thereof, the carbocycle is unsubstituted, i.e. it does not carry any substituent R^{8b}. According to still another embodiment of formula I, it is substituted with R^{8b}.

According to one embodiment, R⁸ is a 3-membered saturated carbocycle, which is unsubstituted such as cyclopropyl.

According to one embodiment, R⁸ is a 3-membered saturated carbocycle, which is substituted with halogen, more specifically by F, such as C₃H₃F₂.

According to one embodiment, R⁸ is a 3-membered saturated carbocycle, which is substituted with halogen. More specifically by Cl, such as C₃H₃Cl₂.

According to one embodiment, R⁸ is a 4-membered saturated carbocycle. According to one embodiment thereof, the carbocycle is unsubstituted, i.e. it does not carry any substituent R^{8b}.

According to still another embodiment of formula I, it is substituted with R^{8b}.

According to one embodiment, R⁸ is a 5-membered saturated carbocycle. According to one embodiment thereof, the carbocycle is unsubstituted, i.e. it does not carry any substituent R^{b}. According to still another embodiment of formula I, it is substituted with R^{8b}.

According to one embodiment, R⁸ is a 6-membered saturated carbocycle. According to one embodiment thereof, the carbocycle is unsubstituted, i.e. it does not carry any substituent R^{8b}. According to still another embodiment of formula I, it is substituted with R^{8b}.

According to still another embodiment of formula I, R⁸ is a partially unsaturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted with substituents R^{8b} as defined below. According to one embodiment thereof, the heterocycle is unsubstituted.

According to still another embodiment of formula I, R⁸ is a saturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted with substituents R^{8b} as defined below. According to one embodiment thereof, the heterocycle is unsubstituted.

According to still another embodiment of formula I, in the embodiments of R⁸ described above, the heterocycle contains preferably one, two or three, more specifically one or two heteroatoms selected from N, O and S. More specifically, the heterocycle contains one heteroatom selected from N, O and S. In particular, the heterocycle contains one or two, in particular one O.

According to one embodiment, R⁸ is a 4-membered saturated heterocycle which contains 1 or 2 heteroatoms, in particular 1 heteroatom, from the group consisting of N, O and S, as ring members. According to one embodiment, the heterocycle contains one O as heteroatom. For example, the formed heterocycle is oxetane. According to one embodiment thereof, the heterocycle is unsubstituted, i.e. it does not carry any substituent R^{b}. According to still another embodiment of formula I, it is substituted with R^{8b}.

According to still another embodiment of formula I, R⁸ is a 5-membered saturated heterocycle which contains 1, 2 or 3, in particular 1 or 2, heteroatoms from the group consisting of N, O and S, as ring members. According to one embodiment, the heterocycle contains one O as heteroatom. According to one embodiment thereof, the heterocycle is unsubstituted, i.e. it does not carry any substituent R^{8b}. According to still another embodiment of formula I, it is substituted with R^{8b}.

According to still another embodiment of formula I, R⁸ is a 6-membered saturated heterocycle which contains 1, 2 or 3, in particular 1 or 2, heteroatoms from the group consisting of N, O and S as ring members. According to one embodiment thereof, the heterocycle is unsubstituted, i.e. it does not carry any substituent R^{8b}. According to still another embodiment of formula I, it is substituted with R^{8b}. According to one specific embodiment thereof, said 6-membered saturated heterocycle contains 1 or 2, in particular 1, heteroatom(s) O. According to one embodiment thereof, the respective 6-membered heterocycle is unsubstituted, i.e. it does not carry any substituent R^{8b}. According to still another embodiment of formula I, it is substituted with R^{8b}.

According to still another embodiment of formula I, R⁸ is phenyl-C₁-C₆-alkyl, such as phenyl-CH₂, wherein the phenyl moiety in each case is unsubstituted or substituted with one, two or three identical or different groups R^{8b} which independently of one another are selected from CN, halogen, C₁-C₂-alkyl, C₁-C₂-alkoxy, C₁-C₂-halogenalkyl, C₁-C₂-halogenalkoxy and S(O)ₙ—C₁-C₆-alkyl, in particular from CN, F, Cl, Br, CH₃, OCH₃, CF₃, CHF₂, OCHF₂, OCF₃ and S(O)₂CH₃.

According to still another embodiment of formula I, R⁸ is aryl, in particular phenyl, wherein the aryl or phenyl moiety in each case is unsubstituted or substituted with identical or different groups R^{8b} which independently of one another are selected from CN, halogen, C₁-C₂-alkyl, C₁-C₂-alkoxy, C₁-C₂-halogenalkyl, C₁-C₂-halogenalkoxy and S(O)ₙ—C₁-C₆-alkyl, in particular from CN, F, Cl, Br, CH₃, OCH₃, CF₃, CHF₂, OCHF₂, OCF₃. According to one embodiment, R⁸ is unsubstituted phenyl. According to another embodiment, R⁸ is phenyl, that is substituted with one, two or three, in particular one, halogen, in particular selected from F, Cl and Br, more specifically selected from F and Cl.

According to still another embodiment of formula I, R⁸ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R⁸ is a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to a further specific embodiment of formula I, R⁸ is C₁-C₆-alkyl, especially CH₂ substituted by a 5-membered saturated heteroaryl which contains 1, 2 or 3, in particular 1 or 2, heteroatoms from the group consisting of N, O and S as ring members. According to one embodiment thereof, the heteroaryl is unsubstituted, i.e. it does not carry any substituent R^{b}. According to still another embodiment of formula I, it is substituted by R^{8b}.

According to a further specific embodiment of formula I, R⁸ is C₁-C₆-alkyl, especially CH₂ substituted by a 5-membered saturated heteroaryl which contains one N as ring member. According to one embodiment thereof, the heteroaryl is unsubstituted, i.e. it does not carry any substituent R^{8b}. According to still another embodiment of formula I, it is substituted by R^{8b}.

According to a further specific embodiment of formula I, R⁸ is C₁-C₆-alkyl, especially CH₂ substituted by a 5-membered saturated heteroaryl which contains two N as ring members. According to one embodiment thereof, the heteroaryl is unsubstituted, i.e. it does not carry any substituent R^{8b}. According to still another embodiment of formula I, it is substituted by R^{8b}.

According to a further specific embodiment of formula I, R⁸ is C₁-C₆-alkyl, especially CH₂ substituted by a 5-membered saturated heteroaryl which contains three N as ring members. According to one embodiment thereof, the heteroaryl is unsubstituted, i.e. it does not carry any substituent R^{8b}. According to still another embodiment of formula I, it is substituted by R^{8b}. According to one specific embodiment thereof, said 5-membered saturated heterocycle contains 1 or 2, in particular 1, heteroatom(s) O.

According to a further specific embodiment of formula I, R⁸ is C₁-C₆-alkyl, especially CH₂ substituted by a 5-membered saturated heteroaryl which contains one S as ring member. According to one embodiment thereof, the heteroaryl is unsubstituted, i.e. it does not carry any substituent R^{8b}. According to still another embodiment of formula I, it is substituted by R^{8b}.

According to a further specific embodiment of formula I, R⁸ is C₁-C₆-alkyl, especially CH₂ substituted by a 5-membered saturated heteroaryl which contains one S and one N as ring members. According to one embodiment thereof, the heteroaryl is unsubstituted, i.e. it does not carry any substituent R^{8b}. According to still another embodiment of formula I, it is substituted by R^{8b}.

According to a further specific embodiment of formula I, R⁸ is C₁-C₆-alkyl, especially CH₂ substituted by a 5-membered saturated heteroaryl which contains one S and two N as ring members. According to one embodiment thereof, the heteroaryl is unsubstituted, i.e. it does not carry any substituent R^{8b}. According to still another embodiment of formula I, it is substituted by R^{8b}.

According to a further specific embodiment of formula I, R⁸ is C₁-C₆-alkyl, especially CH₂ substituted by a 5-membered saturated heteroaryl which contains one oxygen and one N as ring members. According to one embodiment thereof, the heteroaryl is unsubstituted, i.e. it does not carry any substituent R^{8b}. According to still another embodiment of formula I, it is substituted by R^{8b}.

According to a further specific embodiment of formula I, R⁸ is C₁-C₆-alkyl, especially CH₂ substituted by a 5-membered saturated heteroaryl which contains one oxygen and two N as ring members. According to one embodiment thereof, the heteroaryl is unsubstituted, i.e. it does not carry any substituent R^{8b}. According to still another embodiment of formula I, it is substituted by R^{8b}.

According to a further specific embodiment of formula I, R⁸ is C₁-C₆-alkyl, especially CH₂ substituted by a 6-membered saturated heteroaryl which contains 1, 2 or 3, in particular 1 or 2, heteroatoms from the group consisting of N, O and S as ring members. According to one embodiment thereof, the heteroaryl is unsubstituted, i.e. it does not carry any substituent R^{b}. According to still another embodiment of formula I, it is substituted by R^{8b}.

According to a further specific embodiment of formula I, R⁸ is C₁-C₆-alkyl, especially CH₂ substituted by a 6-membered saturated heteroaryl which one N as ring member. According to one embodiment thereof, the heteroaryl is unsubstituted, i.e. it does not carry any substituent R^{8b}. According to still another embodiment of formula I, it is substituted by R^{8b}.

According to a further specific embodiment of formula I, R⁸ is C₁-C₆-alkyl, especially CH₂ substituted by a 6-membered saturated heteroaryl which two N as ring members. According to one embodiment thereof, the heteroaryl is unsubstituted, i.e. it does not carry any substituent R^{b}. According to still another embodiment of formula I, it is substituted by R^{8b}.

According to a further specific embodiment of formula I, R⁸ is C₁-C₆-alkyl, especially CH₂ substituted by a 10-membered saturated heteroaryl which contains 1, 2 or 3, in particular 1 or 2, heteroatoms from the group consisting of N, O and S as ring members. According to one embodiment thereof, the heteroaryl is unsubstituted, i.e. it does not carry any substituent R^{8b}. According to still another embodiment of formula I, it is substituted by R^{8b}. According to one specific embodiment thereof, said 10-membered saturated heterocycle contains 1 or 2, in particular 1, heteroatom(s) N.

According to a further specific embodiment of formula I, R⁸ is C₁-C₆-alkyl, especially CH₂ substituted by a 10-membered saturated heteroaryl which one N as ring members. According to one embodiment thereof, the heteroaryl is unsubstituted, i.e. it does not carry any substituent R^{8b}. According to still another embodiment of formula I, it is substituted by R^{8b}.

According to still another embodiment of formula I, R⁸ is CH₂ substituted by a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R⁸ is CH₂ substituted by a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

According to a further particular embodiment, R⁸ is selected from C₁-C₆-alkyl, C₁-C₆-halogenalkyl, CN, C₂-C₆-alkenyl, C₂-C₆-halogenalkenyl, C₃-C₆-cycloalkenyl, C₂-C₆-alkynyl, C₂-C₆-halogenalkynyl, C₁-C₆-alkoxy, C₃-C₆-cycloalkynyl, C(═O)C₁-C₆-alkyl, C(═O)O(C₁-C₆-alkyl), CR′═NOR″, C₃-C₆-halogencycloalkyl a saturated three-, four-, five-, six-, membered carbocycle or heterocycle, a five- or six-membered heteroaryl or aryl; and C₁-C₆-alkyl substituted by CN, C₁-C₆-alkoxy, C₁-C₄-halogenalkoxy, C₁-C₆-alkylthio, S(O)ₙ—C₁-C₆-alkyl, NH—SO₂—R^{x}, NH(C₁-C₆-alkyl), N(C₁-C₆-alkyl)₂, C(═O)C₁-C₆-alkyl, C(═O)O(C₁-C₆-alkyl), a saturated three-, four-, five-, six-, membered carbocycle, heterocycle, aryl or heteroaryl; wherein the carbocycle, heterocycle, aryl and heteroaryl is unsubstituted or carries one, two, three or four substituents R^{8b} as defined below. According to one embodiment thereof, the carbocycle, heterocycle, heteroaryl and aryl are unsubstituted. In a particular embodiment, R⁸ is selected from CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, a saturated three-, four-, five-, six-, membered carbocycle or heterocycle, a five- or six-membered heteroaryl or aryl; and C₁-C₆-alkyl substituted by a saturated three-, four-, five-, six-, membered carbocycle, heterocycle, aryl or heteroaryl; wherein the carbocycle, heterocycle, aryl and heteroaryl are unsubstituted or carries one, two, three or four substituents R^{8b} as defined below.

According to a further particular embodiment, R⁸ is selected from C₁-C₆-halogenalkyl, CN, C₂-C₆-alkenyl, C₂-C₆-halogenalkenyl, C₃-C₆-cycloalkenyl, C₂-C₆-alkynyl, C₂-C₆-halogenalkynyl, C₁-C₆-alkoxy, C₃-C₆-cycloalkynyl, C(═O)C₁-C₆-alkyl, C(═O)O(C₁-C₆-alkyl), CR′═NOR″, C₃-C₆-halogencycloalkyl a saturated three-, four-, five-, six-, membered carbocycle or heterocycle, a five- or six-membered heteroaryl or aryl; and C₁-C₆-alkyl substituted by CN, C₁-C₆-alkoxy, C₁-C₄-halogenalkoxy, C₁-C₆-alkylthio, S(O)ₙ—C₁-C₆-alkyl, NH—SO₂—R^{x}, NH(C₁-C₆-alkyl), N(C₁-C₆-alkyl)₂, C(═O)C₁-C₆-alkyl, C(═O)O(C₁-C₆-alkyl), a saturated three-, four-, five-, six-, membered carbocycle, heterocycle, aryl or heteroaryl; wherein the carbocycle, heterocycle, aryl and heteroaryl is unsubstituted or carries one, two, three or four substituents R^{8b} as defined below. According to one embodiment thereof, the carbocycle, heterocycle, heteroaryl and aryl are unsubstituted. In a particular embodiment, R⁸ is selected from CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, a saturated three-, four-, five-, six-, membered carbocycle or heterocycle, a five- or six-membered heteroaryl or aryl; and C₁-C₆-alkyl substituted by a saturated three-, four-, five-, six-, membered carbocycle, heterocycle, aryl or heteroaryl; wherein the carbocycle, heterocycle, aryl and heteroaryl are unsubstituted or carries one, two, three or four substituents R^{8b} as defined below.

Particularly preferred embodiments of R⁸ according to the invention are in Table P8 below, wherein each line of lines P8-1 to P8-188 corresponds to one particular embodiment of the invention, wherein P8-1 to P8-188 are also in any combination with one another a preferred embodiment of the present invention. The connection point to the carbon atom, to which R⁸ is bound is marked with “#” in the drawings.

TABLE P8
(py = pyridyl):

| No. | R⁸ |
|---|---|
| P | CH₃ |
| P | CH₂CH₃ |
| P | CF₃ |
| P8-4 | CH₂F |
| P8-5 | CH₂Cl |
| P8-6 | CHF₂ |
| P8-7 | CHCl₂ |
| P8-8 | CH₂CF₃ |
| P8-9 | CH₂CCl₃ |
| P8-10 | CF₂CHF₂ |
| P8-11 | CH₂OCH₃ |
| P8-12 | CH₂OCH₂F |
| P8-13 | CH₂OCHF₂ |
| P8-14 | CH₂OCF₂ |
| P8-15 | CH₂OCF₂CHF₂ |
| P8-16 | CH₂NHMe |
| P8-17 | CH₂SMe |
| P8-18 | CH₂SOMe |
| P8-19 | CH₂SO₂Me |
| P8-20 | CH₂NMe₂ |
| P8-21 | CH₂NSO₂CF₃ |
| P8-22 | CH₂NSO₂CH₃ |
| P8-23 | CN |
| P8-24 | CH₂CN |
| P8-25 | CHO |
| P8-26 | COMe |
| P8-27 | CO₂Me |
| P8-28 | CH₂CHO |
| P8-29 | CH₂COMe |
| P8-30 | CH₂CO₂Me |
| P8-31 | 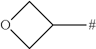 |
| P8-32 | 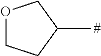 |
| P8-33 | 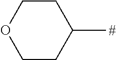 |
| P8-34 | 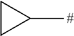 |
| P8-35 | 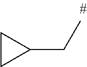 |
| P8-36 | 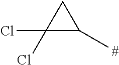 |
| P8-37 | 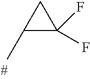 |
| P8-38 | 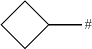 |
| P8-39 | 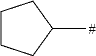 |
| P8-40 | 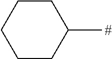 |
| P8-41 | 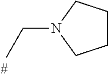 |
| P8-42 | 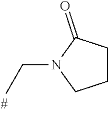 |
| P8-43 | 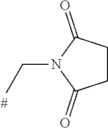 |
| P8-44 | 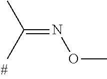 |
| P8-45 | 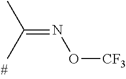 |
| P8-46 | 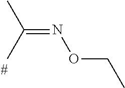 |
| P8-47 | 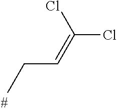 |
| P8-48 | 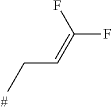 |
| P8-49 | 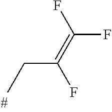 |
| P8-50 | 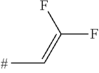 |
| P8-51 | 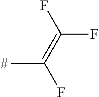 |
| P8-52 | 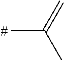 |
| P8-53 | 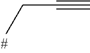 |
| P8-54 | 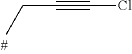 |
| P8-55 | 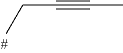 |
| P8-56 | 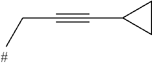 |
| P8-57 | 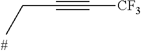 |
| P8-58 | 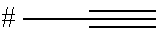 |
| P8-59 | 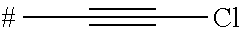 |
| P8-60 | 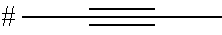 |
| P8-61 | 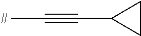 |
| P8-62 | 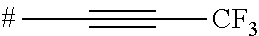 |
| P8-63 | C₆H₅ |
| P8-64 | 4-Cl—C₆H₄ |
| P8-65 | 3-Cl—C₆H₄ |
| P8-66 | 2-Cl—C₆H₄ |
| P8-67 | 2,4-Cl₂ C₆H₃ |
| P8-68 | 4-F—C₆H₄ |
| P8-69 | 3-F—C₆H₄ |
| P8-70 | 2-F—C₆H₄ |
| P8-71 | 2,4-F₂—C₆H₃ |
| P8-72 | 4-MeO—C₆H₄ |
| P8-73 | 3-MeO—C₆H₄ |
| P8-74 | 2-MeO—C₆H₄ |
| P8-75 | 4-MeO₂S—C₆H₄ |
| P8-76 | 3-MeO₂S—C₆H₄ |
| P8-77 | 2-MeO₂S—C₆H₄ |
| P8-78 | —CH₂—C₆H₅ |
| P8-79 | —CH₂—C₆H₄—4-F |
| P8-80 | —CH₂—C₆H₄—4-Cl |
| P8-81 | —CH₂—C₆H₃—2,4-Cl₂ |
| P8-82 | —CH₂—C₆H₄—4-SO₂Me |
| P8-83 | 3-py |
| P8-84 | 2-py |
| P8-85 | 4-py |
| P8-86 | 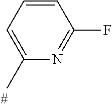 |
| P8-87 | 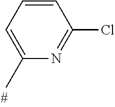 |
| P8-88 | 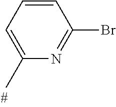 |
| P8-89 | 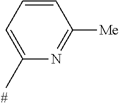 |
| P8-90 | 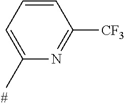 |
| P8-91 | 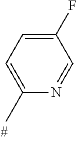 |
| P8-92 | 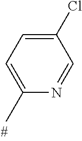 |
| P8-93 | 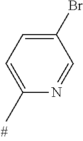 |
| P8-94 | 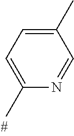 |
| P8-95 | 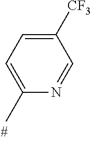 |
| P8-96 | 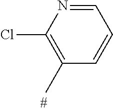 |
| P8-97 | 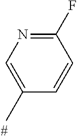 |
| P8-98 | 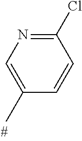 |
| P8-99 | 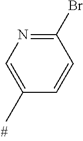 |
| P8-100 | 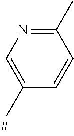 |
| P8-101 | 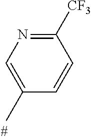 |
| P8-102 | 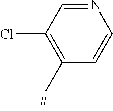 |
| P8-103 | 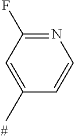 |
| P8-104 | 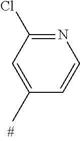 |
| P8-105 | 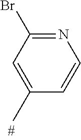 |
| P8-106 | 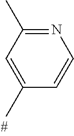 |
| P8-107 | 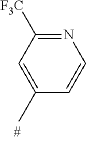 |
| P8-108 | 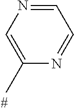 |
| P8-109 | 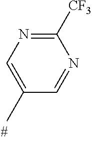 |
| P8-110 | 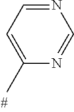 |
| P8-111 | 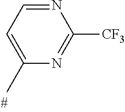 |
| P8-112 | 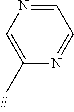 |
| P8-113 | 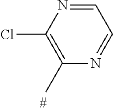 |
| P8-114 | 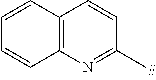 |
| P8-115 | 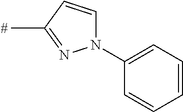 |
| P8-116 | 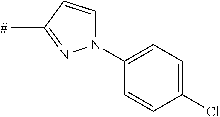 |
| P8-117 | 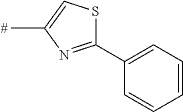 |
| P8-118 | 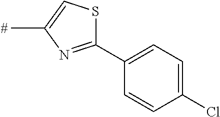 |
| P8-119 | 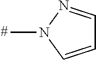 |
| P8-120 | 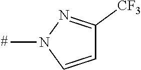 |
| P8-121 | 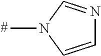 |
| P8-122 | 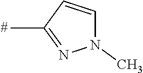 |
| P8-123 | 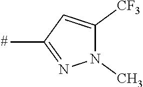 |
| P8-124 | 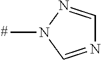 |
| P8-125 | 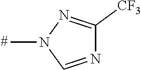 |
| P8-126 | 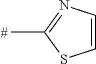 |
| P8-127 | 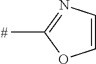 |
| P8-128 | 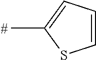 |
| P8-129 | 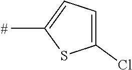 |
| P8-130 | 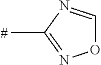 |
| P8-131 | 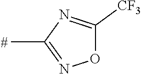 |
| P8-132 | 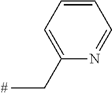 |
| P8-133 | 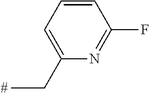 |
| P8-134 | 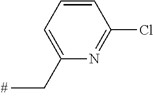 |
| P8-135 | 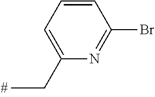 |
| P8-136 | 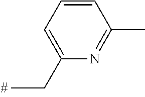 |
| P8-137 | 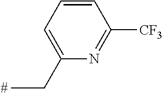 |
| P8-138 | 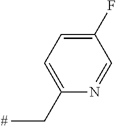 |
| P8-139 | 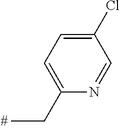 |
| P8-140 | 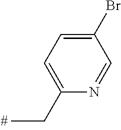 |
| P8-141 | 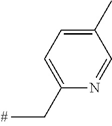 |
| P8-142 | 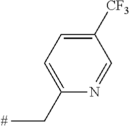 |
| P8-143 | 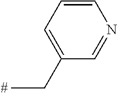 |
| P8-144 | 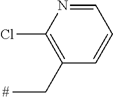 |
| P8-145 | 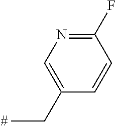 |
| P8-146 | 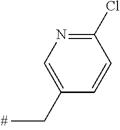 |
| P8-147 | 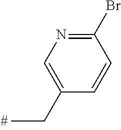 |
| P8-148 | 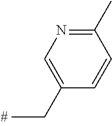 |
| P8-149 | 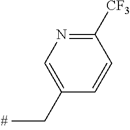 |
| P8-150 | 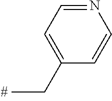 |
| P8-151 | 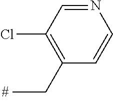 |
| P8-152 | 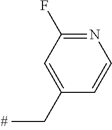 |
| P8-153 | 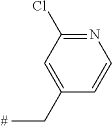 |
| P8-154 | 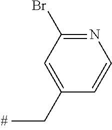 |
| P8-155 | 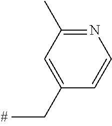 |
| P8-156 | 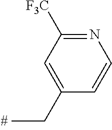 |
| P8-157 | 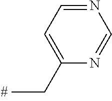 |
| P8-158 | 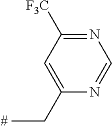 |
| P8-159 | 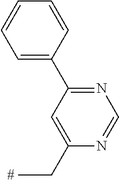 |
| P8-160 | 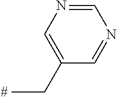 |
| P8-161 | 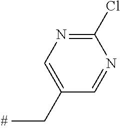 |
| P8-162 | 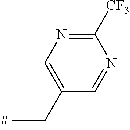 |
| P8-163 | 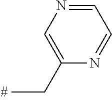 |
| P8-164 | 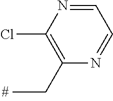 |
| P8-165 | 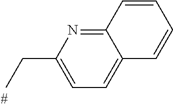 |
| P8-166 | 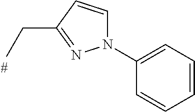 |
| P8-167 | 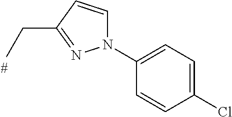 |
| P8-168 | 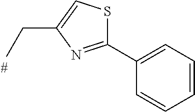 |
| P8-169 | 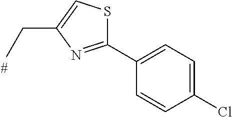 |
| P8-170 | 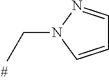 |
| P8-171 | 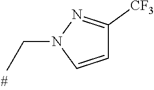 |
| P8-172 | 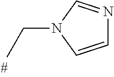 |
| P8-173 | 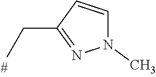 |
| P8-174 | 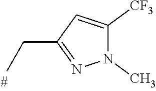 |
| P8-175 | 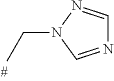 |
| P8-176 | 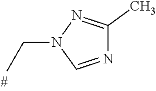 |
| P8-177 | 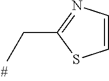 |
| P8-178 | 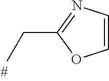 |
| P8-179 | 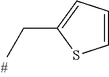 |
| P8-180 | 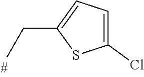 |
| P8-181 | 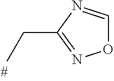 |
| P8-182 | 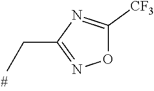 |
| P8-183 | CH₂CH₂CH₃ |
| P8-184 | CH(CH₃)₂ |
| P8-185 | CH₂CH₂CH₂CH₃ |
| P8-186 | CH(CH₃)CH₂CH₃ |
| P8-187 | CH₂CH(CH₃)CH₃ |
| P8-188 | C(CH₃)₃ |

Preferred embodiments of the present invention are the following compounds I.A-1, I.A-2, I.A-3, I.A-4, I.B-1, I.B-2, I.B-3, I.B-4 and I.C-1, I.C-2, I.C-3, I.C-4. In these formulae, the substituents R², R³, R⁶, R⁷ and R⁸ are independently as defined above or preferably defined herein:

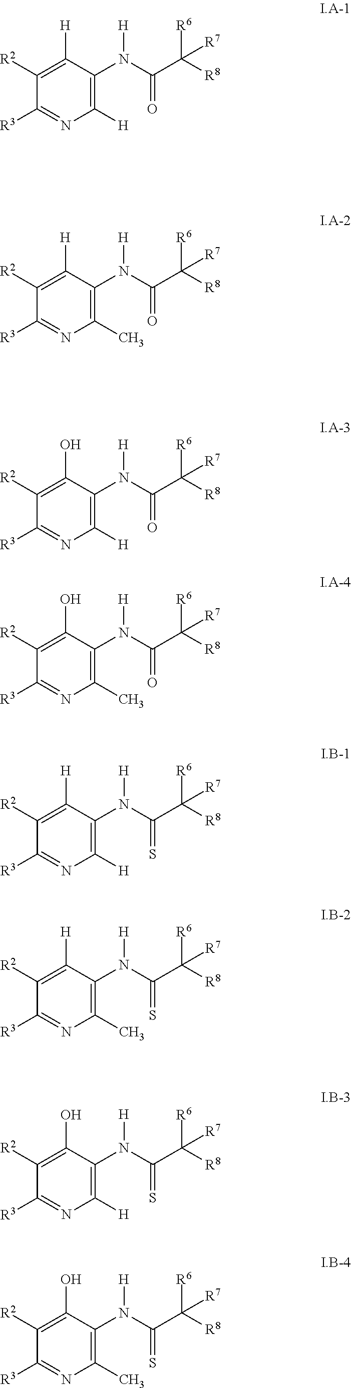

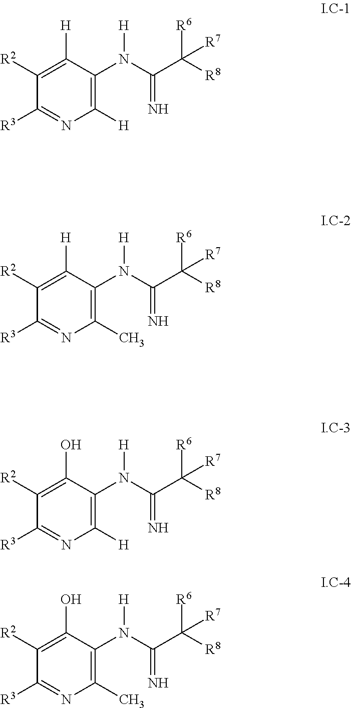

In particular with a view to their use, according to one embodiment, preference is given to the compounds of the formulae I.A-1, I.A-2, I.A-3, I.A-4, I.B-1, I.B-2, I.B-3, I.B-4 and I.C-1, I.C-2, I.C-3, I.C-4 that are compiled in the Tables 1a to 3a. Each of the groups mentioned for a substituent in the tables is furthermore per se, independently of the combination in which it is mentioned, a particularly preferred aspect of the substituent in question.

TABLE B

| No. | R² | R³ | R⁷ | R⁸ |
|---|---|---|---|---|
| B-1 | CH₃ | CH₃ | C₆H₅ | CH₃ |
| B-2 | OCH₃ | CH₃ | C₆H₅ | CH₃ |
| B-3 | CH₂F | CH₃ | C₆H₅ | CH₃ |
| B-4 | CHF₂ | CH₃ | C₆H₅ | CH₃ |
| B-5 | CH₃ | OCH₃ | C₆H₅ | CH₃ |
| B-6 | OCH₃ | OCH₃ | C₆H₅ | CH₃ |
| B-7 | CH₂F | OCH₃ | C₆H₅ | CH₃ |
| B-8 | CHF₂ | OCH₃ | C₆H₅ | CH₃ |
| B-9 | CH₃ | CH₂F | C₆H₅ | CH₃ |
| B-10 | OCH₃ | CH₂F | C₆H₅ | CH₃ |
| B-11 | CH₂F | CH₂F | C₆H₅ | CH₃ |
| B-12 | CHF₂ | CH₂F | C₆H₅ | CH₃ |
| B-13 | CH₃ | CHF₂ | C₆H₅ | CH₃ |
| B-14 | OCH₃ | CHF₂ | C₆H₅ | CH₃ |
| B-15 | CH₂F | CHF₂ | C₆H₅ | CH₃ |
| B-16 | CHF₂ | CHF₂ | C₆H₅ | CH₃ |
| B-17 | CH₃ | CH₃ | —CH₂—C₆H₅ | CH₃ |
| B-18 | OCH₃ | CH₃ | —CH₂—C₆H₅ | CH₃ |
| B-19 | CH₂F | CH₃ | —CH₂—C₆H₅ | CH₃ |
| B-20 | CHF₂ | CH₃ | —CH₂—C₆H₅ | CH₃ |
| B-21 | CH₃ | OCH₃ | —CH₂—C₆H₅ | CH₃ |
| B-22 | OCH₃ | OCH₃ | —CH₂—C₆H₅ | CH₃ |
| B-23 | CH₂F | OCH₃ | —CH₂—C₆H₅ | CH₃ |
| B-24 | CHF₂ | OCH₃ | —CH₂—C₆H₅ | CH₃ |
| B-25 | CH₃ | CH₂F | —CH₂—C₆H₅ | CH₃ |
| B-26 | OCH₃ | CH₂F | —CH₂—C₆H₅ | CH₃ |
| B-27 | CH₂F | CH₂F | —CH₂—C₆H₅ | CH₃ |
| B-28 | CHF₂ | CH₂F | —CH₂—C₆H₅ | CH₃ |
| B-29 | CH₃ | CHF₂ | —CH₂—C₆H₅ | CH₃ |
| B-30 | OCH₃ | CHF₂ | —CH₂—C₆H₅ | CH₃ |
| B-31 | CH₂F | CHF₂ | —CH₂—C₆H₅ | CH₃ |
| B-32 | CHF₂ | CHF₂ | —CH₂—C₆H₅ | CH₃ |
| B-33 | CH₃ | CH₃ | C₆H₅ | CH(CH₃)₂ |
| B-34 | OCH₃ | CH₃ | C₆H₅ | CH(CH₃)₂ |
| B-35 | CH₂F | CH₃ | C₆H₅ | CH(CH₃)₂ |
| B-36 | CHF₂ | CH₃ | C₆H₅ | CH(CH₃)₂ |
| B-37 | CH₃ | OCH₃ | C₆H₅ | CH(CH₃)₂ |
| B-38 | OCH₃ | OCH₃ | C₆H₅ | CH(CH₃)₂ |
| B-39 | CH₂F | OCH₃ | C₆H₅ | CH(CH₃)₂ |
| B-40 | CHF₂ | OCH₃ | C₆H₅ | CH(CH₃)₂ |
| B-41 | CH₃ | CH₂F | C₆H₅ | CH(CH₃)₂ |
| B-42 | OCH₃ | CH₂F | C₆H₅ | CH(CH₃)₂ |
| B-43 | CH₂F | CH₂F | C₆H₅ | CH(CH₃)₂ |
| B-44 | CHF₂ | CH₂F | C₆H₅ | CH(CH₃)₂ |
| B-45 | CH₃ | CHF₂ | C₆H₅ | CH(CH₃)₂ |
| B-46 | OCH₃ | CHF₂ | C₆H₅ | CH(CH₃)₂ |
| B-47 | CH₂F | CHF₂ | C₆H₅ | CH(CH₃)₂ |
| B-48 | CHF₂ | CHF₂ | C₆H₅ | CH(CH₃)₂ |
| B-49 | CH₃ | CH₃ | —CH₂—C₆H₅ | CH(CH₃)₂ |
| B-50 | OCH₃ | CH₃ | —CH₂—C₆H₅ | CH(CH₃)₂ |
| B-51 | CH₂F | CH₃ | —CH₂—C₆H₅ | CH(CH₃)₂ |
| B-52 | CHF₂ | CH₃ | —CH₂—C₆H₅ | CH(CH₃)₂ |
| B-53 | CH₃ | OCH₃ | —CH₂—C₆H₅ | CH(CH₃)₂ |
| B-54 | OCH₃ | OCH₃ | —CH₂—C₆H₅ | CH(CH₃)₂ |
| B-55 | CH₂F | OCH₃ | —CH₂—C₆H₅ | CH(CH₃)₂ |
| B-56 | CHF₂ | OCH₃ | —CH₂—C₆H₅ | CH(CH₃)₂ |
| B-57 | CH₃ | CH₂F | —CH₂—C₆H₅ | CH(CH₃)₂ |
| B-58 | OCH₃ | CH₂F | —CH₂—C₆H₅ | CH(CH₃)₂ |
| B-59 | CH₂F | CH₂F | —CH₂—C₆H₅ | CH(CH₃)₂ |
| B-60 | CHF₂ | CH₂F | —CH₂—C₆H₅ | CH(CH₃)₂ |
| B-61 | CH₃ | CHF₂ | —CH₂—C₆H₅ | CH(CH₃)₂ |
| B-62 | OCH₃ | CHF₂ | —CH₂—C₆H₅ | CH(CH₃)₂ |
| B-63 | CH₂F | CHF₂ | —CH₂—C₆H₅ | CH(CH₃)₂ |
| B-64 | CHF₂ | CHF₂ | —CH₂—C₆H₅ | CH(CH₃)₂ |
| B-65 | CH₃ | CH₃ | C₆H₅ | C(CH₃)₃ |
| B-66 | OCH₃ | CH₃ | C₆H₅ | C(CH₃)₃ |
| B-67 | CH₂F | CH₃ | C₆H₅ | C(CH₃)₃ |
| B-68 | CHF₂ | CH₃ | C₆H₅ | C(CH₃)₃ |
| B-69 | CH₃ | OCH₃ | C₆H₅ | C(CH₃)₃ |
| B-70 | OCH₃ | OCH₃ | C₆H₅ | C(CH₃)₃ |
| B-71 | CH₂F | OCH₃ | C₆H₅ | C(CH₃)₃ |
| B-72 | CHF₂ | OCH₃ | C₆H₅ | C(CH₃)₃ |
| B-73 | CH₃ | CH₂F | C₆H₅ | C(CH₃)₃ |
| B-74 | OCH₃ | CH₂F | C₆H₅ | C(CH₃)₃ |
| B-75 | CH₂F | CH₂F | C₆H₅ | C(CH₃)₃ |
| B-76 | CHF₂ | CH₂F | C₆H₅ | C(CH₃)₃ |
| B-77 | CH₃ | CHF₂ | C₆H₅ | C(CH₃)₃ |
| B-78 | OCH₃ | CHF₂ | C₆H₅ | C(CH₃)₃ |
| B-79 | CH₂F | CHF₂ | C₆H₅ | C(CH₃)₃ |
| B-80 | CHF₂ | CHF₂ | C₆H₅ | C(CH₃)₃ |
| B-81 | CH₃ | CH₃ | —CH₂—C₆H₅ | C(CH₃)₃ |
| B-82 | OCH₃ | CH₃ | —CH₂—C₆H₅ | C(CH₃)₃ |
| B-83 | CH₂F | CH₃ | —CH₂—C₆H₅ | C(CH₃)₃ |
| B-84 | CHF₂ | CH₃ | —CH₂—C₆H₅ | C(CH₃)₃ |
| B-85 | CH₃ | OCH₃ | —CH₂—C₆H₅ | C(CH₃)₃ |
| B-86 | OCH₃ | OCH₃ | —CH₂—C₆H₅ | C(CH₃)₃ |
| B-87 | CH₂F | OCH₃ | —CH₂—C₆H₅ | C(CH₃)₃ |
| B-88 | CHF₂ | OCH₃ | —CH₂—C₆H₅ | C(CH₃)₃ |
| B-89 | CH₃ | CH₂F | —CH₂—C₆H₅ | C(CH₃)₃ |
| B-90 | OCH₃ | CH₂F | —CH₂—C₆H₅ | C(CH₃)₃ |
| B-91 | CH₂F | CH₂F | —CH₂—C₆H₅ | C(CH₃)₃ |
| B-92 | CHF₂ | CH₂F | —CH₂—C₆H₅ | C(CH₃)₃ |
| B-93 | CH₃ | CHF₂ | —CH₂—C₆H₅ | C(CH₃)₃ |
| B-94 | OCH₃ | CHF₂ | —CH₂—C₆H₅ | C(CH₃)₃ |
| B-95 | CH₂F | CHF₂ | —CH₂—C₆H₅ | C(CH₃)₃ |
| B-96 | CHF₂ | CHF₂ | —CH₂—C₆H₅ | C(CH₃)₃ |
| B-97 | CH₃ | CH₃ | C₆H₅ | —CH₂—CH(CH₃)₂ |
| B-98 | OCH₃ | CH₃ | C₆H₅ | —CH₂—CH(CH₃)₂ |
| B-99 | CH₂F | CH₃ | C₆H₅ | —CH₂—CH(CH₃)₂ |
| B-100 | CHF₂ | CH₃ | C₆H₅ | —CH₂—CH(CH₃)₂ |
| B-101 | CH₃ | OCH₃ | C₆H₅ | —CH₂—CH(CH₃)₂ |
| B-102 | OCH₃ | OCH₃ | C₆H₅ | —CH₂—CH(CH₃)₂ |
| B-103 | CH₂F | OCH₃ | C₆H₅ | —CH₂—CH(CH₃)₂ |
| B-104 | CHF₂ | OCH₃ | C₆H₅ | —CH₂—CH(CH₃)₂ |
| B-105 | CH₃ | CH₂F | C₆H₅ | —CH₂—CH(CH₃)₂ |
| B-106 | OCH₃ | CH₂F | C₆H₅ | —CH₂—CH(CH₃)₂ |
| B-107 | CH₂F | CH₂F | C₆H₅ | —CH₂—CH(CH₃)₂ |
| B-108 | CHF₂ | CH₂F | C₆H₅ | —CH₂—CH(CH₃)₂ |
| B-109 | CH₃ | CHF₂ | C₆H₅ | —CH₂—CH(CH₃)₂ |
| B-110 | OCH₃ | CHF₂ | C₆H₅ | —CH₂—CH(CH₃)₂ |
| B-111 | CH₂F | CHF₂ | C₆H₅ | —CH₂—CH(CH₃)₂ |
| B-112 | CHF₂ | CHF₂ | C₆H₅ | —CH₂—CH(CH₃)₂ |
| B-113 | CH₃ | CH₃ | —CH₂—C₆H₅ | —CH₂—CH(CH₃)₂ |
| B-114 | OCH₃ | CH₃ | —CH₂—C₆H₅ | —CH₂—CH(CH₃)₂ |
| B-115 | CH₂F | CH₃ | —CH₂—C₆H₅ | —CH₂—CH(CH₃)₂ |
| B-116 | CHF₂ | CH₃ | —CH₂—C₆H₅ | —CH₂—CH(CH₃)₂ |
| B-117 | CH₃ | OCH₃ | —CH₂—C₆H₅ | —CH₂—CH(CH₃)₂ |
| B-118 | OCH₃ | OCH₃ | —CH₂—C₆H₅ | —CH₂—CH(CH₃)₂ |
| B-119 | CH₂F | OCH₃ | —CH₂—C₆H₅ | —CH₂—CH(CH₃)₂ |
| B-120 | CHF₂ | OCH₃ | —CH₂—C₆H₅ | —CH₂—CH(CH₃)₂ |
| B-121 | CH₃ | CH₂F | —CH₂—C₆H₅ | —CH₂—CH(CH₃)₂ |
| B-122 | OCH₃ | CH₂F | —CH₂—C₆H₅ | —CH₂—CH(CH₃)₂ |
| B-123 | CH₂F | CH₂F | —CH₂—C₆H₅ | —CH₂—CH(CH₃)₂ |
| B-124 | CHF₂ | CH₂F | —CH₂—C₆H₅ | —CH₂—CH(CH₃)₂ |
| B-125 | CH₃ | CHF₂ | —CH₂—C₆H₅ | —CH₂—CH(CH₃)₂ |
| B-126 | OCH₃ | CHF₂ | —CH₂—C₆H₅ | —CH₂—CH(CH₃)₂ |
| B-127 | CH₂F | CHF₂ | —CH₂—C₆H₅ | —CH₂—CH(CH₃)₂ |
| B-128 | CHF₂ | CHF₂ | —CH₂—C₆H₅ | —CH₂—CH(CH₃)₂ |
| B-129 | CH₃ | CH₃ | C₆H₅ | CH₃ |
| B-130 | CH₂F | CH₃ | C₆H₅ | CH₃ |
| B-131 | OCH₃ | CH₃ | C₆H₅ | CH₃ |
| B-132 | CH₃ | CH₂F | C₆H₅ | CH₃ |
| B-133 | CH₂F | CH₂F | C₆H₅ | CH₃ |
| B-134 | OCH₃ | CH₂F | C₆H₅ | CH₃ |
| B-135 | CH₃ | CHF₂ | C₆H₅ | CH₃ |
| B-136 | CH₂F | CHF₂ | C₆H₅ | CH₃ |
| B-137 | OCH₃ | CHF₂ | C₆H₅ | CH₃ |
| B-138 | CH₃ | CF₃ | C₆H₅ | CH₃ |
| B-139 | CH₂F | CF₃ | C₆H₅ | CH₃ |
| B-140 | OCH₃ | CF₃ | C₆H₅ | CH₃ |
| B-141 | CH₃ | CH₃ | —CH₂—C₆H₅ | CH₃ |
| B-142 | CH₂F | CH₃ | —CH₂—C₆H₅ | CH₃ |
| B-143 | OCH₃ | CH₃ | —CH₂—C₆H₅ | CH₃ |
| B-144 | CH₃ | CH₂F | —CH₂—C₆H₅ | CH₃ |
| B-145 | CH₂F | CH₂F | —CH₂—C₆H₅ | CH₃ |
| B-146 | OCH₃ | CH₂F | —CH₂—C₆H₅ | CH₃ |
| B-147 | CH₃ | CHF₂ | —CH₂—C₆H₅ | CH₃ |
| B-148 | CH₂F | CHF₂ | —CH₂—C₆H₅ | CH₃ |
| B-149 | OCH₃ | CHF₂ | —CH₂—C₆H₅ | CH₃ |
| B-150 | CH₃ | CF₃ | —CH₂—C₆H₅ | CH₃ |
| B-151 | CH₂F | CF₃ | —CH₂—C₆H₅ | CH₃ |
| B-152 | OCH₃ | CF₃ | —CH₂—C₆H₅ | CH₃ |
| B-153 | CH₃ | CH₃ | —CH₂-(2-F—C₆H₄) | CH₃ |
| B-154 | CH₂F | CH₃ | —CH₂-(2-F—C₆H₄) | CH₃ |
| B-155 | OCH₃ | CH₃ | —CH₂-(2-F—C₆H₄) | CH₃ |
| B-156 | CH₃ | CH₂F | —CH₂-(2-F—C₆H₄) | CH₃ |
| B-157 | CH₂F | CH₂F | —CH₂-(2-F—C₆H₄) | CH₃ |
| B-158 | OCH₃ | CH₂F | —CH₂-(2-F—C₆H₄) | CH₃ |
| B-159 | CH₃ | CHF₂ | —CH₂-(2-F—C₆H₄) | CH₃ |
| B-160 | CH₂F | CHF₂ | —CH₂-(2-F—C₆H₄) | CH₃ |
| B-161 | OCH₃ | CHF₂ | —CH₂-(2-F—C₆H₄) | CH₃ |
| B-162 | CH₃ | CF₃ | —CH₂-(2-F—C₆H₄) | CH₃ |
| B-163 | CH₂F | CF₃ | —CH₂-(2-F—C₆H₄) | CH₃ |
| B-164 | OCH₃ | CF₃ | —CH₂-(2-F—C₆H₄) | CH₃ |
| B-165 | CH₃ | CH₃ | —CH₂-(3-F—C₆H₄) | CH₃ |
| B-166 | CH₂F | CH₃ | —CH₂-(3-F—C₆H₄) | CH₃ |
| B-167 | OCH₃ | CH₃ | —CH₂-(3-F—C₆H₄) | CH₃ |
| B-168 | CH₃ | CH₂F | —CH₂-(3-F—C₆H₄) | CH₃ |
| B-169 | CH₂F | CH₂F | —CH₂-(3-F—C₆H₄) | CH₃ |
| B-170 | OCH₃ | CH₂F | —CH₂-(3-F—C₆H₄) | CH₃ |
| B-171 | CH₃ | CHF₂ | —CH₂-(3-F—C₆H₄) | CH₃ |
| B-172 | CH₂F | CHF₂ | —CH₂-(3-F—C₆H₄) | CH₃ |
| B-173 | OCH₃ | CHF₂ | —CH₂-(3-F—C₆H₄) | CH₃ |
| B-174 | CH₃ | CF₃ | —CH₂-(3-F—C₆H₄) | CH₃ |
| B-175 | CH₂F | CF₃ | —CH₂-(3-F—C₆H₄) | CH₃ |
| B-176 | OCH₃ | CF₃ | —CH₂-(3-F—C₆H₄) | CH₃ |
| B-177 | CH₃ | CH₃ | —CH₂-(4-F—C₆H₄) | CH₃ |
| B-178 | CH₂F | CH₃ | —CH₂-(4-F—C₆H₄) | CH₃ |
| B-179 | OCH₃ | CH₃ | —CH₂-(4-F—C₆H₄) | CH₃ |
| B-180 | CH₃ | CH₂F | —CH₂-(4-F—C₆H₄) | CH₃ |
| B-181 | CH₂F | CH₂F | —CH₂-(4-F—C₆H₄) | CH₃ |
| B-182 | OCH₃ | CH₂F | —CH₂-(4-F—C₆H₄) | CH₃ |
| B-183 | CH₃ | CHF₂ | —CH₂-(4-F—C₆H₄) | CH₃ |
| B-184 | CH₂F | CHF₂ | —CH₂-(4-F—C₆H₄) | CH₃ |
| B-185 | OCH₃ | CHF₂ | —CH₂-(4-F—C₆H₄) | CH₃ |
| B-186 | CH₃ | CF₃ | —CH₂-(4-F—C₆H₄) | CH₃ |
| B-187 | CH₂F | CF₃ | —CH₂-(4-F—C₆H₄) | CH₃ |
| B-188 | OCH₃ | CF₃ | —CH₂-(4-F—C₆H₄) | CH₃ |
| B-189 | CH₃ | CH₃ | —CH₂-(2,4-F₂—C₆H₃) | CH₃ |
| B-190 | CH₂F | CH₃ | —CH₂-(2,4-F₂—C₆H₃) | CH₃ |
| B-191 | OCH₃ | CH₃ | —CH₂-(2,4-F₂—C₆H₃) | CH₃ |
| B-192 | CH₃ | CH₂F | —CH₂-(2,4-F₂—C₆H₃) | CH₃ |
| B-193 | CH₂F | CH₂F | —CH₂-(2,4-F₂—C₆H₃) | CH₃ |
| B-194 | OCH₃ | CH₂F | —CH₂-(2,4-F₂—C₆H₃) | CH₃ |
| B-195 | CH₃ | CHF₂ | —CH₂-(2,4-F₂—C₆H₃) | CH₃ |
| B-196 | CH₂F | CHF₂ | —CH₂-(2,4-F₂—C₆H₃) | CH₃ |
| B-197 | OCH₃ | CHF₂ | —CH₂-(2,4-F₂—C₆H₃) | CH₃ |
| B-198 | CH₃ | CF₃ | —CH₂-(2,4-F₂—C₆H₃) | CH₃ |
| B-199 | CH₂F | CF₃ | —CH₂-(2,4-F₂—C₆H₃) | CH₃ |
| B-200 | OCH₃ | CF₃ | —CH₂-(2,4-F₂—C₆H₃) | CH₃ |
| B-201 | CH₃ | CH₃ | —CH₂-(2-Cl—C₆H₄) | CH₃ |
| B-202 | CH₂F | CH₃ | —CH₂-(2-Cl—C₆H₄) | CH₃ |
| B-203 | OCH₃ | CH₃ | —CH₂-(2-Cl—C₆H₄) | CH₃ |
| B-204 | CH₃ | CH₂F | —CH₂-(2-Cl—C₆H₄) | CH₃ |
| B-205 | CH₂F | CH₂F | —CH₂-(2-Cl—C₆H₄) | CH₃ |
| B-206 | OCH₃ | CH₂F | —CH₂-(2-Cl—C₆H₄) | CH₃ |
| B-207 | CH₃ | CHF₂ | —CH₂-(2-Cl—C₆H₄) | CH₃ |
| B-208 | CH₂F | CHF₂ | —CH₂-(2-Cl—C₆H₄) | CH₃ |
| B-209 | OCH₃ | CHF₂ | —CH₂-(2-Cl—C₆H₄) | CH₃ |
| B-210 | CH₃ | CF₃ | —CH₂-(2-Cl—C₆H₄) | CH₃ |
| B-211 | CH₂F | CF₃ | —CH₂-(2-Cl—C₆H₄) | CH₃ |
| B-212 | OCH₃ | CF₃ | —CH₂-(2-Cl—C₆H₄) | CH₃ |
| B-213 | CH₃ | CH₃ | —CH₂-(3-Cl—C₆H₄) | CH₃ |
| B-214 | CH₂F | CH₃ | —CH₂-(3-Cl—C₆H₄) | CH₃ |
| B-215 | OCH₃ | CH₃ | —CH₂-(3-Cl—C₆H₄) | CH₃ |
| B-216 | CH₃ | CH₂F | —CH₂-(3-Cl—C₆H₄) | CH₃ |
| B-217 | CH₂F | CH₂F | —CH₂-(3-Cl—C₆H₄) | CH₃ |
| B-218 | OCH₃ | CH₂F | —CH₂-(3-Cl—C₆H₄) | CH₃ |
| B-219 | CH₃ | CHF₂ | —CH₂-(3-Cl—C₆H₄) | CH₃ |
| B-220 | CH₂F | CHF₂ | —CH₂-(3-Cl—C₆H₄) | CH₃ |
| B-221 | OCH₃ | CHF₂ | —CH₂-(3-Cl—C₆H₄) | CH₃ |
| B-222 | CH₃ | CF₃ | —CH₂-(3-Cl—C₆H₄) | CH₃ |
| B-223 | CH₂F | CF₃ | —CH₂-(3-Cl—C₆H₄) | CH₃ |
| B-224 | OCH₃ | CF₃ | —CH₂-(3-Cl—C₆H₄) | CH₃ |
| B-225 | CH₃ | CH₃ | —CH₂-(4-Cl—C₆H₄) | CH₃ |
| B-226 | CH₂F | CH₃ | —CH₂-(4-Cl—C₆H₄) | CH₃ |
| B-227 | OCH₃ | CH₃ | —CH₂-(4-Cl—C₆H₄) | CH₃ |
| B-228 | CH₃ | CH₂F | —CH₂-(4-Cl—C₆H₄) | CH₃ |
| B-229 | CH₂F | CH₂F | —CH₂-(4-Cl—C₆H₄) | CH₃ |
| B-230 | OCH₃ | CH₂F | —CH₂-(4-Cl—C₆H₄) | CH₃ |
| B-231 | CH₃ | CHF₂ | —CH₂-(4-Cl—C₆H₄) | CH₃ |
| B-232 | CH₂F | CHF₂ | —CH₂-(4-Cl—C₆H₄) | CH₃ |
| B-233 | OCH₃ | CHF₂ | —CH₂-(4-Cl—C₆H₄) | CH₃ |
| B-234 | CH₃ | CF₃ | —CH₂-(4-Cl—C₆H₄) | CH₃ |
| B-235 | CH₂F | CF₃ | —CH₂-(4-Cl—C₆H₄) | CH₃ |
| B-236 | OCH₃ | CF₃ | —CH₂-(4-Cl—C₆H₄) | CH₃ |
| B-237 | CH₃ | CH₃ | —CH₂-(2,4-Cl₂—C₆H₃) | CH₃ |
| B-238 | CH₂F | CH₃ | —CH₂-(2,4-Cl₂—C₆H₃) | CH₃ |
| B-239 | OCH₃ | CH₃ | —CH₂-(2,4-Cl₂—C₆H₃) | CH₃ |
| B-240 | CH₃ | CH₂F | —CH₂-(2,4-Cl₂—C₆H₃) | CH₃ |
| B-241 | CH₂F | CH₂F | —CH₂-(2,4-Cl₂—C₆H₃) | CH₃ |
| B-242 | OCH₃ | CH₂F | —CH₂-(2,4-Cl₂—C₆H₃) | CH₃ |
| B-243 | CH₃ | CHF₂ | —CH₂-(2,4-Cl₂—C₆H₃) | CH₃ |
| B-244 | CH₂F | CHF₂ | —CH₂-(2,4-Cl₂—C₆H₃) | CH₃ |
| B-245 | OCH₃ | CHF₂ | —CH₂-(2,4-Cl₂—C₆H₃) | CH₃ |
| B-246 | CH₃ | CF₃ | —CH₂-(2,4-Cl₂—C₆H₃) | CH₃ |
| B-247 | CH₂F | CF₃ | —CH₂-(2,4-Cl₂—C₆H₃) | CH₃ |
| B-248 | OCH₃ | CF₃ | —CH₂-(2,4-Cl₂—C₆H₃) | CH₃ |
| B-249 | CH₃ | CH₃ | —CH₂—CH₂—C₆H₅ | CH₃ |
| B-250 | CH₂F | CH₃ | —CH₂—CH₂—C₆H₅ | CH₃ |
| B-251 | OCH₃ | CH₃ | —CH₂—CH₂—C₆H₅ | CH₃ |
| B-252 | CH₃ | CH₂F | —CH₂—CH₂—C₆H₅ | CH₃ |
| B-253 | CH₂F | CH₂F | —CH₂—CH₂—C₆H₅ | CH₃ |
| B-254 | OCH₃ | CH₂F | —CH₂—CH₂—C₆H₅ | CH₃ |
| B-255 | CH₃ | CHF₂ | —CH₂—CH₂—C₆H₅ | CH₃ |
| B-256 | CH₂F | CHF₂ | —CH₂—CH₂—C₆H₅ | CH₃ |
| B-257 | OCH₃ | CHF₂ | —CH₂—CH₂—C₆H₅ | CH₃ |
| B-258 | CH₃ | CF₃ | —CH₂—CH₂—C₆H₅ | CH₃ |
| B-259 | CH₂F | CF₃ | —CH₂—CH₂—C₆H₅ | CH₃ |
| B-260 | OCH₃ | CF₃ | —CH₂—CH₂—C₆H₅ | CH₃ |
| B-261 | CH₃ | CH₃ | C₆H₅ | CH₂—CH₃ |
| B-262 | CH₂F | CH₃ | C₆H₅ | CH₂—CH₃ |
| B-263 | OCH₃ | CH₃ | C₆H₅ | CH₂—CH₃ |
| B-264 | CH₃ | CH₂F | C₆H₅ | CH₂—CH₃ |
| B-265 | CH₂F | CH₂F | C₆H₅ | CH₂—CH₃ |
| B-266 | OCH₃ | CH₂F | C₆H₅ | CH₂—CH₃ |
| B-267 | CH₃ | CHF₂ | C₆H₅ | CH₂—CH₃ |
| B-268 | CH₂F | CHF₂ | C₆H₅ | CH₂—CH₃ |
| B-269 | OCH₃ | CHF₂ | C₆H₅ | CH₂—CH₃ |
| B-270 | CH₃ | CF₃ | C₆H₅ | CH₂—CH₃ |
| B-271 | CH₂F | CF₃ | C₆H₅ | CH₂—CH₃ |
| B-272 | OCH₃ | CF₃ | C₆H₅ | CH₂—CH₃ |
| B-273 | CH₃ | CH₃ | —CH₂—C₆H₅ | CH₂—CH₃ |
| B-274 | CH₂F | CH₃ | —CH₂—C₆H₅ | CH₂—CH₃ |
| B-275 | OCH₃ | CH₃ | —CH₂—C₆H₅ | CH₂—CH₃ |
| B-276 | CH₃ | CH₂F | —CH₂—C₆H₅ | CH₂—CH₃ |
| B-277 | CH₂F | CH₂F | —CH₂—C₆H₅ | CH₂—CH₃ |
| B-278 | OCH₃ | CH₂F | —CH₂—C₆H₅ | CH₂—CH₃ |
| B-279 | CH₃ | CHF₂ | —CH₂—C₆H₅ | CH₂—CH₃ |
| B-280 | CH₂F | CHF₂ | —CH₂—C₆H₅ | CH₂—CH₃ |
| B-281 | OCH₃ | CHF₂ | —CH₂—C₆H₅ | CH₂—CH₃ |
| B-282 | CH₃ | CF₃ | —CH₂—C₆H₅ | CH₂—CH₃ |
| B-283 | CH₂F | CF₃ | —CH₂—C₆H₅ | CH₂—CH₃ |
| B-284 | OCH₃ | CF₃ | —CH₂—C₆H₅ | CH₂—CH₃ |
| B-285 | CH₃ | CH₃ | —CH₂-(2-F—C₆H₄) | CH₂—CH₃ |
| B-286 | CH₂F | CH₃ | —CH₂-(2-F—C₆H₄) | CH₂—CH₃ |
| B-287 | OCH₃ | CH₃ | —CH₂-(2-F—C₆H₄) | CH₂—CH₃ |
| B-288 | CH₃ | CH₂F | —CH₂-(2-F—C₆H₄) | CH₂—CH₃ |
| B-289 | CH₂F | CH₂F | —CH₂-(2-F—C₆H₄) | CH₂—CH₃ |
| B-290 | OCH₃ | CH₂F | —CH₂-(2-F—C₆H₄) | CH₂—CH₃ |
| B-291 | CH₃ | CHF₂ | —CH₂-(2-F—C₆H₄) | CH₂—CH₃ |
| B-292 | CH₂F | CHF₂ | —CH₂-(2-F—C₆H₄) | CH₂—CH₃ |
| B-293 | OCH₃ | CHF₂ | —CH₂-(2-F—C₆H₄) | CH₂—CH₃ |
| B-294 | CH₃ | CF₃ | —CH₂-(2-F—C₆H₄) | CH₂—CH₃ |
| B-295 | CH₂F | CF₃ | —CH₂-(2-F—C₆H₄) | CH₂—CH₃ |
| B-296 | OCH₃ | CF₃ | —CH₂-(2-F—C₆H₄) | CH₂—CH₃ |
| B-297 | CH₃ | CH₃ | —CH₂-(3-F—C₆H₄) | CH₂—CH₃ |
| B-298 | CH₂F | CH₃ | —CH₂-(3-F—C₆H₄) | CH₂—CH₃ |
| B-299 | OCH₃ | CH₃ | —CH₂-(3-F—C₆H₄) | CH₂—CH₃ |
| B-300 | CH₃ | CH₂F | —CH₂-(3-F—C₆H₄) | CH₂—CH₃ |
| B-301 | CH₂F | CH₂F | —CH₂-(3-F—C₆H₄) | CH₂—CH₃ |
| B-302 | OCH₃ | CH₂F | —CH₂-(3-F—C₆H₄) | CH₂—CH₃ |
| B-303 | CH₃ | CHF₂ | —CH₂-(3-F—C₆H₄) | CH₂—CH₃ |
| B-304 | CH₂F | CHF₂ | —CH₂-(3-F—C₆H₄) | CH₂—CH₃ |
| B-305 | OCH₃ | CHF₂ | —CH₂-(3-F—C₆H₄) | CH₂—CH₃ |
| B-306 | CH₃ | CF₃ | —CH₂-(3-F—C₆H₄) | CH₂—CH₃ |
| B-307 | CH₂F | CF₃ | —CH₂-(3-F—C₆H₄) | CH₂—CH₃ |
| B-308 | OCH₃ | CF₃ | —CH₂-(3-F—C₆H₄) | CH₂—CH₃ |
| B-309 | CH₃ | CH₃ | —CH₂-(4-F—C₆H₄) | CH₂—CH₃ |
| B-310 | CH₂F | CH₃ | —CH₂-(4-F—C₆H₄) | CH₂—CH₃ |
| B-311 | OCH₃ | CH₃ | —CH₂-(4-F—C₆H₄) | CH₂—CH₃ |
| B-312 | CH₃ | CH₂F | —CH₂-(4-F—C₆H₄) | CH₂—CH₃ |
| B-313 | CH₂F | CH₂F | —CH₂-(4-F—C₆H₄) | CH₂—CH₃ |
| B-314 | OCH₃ | CH₂F | —CH₂-(4-F—C₆H₄) | CH₂—CH₃ |
| B-315 | CH₃ | CHF₂ | —CH₂-(4-F—C₆H₄) | CH₂—CH₃ |
| B-316 | CH₂F | CHF₂ | —CH₂-(4-F—C₆H₄) | CH₂—CH₃ |
| B-317 | OCH₃ | CHF₂ | —CH₂-(4-F—C₆H₄) | CH₂—CH₃ |
| B-318 | CH₃ | CF₃ | —CH₂-(4-F—C₆H₄) | CH₂—CH₃ |
| B-319 | CH₂F | CF₃ | —CH₂-(4-F—C₆H₄) | CH₂—CH₃ |
| B-320 | OCH₃ | CF₃ | —CH₂-(4-F—C₆H₄) | CH₂—CH₃ |
| B-321 | CH₃ | CH₃ | —CH₂-(2,4-F₂—C₆H₃) | CH₂—CH₃ |
| B-322 | CH₂F | CH₃ | —CH₂-(2,4-F₂—C₆H₃) | CH₂—CH₃ |
| B-323 | OCH₃ | CH₃ | —CH₂-(2,4-F₂—C₆H₃) | CH₂—CH₃ |
| B-324 | CH₃ | CH₂F | —CH₂-(2,4-F₂—C₆H₃) | CH₂—CH₃ |
| B-325 | CH₂F | CH₂F | —CH₂-(2,4-F₂—C₆H₃) | CH₂—CH₃ |
| B-326 | OCH₃ | CH₂F | —CH₂-(2,4-F₂—C₆H₃) | CH₂—CH₃ |
| B-327 | CH₃ | CHF₂ | —CH₂-(2,4-F₂—C₆H₃) | CH₂—CH₃ |
| B-328 | CH₂F | CHF₂ | —CH₂-(2,4-F₂—C₆H₃) | CH₂—CH₃ |
| B-329 | OCH₃ | CHF₂ | —CH₂-(2,4-F₂—C₆H₃) | CH₂—CH₃ |
| B-330 | CH₃ | CF₃ | —CH₂-(2,4-F₂—C₆H₃) | CH₂—CH₃ |
| B-331 | CH₂F | CF₃ | —CH₂-(2,4-F₂—C₆H₃) | CH₂—CH₃ |
| B-332 | OCH₃ | CF₃ | —CH₂-(2,4-F₂—C₆H₃) | CH₂—CH₃ |
| B-333 | CH₃ | CH₃ | —CH₂-(2-Cl—C₆H₄) | CH₂—CH₃ |
| B-334 | CH₂F | CH₃ | —CH₂-(2-Cl—C₆H₄) | CH₂—CH₃ |
| B-335 | OCH₃ | CH₃ | —CH₂-(2-Cl—C₆H₄) | CH₂—CH₃ |
| B-336 | CH₃ | CH₂F | —CH₂-(2-Cl—C₆H₄) | CH₂—CH₃ |
| B-337 | CH₂F | CH₂F | —CH₂-(2-Cl—C₆H₄) | CH₂—CH₃ |
| B-338 | OCH₃ | CH₂F | —CH₂-(2-Cl—C₆H₄) | CH₂—CH₃ |
| B-339 | CH₃ | CHF₂ | —CH₂-(2-Cl—C₆H₄) | CH₂—CH₃ |
| B-340 | CH₂F | CHF₂ | —CH₂-(2-Cl—C₆H₄) | CH₂—CH₃ |
| B-341 | OCH₃ | CHF₂ | —CH₂-(2-Cl—C₆H₄) | CH₂—CH₃ |
| B-342 | CH₃ | CF₃ | —CH₂-(2-Cl—C₆H₄) | CH₂—CH₃ |
| B-343 | CH₂F | CF₃ | —CH₂-(2-Cl—C₆H₄) | CH₂—CH₃ |
| B-344 | OCH₃ | CF₃ | —CH₂-(2-Cl—C₆H₄) | CH₂—CH₃ |
| B-345 | CH₃ | CH₃ | —CH₂-(3-Cl—C₆H₄) | CH₂—CH₃ |
| B-346 | CH₂F | CH₃ | —CH₂-(3-Cl—C₆H₄) | CH₂—CH₃ |
| B-347 | OCH₃ | CH₃ | —CH₂-(3-Cl—C₆H₄) | CH₂—CH₃ |
| B-348 | CH₃ | CH₂F | —CH₂-(3-Cl—C₆H₄) | CH₂—CH₃ |
| B-349 | CH₂F | CH₂F | —CH₂-(3-Cl—C₆H₄) | CH₂—CH₃ |
| B-350 | OCH₃ | CH₂F | —CH₂-(3-Cl—C₆H₄) | CH₂—CH₃ |
| B-351 | CH₃ | CHF₂ | —CH₂-(3-Cl—C₆H₄) | CH₂—CH₃ |
| B-352 | CH₂F | CHF₂ | —CH₂-(3-Cl—C₆H₄) | CH₂—CH₃ |
| B-353 | OCH₃ | CHF₂ | —CH₂-(3-Cl—C₆H₄) | CH₂—CH₃ |
| B-354 | CH₃ | CF₃ | —CH₂-(3-Cl—C₆H₄) | CH₂—CH₃ |
| B-355 | CH₂F | CF₃ | —CH₂-(3-Cl—C₆H₄) | CH₂—CH₃ |
| B-356 | OCH₃ | CF₃ | —CH₂-(3-Cl—C₆H₄) | CH₂—CH₃ |
| B-357 | CH₃ | CH₃ | —CH₂-(4-Cl—C₆H₄) | CH₂—CH₃ |
| B-358 | CH₂F | CH₃ | —CH₂-(4-Cl—C₆H₄) | CH₂—CH₃ |
| B-359 | OCH₃ | CH₃ | —CH₂-(4-Cl—C₆H₄) | CH₂—CH₃ |
| B-360 | CH₃ | CH₂F | —CH₂-(4-Cl—C₆H₄) | CH₂—CH₃ |
| B-361 | CH₂F | CH₂F | —CH₂-(4-Cl—C₆H₄) | CH₂—CH₃ |
| B-362 | OCH₃ | CH₂F | —CH₂-(4-Cl—C₆H₄) | CH₂—CH₃ |
| B-363 | CH₃ | CHF₂ | —CH₂-(4-Cl—C₆H₄) | CH₂—CH₃ |
| B-364 | CH₂F | CHF₂ | —CH₂-(4-Cl—C₆H₄) | CH₂—CH₃ |
| B-365 | OCH₃ | CHF₂ | —CH₂-(4-Cl—C₆H₄) | CH₂—CH₃ |
| B-366 | CH₃ | CF₃ | —CH₂-(4-Cl—C₆H₄) | CH₂—CH₃ |
| B-367 | CH₂F | CF₃ | —CH₂-(4-Cl—C₆H₄) | CH₂—CH₃ |
| B-368 | OCH₃ | CF₃ | —CH₂-(4-Cl—C₆H₄) | CH₂—CH₃ |
| B-369 | CH₃ | CH₃ | —CH₂-(2,4-Cl₂—C₆H₃) | CH₂—CH₃ |
| B-370 | CH₂F | CH₃ | —CH₂-(2,4-Cl₂—C₆H₃) | CH₂—CH₃ |
| B-371 | OCH₃ | CH₃ | —CH₂-(2,4-Cl₂—C₆H₃) | CH₂—CH₃ |
| B-372 | CH₃ | CH₂F | —CH₂-(2,4-Cl₂—C₆H₃) | CH₂—CH₃ |
| B-373 | CH₂F | CH₂F | —CH₂-(2,4-Cl₂—C₆H₃) | CH₂—CH₃ |
| B-374 | OCH₃ | CH₂F | —CH₂-(2,4-Cl₂—C₆H₃) | CH₂—CH₃ |
| B-375 | CH₃ | CHF₂ | —CH₂-(2,4-Cl₂—C₆H₃) | CH₂—CH₃ |
| B-376 | CH₂F | CHF₂ | —CH₂-(2,4-Cl₂—C₆H₃) | CH₂—CH₃ |
| B-377 | OCH₃ | CHF₂ | —CH₂-(2,4-Cl₂—C₆H₃) | CH₂—CH₃ |
| B-378 | CH₃ | CF₃ | —CH₂-(2,4-Cl₂—C₆H₃) | CH₂—CH₃ |
| B-379 | CH₂F | CF₃ | —CH₂-(2,4-Cl₂—C₆H₃) | CH₂—CH₃ |
| B-380 | OCH₃ | CF₃ | —CH₂-(2,4-Cl₂—C₆H₃) | CH₂—CH₃ |
| B-381 | CH₃ | CH₃ | —CH₂—CH₂—C₆H₅ | CH₂—CH₃ |
| B-382 | CH₂F | CH₃ | —CH₂—CH₂—C₆H₅ | CH₂—CH₃ |
| B-383 | OCH₃ | CH₃ | —CH₂—CH₂—C₆H₅ | CH₂—CH₃ |
| B-384 | CH₃ | CH₂F | —CH₂—CH₂—C₆H₅ | CH₂—CH₃ |
| B-385 | CH₂F | CH₂F | —CH₂—CH₂—C₆H₅ | CH₂—CH₃ |
| B-386 | OCH₃ | CH₂F | —CH₂—CH₂—C₆H₅ | CH₂—CH₃ |
| B-387 | CH₃ | CHF₂ | —CH₂—CH₂—C₆H₅ | CH₂—CH₃ |
| B-388 | CH₂F | CHF₂ | —CH₂—CH₂—C₆H₅ | CH₂—CH₃ |
| B-389 | OCH₃ | CHF₂ | —CH₂—CH₂—C₆H₅ | CH₂—CH₃ |
| B-390 | CH₃ | CF₃ | —CH₂—CH₂—C₆H₅ | CH₂—CH₃ |
| B-391 | CH₂F | CF₃ | —CH₂—CH₂—C₆H₅ | CH₂—CH₃ |
| B-392 | OCH₃ | CF₃ | —CH₂—CH₂—C₆H₅ | CH₂—CH₃ |
| B-393 | CH₃ | CH₃ | C₆H₅ | CH₂—CH₂—CH₃ |
| B-394 | CH₂F | CH₃ | C₆H₅ | CH₂—CH₂—CH₃ |
| B-395 | OCH₃ | CH₃ | C₆H₅ | CH₂—CH₂—CH₃ |
| B-396 | CH₃ | CH₂F | C₆H₅ | CH₂—CH₂—CH₃ |
| B-397 | CH₂F | CH₂F | C₆H₅ | CH₂—CH₂—CH₃ |
| B-398 | OCH₃ | CH₂F | C₆H₅ | CH₂—CH₂—CH₃ |
| B-399 | CH₃ | CHF₂ | C₆H₅ | CH₂—CH₂—CH₃ |
| B-400 | CH₂F | CHF₂ | C₆H₅ | CH₂—CH₂—CH₃ |
| B-401 | OCH₃ | CHF₂ | C₆H₅ | CH₂—CH₂—CH₃ |
| B-402 | CH₃ | CF₃ | C₆H₅ | CH₂—CH₂—CH₃ |
| B-403 | CH₂F | CF₃ | C₆H₅ | CH₂—CH₂—CH₃ |
| B-404 | OCH₃ | CF₃ | C₆H₅ | CH₂—CH₂—CH₃ |
| B-405 | CH₃ | CH₃ | —CH₂—C₆H₅ | CH₂—CH₂—CH₃ |
| B-406 | CH₂F | CH₃ | —CH₂—C₆H₅ | CH₂—CH₂—CH₃ |
| B-407 | OCH₃ | CH₃ | —CH₂—C₆H₅ | CH₂—CH₂—CH₃ |
| B-408 | CH₃ | CH₂F | —CH₂—C₆H₅ | CH₂—CH₂—CH₃ |
| B-409 | CH₂F | CH₂F | —CH₂—C₆H₅ | CH₂—CH₂—CH₃ |
| B-410 | OCH₃ | CH₂F | —CH₂—C₆H₅ | CH₂—CH₂—CH₃ |
| B-411 | CH₃ | CHF₂ | —CH₂—C₆H₅ | CH₂—CH₂—CH₃ |
| B-412 | CH₂F | CHF₂ | —CH₂—C₆H₅ | CH₂—CH₂—CH₃ |
| B-413 | OCH₃ | CHF₂ | —CH₂—C₆H₅ | CH₂—CH₂—CH₃ |
| B-414 | CH₃ | CF₃ | —CH₂—C₆H₅ | CH₂—CH₂—CH₃ |
| B-415 | CH₂F | CF₃ | —CH₂—C₆H₅ | CH₂—CH₂—CH₃ |
| B-416 | OCH₃ | CF₃ | —CH₂—C₆H₅ | CH₂—CH₂—CH₃ |
| B-417 | CH₃ | CH₃ | —CH₂-(2-F—C₆H₄) | CH₂—CH₂—CH₃ |
| B-418 | CH₂F | CH₃ | —CH₂-(2-F—C₆H₄) | CH₂—CH₂—CH₃ |
| B-419 | OCH₃ | CH₃ | —CH₂-(2-F—C₆H₄) | CH₂—CH₂—CH₃ |
| B-420 | CH₃ | CH₂F | —CH₂-(2-F—C₆H₄) | CH₂—CH₂—CH₃ |
| B-421 | CH₂F | CH₂F | —CH₂-(2-F—C₆H₄) | CH₂—CH₂—CH₃ |
| B-422 | OCH₃ | CH₂F | —CH₂-(2-F—C₆H₄) | CH₂—CH₂—CH₃ |
| B-423 | CH₃ | CHF₂ | —CH₂-(2-F—C₆H₄) | CH₂—CH₂—CH₃ |
| B-424 | CH₂F | CHF₂ | —CH₂-(2-F—C₆H₄) | CH₂—CH₂—CH₃ |
| B-425 | OCH₃ | CHF₂ | —CH₂-(2-F—C₆H₄) | CH₂—CH₂—CH₃ |
| B-426 | CH₃ | CF₃ | —CH₂-(2-F—C₆H₄) | CH₂—CH₂—CH₃ |
| B-427 | CH₂F | CF₃ | —CH₂-(2-F—C₆H₄) | CH₂—CH₂—CH₃ |
| B-428 | OCH₃ | CF₃ | —CH₂-(2-F—C₆H₄) | CH₂—CH₂—CH₃ |
| B-429 | CH₃ | CH₃ | —CH₂-(3-F—C₆H₄) | CH₂—CH₂—CH₃ |
| B-430 | CH₂F | CH₃ | —CH₂-(3-F—C₆H₄) | CH₂—CH₂—CH₃ |
| B-431 | OCH₃ | CH₃ | —CH₂-(3-F—C₆H₄) | CH₂—CH₂—CH₃ |
| B-432 | CH₃ | CH₂F | —CH₂-(3-F—C₆H₄) | CH₂—CH₂—CH₃ |
| B-433 | CH₂F | CH₂F | —CH₂-(3-F—C₆H₄) | CH₂—CH₂—CH₃ |
| B-434 | OCH₃ | CH₂F | —CH₂-(3-F—C₆H₄) | CH₂—CH₂—CH₃ |
| B-435 | CH₃ | CHF₂ | —CH₂-(3-F—C₆H₄) | CH₂—CH₂—CH₃ |
| B-436 | CH₂F | CHF₂ | —CH₂-(3-F—C₆H₄) | CH₂—CH₂—CH₃ |
| B-437 | OCH₃ | CHF₂ | —CH₂-(3-F—C₆H₄) | CH₂—CH₂—CH₃ |
| B-438 | CH₃ | CF₃ | —CH₂-(3-F—C₆H₄) | CH₂—CH₂—CH₃ |
| B-439 | CH₂F | CF₃ | —CH₂-(3-F—C₆H₄) | CH₂—CH₂—CH₃ |
| B-440 | OCH₃ | CF₃ | —CH₂-(3-F—C₆H₄) | CH₂—CH₂—CH₃ |
| B-441 | CH₃ | CH₃ | —CH₂-(4-F—C₆H₄) | CH₂—CH₂—CH₃ |
| B-442 | CH₂F | CH₃ | —CH₂-(4-F—C₆H₄) | CH₂—CH₂—CH₃ |
| B-443 | OCH₃ | CH₃ | —CH₂-(4-F—C₆H₄) | CH₂—CH₂—CH₃ |
| B-444 | CH₃ | CH₂F | —CH₂-(4-F—C₆H₄) | CH₂—CH₂—CH₃ |
| B-445 | CH₂F | CH₂F | —CH₂-(4-F—C₆H₄) | CH₂—CH₂—CH₃ |
| B-446 | OCH₃ | CH₂F | —CH₂-(4-F—C₆H₄) | CH₂—CH₂—CH₃ |
| B-447 | CH₃ | CHF₂ | —CH₂-(4-F—C₆H₄) | CH₂—CH₂—CH₃ |
| B-448 | CH₂F | CHF₂ | —CH₂-(4-F—C₆H₄) | CH₂—CH₂—CH₃ |
| B-449 | OCH₃ | CHF₂ | —CH₂-(4-F—C₆H₄) | CH₂—CH₂—CH₃ |
| B-450 | CH₃ | CF₃ | —CH₂-(4-F—C₆H₄) | CH₂—CH₂—CH₃ |
| B-451 | CH₂F | CF₃ | —CH₂-(4-F—C₆H₄) | CH₂—CH₂—CH₃ |
| B-452 | OCH₃ | CF₃ | —CH₂-(4-F—C₆H₄) | CH₂—CH₂—CH₃ |
| B-453 | CH₃ | CH₃ | —CH₂-(2,4-F₂—C₆H₃) | CH₂—CH₂—CH₃ |
| B-454 | CH₂F | CH₃ | —CH₂-(2,4-F₂—C₆H₃) | CH₂—CH₂—CH₃ |
| B-455 | OCH₃ | CH₃ | —CH₂-(2,4-F₂—C₆H₃) | CH₂—CH₂—CH₃ |
| B-456 | CH₃ | CH₂F | —CH₂-(2,4-F₂—C₆H₃) | CH₂—CH₂—CH₃ |
| B-457 | CH₂F | CH₂F | —CH₂-(2,4-F₂—C₆H₃) | CH₂—CH₂—CH₃ |
| B-458 | OCH₃ | CH₂F | —CH₂-(2,4-F₂—C₆H₃) | CH₂—CH₂—CH₃ |
| B-459 | CH₃ | CHF₂ | —CH₂-(2,4-F₂—C₆H₃) | CH₂—CH₂—CH₃ |
| B-460 | CH₂F | CHF₂ | —CH₂-(2,4-F₂—C₆H₃) | CH₂—CH₂—CH₃ |
| B-461 | OCH₃ | CHF₂ | —CH₂-(2,4-F₂—C₆H₃) | CH₂—CH₂—CH₃ |
| B-462 | CH₃ | CF₃ | —CH₂-(2,4-F₂—C₆H₃) | CH₂—CH₂—CH₃ |
| B-463 | CH₂F | CF₃ | —CH₂-(2,4-F₂—C₆H₃) | CH₂—CH₂—CH₃ |
| B-464 | OCH₃ | CF₃ | —CH₂-(2,4-F₂—C₆H₃) | CH₂—CH₂—CH₃ |
| B-465 | CH₃ | CH₃ | —CH₂-(2-Cl—C₆H₄) | CH₂—CH₂—CH₃ |
| B-466 | CH₂F | CH₃ | —CH₂-(2-Cl—C₆H₄) | CH₂—CH₂—CH₃ |
| B-467 | OCH₃ | CH₃ | —CH₂-(2-Cl—C₆H₄) | CH₂—CH₂—CH₃ |
| B-468 | CH₃ | CH₂F | —CH₂-(2-Cl—C₆H₄) | CH₂—CH₂—CH₃ |
| B-469 | CH₂F | CH₂F | —CH₂-(2-Cl—C₆H₄) | CH₂—CH₂—CH₃ |
| B-470 | OCH₃ | CH₂F | —CH₂-(2-Cl—C₆H₄) | CH₂—CH₂—CH₃ |
| B-471 | CH₃ | CHF₂ | —CH₂-(2-Cl—C₆H₄) | CH₂—CH₂—CH₃ |
| B-472 | CH₂F | CHF₂ | —CH₂-(2-Cl—C₆H₄) | CH₂—CH₂—CH₃ |
| B-473 | OCH₃ | CHF₂ | —CH₂-(2-Cl—C₆H₄) | CH₂—CH₂—CH₃ |
| B-474 | CH₃ | CF₃ | —CH₂-(2-Cl—C₆H₄) | CH₂—CH₂—CH₃ |
| B-475 | CH₂F | CF₃ | —CH₂-(2-Cl—C₆H₄) | CH₂—CH₂—CH₃ |
| B-476 | OCH₃ | CF₃ | —CH₂-(2-Cl—C₆H₄) | CH₂—CH₂—CH₃ |
| B-477 | CH₃ | CH₃ | —CH₂-(3-Cl—C₆H₄) | CH₂—CH₂—CH₃ |
| B-478 | CH₂F | CH₃ | —CH₂-(3-Cl—C₆H₄) | CH₂—CH₂—CH₃ |
| B-479 | OCH₃ | CH₃ | —CH₂-(3-Cl—C₆H₄) | CH₂—CH₂—CH₃ |
| B-480 | CH₃ | CH₂F | —CH₂-(3-Cl—C₆H₄) | CH₂—CH₂—CH₃ |
| B-481 | CH₂F | CH₂F | —CH₂-(3-Cl—C₆H₄) | CH₂—CH₂—CH₃ |
| B-482 | OCH₃ | CH₂F | —CH₂-(3-Cl—C₆H₄) | CH₂—CH₂—CH₃ |
| B-483 | CH₃ | CHF₂ | —CH₂-(3-Cl—C₆H₄) | CH₂—CH₂—CH₃ |
| B-484 | CH₂F | CHF₂ | —CH₂-(3-Cl—C₆H₄) | CH₂—CH₂—CH₃ |
| B-485 | OCH₃ | CHF₂ | —CH₂-(3-Cl—C₆H₄) | CH₂—CH₂—CH₃ |
| B-486 | CH₃ | CF₃ | —CH₂-(3-Cl—C₆H₄) | CH₂—CH₂—CH₃ |
| B-487 | CH₂F | CF₃ | —CH₂-(3-Cl—C₆H₄) | CH₂—CH₂—CH₃ |
| B-488 | OCH₃ | CF₃ | —CH₂-(3-Cl—C₆H₄) | CH₂—CH₂—CH₃ |
| B-489 | CH₃ | CH₃ | —CH₂-(4-Cl—C₆H₄) | CH₂—CH₂—CH₃ |
| B-490 | CH₂F | CH₃ | —CH₂-(4-Cl—C₆H₄) | CH₂—CH₂—CH₃ |
| B-491 | OCH₃ | CH₃ | —CH₂-(4-Cl—C₆H₄) | CH₂—CH₂—CH₃ |
| B-492 | CH₃ | CH₂F | —CH₂-(4-Cl—C₆H₄) | CH₂—CH₂—CH₃ |
| B-493 | CH₂F | CH₂F | —CH₂-(4-Cl—C₆H₄) | CH₂—CH₂—CH₃ |
| B-494 | OCH₃ | CH₂F | —CH₂-(4-Cl—C₆H₄) | CH₂—CH₂—CH₃ |
| B-495 | CH₃ | CHF₂ | —CH₂-(4-Cl—C₆H₄) | CH₂—CH₂—CH₃ |
| B-496 | CH₂F | CHF₂ | —CH₂-(4-Cl—C₆H₄) | CH₂—CH₂—CH₃ |
| B-497 | OCH₃ | CHF₂ | —CH₂-(4-Cl—C₆H₄) | CH₂—CH₂—CH₃ |
| B-498 | CH₃ | CF₃ | —CH₂-(4-Cl—C₆H₄) | CH₂—CH₂—CH₃ |
| B-499 | CH₂F | CF₃ | —CH₂-(4-Cl—C₆H₄) | CH₂—CH₂—CH₃ |
| B-500 | OCH₃ | CF₃ | —CH₂-(4-Cl—C₆H₄) | CH₂—CH₂—CH₃ |
| B-501 | CH₃ | CH₃ | —CH₂-(2,4-Cl₂—C₆H₃) | CH₂—CH₂—CH₃ |
| B-502 | CH₂F | CH₃ | —CH₂-(2,4-Cl₂—C₆H₃) | CH₂—CH₂—CH₃ |
| B-503 | OCH₃ | CH₃ | —CH₂-(2,4-Cl₂—C₆H₃) | CH₂—CH₂—CH₃ |
| B-504 | CH₃ | CH₂F | —CH₂-(2,4-Cl₂—C₆H₃) | CH₂—CH₂—CH₃ |
| B-505 | CH₂F | CH₂F | —CH₂-(2,4-Cl₂—C₆H₃) | CH₂—CH₂—CH₃ |
| B-506 | OCH₃ | CH₂F | —CH₂-(2,4-Cl₂—C₆H₃) | CH₂—CH₂—CH₃ |
| B-507 | CH₃ | CHF₂ | —CH₂-(2,4-Cl₂—C₆H₃) | CH₂—CH₂—CH₃ |
| B-508 | CH₂F | CHF₂ | —CH₂-(2,4-Cl₂—C₆H₃) | CH₂—CH₂—CH₃ |
| B-509 | OCH₃ | CHF₂ | —CH₂-(2,4-Cl₂—C₆H₃) | CH₂—CH₂—CH₃ |
| B-510 | CH₃ | CF₃ | —CH₂-(2,4-Cl₂—C₆H₃) | CH₂—CH₂—CH₃ |
| B-511 | CH₂F | CF₃ | —CH₂-(2,4-Cl₂—C₆H₃) | CH₂—CH₂—CH₃ |
| B-512 | OCH₃ | CF₃ | —CH₂-(2,4-Cl₂—C₆H₃) | CH₂—CH₂—CH₃ |
| B-513 | CH₃ | CH₃ | —CH₂—CH₂—C₆H₅ | CH₂—CH₂—CH₃ |
| B-514 | CH₂F | CH₃ | —CH₂—CH₂—C₆H₅ | CH₂—CH₂—CH₃ |
| B-515 | OCH₃ | CH₃ | —CH₂—CH₂—C₆H₅ | CH₂—CH₂—CH₃ |
| B-516 | CH₃ | CH₂F | —CH₂—CH₂—C₆H₅ | CH₂—CH₂—CH₃ |
| B-517 | CH₂F | CH₂F | —CH₂—CH₂—C₆H₅ | CH₂—CH₂—CH₃ |
| B-518 | OCH₃ | CH₂F | —CH₂—CH₂—C₆H₅ | CH₂—CH₂—CH₃ |
| B-519 | CH₃ | CHF₂ | —CH₂—CH₂—C₆H₅ | CH₂—CH₂—CH₃ |
| B-520 | CH₂F | CHF₂ | —CH₂—CH₂—C₆H₅ | CH₂—CH₂—CH₃ |
| B-521 | OCH₃ | CHF₂ | —CH₂—CH₂—C₆H₅ | CH₂—CH₂—CH₃ |
| B-522 | CH₃ | CF₃ | —CH₂—CH₂—C₆H₅ | CH₂—CH₂—CH₃ |
| B-523 | CH₂F | CF₃ | —CH₂—CH₂—C₆H₅ | CH₂—CH₂—CH₃ |
| B-524 | OCH₃ | CF₃ | —CH₂—CH₂—C₆H₅ | CH₂—CH₂—CH₃ |
| B-525 | CH₃ | CH₃ | C₆H₅ | —CH(CH₃)₂ |
| B-526 | CH₂F | CH₃ | C₆H₅ | —CH(CH₃)₂ |
| B-527 | OCH₃ | CH₃ | C₆H₅ | —CH(CH₃)₂ |
| B-528 | CH₃ | CH₂F | C₆H₅ | —CH(CH₃)₂ |
| B-529 | CH₂F | CH₂F | C₆H₅ | —CH(CH₃)₂ |
| B-530 | OCH₃ | CH₂F | C₆H₅ | —CH(CH₃)₂ |
| B-531 | CH₃ | CHF₂ | C₆H₅ | —CH(CH₃)₂ |
| B-532 | CH₂F | CHF₂ | C₆H₅ | —CH(CH₃)₂ |
| B-533 | OCH₃ | CHF₂ | C₆H₅ | —CH(CH₃)₂ |
| B-534 | CH₃ | CF₃ | C₆H₅ | —CH(CH₃)₂ |
| B-535 | CH₂F | CF₃ | C₆H₅ | —CH(CH₃)₂ |
| B-536 | OCH₃ | CF₃ | C₆H₅ | —CH(CH₃)₂ |
| B-537 | CH₃ | CH₃ | —CH₂—C₆H₅ | —CH(CH₃)₂ |
| B-538 | CH₂F | CH₃ | —CH₂—C₆H₅ | —CH(CH₃)₂ |
| B-539 | OCH₃ | CH₃ | —CH₂—C₆H₅ | —CH(CH₃)₂ |
| B-540 | CH₃ | CH₂F | —CH₂—C₆H₅ | —CH(CH₃)₂ |
| B-541 | CH₂F | CH₂F | —CH₂—C₆H₅ | —CH(CH₃)₂ |
| B-542 | OCH₃ | CH₂F | —CH₂—C₆H₅ | —CH(CH₃)₂ |
| B-543 | CH₃ | CHF₂ | —CH₂—C₆H₅ | —CH(CH₃)₂ |
| B-544 | CH₂F | CHF₂ | —CH₂—C₆H₅ | —CH(CH₃)₂ |
| B-545 | OCH₃ | CHF₂ | —CH₂—C₆H₅ | —CH(CH₃)₂ |
| B-546 | CH₃ | CF₃ | —CH₂—C₆H₅ | —CH(CH₃)₂ |
| B-547 | CH₂F | CF₃ | —CH₂—C₆H₅ | —CH(CH₃)₂ |
| B-548 | OCH₃ | CF₃ | —CH₂—C₆H₅ | —CH(CH₃)₂ |
| B-549 | CH₃ | CH₃ | —CH₂-(2-F—C₆H₄) | —CH(CH₃)₂ |
| B-550 | CH₂F | CH₃ | —CH₂-(2-F—C₆H₄) | —CH(CH₃)₂ |
| B-551 | OCH₃ | CH₃ | —CH₂-(2-F—C₆H₄) | —CH(CH₃)₂ |
| B-552 | CH₃ | CH₂F | —CH₂-(2-F—C₆H₄) | —CH(CH₃)₂ |
| B-553 | CH₂F | CH₂F | —CH₂-(2-F—C₆H₄) | —CH(CH₃)₂ |
| B-554 | OCH₃ | CH₂F | —CH₂-(2-F—C₆H₄) | —CH(CH₃)₂ |
| B-555 | CH₃ | CHF₂ | —CH₂-(2-F—C₆H₄) | —CH(CH₃)₂ |
| B-556 | CH₂F | CHF₂ | —CH₂-(2-F—C₆H₄) | —CH(CH₃)₂ |
| B-557 | OCH₃ | CHF₂ | —CH₂-(2-F—C₆H₄) | —CH(CH₃)₂ |
| B-558 | CH₃ | CF₃ | —CH₂-(2-F—C₆H₄) | —CH(CH₃)₂ |
| B-559 | CH₂F | CF₃ | —CH₂-(2-F—C₆H₄) | —CH(CH₃)₂ |
| B-560 | OCH₃ | CF₃ | —CH₂-(2-F—C₆H₄) | —CH(CH₃)₂ |
| B-561 | CH₃ | CH₃ | —CH₂-(3-F—C₆H₄) | —CH(CH₃)₂ |
| B-562 | CH₂F | CH₃ | —CH₂-(3-F—C₆H₄) | —CH(CH₃)₂ |
| B-563 | OCH₃ | CH₃ | —CH₂-(3-F—C₆H₄) | —CH(CH₃)₂ |
| B-564 | CH₃ | CH₂F | —CH₂-(3-F—C₆H₄) | —CH(CH₃)₂ |
| B-565 | CH₂F | CH₂F | —CH₂-(3-F—C₆H₄) | —CH(CH₃)₂ |
| B-566 | OCH₃ | CH₂F | —CH₂-(3-F—C₆H₄) | —CH(CH₃)₂ |
| B-567 | CH₃ | CHF₂ | —CH₂-(3-F—C₆H₄) | —CH(CH₃)₂ |
| B-568 | CH₂F | CHF₂ | —CH₂-(3-F—C₆H₄) | —CH(CH₃)₂ |
| B-569 | OCH₃ | CHF₂ | —CH₂-(3-F—C₆H₄) | —CH(CH₃)₂ |
| B-570 | CH₃ | CF₃ | —CH₂-(3-F—C₆H₄) | —CH(CH₃)₂ |
| B-571 | CH₂F | CF₃ | —CH₂-(3-F—C₆H₄) | —CH(CH₃)₂ |
| B-572 | OCH₃ | CF₃ | —CH₂-(3-F—C₆H₄) | —CH(CH₃)₂ |
| B-573 | CH₃ | CH₃ | —CH₂-(4-F—C₆H₄) | —CH(CH₃)₂ |
| B-574 | CH₂F | CH₃ | —CH₂-(4-F—C₆H₄) | —CH(CH₃)₂ |
| B-575 | OCH₃ | CH₃ | —CH₂-(4-F—C₆H₄) | —CH(CH₃)₂ |
| B-576 | CH₃ | CH₂F | —CH₂-(4-F—C₆H₄) | —CH(CH₃)₂ |
| B-577 | CH₂F | CH₂F | —CH₂-(4-F—C₆H₄) | —CH(CH₃)₂ |
| B-578 | OCH₃ | CH₂F | —CH₂-(4-F—C₆H₄) | —CH(CH₃)₂ |
| B-579 | CH₃ | CHF₂ | —CH₂-(4-F—C₆H₄) | —CH(CH₃)₂ |
| B-580 | CH₂F | CHF₂ | —CH₂-(4-F—C₆H₄) | —CH(CH₃)₂ |
| B-581 | OCH₃ | CHF₂ | —CH₂-(4-F—C₆H₄) | —CH(CH₃)₂ |
| B-582 | CH₃ | CF₃ | —CH₂-(4-F—C₆H₄) | —CH(CH₃)₂ |
| B-583 | CH₂F | CF₃ | —CH₂-(4-F—C₆H₄) | —CH(CH₃)₂ |
| B-584 | OCH₃ | CF₃ | —CH₂-(4-F—C₆H₄) | —CH(CH₃)₂ |
| B-585 | CH₃ | CH₃ | —CH₂-(2,4-F₂—C₆H₃) | —CH(CH₃)₂ |
| B-586 | CH₂F | CH₃ | —CH₂-(2,4-F₂—C₆H₃) | —CH(CH₃)₂ |
| B-587 | OCH₃ | CH₃ | —CH₂-(2,4-F₂—C₆H₃) | —CH(CH₃)₂ |
| B-588 | CH₃ | CH₂F | —CH₂-(2,4-F₂—C₆H₃) | —CH(CH₃)₂ |
| B-589 | CH₂F | CH₂F | —CH₂-(2,4-F₂—C₆H₃) | —CH(CH₃)₂ |
| B-590 | OCH₃ | CH₂F | —CH₂-(2,4-F₂—C₆H₃) | —CH(CH₃)₂ |
| B-591 | CH₃ | CHF₂ | —CH₂-(2,4-F₂—C₆H₃) | —CH(CH₃)₂ |
| B-592 | CH₂F | CHF₂ | —CH₂-(2,4-F₂—C₆H₃) | —CH(CH₃)₂ |
| B-593 | OCH₃ | CHF₂ | —CH₂-(2,4-F₂—C₆H₃) | —CH(CH₃)₂ |
| B-594 | CH₃ | CF₃ | —CH₂-(2,4-F₂—C₆H₃) | —CH(CH₃)₂ |
| B-595 | CH₂F | CF₃ | —CH₂-(2,4-F₂—C₆H₃) | —CH(CH₃)₂ |
| B-596 | OCH₃ | CF₃ | —CH₂-(2,4-F₂—C₆H₃) | —CH(CH₃)₂ |
| B-597 | CH₃ | CH₃ | —CH₂-(2-Cl—C₆H₄) | —CH(CH₃)₂ |
| B-598 | CH₂F | CH₃ | —CH₂-(2-Cl—C₆H₄) | —CH(CH₃)₂ |
| B-599 | OCH₃ | CH₃ | —CH₂-(2-Cl—C₆H₄) | —CH(CH₃)₂ |
| B-600 | CH₃ | CH₂F | —CH₂-(2-Cl—C₆H₄) | —CH(CH₃)₂ |
| B-601 | CH₂F | CH₂F | —CH₂-(2-Cl—C₆H₄) | —CH(CH₃)₂ |
| B-602 | OCH₃ | CH₂F | —CH₂-(2-Cl—C₆H₄) | —CH(CH₃)₂ |
| B-603 | CH₃ | CHF₂ | —CH₂-(2-Cl—C₆H₄) | —CH(CH₃)₂ |
| B-604 | CH₂F | CHF₂ | —CH₂-(2-Cl—C₆H₄) | —CH(CH₃)₂ |
| B-605 | OCH₃ | CHF₂ | —CH₂-(2-Cl—C₆H₄) | —CH(CH₃)₂ |
| B-606 | CH₃ | CF₃ | —CH₂-(2-Cl—C₆H₄) | —CH(CH₃)₂ |
| B-607 | CH₂F | CF₃ | —CH₂-(2-Cl—C₆H₄) | —CH(CH₃)₂ |
| B-608 | OCH₃ | CF₃ | —CH₂-(2-Cl—C₆H₄) | —CH(CH₃)₂ |
| B-609 | CH₃ | CH₃ | —CH₂-(3-Cl—C₆H₄) | —CH(CH₃)₂ |
| B-610 | CH₂F | CH₃ | —CH₂-(3-Cl—C₆H₄) | —CH(CH₃)₂ |
| B-611 | OCH₃ | CH₃ | —CH₂-(3-Cl—C₆H₄) | —CH(CH₃)₂ |
| B-612 | CH₃ | CH₂F | —CH₂-(3-Cl—C₆H₄) | —CH(CH₃)₂ |
| B-613 | CH₂F | CH₂F | —CH₂-(3-Cl—C₆H₄) | —CH(CH₃)₂ |
| B-614 | OCH₃ | CH₂F | —CH₂-(3-Cl—C₆H₄) | —CH(CH₃)₂ |
| B-615 | CH₃ | CHF₂ | —CH₂-(3-Cl—C₆H₄) | —CH(CH₃)₂ |
| B-616 | CH₂F | CHF₂ | —CH₂-(3-Cl—C₆H₄) | —CH(CH₃)₂ |
| B-617 | OCH₃ | CHF₂ | —CH₂-(3-Cl—C₆H₄) | —CH(CH₃)₂ |
| B-618 | CH₃ | CF₃ | —CH₂-(3-Cl—C₆H₄) | —CH(CH₃)₂ |
| B-619 | CH₂F | CF₃ | —CH₂-(3-Cl—C₆H₄) | —CH(CH₃)₂ |
| B-620 | OCH₃ | CF₃ | —CH₂-(3-Cl—C₆H₄) | —CH(CH₃)₂ |
| B-621 | CH₃ | CH₃ | —CH₂-(4-Cl—C₆H₄) | —CH(CH₃)₂ |
| B-622 | CH₂F | CH₃ | —CH₂-(4-Cl—C₆H₄) | —CH(CH₃)₂ |
| B-623 | OCH₃ | CH₃ | —CH₂-(4-Cl—C₆H₄) | —CH(CH₃)₂ |
| B-624 | CH₃ | CH₂F | —CH₂-(4-Cl—C₆H₄) | —CH(CH₃)₂ |
| B-625 | CH₂F | CH₂F | —CH₂-(4-Cl—C₆H₄) | —CH(CH₃)₂ |
| B-626 | OCH₃ | CH₂F | —CH₂-(4-Cl—C₆H₄) | —CH(CH₃)₂ |
| B-627 | CH₃ | CHF₂ | —CH₂-(4-Cl—C₆H₄) | —CH(CH₃)₂ |
| B-628 | CH₂F | CHF₂ | —CH₂-(4-Cl—C₆H₄) | —CH(CH₃)₂ |
| B-629 | OCH₃ | CHF₂ | —CH₂-(4-Cl—C₆H₄) | —CH(CH₃)₂ |
| B-630 | CH₃ | CF₃ | —CH₂-(4-Cl—C₆H₄) | —CH(CH₃)₂ |
| B-631 | CH₂F | CF₃ | —CH₂-(4-Cl—C₆H₄) | —CH(CH₃)₂ |
| B-632 | OCH₃ | CF₃ | —CH₂-(4-Cl—C₆H₄) | —CH(CH₃)₂ |
| B-633 | CH₃ | CH₃ | —CH₂-(2,4-Cl₂—C₆H₃) | —CH(CH₃)₂ |
| B-634 | CH₂F | CH₃ | —CH₂-(2,4-Cl₂—C₆H₃) | —CH(CH₃)₂ |
| B-635 | OCH₃ | CH₃ | —CH₂-(2,4-Cl₂—C₆H₃) | —CH(CH₃)₂ |
| B-636 | CH₃ | CH₂F | —CH₂-(2,4-Cl₂—C₆H₃) | —CH(CH₃)₂ |
| B-637 | CH₂F | CH₂F | —CH₂-(2,4-Cl₂—C₆H₃) | —CH(CH₃)₂ |
| B-638 | OCH₃ | CH₂F | —CH₂-(2,4-Cl₂—C₆H₃) | —CH(CH₃)₂ |
| B-639 | CH₃ | CHF₂ | —CH₂-(2,4-Cl₂—C₆H₃) | —CH(CH₃)₂ |
| B-640 | CH₂F | CHF₂ | —CH₂-(2,4-Cl₂—C₆H₃) | —CH(CH₃)₂ |
| B-641 | OCH₃ | CHF₂ | —CH₂-(2,4-Cl₂—C₆H₃) | —CH(CH₃)₂ |
| B-642 | CH₃ | CF₃ | —CH₂-(2,4-Cl₂—C₆H₃) | —CH(CH₃)₂ |
| B-643 | CH₂F | CF₃ | —CH₂-(2,4-Cl₂—C₆H₃) | —CH(CH₃)₂ |
| B-644 | OCH₃ | CF₃ | —CH₂-(2,4-Cl₂—C₆H₃) | —CH(CH₃)₂ |
| B-645 | CH₃ | CH₃ | —CH₂—CH₂—C₆H₅ | —CH(CH₃)₂ |
| B-646 | CH₂F | CH₃ | —CH₂—CH₂—C₆H₅ | —CH(CH₃)₂ |
| B-647 | OCH₃ | CH₃ | —CH₂—CH₂—C₆H₅ | —CH(CH₃)₂ |
| B-648 | CH₃ | CH₂F | —CH₂—CH₂—C₆H₅ | —CH(CH₃)₂ |
| B-649 | CH₂F | CH₂F | —CH₂—CH₂—C₆H₅ | —CH(CH₃)₂ |
| B-650 | OCH₃ | CH₂F | —CH₂—CH₂—C₆H₅ | —CH(CH₃)₂ |
| B-651 | CH₃ | CHF₂ | —CH₂—CH₂—C₆H₅ | —CH(CH₃)₂ |
| B-652 | CH₂F | CHF₂ | —CH₂—CH₂—C₆H₅ | —CH(CH₃)₂ |
| B-653 | OCH₃ | CHF₂ | —CH₂—CH₂—C₆H₅ | —CH(CH₃)₂ |
| B-654 | CH₃ | CF₃ | —CH₂—CH₂—C₆H₅ | —CH(CH₃)₂ |
| B-655 | CH₂F | CF₃ | —CH₂—CH₂—C₆H₅ | —CH(CH₃)₂ |
| B-656 | OCH₃ | CF₃ | —CH₂—CH₂—C₆H₅ | —CH(CH₃)₂ |
| B-657 | CH₃ | CH₃ | C₆H₅ | —(CH₂)₃—CH₃ |
| B-658 | CH₂F | CH₃ | C₆H₅ | —(CH₂)₃—CH₃ |
| B-659 | OCH₃ | CH₃ | C₆H₅ | —(CH₂)₃—CH₃ |
| B-660 | CH₃ | CH₂F | C₆H₅ | —(CH₂)₃—CH₃ |
| B-661 | CH₂F | CH₂F | C₆H₅ | —(CH₂)₃—CH₃ |
| B-662 | OCH₃ | CH₂F | C₆H₅ | —(CH₂)₃—CH₃ |
| B-663 | CH₃ | CHF₂ | C₆H₅ | —(CH₂)₃—CH₃ |
| B-664 | CH₂F | CHF₂ | C₆H₅ | —(CH₂)₃—CH₃ |
| B-665 | OCH₃ | CHF₂ | C₆H₅ | —(CH₂)₃—CH₃ |
| B-666 | CH₃ | CF₃ | C₆H₅ | —(CH₂)₃—CH₃ |
| B-667 | CH₂F | CF₃ | C₆H₅ | —(CH₂)₃—CH₃ |
| B-668 | OCH₃ | CF₃ | C₆H₅ | —(CH₂)₃—CH₃ |
| B-669 | CH₃ | CH₃ | —CH₂—C₆H₅ | —(CH₂)₃—CH₃ |
| B-670 | CH₂F | CH₃ | —CH₂—C₆H₅ | —(CH₂)₃—CH₃ |
| B-671 | OCH₃ | CH₃ | —CH₂—C₆H₅ | —(CH₂)₃—CH₃ |
| B-672 | CH₃ | CH₂F | —CH₂—C₆H₅ | —(CH₂)₃—CH₃ |
| B-673 | CH₂F | CH₂F | —CH₂—C₆H₅ | —(CH₂)₃—CH₃ |
| B-674 | OCH₃ | CH₂F | —CH₂—C₆H₅ | —(CH₂)₃—CH₃ |
| B-675 | CH₃ | CHF₂ | —CH₂—C₆H₅ | —(CH₂)₃—CH₃ |
| B-676 | CH₂F | CHF₂ | —CH₂—C₆H₅ | —(CH₂)₃—CH₃ |
| B-677 | OCH₃ | CHF₂ | —CH₂—C₆H₅ | —(CH₂)₃—CH₃ |
| B-678 | CH₃ | CF₃ | —CH₂—C₆H₅ | —(CH₂)₃—CH₃ |
| B-679 | CH₂F | CF₃ | —CH₂—C₆H₅ | —(CH₂)₃—CH₃ |
| B-680 | OCH₃ | CF₃ | —CH₂—C₆H₅ | —(CH₂)₃—CH₃ |
| B-681 | CH₃ | CH₃ | —CH₂-(2-F—C₆H₄) | —(CH₂)₃—CH₃ |
| B-682 | CH₂F | CH₃ | —CH₂-(2-F—C₆H₄) | —(CH₂)₃—CH₃ |
| B-683 | OCH₃ | CH₃ | —CH₂-(2-F—C₆H₄) | —(CH₂)₃—CH₃ |
| B-684 | CH₃ | CH₂F | —CH₂-(2-F—C₆H₄) | —(CH₂)₃—CH₃ |
| B-685 | CH₂F | CH₂F | —CH₂-(2-F—C₆H₄) | —(CH₂)₃—CH₃ |
| B-686 | OCH₃ | CH₂F | —CH₂-(2-F—C₆H₄) | —(CH₂)₃—CH₃ |
| B-687 | CH₃ | CHF₂ | —CH₂-(2-F—C₆H₄) | —(CH₂)₃—CH₃ |
| B-688 | CH₂F | CHF₂ | —CH₂-(2-F—C₆H₄) | —(CH₂)₃—CH₃ |
| B-689 | OCH₃ | CHF₂ | —CH₂-(2-F—C₆H₄) | —(CH₂)₃—CH₃ |
| B-690 | CH₃ | CF₃ | —CH₂-(2-F—C₆H₄) | —(CH₂)₃—CH₃ |
| B-691 | CH₂F | CF₃ | —CH₂-(2-F—C₆H₄) | —(CH₂)₃—CH₃ |
| B-692 | OCH₃ | CF₃ | —CH₂-(2-F—C₆H₄) | —(CH₂)₃—CH₃ |
| B-693 | CH₃ | CH₃ | —CH₂-(3-F—C₆H₄) | —(CH₂)₃—CH₃ |
| B-694 | CH₂F | CH₃ | —CH₂-(3-F—C₆H₄) | —(CH₂)₃—CH₃ |
| B-695 | OCH₃ | CH₃ | —CH₂-(3-F—C₆H₄) | —(CH₂)₃—CH₃ |
| B-696 | CH₃ | CH₂F | —CH₂-(3-F—C₆H₄) | —(CH₂)₃—CH₃ |
| B-697 | CH₂F | CH₂F | —CH₂-(3-F—C₆H₄) | —(CH₂)₃—CH₃ |
| B-698 | OCH₃ | CH₂F | —CH₂-(3-F—C₆H₄) | —(CH₂)₃—CH₃ |
| B-699 | CH₃ | CHF₂ | —CH₂-(3-F—C₆H₄) | —(CH₂)₃—CH₃ |
| B-700 | CH₂F | CHF₂ | —CH₂-(3-F—C₆H₄) | —(CH₂)₃—CH₃ |
| B-701 | OCH₃ | CHF₂ | —CH₂-(3-F—C₆H₄) | —(CH₂)₃—CH₃ |
| B-702 | CH₃ | CF₃ | —CH₂-(3-F—C₆H₄) | —(CH₂)₃—CH₃ |
| B-703 | CH₂F | CF₃ | —CH₂-(3-F—C₆H₄) | —(CH₂)₃—CH₃ |
| B-704 | OCH₃ | CF₃ | —CH₂-(3-F—C₆H₄) | —(CH₂)₃—CH₃ |
| B-705 | CH₃ | CH₃ | —CH₂-(4-F—C₆H₄) | —(CH₂)₃—CH₃ |
| B-706 | CH₂F | CH₃ | —CH₂-(4-F—C₆H₄) | —(CH₂)₃—CH₃ |
| B-707 | OCH₃ | CH₃ | —CH₂-(4-F—C₆H₄) | —(CH₂)₃—CH₃ |
| B-708 | CH₃ | CH₂F | —CH₂-(4-F—C₆H₄) | —(CH₂)₃—CH₃ |
| B-709 | CH₂F | CH₂F | —CH₂-(4-F—C₆H₄) | —(CH₂)₃—CH₃ |
| B-710 | OCH₃ | CH₂F | —CH₂-(4-F—C₆H₄) | —(CH₂)₃—CH₃ |
| B-711 | CH₃ | CHF₂ | —CH₂-(4-F—C₆H₄) | —(CH₂)₃—CH₃ |
| B-712 | CH₂F | CHF₂ | —CH₂-(4-F—C₆H₄) | —(CH₂)₃—CH₃ |
| B-713 | OCH₃ | CHF₂ | —CH₂-(4-F—C₆H₄) | —(CH₂)₃—CH₃ |
| B-714 | CH₃ | CF₃ | —CH₂-(4-F—C₆H₄) | —(CH₂)₃—CH₃ |
| B-715 | CH₂F | CF₃ | —CH₂-(4-F—C₆H₄) | —(CH₂)₃—CH₃ |
| B-716 | OCH₃ | CF₃ | —CH₂-(4-F—C₆H₄) | —(CH₂)₃—CH₃ |
| B-717 | CH₃ | CH₃ | —CH₂-(2,4-F₂—C₆H₃) | —(CH₂)₃—CH₃ |
| B-718 | CH₂F | CH₃ | —CH₂-(2,4-F₂—C₆H₃) | —(CH₂)₃—CH₃ |
| B-719 | OCH₃ | CH₃ | —CH₂-(2,4-F₂—C₆H₃) | —(CH₂)₃—CH₃ |
| B-720 | CH₃ | CH₂F | —CH₂-(2,4-F₂—C₆H₃) | —(CH₂)₃—CH₃ |
| B-721 | CH₂F | CH₂F | —CH₂-(2,4-F₂—C₆H₃) | —(CH₂)₃—CH₃ |
| B-722 | OCH₃ | CH₂F | —CH₂-(2,4-F₂—C₆H₃) | —(CH₂)₃—CH₃ |
| B-723 | CH₃ | CHF₂ | —CH₂-(2,4-F₂—C₆H₃) | —(CH₂)₃—CH₃ |
| B-724 | CH₂F | CHF₂ | —CH₂-(2,4-F₂—C₆H₃) | —(CH₂)₃—CH₃ |
| B-725 | OCH₃ | CHF₂ | —CH₂-(2,4-F₂—C₆H₃) | —(CH₂)₃—CH₃ |
| B-726 | CH₃ | CF₃ | —CH₂-(2,4-F₂—C₆H₃) | —(CH₂)₃—CH₃ |
| B-727 | CH₂F | CF₃ | —CH₂-(2,4-F₂—C₆H₃) | —(CH₂)₃—CH₃ |
| B-728 | OCH₃ | CF₃ | —CH₂-(2,4-F₂—C₆H₃) | —(CH₂)₃—CH₃ |
| B-729 | CH₃ | CH₃ | —CH₂-(2-Cl—C₆H₄) | —(CH₂)₃—CH₃ |
| B-730 | CH₂F | CH₃ | —CH₂-(2-Cl—C₆H₄) | —(CH₂)₃—CH₃ |
| B-731 | OCH₃ | CH₃ | —CH₂-(2-Cl—C₆H₄) | —(CH₂)₃—CH₃ |
| B-732 | CH₃ | CH₂F | —CH₂-(2-Cl—C₆H₄) | —(CH₂)₃—CH₃ |
| B-733 | CH₂F | CH₂F | —CH₂-(2-Cl—C₆H₄) | —(CH₂)₃—CH₃ |
| B-734 | OCH₃ | CH₂F | —CH₂-(2-Cl—C₆H₄) | —(CH₂)₃—CH₃ |
| B-735 | CH₃ | CHF₂ | —CH₂-(2-Cl—C₆H₄) | —(CH₂)₃—CH₃ |
| B-736 | CH₂F | CHF₂ | —CH₂-(2-Cl—C₆H₄) | —(CH₂)₃—CH₃ |
| B-737 | OCH₃ | CHF₂ | —CH₂-(2-Cl—C₆H₄) | —(CH₂)₃—CH₃ |
| B-738 | CH₃ | CF₃ | —CH₂-(2-Cl—C₆H₄) | —(CH₂)₃—CH₃ |
| B-739 | CH₂F | CF₃ | —CH₂-(2-Cl—C₆H₄) | —(CH₂)₃—CH₃ |
| B-740 | OCH₃ | CF₃ | —CH₂-(2-Cl—C₆H₄) | —(CH₂)₃—CH₃ |
| B-741 | CH₃ | CH₃ | —CH₂-(3-Cl—C₆H₄) | —(CH₂)₃—CH₃ |
| B-742 | CH₂F | CH₃ | —CH₂-(3-Cl—C₆H₄) | —(CH₂)₃—CH₃ |
| B-743 | OCH₃ | CH₃ | —CH₂-(3-Cl—C₆H₄) | —(CH₂)₃—CH₃ |
| B-744 | CH₃ | CH₂F | —CH₂-(3-Cl—C₆H₄) | —(CH₂)₃—CH₃ |
| B-745 | CH₂F | CH₂F | —CH₂-(3-Cl—C₆H₄) | —(CH₂)₃—CH₃ |
| B-746 | OCH₃ | CH₂F | —CH₂-(3-Cl—C₆H₄) | —(CH₂)₃—CH₃ |
| B-747 | CH₃ | CHF₂ | —CH₂-(3-Cl—C₆H₄) | —(CH₂)₃—CH₃ |
| B-748 | CH₂F | CHF₂ | —CH₂-(3-Cl—C₆H₄) | —(CH₂)₃—CH₃ |
| B-749 | OCH₃ | CHF₂ | —CH₂-(3-Cl—C₆H₄) | —(CH₂)₃—CH₃ |
| B-750 | CH₃ | CF₃ | —CH₂-(3-Cl—C₆H₄) | —(CH₂)₃—CH₃ |
| B-751 | CH₂F | CF₃ | —CH₂-(3-Cl—C₆H₄) | —(CH₂)₃—CH₃ |
| B-752 | OCH₃ | CF₃ | —CH₂-(3-Cl—C₆H₄) | —(CH₂)₃—CH₃ |
| B-753 | CH₃ | CH₃ | —CH₂-(4-Cl—C₆H₄) | —(CH₂)₃—CH₃ |
| B-754 | CH₂F | CH₃ | —CH₂-(4-Cl—C₆H₄) | —(CH₂)₃—CH₃ |
| B-755 | OCH₃ | CH₃ | —CH₂-(4-Cl—C₆H₄) | —(CH₂)₃—CH₃ |
| B-756 | CH₃ | CH₂F | —CH₂-(4-Cl—C₆H₄) | —(CH₂)₃—CH₃ |
| B-757 | CH₂F | CH₂F | —CH₂-(4-Cl—C₆H₄) | —(CH₂)₃—CH₃ |
| B-758 | OCH₃ | CH₂F | —CH₂-(4-Cl—C₆H₄) | —(CH₂)₃—CH₃ |
| B-759 | CH₃ | CHF₂ | —CH₂-(4-Cl—C₆H₄) | —(CH₂)₃—CH₃ |
| B-760 | CH₂F | CHF₂ | —CH₂-(4-Cl—C₆H₄) | —(CH₂)₃—CH₃ |
| B-761 | OCH₃ | CHF₂ | —CH₂-(4-Cl—C₆H₄) | —(CH₂)₃—CH₃ |
| B-762 | CH₃ | CF₃ | —CH₂-(4-Cl—C₆H₄) | —(CH₂)₃—CH₃ |
| B-763 | CH₂F | CF₃ | —CH₂-(4-Cl—C₆H₄) | —(CH₂)₃—CH₃ |
| B-764 | OCH₃ | CF₃ | —CH₂-(4-Cl—C₆H₄) | —(CH₂)₃—CH₃ |
| B-765 | CH₃ | CH₃ | —CH₂-(2,4-Cl₂—C₆H₃) | —(CH₂)₃—CH₃ |
| B-766 | CH₂F | CH₃ | —CH₂-(2,4-Cl₂—C₆H₃) | —(CH₂)₃—CH₃ |
| B-767 | OCH₃ | CH₃ | —CH₂-(2,4-Cl₂—C₆H₃) | —(CH₂)₃—CH₃ |
| B-768 | CH₃ | CH₂F | —CH₂-(2,4-Cl₂—C₆H₃) | —(CH₂)₃—CH₃ |
| B-769 | CH₂F | CH₂F | —CH₂-(2,4-Cl₂—C₆H₃) | —(CH₂)₃—CH₃ |
| B-770 | OCH₃ | CH₂F | —CH₂-(2,4-Cl₂—C₆H₃) | —(CH₂)₃—CH₃ |
| B-771 | CH₃ | CHF₂ | —CH₂-(2,4-Cl₂—C₆H₃) | —(CH₂)₃—CH₃ |
| B-772 | CH₂F | CHF₂ | —CH₂-(2,4-Cl₂—C₆H₃) | —(CH₂)₃—CH₃ |
| B-773 | OCH₃ | CHF₂ | —CH₂-(2,4-Cl₂—C₆H₃) | —(CH₂)₃—CH₃ |
| B-774 | CH₃ | CF₃ | —CH₂-(2,4-Cl₂—C₆H₃) | —(CH₂)₃—CH₃ |
| B-775 | CH₂F | CF₃ | —CH₂-(2,4-Cl₂—C₆H₃) | —(CH₂)₃—CH₃ |
| B-776 | OCH₃ | CF₃ | —CH₂-(2,4-Cl₂—C₆H₃) | —(CH₂)₃—CH₃ |
| B-777 | CH₃ | CH₃ | —CH₂—CH₂—C₆H₅ | —(CH₂)₃—CH₃ |
| B-778 | CH₂F | CH₃ | —CH₂—CH₂—C₆H₅ | —(CH₂)₃—CH₃ |
| B-779 | OCH₃ | CH₃ | —CH₂—CH₂—C₆H₅ | —(CH₂)₃—CH₃ |
| B-780 | CH₃ | CH₂F | —CH₂—CH₂—C₆H₅ | —(CH₂)₃—CH₃ |
| B-781 | CH₂F | CH₂F | —CH₂—CH₂—C₆H₅ | —(CH₂)₃—CH₃ |
| B-782 | OCH₃ | CH₂F | —CH₂—CH₂—C₆H₅ | —(CH₂)₃—CH₃ |
| B-783 | CH₃ | CHF₂ | —CH₂—CH₂—C₆H₅ | —(CH₂)₃—CH₃ |
| B-784 | CH₂F | CHF₂ | —CH₂—CH₂—C₆H₅ | —(CH₂)₃—CH₃ |
| B-785 | OCH₃ | CHF₂ | —CH₂—CH₂—C₆H₅ | —(CH₂)₃—CH₃ |
| B-786 | CH₃ | CF₃ | —CH₂—CH₂—C₆H₅ | —(CH₂)₃—CH₃ |
| B-787 | CH₂F | CF₃ | —CH₂—CH₂—C₆H₅ | —(CH₂)₃—CH₃ |
| B-788 | OCH₃ | CF₃ | —CH₂—CH₂—C₆H₅ | —(CH₂)₃—CH₃ |
| B-789 | CH₃ | CH₃ | C₆H₅ | —CH—CH(CH₃)₂ |
| B-790 | CH₂F | CH₃ | C₆H₅ | —CH—CH(CH₃)₂ |
| B-791 | OCH₃ | CH₃ | C₆H₅ | —CH—CH(CH₃)₂ |
| B-792 | CH₃ | CH₂F | C₆H₅ | —CH—CH(CH₃)₂ |
| B-793 | CH₂F | CH₂F | C₆H₅ | —CH—CH(CH₃)₂ |
| B-794 | OCH₃ | CH₂F | C₆H₅ | —CH—CH(CH₃)₂ |
| B-795 | CH₃ | CHF₂ | C₆H₅ | —CH—CH(CH₃)₂ |
| B-796 | CH₂F | CHF₂ | C₆H₅ | —CH—CH(CH₃)₂ |
| B-797 | OCH₃ | CHF₂ | C₆H₅ | —CH—CH(CH₃)₂ |
| B-798 | CH₃ | CF₃ | C₆H₅ | —CH—CH(CH₃)₂ |
| B-799 | CH₂F | CF₃ | C₆H₅ | —CH—CH(CH₃)₂ |
| B-800 | OCH₃ | CF₃ | C₆H₅ | —CH—CH(CH₃)₂ |
| B-801 | CH₃ | CH₃ | —CH₂—C₆H₅ | —CH—CH(CH₃)₂ |
| B-802 | CH₂F | CH₃ | —CH₂—C₆H₅ | —CH—CH(CH₃)₂ |
| B-803 | OCH₃ | CH₃ | —CH₂—C₆H₅ | —CH—CH(CH₃)₂ |
| B-804 | CH₃ | CH₂F | —CH₂—C₆H₅ | —CH—CH(CH₃)₂ |
| B-805 | CH₂F | CH₂F | —CH₂—C₆H₅ | —CH—CH(CH₃)₂ |
| B-806 | OCH₃ | CH₂F | —CH₂—C₆H₅ | —CH—CH(CH₃)₂ |
| B-807 | CH₃ | CHF₂ | —CH₂—C₆H₅ | —CH—CH(CH₃)₂ |
| B-808 | CH₂F | CHF₂ | —CH₂—C₆H₅ | —CH—CH(CH₃)₂ |
| B-809 | OCH₃ | CHF₂ | —CH₂—C₆H₅ | —CH—CH(CH₃)₂ |
| B-810 | CH₃ | CF₃ | —CH₂—C₆H₅ | —CH—CH(CH₃)₂ |
| B-811 | CH₂F | CF₃ | —CH₂—C₆H₅ | —CH—CH(CH₃)₂ |
| B-812 | OCH₃ | CF₃ | —CH₂—C₆H₅ | —CH—CH(CH₃)₂ |
| B-813 | CH₃ | CH₃ | —CH₂-(2-F—C₆H₄) | —CH—CH(CH₃)₂ |
| B-814 | CH₂F | CH₃ | —CH₂-(2-F—C₆H₄) | —CH—CH(CH₃)₂ |
| B-815 | OCH₃ | CH₃ | —CH₂-(2-F—C₆H₄) | —CH—CH(CH₃)₂ |
| B-816 | CH₃ | CH₂F | —CH₂-(2-F—C₆H₄) | —CH—CH(CH₃)₂ |
| B-817 | CH₂F | CH₂F | —CH₂-(2-F—C₆H₄) | —CH—CH(CH₃)₂ |
| B-818 | OCH₃ | CH₂F | —CH₂-(2-F—C₆H₄) | —CH—CH(CH₃)₂ |
| B-819 | CH₃ | CHF₂ | —CH₂-(2-F—C₆H₄) | —CH—CH(CH₃)₂ |
| B-820 | CH₂F | CHF₂ | —CH₂-(2-F—C₆H₄) | —CH—CH(CH₃)₂ |
| B-821 | OCH₃ | CHF₂ | —CH₂-(2-F—C₆H₄) | —CH—CH(CH₃)₂ |
| B-822 | CH₃ | CF₃ | —CH₂-(2-F—C₆H₄) | —CH—CH(CH₃)₂ |
| B-823 | CH₂F | CF₃ | —CH₂-(2-F—C₆H₄) | —CH—CH(CH₃)₂ |
| B-824 | OCH₃ | CF₃ | —CH₂-(2-F—C₆H₄) | —CH—CH(CH₃)₂ |
| B-825 | CH₃ | CH₃ | —CH₂-(3-F—C₆H₄) | —CH—CH(CH₃)₂ |
| B-826 | CH₂F | CH₃ | —CH₂-(3-F—C₆H₄) | —CH—CH(CH₃)₂ |
| B-827 | OCH₃ | CH₃ | —CH₂-(3-F—C₆H₄) | —CH—CH(CH₃)₂ |
| B-828 | CH₃ | CH₂F | —CH₂-(3-F—C₆H₄) | —CH—CH(CH₃)₂ |
| B-829 | CH₂F | CH₂F | —CH₂-(3-F—C₆H₄) | —CH—CH(CH₃)₂ |
| B-830 | OCH₃ | CH₂F | —CH₂-(3-F—C₆H₄) | —CH—CH(CH₃)₂ |
| B-831 | CH₃ | CHF₂ | —CH₂-(3-F—C₆H₄) | —CH—CH(CH₃)₂ |
| B-832 | CH₂F | CHF₂ | —CH₂-(3-F—C₆H₄) | —CH—CH(CH₃)₂ |
| B-833 | OCH₃ | CHF₂ | —CH₂-(3-F—C₆H₄) | —CH—CH(CH₃)₂ |
| B-834 | CH₃ | CF₃ | —CH₂-(3-F—C₆H₄) | —CH—CH(CH₃)₂ |
| B-835 | CH₂F | CF₃ | —CH₂-(3-F—C₆H₄) | —CH—CH(CH₃)₂ |
| B-836 | OCH₃ | CF₃ | —CH₂-(3-F—C₆H₄) | —CH—CH(CH₃)₂ |
| B-837 | CH₃ | CH₃ | —CH₂-(4-F—C₆H₄) | —CH—CH(CH₃)₂ |
| B-838 | CH₂F | CH₃ | —CH₂-(4-F—C₆H₄) | —CH—CH(CH₃)₂ |
| B-839 | OCH₃ | CH₃ | —CH₂-(4-F—C₆H₄) | —CH—CH(CH₃)₂ |
| B-840 | CH₃ | CH₂F | —CH₂-(4-F—C₆H₄) | —CH—CH(CH₃)₂ |
| B-841 | CH₂F | CH₂F | —CH₂-(4-F—C₆H₄) | —CH—CH(CH₃)₂ |
| B-842 | OCH₃ | CH₂F | —CH₂-(4-F—C₆H₄) | —CH—CH(CH₃)₂ |
| B-843 | CH₃ | CHF₂ | —CH₂-(4-F—C₆H₄) | —CH—CH(CH₃)₂ |
| B-844 | CH₂F | CHF₂ | —CH₂-(4-F—C₆H₄) | —CH—CH(CH₃)₂ |
| B-845 | OCH₃ | CHF₂ | —CH₂-(4-F—C₆H₄) | —CH—CH(CH₃)₂ |
| B-846 | CH₃ | CF₃ | —CH₂-(4-F—C₆H₄) | —CH—CH(CH₃)₂ |
| B-847 | CH₂F | CF₃ | —CH₂-(4-F—C₆H₄) | —CH—CH(CH₃)₂ |
| B-848 | OCH₃ | CF₃ | —CH₂-(4-F—C₆H₄) | —CH—CH(CH₃)₂ |
| B-849 | CH₃ | CH₃ | —CH₂-(2,4-F₂—C₆H₃) | —CH—CH(CH₃)₂ |
| B-850 | CH₂F | CH₃ | —CH₂-(2,4-F₂—C₆H₃) | —CH—CH(CH₃)₂ |
| B-851 | OCH₃ | CH₃ | —CH₂-(2,4-F₂—C₆H₃) | —CH—CH(CH₃)₂ |
| B-852 | CH₃ | CH₂F | —CH₂-(2,4-F₂—C₆H₃) | —CH—CH(CH₃)₂ |
| B-853 | CH₂F | CH₂F | —CH₂-(2,4-F₂—C₆H₃) | —CH—CH(CH₃)₂ |
| B-854 | OCH₃ | CH₂F | —CH₂-(2,4-F₂—C₆H₃) | —CH—CH(CH₃)₂ |
| B-855 | CH₃ | CHF₂ | —CH₂-(2,4-F₂—C₆H₃) | —CH—CH(CH₃)₂ |
| B-856 | CH₂F | CHF₂ | —CH₂-(2,4-F₂—C₆H₃) | —CH—CH(CH₃)₂ |
| B-857 | OCH₃ | CHF₂ | —CH₂-(2,4-F₂—C₆H₃) | —CH—CH(CH₃)₂ |
| B-858 | CH₃ | CF₃ | —CH₂-(2,4-F₂—C₆H₃) | —CH—CH(CH₃)₂ |
| B-859 | CH₂F | CF₃ | —CH₂-(2,4-F₂—C₆H₃) | —CH—CH(CH₃)₂ |
| B-860 | OCH₃ | CF₃ | —CH₂-(2,4-F₂—C₆H₃) | —CH—CH(CH₃)₂ |
| B-861 | CH₃ | CH₃ | —CH₂-(2-Cl—C₆H₄) | —CH—CH(CH₃)₂ |
| B-862 | CH₂F | CH₃ | —CH₂-(2-Cl—C₆H₄) | —CH—CH(CH₃)₂ |
| B-863 | OCH₃ | CH₃ | —CH₂-(2-Cl—C₆H₄) | —CH—CH(CH₃)₂ |
| B-864 | CH₃ | CH₂F | —CH₂-(2-Cl—C₆H₄) | —CH—CH(CH₃)₂ |
| B-865 | CH₂F | CH₂F | —CH₂-(2-Cl—C₆H₄) | —CH—CH(CH₃)₂ |
| B-866 | OCH₃ | CH₂F | —CH₂-(2-Cl—C₆H₄) | —CH—CH(CH₃)₂ |
| B-867 | CH₃ | CHF₂ | —CH₂-(2-Cl—C₆H₄) | —CH—CH(CH₃)₂ |
| B-868 | CH₂F | CHF₂ | —CH₂-(2-Cl—C₆H₄) | —CH—CH(CH₃)₂ |
| B-869 | OCH₃ | CHF₂ | —CH₂-(2-Cl—C₆H₄) | —CH—CH(CH₃)₂ |
| B-870 | CH₃ | CF₃ | —CH₂-(2-Cl—C₆H₄) | —CH—CH(CH₃)₂ |
| B-871 | CH₂F | CF₃ | —CH₂-(2-Cl—C₆H₄) | —CH—CH(CH₃)₂ |
| B-872 | OCH₃ | CF₃ | —CH₂-(2-Cl—C₆H₄) | —CH—CH(CH₃)₂ |
| B-873 | CH₃ | CH₃ | —CH₂-(3-Cl—C₆H₄) | —CH—CH(CH₃)₂ |
| B-874 | CH₂F | CH₃ | —CH₂-(3-Cl—C₆H₄) | —CH—CH(CH₃)₂ |
| B-875 | OCH₃ | CH₃ | —CH₂-(3-Cl—C₆H₄) | —CH—CH(CH₃)₂ |
| B-876 | CH₃ | CH₂F | —CH₂-(3-Cl—C₆H₄) | —CH—CH(CH₃)₂ |
| B-877 | CH₂F | CH₂F | —CH₂-(3-Cl—C₆H₄) | —CH—CH(CH₃)₂ |
| B-878 | OCH₃ | CH₂F | —CH₂-(3-Cl—C₆H₄) | —CH—CH(CH₃)₂ |
| B-879 | CH₃ | CHF₂ | —CH₂-(3-Cl—C₆H₄) | —CH—CH(CH₃)₂ |
| B-880 | CH₂F | CHF₂ | —CH₂-(3-Cl—C₆H₄) | —CH—CH(CH₃)₂ |
| B-881 | OCH₃ | CHF₂ | —CH₂-(3-Cl—C₆H₄) | —CH—CH(CH₃)₂ |
| B-882 | CH₃ | CF₃ | —CH₂-(3-Cl—C₆H₄) | —CH—CH(CH₃)₂ |
| B-883 | CH₂F | CF₃ | —CH₂-(3-Cl—C₆H₄) | —CH—CH(CH₃)₂ |
| B-884 | OCH₃ | CF₃ | —CH₂-(3-Cl—C₆H₄) | —CH—CH(CH₃)₂ |
| B-885 | CH₃ | CH₃ | —CH₂-(4-Cl—C₆H₄) | —CH—CH(CH₃)₂ |
| B-886 | CH₂F | CH₃ | —CH₂-(4-Cl—C₆H₄) | —CH—CH(CH₃)₂ |
| B-887 | OCH₃ | CH₃ | —CH₂-(4-Cl—C₆H₄) | —CH—CH(CH₃)₂ |
| B-888 | CH₃ | CH₂F | —CH₂-(4-Cl—C₆H₄) | —CH—CH(CH₃)₂ |
| B-889 | CH₂F | CH₂F | —CH₂-(4-Cl—C₆H₄) | —CH—CH(CH₃)₂ |
| B-890 | OCH₃ | CH₂F | —CH₂-(4-Cl—C₆H₄) | —CH—CH(CH₃)₂ |
| B-891 | CH₃ | CHF₂ | —CH₂-(4-Cl—C₆H₄) | —CH—CH(CH₃)₂ |
| B-892 | CH₂F | CHF₂ | —CH₂-(4-Cl—C₆H₄) | —CH—CH(CH₃)₂ |
| B-893 | OCH₃ | CHF₂ | —CH₂-(4-Cl—C₆H₄) | —CH—CH(CH₃)₂ |
| B-894 | CH₃ | CF₃ | —CH₂-(4-Cl—C₆H₄) | —CH—CH(CH₃)₂ |
| B-895 | CH₂F | CF₃ | —CH₂-(4-Cl—C₆H₄) | —CH—CH(CH₃)₂ |
| B-896 | OCH₃ | CF₃ | —CH₂-(4-Cl—C₆H₄) | —CH—CH(CH₃)₂ |
| B-897 | CH₃ | CH₃ | —CH₂-(2,4-Cl₂—C₆H₃) | —CH—CH(CH₃)₂ |
| B-898 | CH₂F | CH₃ | —CH₂-(2,4-Cl₂—C₆H₃) | —CH—CH(CH₃)₂ |
| B-899 | OCH₃ | CH₃ | —CH₂-(2,4-Cl₂—C₆H₃) | —CH—CH(CH₃)₂ |
| B-900 | CH₃ | CH₂F | —CH₂-(2,4-Cl₂—C₆H₃) | —CH—CH(CH₃)₂ |
| B-901 | CH₂F | CH₂F | —CH₂-(2,4-Cl₂—C₆H₃) | —CH—CH(CH₃)₂ |
| B-902 | OCH₃ | CH₂F | —CH₂-(2,4-Cl₂—C₆H₃) | —CH—CH(CH₃)₂ |
| B-903 | CH₃ | CHF₂ | —CH₂-(2,4-Cl₂—C₆H₃) | —CH—CH(CH₃)₂ |
| B-904 | CH₂F | CHF₂ | —CH₂-(2,4-Cl₂—C₆H₃) | —CH—CH(CH₃)₂ |
| B-905 | OCH₃ | CHF₂ | —CH₂-(2,4-Cl₂—C₆H₃) | —CH—CH(CH₃)₂ |
| B-906 | CH₃ | CF₃ | —CH₂-(2,4-Cl₂—C₆H₃) | —CH—CH(CH₃)₂ |
| B-907 | CH₂F | CF₃ | —CH₂-(2,4-Cl₂—C₆H₃) | —CH—CH(CH₃)₂ |
| B-908 | OCH₃ | CF₃ | —CH₂-(2,4-Cl₂—C₆H₃) | —CH—CH(CH₃)₂ |
| B-909 | CH₃ | CH₃ | —CH₂—CH₂—C₆H₅ | —CH—CH(CH₃)₂ |
| B-910 | CH₂F | CH₃ | —CH₂—CH₂—C₆H₅ | —CH—CH(CH₃)₂ |
| B-911 | OCH₃ | CH₃ | —CH₂—CH₂—C₆H₅ | —CH—CH(CH₃)₂ |
| B-912 | CH₃ | CH₂F | —CH₂—CH₂—C₆H₅ | —CH—CH(CH₃)₂ |
| B-913 | CH₂F | CH₂F | —CH₂—CH₂—C₆H₅ | —CH—CH(CH₃)₂ |
| B-914 | OCH₃ | CH₂F | —CH₂—CH₂—C₆H₅ | —CH—CH(CH₃)₂ |
| B-915 | CH₃ | CHF₂ | —CH₂—CH₂—C₆H₅ | —CH—CH(CH₃)₂ |
| B-916 | CH₂F | CHF₂ | —CH₂—CH₂—C₆H₅ | —CH—CH(CH₃)₂ |
| B-917 | OCH₃ | CHF₂ | —CH₂—CH₂—C₆H₅ | —CH—CH(CH₃)₂ |
| B-918 | CH₃ | CF₃ | —CH₂—CH₂—C₆H₅ | —CH—CH(CH₃)₂ |
| B-919 | CH₂F | CF₃ | —CH₂—CH₂—C₆H₅ | —CH—CH(CH₃)₂ |
| B-920 | OCH₃ | CF₃ | —CH₂—CH₂—C₆H₅ | —CH—CH(CH₃)₂ |
| B-921 | CH₃ | CH₃ | C₆H₅ | —CH(CH₃)—CH₂—CH₃ |
| B-922 | CH₂F | CH₃ | C₆H₅ | —CH(CH₃)—CH₂—CH₃ |
| B-923 | OCH₃ | CH₃ | C₆H₅ | —CH(CH₃)—CH₂—CH₃ |
| B-924 | CH₃ | CH₂F | C₆H₅ | —CH(CH₃)—CH₂—CH₃ |
| B-925 | CH₂F | CH₂F | C₆H₅ | —CH(CH₃)—CH₂—CH₃ |
| B-926 | OCH₃ | CH₂F | C₆H₅ | —CH(CH₃)—CH₂—CH₃ |
| B-927 | CH₃ | CHF₂ | C₆H₅ | —CH(CH₃)—CH₂—CH₃ |
| B-928 | CH₂F | CHF₂ | C₆H₅ | —CH(CH₃)—CH₂—CH₃ |
| B-929 | OCH₃ | CHF₂ | C₆H₅ | —CH(CH₃)—CH₂—CH₃ |
| B-930 | CH₃ | CF₃ | C₆H₅ | —CH(CH₃)—CH₂—CH₃ |
| B-931 | CH₂F | CF₃ | C₆H₅ | —CH(CH₃)—CH₂—CH₃ |
| B-932 | OCH₃ | CF₃ | C₆H₅ | —CH(CH₃)—CH₂—CH₃ |
| B-933 | CH₃ | CH₃ | —CH₂—C₆H₅ | —CH(CH₃)—CH₂—CH₃ |
| B-934 | CH₂F | CH₃ | —CH₂—C₆H₅ | —CH(CH₃)—CH₂—CH₃ |
| B-935 | OCH₃ | CH₃ | —CH₂—C₆H₅ | —CH(CH₃)—CH₂—CH₃ |
| B-936 | CH₃ | CH₂F | —CH₂—C₆H₅ | —CH(CH₃)—CH₂—CH₃ |
| B-937 | CH₂F | CH₂F | —CH₂—C₆H₅ | —CH(CH₃)—CH₂—CH₃ |
| B-938 | OCH₃ | CH₂F | —CH₂—C₆H₅ | —CH(CH₃)—CH₂—CH₃ |
| B-939 | CH₃ | CHF₂ | —CH₂—C₆H₅ | —CH(CH₃)—CH₂—CH₃ |
| B-940 | CH₂F | CHF₂ | —CH₂—C₆H₅ | —CH(CH₃)—CH₂—CH₃ |
| B-941 | OCH₃ | CHF₂ | —CH₂—C₆H₅ | —CH(CH₃)—CH₂—CH₃ |
| B-942 | CH₃ | CF₃ | —CH₂—C₆H₅ | —CH(CH₃)—CH₂—CH₃ |
| B-943 | CH₂F | CF₃ | —CH₂—C₆H₅ | —CH(CH₃)—CH₂—CH₃ |
| B-944 | OCH₃ | CF₃ | —CH₂—C₆H₅ | —CH(CH₃)—CH₂—CH₃ |
| B-945 | CH₃ | CH₃ | —CH₂-(2-F—C₆H₄) | —CH(CH₃)—CH₂—CH₃ |
| B-946 | CH₂F | CH₃ | —CH₂-(2-F—C₆H₄) | —CH(CH₃)—CH₂—CH₃ |
| B-947 | OCH₃ | CH₃ | —CH₂-(2-F—C₆H₄) | —CH(CH₃)—CH₂—CH₃ |
| B-948 | CH₃ | CH₂F | —CH₂-(2-F—C₆H₄) | —CH(CH₃)—CH₂—CH₃ |
| B-949 | CH₂F | CH₂F | —CH₂-(2-F—C₆H₄) | —CH(CH₃)—CH₂—CH₃ |
| B-950 | OCH₃ | CH₂F | —CH₂-(2-F—C₆H₄) | —CH(CH₃)—CH₂—CH₃ |
| B-951 | CH₃ | CHF₂ | —CH₂-(2-F—C₆H₄) | —CH(CH₃)—CH₂—CH₃ |
| B-952 | CH₂F | CHF₂ | —CH₂-(2-F—C₆H₄) | —CH(CH₃)—CH₂—CH₃ |
| B-953 | OCH₃ | CHF₂ | —CH₂-(2-F—C₆H₄) | —CH(CH₃)—CH₂—CH₃ |
| B-954 | CH₃ | CF₃ | —CH₂-(2-F—C₆H₄) | —CH(CH₃)—CH₂—CH₃ |
| B-955 | CH₂F | CF₃ | —CH₂-(2-F—C₆H₄) | —CH(CH₃)—CH₂—CH₃ |
| B-956 | OCH₃ | CF₃ | —CH₂-(2-F—C₆H₄) | —CH(CH₃)—CH₂—CH₃ |
| B-957 | CH₃ | CH₃ | —CH₂-(3-F—C₆H₄) | —CH(CH₃)—CH₂—CH₃ |
| B-958 | CH₂F | CH₃ | —CH₂-(3-F—C₆H₄) | —CH(CH₃)—CH₂—CH₃ |
| B-959 | OCH₃ | CH₃ | —CH₂-(3-F—C₆H₄) | —CH(CH₃)—CH₂—CH₃ |
| B-960 | CH₃ | CH₂F | —CH₂-(3-F—C₆H₄) | —CH(CH₃)—CH₂—CH₃ |
| B-961 | CH₂F | CH₂F | —CH₂-(3-F—C₆H₄) | —CH(CH₃)—CH₂—CH₃ |
| B-962 | OCH₃ | CH₂F | —CH₂-(3-F—C₆H₄) | —CH(CH₃)—CH₂—CH₃ |
| B-963 | CH₃ | CHF₂ | —CH₂-(3-F—C₆H₄) | —CH(CH₃)—CH₂—CH₃ |
| B-964 | CH₂F | CHF₂ | —CH₂-(3-F—C₆H₄) | —CH(CH₃)—CH₂—CH₃ |
| B-965 | OCH₃ | CHF₂ | —CH₂-(3-F—C₆H₄) | —CH(CH₃)—CH₂—CH₃ |
| B-966 | CH₃ | CF₃ | —CH₂-(3-F—C₆H₄) | —CH(CH₃)—CH₂—CH₃ |
| B-967 | CH₂F | CF₃ | —CH₂-(3-F—C₆H₄) | —CH(CH₃)—CH₂—CH₃ |
| B-968 | OCH₃ | CF₃ | —CH₂-(3-F—C₆H₄) | —CH(CH₃)—CH₂—CH₃ |
| B-969 | CH₃ | CH₃ | —CH₂-(4-F—C₆H₄) | —CH(CH₃)—CH₂—CH₃ |
| B-970 | CH₂F | CH₃ | —CH₂-(4-F—C₆H₄) | —CH(CH₃)—CH₂—CH₃ |
| B-971 | OCH₃ | CH₃ | —CH₂-(4-F—C₆H₄) | —CH(CH₃)—CH₂—CH₃ |
| B-972 | CH₃ | CH₂F | —CH₂-(4-F—C₆H₄) | —CH(CH₃)—CH₂—CH₃ |
| B-973 | CH₂F | CH₂F | —CH₂-(4-F—C₆H₄) | —CH(CH₃)—CH₂—CH₃ |
| B-974 | OCH₃ | CH₂F | —CH₂-(4-F—C₆H₄) | —CH(CH₃)—CH₂—CH₃ |
| B-975 | CH₃ | CHF₂ | —CH₂-(4-F—C₆H₄) | —CH(CH₃)—CH₂—CH₃ |
| B-976 | CH₂F | CHF₂ | —CH₂-(4-F—C₆H₄) | —CH(CH₃)—CH₂—CH₃ |
| B-977 | OCH₃ | CHF₂ | —CH₂-(4-F—C₆H₄) | —CH(CH₃)—CH₂—CH₃ |
| B-978 | CH₃ | CF₃ | —CH₂-(4-F—C₆H₄) | —CH(CH₃)—CH₂—CH₃ |
| B-979 | CH₂F | CF₃ | —CH₂-(4-F—C₆H₄) | —CH(CH₃)—CH₂—CH₃ |
| B-980 | OCH₃ | CF₃ | —CH₂-(4-F—C₆H₄) | —CH(CH₃)—CH₂—CH₃ |
| B-981 | CH₃ | CH₃ | —CH₂-(2,4-F₂—C₆H₃) | —CH(CH₃)—CH₂—CH₃ |
| B-982 | CH₂F | CH₃ | —CH₂-(2,4-F₂—C₆H₃) | —CH(CH₃)—CH₂—CH₃ |
| B-983 | OCH₃ | CH₃ | —CH₂-(2,4-F₂—C₆H₃) | —CH(CH₃)—CH₂—CH₃ |
| B-984 | CH₃ | CH₂F | —CH₂-(2,4-F₂—C₆H₃) | —CH(CH₃)—CH₂—CH₃ |
| B-985 | CH₂F | CH₂F | —CH₂-(2,4-F₂—C₆H₃) | —CH(CH₃)—CH₂—CH₃ |
| B-986 | OCH₃ | CH₂F | —CH₂-(2,4-F₂—C₆H₃) | —CH(CH₃)—CH₂—CH₃ |
| B-987 | CH₃ | CHF₂ | —CH₂-(2,4-F₂—C₆H₃) | —CH(CH₃)—CH₂—CH₃ |
| B-988 | CH₂F | CHF₂ | —CH₂-(2,4-F₂—C₆H₃) | —CH(CH₃)—CH₂—CH₃ |
| B-989 | OCH₃ | CHF₂ | —CH₂-(2,4-F₂—C₆H₃) | —CH(CH₃)—CH₂—CH₃ |
| B-990 | CH₃ | CF₃ | —CH₂-(2,4-F₂—C₆H₃) | —CH(CH₃)—CH₂—CH₃ |
| B-991 | CH₂F | CF₃ | —CH₂-(2,4-F₂—C₆H₃) | —CH(CH₃)—CH₂—CH₃ |
| B-992 | OCH₃ | CF₃ | —CH₂-(2,4-F₂—C₆H₃) | —CH(CH₃)—CH₂—CH₃ |
| B-993 | CH₃ | CH₃ | —CH₂-(2-Cl—C₆H₄) | —CH(CH₃)—CH₂—CH₃ |
| B-994 | CH₂F | CH₃ | —CH₂-(2-Cl—C₆H₄) | —CH(CH₃)—CH₂—CH₃ |
| B-995 | OCH₃ | CH₃ | —CH₂-(2-Cl—C₆H₄) | —CH(CH₃)—CH₂—CH₃ |
| B-996 | CH₃ | CH₂F | —CH₂-(2-Cl—C₆H₄) | —CH(CH₃)—CH₂—CH₃ |
| B-997 | CH₂F | CH₂F | —CH₂-(2-Cl—C₆H₄) | —CH(CH₃)—CH₂—CH₃ |
| B-998 | OCH₃ | CH₂F | —CH₂-(2-Cl—C₆H₄) | —CH(CH₃)—CH₂—CH₃ |
| B-999 | CH₃ | CHF₂ | —CH₂-(2-Cl—C₆H₄) | —CH(CH₃)—CH₂—CH₃ |
| B-1000 | CH₂F | CHF₂ | —CH₂-(2-Cl—C₆H₄) | —CH(CH₃)—CH₂—CH₃ |
| B-1001 | OCH₃ | CHF₂ | —CH₂-(2-Cl—C₆H₄) | —CH(CH₃)—CH₂—CH₃ |
| B-1002 | CH₃ | CF₃ | —CH₂-(2-Cl—C₆H₄) | —CH(CH₃)—CH₂—CH₃ |
| B-1003 | CH₂F | CF₃ | —CH₂-(2-Cl—C₆H₄) | —CH(CH₃)—CH₂—CH₃ |
| B-1004 | OCH₃ | CF₃ | —CH₂-(2-Cl—C₆H₄) | —CH(CH₃)—CH₂—CH₃ |
| B-1005 | CH₃ | CH₃ | —CH₂-(3-Cl—C₆H₄) | —CH(CH₃)—CH₂—CH₃ |
| B-1006 | CH₂F | CH₃ | —CH₂-(3-Cl—C₆H₄) | —CH(CH₃)—CH₂—CH₃ |
| B-1007 | OCH₃ | CH₃ | —CH₂-(3-Cl—C₆H₄) | —CH(CH₃)—CH₂—CH₃ |
| B-1008 | CH₃ | CH₂F | —CH₂-(3-Cl—C₆H₄) | —CH(CH₃)—CH₂—CH₃ |
| B-1009 | CH₂F | CH₂F | —CH₂-(3-Cl—C₆H₄) | —CH(CH₃)—CH₂—CH₃ |
| B-1010 | OCH₃ | CH₂F | —CH₂-(3-Cl—C₆H₄) | —CH(CH₃)—CH₂—CH₃ |
| B-1011 | CH₃ | CHF₂ | —CH₂-(3-Cl—C₆H₄) | —CH(CH₃)—CH₂—CH₃ |
| B-1012 | CH₂F | CHF₂ | —CH₂-(3-Cl—C₆H₄) | —CH(CH₃)—CH₂—CH₃ |
| B-1013 | OCH₃ | CHF₂ | —CH₂-(3-Cl—C₆H₄) | —CH(CH₃)—CH₂—CH₃ |
| B-1014 | CH₃ | CF₃ | —CH₂-(3-Cl—C₆H₄) | —CH(CH₃)—CH₂—CH₃ |
| B-1015 | CH₂F | CF₃ | —CH₂-(3-Cl—C₆H₄) | —CH(CH₃)—CH₂—CH₃ |
| B-1016 | OCH₃ | CF₃ | —CH₂-(3-Cl—C₆H₄) | —CH(CH₃)—CH₂—CH₃ |
| B-1017 | CH₃ | CH₃ | —CH₂-(4-Cl—C₆H₄) | —CH(CH₃)—CH₂—CH₃ |
| B-1018 | CH₂F | CH₃ | —CH₂-(4-Cl—C₆H₄) | —CH(CH₃)—CH₂—CH₃ |
| B-1019 | OCH₃ | CH₃ | —CH₂-(4-Cl—C₆H₄) | —CH(CH₃)—CH₂—CH₃ |
| B-1020 | CH₃ | CH₂F | —CH₂-(4-Cl—C₆H₄) | —CH(CH₃)—CH₂—CH₃ |
| B-1021 | CH₂F | CH₂F | —CH₂-(4-Cl—C₆H₄) | —CH(CH₃)—CH₂—CH₃ |
| B-1022 | OCH₃ | CH₂F | —CH₂-(4-Cl—C₆H₄) | —CH(CH₃)—CH₂—CH₃ |
| B-1023 | CH₃ | CHF₂ | —CH₂-(4-Cl—C₆H₄) | —CH(CH₃)—CH₂—CH₃ |
| B-1024 | CH₂F | CHF₂ | —CH₂-(4-Cl—C₆H₄) | —CH(CH₃)—CH₂—CH₃ |
| B-1025 | OCH₃ | CHF₂ | —CH₂-(4-Cl—C₆H₄) | —CH(CH₃)—CH₂—CH₃ |
| B-1026 | CH₃ | CF₃ | —CH₂-(4-Cl—C₆H₄) | —CH(CH₃)—CH₂—CH₃ |
| B-1027 | CH₂F | CF₃ | —CH₂-(4-Cl—C₆H₄) | —CH(CH₃)—CH₂—CH₃ |
| B-1028 | OCH₃ | CF₃ | —CH₂-(4-Cl—C₆H₄) | —CH(CH₃)—CH₂—CH₃ |
| B-1029 | CH₃ | CH₃ | —CH₂-(2,4-Cl₂—C₆H₃) | —CH(CH₃)—CH₂—CH₃ |
| B-1030 | CH₂F | CH₃ | —CH₂-(2,4-Cl₂—C₆H₃) | —CH(CH₃)—CH₂—CH₃ |
| B-1031 | OCH₃ | CH₃ | —CH₂-(2,4-Cl₂—C₆H₃) | —CH(CH₃)—CH₂—CH₃ |
| B-1032 | CH₃ | CH₂F | —CH₂-(2,4-Cl₂—C₆H₃) | —CH(CH₃)—CH₂—CH₃ |
| B-1033 | CH₂F | CH₂F | —CH₂-(2,4-Cl₂—C₆H₃) | —CH(CH₃)—CH₂—CH₃ |
| B-1034 | OCH₃ | CH₂F | —CH₂-(2,4-Cl₂—C₆H₃) | —CH(CH₃)—CH₂—CH₃ |
| B-1035 | CH₃ | CHF₂ | —CH₂-(2,4-Cl₂—C₆H₃) | —CH(CH₃)—CH₂—CH₃ |
| B-1036 | CH₂F | CHF₂ | —CH₂-(2,4-Cl₂—C₆H₃) | —CH(CH₃)—CH₂—CH₃ |
| B-1037 | OCH₃ | CHF₂ | —CH₂-(2,4-Cl₂—C₆H₃) | —CH(CH₃)—CH₂—CH₃ |
| B-1038 | CH₃ | CF₃ | —CH₂-(2,4-Cl₂—C₆H₃) | —CH(CH₃)—CH₂—CH₃ |
| B-1039 | CH₂F | CF₃ | —CH₂-(2,4-Cl₂—C₆H₃) | —CH(CH₃)—CH₂—CH₃ |
| B-1040 | OCH₃ | CF₃ | —CH₂-(2,4-Cl₂—C₆H₃) | —CH(CH₃)—CH₂—CH₃ |
| B-1041 | CH₃ | CH₃ | —CH₂—CH₂—C₆H₅ | —CH(CH₃)—CH₂—CH₃ |
| B-1042 | CH₂F | CH₃ | —CH₂—CH₂—C₆H₅ | —CH(CH₃)—CH₂—CH₃ |
| B-1043 | OCH₃ | CH₃ | —CH₂—CH₂—C₆H₅ | —CH(CH₃)—CH₂—CH₃ |
| B-1044 | CH₃ | CH₂F | —CH₂—CH₂—C₆H₅ | —CH(CH₃)—CH₂—CH₃ |
| B-1045 | CH₂F | CH₂F | —CH₂—CH₂—C₆H₅ | —CH(CH₃)—CH₂—CH₃ |
| B-1046 | OCH₃ | CH₂F | —CH₂—CH₂—C₆H₅ | —CH(CH₃)—CH₂—CH₃ |
| B-1047 | CH₃ | CHF₂ | —CH₂—CH₂—C₆H₅ | —CH(CH₃)—CH₂—CH₃ |
| B-1048 | CH₂F | CHF₂ | —CH₂—CH₂—C₆H₅ | —CH(CH₃)—CH₂—CH₃ |
| B-1049 | OCH₃ | CHF₂ | —CH₂—CH₂—C₆H₅ | —CH(CH₃)—CH₂—CH₃ |
| B-1050 | CH₃ | CF₃ | —CH₂—CH₂—C₆H₅ | —CH(CH₃)—CH₂—CH₃ |
| B-1051 | CH₂F | CF₃ | —CH₂—CH₂—C₆H₅ | —CH(CH₃)—CH₂—CH₃ |
| B-1052 | OCH₃ | CF₃ | —CH₂—CH₂—C₆H₅ | —CH(CH₃)—CH₂—CH₃ |
| B-1053 | CH₃ | CH₃ | C₆H₅ | —C(CH₃)₃ |
| B-1054 | CH₂F | CH₃ | C₆H₅ | —C(CH₃)₃ |
| B-1055 | OCH₃ | CH₃ | C₆H₅ | —C(CH₃)₃ |
| B-1056 | CH₃ | CH₂F | C₆H₅ | —C(CH₃)₃ |
| B-1057 | CH₂F | CH₂F | C₆H₅ | —C(CH₃)₃ |
| B-1058 | OCH₃ | CH₂F | C₆H₅ | —C(CH₃)₃ |
| B-1059 | CH₃ | CHF₂ | C₆H₅ | —C(CH₃)₃ |
| B-1060 | CH₂F | CHF₂ | C₆H₅ | —C(CH₃)₃ |
| B-1061 | OCH₃ | CHF₂ | C₆H₅ | —C(CH₃)₃ |
| B-1062 | CH₃ | CF₃ | C₆H₅ | —C(CH₃)₃ |
| B-1063 | CH₂F | CF₃ | C₆H₅ | —C(CH₃)₃ |
| B-1064 | OCH₃ | CF₃ | C₆H₅ | —C(CH₃)₃ |
| B-1065 | CH₃ | CH₃ | —CH₂—C₆H₅ | —C(CH₃)₃ |
| B-1066 | CH₂F | CH₃ | —CH₂—C₆H₅ | —C(CH₃)₃ |
| B-1067 | OCH₃ | CH₃ | —CH₂—C₆H₅ | —C(CH₃)₃ |
| B-1068 | CH₃ | CH₂F | —CH₂—C₆H₅ | —C(CH₃)₃ |
| B-1069 | CH₂F | CH₂F | —CH₂—C₆H₅ | —C(CH₃)₃ |
| B-1070 | OCH₃ | CH₂F | —CH₂—C₆H₅ | —C(CH₃)₃ |
| B-1071 | CH₃ | CHF₂ | —CH₂—C₆H₅ | —C(CH₃)₃ |
| B-1072 | CH₂F | CHF₂ | —CH₂—C₆H₅ | —C(CH₃)₃ |
| B-1073 | OCH₃ | CHF₂ | —CH₂—C₆H₅ | —C(CH₃)₃ |
| B-1074 | CH₃ | CF₃ | —CH₂—C₆H₅ | —C(CH₃)₃ |
| B-1075 | CH₂F | CF₃ | —CH₂—C₆H₅ | —C(CH₃)₃ |
| B-1076 | OCH₃ | CF₃ | —CH₂—C₆H₅ | —C(CH₃)₃ |
| B-1077 | CH₃ | CH₃ | —CH₂-(2-F—C₆H₄) | —C(CH₃)₃ |
| B-1078 | CH₂F | CH₃ | —CH₂-(2-F—C₆H₄) | —C(CH₃)₃ |
| B-1079 | OCH₃ | CH₃ | —CH₂-(2-F—C₆H₄) | —C(CH₃)₃ |
| B-1080 | CH₃ | CH₂F | —CH₂-(2-F—C₆H₄) | —C(CH₃)₃ |
| B-1081 | CH₂F | CH₂F | —CH₂-(2-F—C₆H₄) | —C(CH₃)₃ |
| B-1082 | OCH₃ | CH₂F | —CH₂-(2-F—C₆H₄) | —C(CH₃)₃ |
| B-1083 | CH₃ | CHF₂ | —CH₂-(2-F—C₆H₄) | —C(CH₃)₃ |
| B-1084 | CH₂F | CHF₂ | —CH₂-(2-F—C₆H₄) | —C(CH₃)₃ |
| B-1085 | OCH₃ | CHF₂ | —CH₂-(2-F—C₆H₄) | —C(CH₃)₃ |
| B-1086 | CH₃ | CF₃ | —CH₂-(2-F—C₆H₄) | —C(CH₃)₃ |
| B-1087 | CH₂F | CF₃ | —CH₂-(2-F—C₆H₄) | —C(CH₃)₃ |
| B-1088 | OCH₃ | CF₃ | —CH₂-(2-F—C₆H₄) | —C(CH₃)₃ |
| B-1089 | CH₃ | CH₃ | —CH₂-(3-F—C₆H₄) | —C(CH₃)₃ |
| B-1090 | CH₂F | CH₃ | —CH₂-(3-F—C₆H₄) | —C(CH₃)₃ |
| B-1091 | OCH₃ | CH₃ | —CH₂-(3-F—C₆H₄) | —C(CH₃)₃ |
| B-1092 | CH₃ | CH₂F | —CH₂-(3-F—C₆H₄) | —C(CH₃)₃ |
| B-1093 | CH₂F | CH₂F | —CH₂-(3-F—C₆H₄) | —C(CH₃)₃ |
| B-1094 | OCH₃ | CH₂F | —CH₂-(3-F—C₆H₄) | —C(CH₃)₃ |
| B-1095 | CH₃ | CHF₂ | —CH₂-(3-F—C₆H₄) | —C(CH₃)₃ |
| B-1096 | CH₂F | CHF₂ | —CH₂-(3-F—C₆H₄) | —C(CH₃)₃ |
| B-1097 | OCH₃ | CHF₂ | —CH₂-(3-F—C₆H₄) | —C(CH₃)₃ |
| B-1098 | CH₃ | CF₃ | —CH₂-(3-F—C₆H₄) | —C(CH₃)₃ |
| B-1099 | CH₂F | CF₃ | —CH₂-(3-F—C₆H₄) | —C(CH₃)₃ |
| B-1100 | OCH₃ | CF₃ | —CH₂-(3-F—C₆H₄) | —C(CH₃)₃ |
| B-1101 | CH₃ | CH₃ | —CH₂-(4-F—C₆H₄) | —C(CH₃)₃ |
| B-1102 | CH₂F | CH₃ | —CH₂-(4-F—C₆H₄) | —C(CH₃)₃ |
| B-1103 | OCH₃ | CH₃ | —CH₂-(4-F—C₆H₄) | —C(CH₃)₃ |
| B-1104 | CH₃ | CH₂F | —CH₂-(4-F—C₆H₄) | —C(CH₃)₃ |
| B-1105 | CH₂F | CH₂F | —CH₂-(4-F—C₆H₄) | —C(CH₃)₃ |
| B-1106 | OCH₃ | CH₂F | —CH₂-(4-F—C₆H₄) | —C(CH₃)₃ |
| B-1107 | CH₃ | CHF₂ | —CH₂-(4-F—C₆H₄) | —C(CH₃)₃ |
| B-1108 | CH₂F | CHF₂ | —CH₂-(4-F—C₆H₄) | —C(CH₃)₃ |
| B-1109 | OCH₃ | CHF₂ | —CH₂-(4-F—C₆H₄) | —C(CH₃)₃ |
| B-1110 | CH₃ | CF₃ | —CH₂-(4-F—C₆H₄) | —C(CH₃)₃ |
| B-1111 | CH₂F | CF₃ | —CH₂-(4-F—C₆H₄) | —C(CH₃)₃ |
| B-1112 | OCH₃ | CF₃ | —CH₂-(4-F—C₆H₄) | —C(CH₃)₃ |
| B-1113 | CH₃ | CH₃ | —CH₂-(2,4-F₂—C₆H₃) | —C(CH₃)₃ |
| B-1114 | CH₂F | CH₃ | —CH₂-(2,4-F₂—C₆H₃) | —C(CH₃)₃ |
| B-1115 | OCH₃ | CH₃ | —CH₂-(2,4-F₂—C₆H₃) | —C(CH₃)₃ |
| B-1116 | CH₃ | CH₂F | —CH₂-(2,4-F₂—C₆H₃) | —C(CH₃)₃ |
| B-1117 | CH₂F | CH₂F | —CH₂-(2,4-F₂—C₆H₃) | —C(CH₃)₃ |
| B-1118 | OCH₃ | CH₂F | —CH₂-(2,4-F₂—C₆H₃) | —C(CH₃)₃ |
| B-1119 | CH₃ | CHF₂ | —CH₂-(2,4-F₂—C₆H₃) | —C(CH₃)₃ |
| B-1120 | CH₂F | CHF₂ | —CH₂-(2,4-F₂—C₆H₃) | —C(CH₃)₃ |
| B-1121 | OCH₃ | CHF₂ | —CH₂-(2,4-F₂—C₆H₃) | —C(CH₃)₃ |
| B-1122 | CH₃ | CF₃ | —CH₂-(2,4-F₂—C₆H₃) | —C(CH₃)₃ |
| B-1123 | CH₂F | CF₃ | —CH₂-(2,4-F₂—C₆H₃) | —C(CH₃)₃ |
| B-1124 | OCH₃ | CF₃ | —CH₂-(2,4-F₂—C₆H₃) | —C(CH₃)₃ |
| B-1125 | CH₃ | CH₃ | —CH₂-(2-Cl—C₆H₄) | —C(CH₃)₃ |
| B-1126 | CH₂F | CH₃ | —CH₂-(2-Cl—C₆H₄) | —C(CH₃)₃ |
| B-1127 | OCH₃ | CH₃ | —CH₂-(2-Cl—C₆H₄) | —C(CH₃)₃ |
| B-1128 | CH₃ | CH₂F | —CH₂-(2-Cl—C₆H₄) | —C(CH₃)₃ |
| B-1129 | CH₂F | CH₂F | —CH₂-(2-Cl—C₆H₄) | —C(CH₃)₃ |
| B-1130 | OCH₃ | CH₂F | —CH₂-(2-Cl—C₆H₄) | —C(CH₃)₃ |
| B-1131 | CH₃ | CHF₂ | —CH₂-(2-Cl—C₆H₄) | —C(CH₃)₃ |
| B-1132 | CH₂F | CHF₂ | —CH₂-(2-Cl—C₆H₄) | —C(CH₃)₃ |
| B-1133 | OCH₃ | CHF₂ | —CH₂-(2-Cl—C₆H₄) | —C(CH₃)₃ |
| B-1134 | CH₃ | CF₃ | —CH₂-(2-Cl—C₆H₄) | —C(CH₃)₃ |
| B-1135 | CH₂F | CF₃ | —CH₂-(2-Cl—C₆H₄) | —C(CH₃)₃ |
| B-1136 | OCH₃ | CF₃ | —CH₂-(2-Cl—C₆H₄) | —C(CH₃)₃ |
| B-1137 | CH₃ | CH₃ | —CH₂-(3-Cl—C₆H₄) | —C(CH₃)₃ |
| B-1138 | CH₂F | CH₃ | —CH₂-(3-Cl—C₆H₄) | —C(CH₃)₃ |
| B-1139 | OCH₃ | CH₃ | —CH₂-(3-Cl—C₆H₄) | —C(CH₃)₃ |
| B-1140 | CH₃ | CH₂F | —CH₂-(3-Cl—C₆H₄) | —C(CH₃)₃ |
| B-1141 | CH₂F | CH₂F | —CH₂-(3-Cl—C₆H₄) | —C(CH₃)₃ |
| B-1142 | OCH₃ | CH₂F | —CH₂-(3-Cl—C₆H₄) | —C(CH₃)₃ |
| B-1143 | CH₃ | CHF₂ | —CH₂-(3-Cl—C₆H₄) | —C(CH₃)₃ |
| B-1144 | CH₂F | CHF₂ | —CH₂-(3-Cl—C₆H₄) | —C(CH₃)₃ |
| B-1145 | OCH₃ | CHF₂ | —CH₂-(3-Cl—C₆H₄) | —C(CH₃)₃ |
| B-1146 | CH₃ | CF₃ | —CH₂-(3-Cl—C₆H₄) | —C(CH₃)₃ |
| B-1147 | CH₂F | CF₃ | —CH₂-(3-Cl—C₆H₄) | —C(CH₃)₃ |
| B-1148 | OCH₃ | CF₃ | —CH₂-(3-Cl—C₆H₄) | —C(CH₃)₃ |
| B-1149 | CH₃ | CH₃ | —CH₂-(4-Cl—C₆H₄) | —C(CH₃)₃ |
| B-1150 | CH₂F | CH₃ | —CH₂-(4-Cl—C₆H₄) | —C(CH₃)₃ |
| B-1151 | OCH₃ | CH₃ | —CH₂-(4-Cl—C₆H₄) | —C(CH₃)₃ |
| B-1152 | CH₃ | CH₂F | —CH₂-(4-Cl—C₆H₄) | —C(CH₃)₃ |
| B-1153 | CH₂F | CH₂F | —CH₂-(4-Cl—C₆H₄) | —C(CH₃)₃ |
| B-1154 | OCH₃ | CH₂F | —CH₂-(4-Cl—C₆H₄) | —C(CH₃)₃ |
| B-1155 | CH₃ | CHF₂ | —CH₂-(4-Cl—C₆H₄) | —C(CH₃)₃ |
| B-1156 | CH₂F | CHF₂ | —CH₂-(4-Cl—C₆H₄) | —C(CH₃)₃ |
| B-1157 | OCH₃ | CHF₂ | —CH₂-(4-Cl—C₆H₄) | —C(CH₃)₃ |
| B-1158 | CH₃ | CF₃ | —CH₂-(4-Cl—C₆H₄) | —C(CH₃)₃ |
| B-1159 | CH₂F | CF₃ | —CH₂-(4-Cl—C₆H₄) | —C(CH₃)₃ |
| B-1160 | OCH₃ | CF₃ | —CH₂-(4-Cl—C₆H₄) | —C(CH₃)₃ |
| B-1161 | CH₃ | CH₃ | —CH₂-(2,4-Cl₂—C₆H₃) | —C(CH₃)₃ |
| B-1162 | CH₂F | CH₃ | —CH₂-(2,4-Cl₂—C₆H₃) | —C(CH₃)₃ |
| B-1163 | OCH₃ | CH₃ | —CH₂-(2,4-Cl₂—C₆H₃) | —C(CH₃)₃ |
| B-1164 | CH₃ | CH₂F | —CH₂-(2,4-Cl₂—C₆H₃) | —C(CH₃)₃ |
| B-1165 | CH₂F | CH₂F | —CH₂-(2,4-Cl₂—C₆H₃) | —C(CH₃)₃ |
| B-1166 | OCH₃ | CH₂F | —CH₂-(2,4-Cl₂—C₆H₃) | —C(CH₃)₃ |
| B-1167 | CH₃ | CHF₂ | —CH₂-(2,4-Cl₂—C₆H₃) | —C(CH₃)₃ |
| B-1168 | CH₂F | CHF₂ | —CH₂-(2,4-Cl₂—C₆H₃) | —C(CH₃)₃ |
| B-1169 | OCH₃ | CHF₂ | —CH₂-(2,4-Cl₂—C₆H₃) | —C(CH₃)₃ |
| B-1170 | CH₃ | CF₃ | —CH₂-(2,4-Cl₂—C₆H₃) | —C(CH₃)₃ |
| B-1171 | CH₂F | CF₃ | —CH₂-(2,4-Cl₂—C₆H₃) | —C(CH₃)₃ |
| B-1172 | OCH₃ | CF₃ | —CH₂-(2,4-Cl₂—C₆H₃) | —C(CH₃)₃ |
| B-1173 | CH₃ | CH₃ | —CH₂—CH₂—C₆H₅ | —C(CH₃)₃ |
| B-1174 | CH₂F | CH₃ | —CH₂—CH₂—C₆H₅ | —C(CH₃)₃ |
| B-1175 | OCH₃ | CH₃ | —CH₂—CH₂—C₆H₅ | —C(CH₃)₃ |
| B-1176 | CH₃ | CH₂F | —CH₂—CH₂—C₆H₆ | —C(CH₃)₃ |
| B-1177 | CH₂F | CH₂F | —CH₂—CH₂—C₆H₆ | —C(CH₃)₃ |
| B-1178 | OCH₃ | CH₂F | —CH₂—CH₂—C₆H₅ | —C(CH₃)₃ |
| B-1179 | CH₃ | CHF₂ | —CH₂—CH₂—C₆H₆ | —C(CH₃)₃ |
| B-1180 | CH₂F | CHF₂ | —CH₂—CH₂—C₆H₅ | —C(CH₃)₃ |
| B-1181 | OCH₃ | CHF₂ | —CH₂—CH₂—C₆H₅ | —C(CH₃)₃ |
| B-1182 | CH₃ | CF₃ | —CH₂—CH₂—C₆H₅ | —C(CH₃)₃ |
| B-1183 | CH₂F | CF₃ | —CH₂—CH₂—C₆H₅ | —C(CH₃)₃ |
| B-1184 | OCH₃ | CF₃ | —CH₂—CH₂—C₆H₅ | —C(CH₃)₃ |

Table 1a Compounds of the formula I.A-1, I.A-2, I.A-3, I.A-4, I.B-1, I.B-2, I.B-3, I.B-4 and I.C-1, I.C-2, I.C-3, I.C-4 in which R⁶ is H and the meaning for the combination of R², R³, R⁷ and R⁸ for each individual compound corresponds in each case to one line of Table B (compounds I.A-1.1a.B-1 to I.A-1.1a.B-1184, I.A-2.1a.B-1 to I.A-2.1a.B-1184, I.A-3.1a.B-1 to I.A-3.1a.B-1184, I.A-4.1a.B-1 to I.A-4.1a.B-1184; I.B-1.1a.B-1 to 1.6-1.1a.B-1184, I.B-2.1a.B-1 to 1.6-2.1a.B-1184, I.B-3.1a.B-1 to 1.6-3.1a.B-1184, I.B-4.1a.B-1 to 1.6-4.1a.B-1184; I.C-1.1a.B-1 to I.C-1.1a.B-1184, I.C-2.1a.B-1 to I.C-2.1a.B-1184, I.C-3.1a.B-1 to I.C-3.1a.B-1184, I.C-4.1a.B-1 to I.C-4.1a.B-1184).

Table 2a Compounds of the formula I.A-1, I.A-2, I.A-3, I.A-4, I.B-1, I.B-2, I.B-3, I.B-4 and I.C-1, I.C-2, I.C-3, I.C-4 in which R⁶ is CH₃ and the meaning for the combination of R², R³, R⁷ and R⁸ for each individual compound corresponds in each case to one line of Table B (compounds I.A-1.1b.B-1 to I.A-1.1b.B-1184, I.A-2.1b.B-1 to I.A-2.1b.B-1184, I.A-3.1b.B-1 to I.A-3.1b.B-1184, I.A-4.1b.B-1 to I.A-4.1b.B-1184;I.B-1.1b.B-1 to 1.6-1.1b.B-1184, I.B-2.1b.B-1 to 1.6-2.1b.B-1184, I.B-3.1b.B-1 to I.B-3.1b.B-1184, I.B-4.1b.B-1 to I.B-4.1b.B-1184; I.C-1.1b.B-1 to I.C-1.1b.B-1184, I.C-2.1b.B-1 to I.C-2.1b.B-1184, I.C-3.1b.B-1 to I.C-3.1b.B-1184, I.C-4.1b.B-1 to I.C-4.1b.B-1184).

Table 3a Compounds of the formula I.A-1, I.A-2, I.A-3, I.A-4, I.B-1, I.B-2, I.B-3, I.B-4 and I.C-1, I.C-2, I.C-3, I.C-4 in which R⁶ is —CH₂—CH₃ and the meaning for the combination of R², R³, R⁷ and R⁸ for each individual compound corresponds in each case to one line of Table B (compounds I.A-1.1b.B-1 to I.A-1.1b.B-1184, I.A-2.1b.B-1 to I.A-2.1b.B-1184, I.A-3.1b.B-1 to I.A-3.1b.B-1184, I.A-4.1b.B-1 to I.A-4.1b.B-1184; I.B-1.1b.B-1 to I.B-1.1b.B-1184, I.B-2.1b.B-1 to I.B-2.1b.B-1184, I.B-3.1b.B-1 to I.B-3.1b.B-1184, I.B-4.1b.B-1 to I.B-4.1b.B-1184; I.C-1.1b.B-1 to I.C-1.1b.B-1184, I.C-2.1b.B-1 to I.C-2.1b.B-1184, I.C-3.1b.B-1 to I.C-3.1b.B-1184, I.C-4.1b.B-1 to I.C-4.1b.B-1184).

The compounds I and the compositions according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes, and Deuteromycetes (syn. Fungi imperfecti). Some are systemically effective and they can be used in crop protection as foliar fungicides, fungicides for seed dressing, and soil fungicides. Moreover, they are suitable for controlling harmful fungi, which inter alia occur in wood or roots of plants.

The compounds I and the compositions according to the invention are particularly important in the control of a multitude of phytopathogenic fungi on various cultivated plants, such as cereals, e. g. wheat, rye, barley, triticale, oats, or rice; beet, e. g. sugar beet or fodder beet; fruits, e. g. pomes (apples, pears, etc.), stone fruits (plums, peaches, almonds, cherries, etc.), or soft fruits, which are also called berries (strawberries, raspberries, blackberries, gooseberries, etc.); leguminous plants, e. g. lentils, peas, alfalfa, or soybeans; oil plants, e. g. rape, mustard, olives, sunflowers, coconut, cocoa beans, castor oil plants, oil palms, ground nuts, or soybeans; cucurbits, e. g. squashes, cucumber, or melons; fiber plants, e. g. cotton, flax, hemp, or jute; citrus fruits, e. g. oranges, lemons, grapefruits, or mandarins; vegetables, e. g. spinach, lettuce, asparagus, cabbages, carrots, onions, tomatoes, potatoes, cucurbits, or paprika; lauraceous plants, e. g. avocados, cinnamon, or camphor; energy and raw material plants, e. g. corn, soybean, rape, sugar cane, or oil palm; corn; tobacco; nuts; coffee; tea; bananas; vines (table grapes and grape juice grape vines); hop; turf; sweet leaf (also called Stevia); natural rubber plants; or ornamental and forestry plants, e. g. flowers, shrubs, broad-leaved trees, or evergreens (conifers, eucalyptus, etc.); and on the plant propagation material, such as seeds; and on the crop material of these plants.

Preferably, compounds I and compositions thereof, respectively are used for controlling a multitude of fungi on field crops, such as potatoes, sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant, such as seeds; and vegetative plant materials, such as cuttings and tubers (e. g. potatoes), which can be used for the multiplication of the plant. This includes seeds, roots, fruits, tubers, bulbs, rhizomes, shoots, sprouts and other parts of plants; including seedlings and young plants, which are to be transplanted after germination or after emergence from soil. These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by mutagenesis or genetic engineering to provide a new trait to a plant or to modify an already present trait. Mutagenesis includes techniques of random mutagenesis using X-rays or mutagenic chemicals, but also techniques of targeted mutagenesis, to create mutations at a specific locus of a plant genome. Targeted mutagenesis techniques frequently use oligonucleotides or proteins like CRISPR/Cas, zinc-finger nucleases, TALENs or meganucleases to achieve the targeting effect. Genetic engineering usually uses recombinant DNA techniques to create modifications in a plant genome which under natural circumstances cannot readily be obtained by cross breeding, mutagenesis or natural recombination. Typically, one or more genes are integrated into the genome of a plant to add a trait or improve a trait. These integrated genes are also referred to as transgenes in the art, while plant comprising such transgenes are referred to as transgenic plants. The process of plant transformation usually produces several transformation events, which differ in the genomic locus in which a transgene has been integrated. Plants comprising a specific transgene on a specific genomic locus are usually described as comprising a specific “event”, which is referred to by a specific event name. Traits which have been introduced in plants or have been modified include herbicide tolerance, insect resistance, increased yield and tolerance to abiotic conditions, like drought.

Herbicide tolerance has been created by using mutagenesis as well as using genetic engineering. Plants which have been rendered tolerant to acetolactate synthase (ALS) inhibitor herbicides by mutagenesis and breeding comprise plant varieties commercially available under the name Clearfield®.

Herbicide tolerance has been created via the use of transgenes to glyphosate, glufosinate, 2,4-D, dicamba, oxynil herbicides, like bromoxynil and ioxynil, sulfonylurea herbicides, ALS inhibitors and 4-hydroxyphenylpyruvate dioxygenase (HPPD) inhibitors, like isoxaflutole and mesotrione.

Transgenes which have been used to provide herbicide tolerance traits comprise: for tolerance to glyphosate: cp4 epsps, epsps grg23ace5, mepsps, 2mepsps, gat4601, gat4621, goxv247; for tolerance to glufosinate: pat and bar, for tolerance to 2,4-D: aad-1, aad-12; for tolerance to dicamba: dmo; for tolerance to oxynil herbicies: bxn; for tolerance to sulfonylurea herbicides: zm-hra, csr1-2, gm-hra, S4-HrA; for tolerance to ALS inhibitors: csr1-2; and for tolerance to HPPD inhibitors: hppdPF, W336, avhppd-03.

Transgenic corn events comprising herbicide tolerance genes include, but are not limited to, DAS40278, MON801, MON802, MON809, MON810, MON832, MON87411, MON87419, MON87427, MON88017, MON89034, NK603, GA21, MZHGOJG, HCEM485, VCO-01981-5, 676, 678, 680, 33121, 4114, 59122, 98140, Bt10, Bt176, CBH-351, DBT418, DLL25, MS3, MS6, MZIR098, T25, TC1507 and TC6275. Transgenic soybean events comprising herbicide tolerance genes include, but are not limited to, GTS 40-3-2, MON87705, MON87708, MON87712, MON87769, MON89788, A2704-12, A2704-21, A5547-127, A5547-35, DP356043, DAS44406-6, DAS68416-4, DAS-81419-2, GU262, SYHTOH2, W62, W98, FG72 and CV127. Transgenic cotton events comprising herbicide tolerance genes include, but are not limited to, 19-51a, 31707, 42317, 81910, 281-24-236, 3006-210-23, BXN10211, BXN10215, BXN10222, BXN10224, MON1445, MON1698, MON88701, MON88913, GHB119, GHB614, LLCotton25, T303-3 and T304-40. Transgenic canola events comprising herbicide tolerance genes are for example, but not excluding others, MON88302, HCR-1, HCN10, HCN28, HCN92, MS1, MS8, PHY14, PHY23, PHY35, PHY36, RF1, RF2 and RF3.

Insect resistance has mainly been created by transferring bacterial genes for insecticidal proteins to plants: Transgenes which have most frequently been used are toxin genes of Bacillus spp. and synthetic variants thereof, like cry1A, cry1Ab, cry1Ab-Ac, cry1Ac, cry1A.105, cry1F, cry1Fa2, cry2Ab2, cry2Ae, mcry3A, ecry3.1Ab, cry3Bb1, cry34Ab1, cry35Ab1, cry9C, vip3A(a), vip3Aa20. However, also genes of plant origin, such as genes coding for protease inhibitors, like CpTI and pinII, have been transferred to other plants. A further approach uses transgenes such as dvsnf7 to produce double-stranded RNA in plants.

Transgenic corn events comprising genes for insecticidal proteins or double stranded RNA include, but are not limited to, Bt10, Bt11, Bt176, MON801, MON802, MON809, MON810, MON863, MON87411, MON88017, MON89034, 33121, 4114, 5307, 59122, TC1507, TC6275, CBH-351, MIR162, DBT418 and MZIR098. Transgenic soybean events comprising genes for insecticidal proteins include, but are not limited to, MON87701, MON87751 and DAS-81419. Transgenic cotton events comprising genes for insecticidal proteins include, but are not limited to, SGK321, MON531, MON757, MON1076, MON15985, 31707, 31803, 31807, 31808, 42317, BNLA-601, Event1, COT67B, COT102, T303-3, T304-40, GFM Cry1A, GK12, MLS 9124, 281-24-236, 3006-210-23, GHB119 and SGK321.

Increased yield has been created by using the transgene athb17, being present for example in corn event MON87403, or by using the transgene bbx32, being present for example in the soybean event MON87712.

Cultivated plants comprising a modified oil content have been created by using the transgenes: gm-fad2-1, Pj.D6D, Nc.Fad3, fad2-1A and fatb1-A. Soybean events comprising at least one of these genes are: 260-05, MON87705 and MON87769.

Tolerance to abiotic conditions, such as drought, has been created by using the transgene cspB, comprised by the corn event MON87460 and by using the transgene Hahb-4, comprised by soybean event IND-00410-5.

Traits are frequently combined by combining genes in a transformation event or by combining different events during the breeding process resulting in a cultivated plant with stacked traits. Preferred combinations of traits are combinations of herbicide tolerance traits to different groups of herbicides, combinations of insect tolerance to different kind of insects, in particular tolerance to lepidopteran and coleopteran insects, combinations of herbicide tolerance with one or several types of insect resistance, combinations of herbicide tolerance with increased yield as well as combinations of herbicide tolerance and tolerance to abiotic conditions.

Plants comprising singular or stacked traits as well as the genes and events providing these traits are well known in the art. For example, detailed information as to the mutagenized or integrated genes and the respective events are available from websites of the organizations “International Service for the Acquisition of Agri-biotech Applications (ISAAA)” (http://www.isaaa.org/gmapprovaldatabase) and the “Center for Environmental Risk Assessment (CERA)” (http://cera-gmc.org/GMCropDatabase). Further information on specific events and methods to detect them can be found for canola events MS1, MS8, RF3, GT73, MON88302, KK179 in WO01/031042, WO01/041558, WO01/041558, WO02/036831, WO11/153186, WO13/003558, for cotton events MON1445, MON15985, MON531 (MON15985), LLCotton25, MON88913, COT102, 281-24-236, 3006-210-23, COT67B, GHB614, T304-40, GHB119, MON88701, 81910 in WO02/034946, WO02/100163, WO02/100163, WO03/013224, WO04/072235, WO04/039986, WO05/103266, WO05/103266, WO06/128573, WO07/017186, WO08/122406, WO08/151780, WO12/134808, WO13/112527; for corn events GA21, MON810, DLL25, TC1507, MON863, MIR604, LY038, MON88017, 3272, 59122, NK603, MIR162, MON89034, 98140, 32138, MON87460, 5307, 4114, MON87427, DAS40278, MON87411, 33121, MON87403, MON87419 in WO98/044140, U.S. Ser. No. 02/102,582, U.S. Ser. No. 03/126,634, WO04/099447, WO04/011601, WO05/103301, WO05/061720, WO05/059103, WO06/098952, WO06/039376, US2007/292854, WO07/142840, WO07/140256, WO08/112019, WO09/103049, WO09/111263, WO10/077816, WO11/084621, WO11/062904, WO11/022469, WO13/169923, WO14/116854, WO15/053998, WO15/142571; for potato events E12, F10, J3, J55, V11, X17, Y9 in WO14/178910, WO14/178913, WO14/178941, WO14/179276, WO16/183445, WO17/062831, WO17/062825; for rice events LLRICE06, LLRICE601, LLRICE62 in WO00/026345, WO00/026356, WO00/026345; and for soybean events H7-1, MON89788, A2704-12, A5547-127, DP305423, DP356043, MON87701, MON87769, CV127, MON87705, DAS68416-4, MON87708, MON87712, SYHTOH2, DAS81419, DAS81419×DAS44406-6, MON87751 in WO04/074492, WO06/130436, WO06/108674, WO06/108675, WO08/054747, WO08/002872, WO09/064652, WO09/102873, WO10/080829, WO10/037016, WO11/066384, WO11/034704, WO12/051199, WO12/082548, WO13/016527, WO13/016516, WO14/201235.

The use of compounds I and compositions according to the invention, respectively, on cultivated plants may result in effects which are specific to a cultivated plant comprising a certain gene or event. These effects might involve changes in growth behavior or changed resistance to biotic or abiotic stress factors. Such effects may in particular comprise enhanced yield, enhanced resistance or tolerance to insects, nematodes, fungal, bacterial, mycoplasma, viral or viroid pathogens as well as early vigour, early or delayed ripening, cold or heat tolerance as well as changed amino acid or fatty acid spectrum or content.

The compounds I and compositions thereof, respectively, are particularly suitable for controlling the following plant diseases:

Albugo spp. (white rust) on ornamentals, vegetables (e. g. A. candida) and sunflowers (e. g. A. tragopogonis); Alternaria spp. (Alternaria leaf spot) on vegetables, rape (A. brassicola or brassicae), sugar beets (A. tenuis), fruits, rice, soybeans, potatoes (e. g. A. solani or A. alternata), tomatoes (e. g. A. solani or A. alternata) and wheat; Aphanomyces spp. on sugar beets and vegetables; Ascochyta spp. on cereals and vegetables, e. g. A. tritici (anthracnose) on wheat and A. hordei on barley; Bipolaris and Drechslera spp. (teleomorph: Cochliobolus spp.), e. g. Southern leaf blight (D. maydis) or Northern leaf blight (B. zeicola) on corn, e. g. spot blotch (B. sorokiniana) on cereals and e. g. B. oryzae on rice and turfs; Blumeria (formerly Erysphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e. g. C. ulmi (Dutch elm disease) on elms; Cercospora spp. (Cercospora leaf spots) on corn (e. g. Gray leaf spot: C. zeae-maydis), rice, sugar beets (e. g. C. beticola), sugarcane, vegetables, coffee, soybeans (e. g. C. sojina or C. kikuchii) and rice; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypi), corn (e. g. C. graminicola: Anthracnose stalk rot), soft fruits, potatoes (e. g. C. coccodes: black dot), beans (e. g. C. lindemuthianum) and soybeans (e. g. C. truncatum or C. gloeosporioides); Corticium spp., e. g. C. sasakii (sheath blight) on rice; Corynespora cassiicola (leaf spots) on soybeans and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri, teleomorph: Neonectria liriodendri Black Foot Disease) and ornamentals; Dematophora (teleomorph: Rosellinia) necatrx (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiohe spp. (powdery mildew) on sugar beets (E. betae), vegetables (e. g. E. pisi), such as cucurbits (e. g. E. cichoracearum), cabbages, rape (e. g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani (f. sp. glycines now syn. F. virguliforme) and F. tucumaniae and F. braslilense each causing sudden death syndrome on soybeans, and F. verticillioides on corn; Gaeumannomyces graminis (take-all) on cereals (e. g. wheat or barley) and corn; Gibberella spp. on cereals (e. g. G. zeae) and rice (e. g. G. fujikuroi: Bakanae disease); Glomerella cingulata on vines, pome fruits and other plants and G. gossypi on cotton; Grainstaining complex on rice; Guignardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals and rice; Hemleia spp., e. g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (bloom and twig blight, brown rot) on stone fruits and other rosaceous plants; Mycosphaerella spp. on cereals, bananas, soft fruits and ground nuts, such as e. g. M. graminicola (anamorph: Septoria tritici, Septoria blotch) on wheat or M. fliensis (black Sigatoka disease) on bananas; Peronospora spp. (downy mildew) on cabbage (e. g. P. brassicae), rape (e. g. P. parasitica), onions (e. g. P. destructor), tobacco (P. tabacina) and soybeans (e. g. P. manshurica); Phakopsora pachyrhlzi and P. meibomiae (soybean rust) on soybeans; Phialophora spp. e. g. on vines (e. g. P. trachephila and P. tetraspora) and soybeans (e. g. P. gregata: stem rot); Phoma lingam (root and stem rot) on rape and cabbage and P. betae (root rot, leaf spot and damping-off) on sugar beets; Phomopsis spp. on sunflowers, vines (e. g. P. viticola: can and leaf spot) and soybeans (e. g. stem rot: P. phaseol, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsic), soybeans (e. g. P. megasperma, syn. P. sojae), potatoes and tomatoes (e. g. P. infestans: late blight) and broad-leaved trees (e. g. P. ramorum: sudden oak death); Plasmodiophora brassicae (club root) on cabbage, rape, radish and other plants; Plasmopara spp., e. g. P. viticola (grapevine downy mildew) on vines and P. halstedii on sunflowers; Podosphaera spp. (powdery mildew) on rosaceous plants, hop, pome and soft fruits, e. g. P. leucotricha on apples; Polymyxa spp., e. g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula trachephila (red fire disease or ‘rotbrenner’, anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, P. kuehnii (orange rust) on sugar cane and P. asparagi on asparagus; Pyrenophora (anamorph: Drechslera) tritici-repentis (tan spot) on wheat or P. teres (net blotch) on barley; Pyricularia spp., e. g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e. g. P. ultimum or P. aphanidermatum); Ramularia spp., e. g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secals (scald) on barley, rye and triticale; Sarocladium oryzae and S. attenuatum (sheath rot) on rice; Sclerotinia spp. (stem rot or white mold) on vegetables and field crops, such as rape, sunflowers (e. g. S. sclerotiorum) and soybeans (e. g. S. rolfsii or S. sclerotiorum); Septoria spp. on various plants, e. g. S. glycines (brown spot) on soybeans, S. tritici (Septoria blotch) on wheat and S. (syn. Stagonospora) nodorum (Stagonospora blotch) on cereals; Uncinula (syn. Erysphe) necator (powdery mildew, anamorph: Oidium tuckeri) on vines; Setospaeria Spp. (Leaf Blight) on Corn (e. g. S. turcicum, Syn. Helminthosporium turcicum) and turf; Sphacelotheca spp. (smut) on corn, (e. g. S. reliana: head smut), sorghum und sugar cane; Sphaerotheca fuliginea (powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tietia spp. (common bunt or stinking smut) on cereals, such as e. g. T. tritici (syn. T caries, wheat bunt) and T. controversa (dwarf bunt) on wheat; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli) and sugar beets (e. g. U. betae); Ustilago spp. (loose smut) on cereals (e. g. U. nuda and U. avaenae), corn (e. g. U. maydis: corn smut) and sugar cane; Venturia spp. (scab) on apples (e. g. V. inaequais) and pears; and Verticillium spp. (wilt) on various plants, such as fruits and ornamentals, vines, soft fruits, vegetables and field crops, e. g. V. dahliae on strawberries, rape, potatoes and tomatoes.

The compounds I and compositions thereof, respectively, are also suitable for controlling harmful microorganisms in the protection of stored products or harvest, and in the protection of materials.

The term “stored products or harvest” is understood to denote natural substances of plant or animal origin and their processed forms, which have been taken from the natural life cycle and for which long-term protection is desired. Stored products of crop plant origin, such as plants or parts thereof, for example stalks, leafs, tubers, seeds, fruits or grains, can be protected in the freshly harvested state or in processed form, such as pre-dried, moistened, comminuted, ground, pressed or roasted, which process is also known as post-harvest treatment. Also falling under the definition of stored products is timber, whether in the form of crude timber, such as construction timber, electricity pylons and barriers, or in the form of finished articles, such as furniture or objects made from wood. Stored products of animal origin are hides, leather, furs, hairs and the like. Preferably, “stored products” is understood to denote natural substances of plant origin and their processed forms, more preferably fruits and their processed forms, such as pomes, stone fruits, soft fruits and citrus fruits and their processed forms. The compounds I and compositions thereof according the present invention can prevent disadvantageous effects such as decay, discoloration or mold.

The term “protection of materials” is to be understood to denote the protection of technical and non-living materials, such as adhesives, glues, wood, paper, paperboard, textiles, leather, paint dispersions, plastics, cooling lubricants, fiber, or fabrics; against the infestation and destruction by harmful microorganisms, such as fungi and bacteria. As to the protection of materials, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp.; Deuteromycetes, such as Aspergillus spp., Cladosporium spp., Penicillium spp., Trichoderma spp., Alternaria spp., Paecilomyces spp.; and Zygomycetes, such as Mucor spp.

In the protection of stored products and harvest in addition the following yeast fungi are worthy of note: Candida spp. and Saccharomyces cerevisae.

The compounds I and compositions thereof, respectively, may be used for improving the health of a plant. The invention also relates to a method for improving plant health by treating a plant, its propagation material, and/or the locus where the plant is growing or is to grow with an effective amount of compounds I and compositions thereof, respectively.

The term “plant health” is to be understood to denote a condition of the plant and/or its products which is determined by several indicators alone or in combination with each other, such as yield (e. g. increased biomass and/or increased content of valuable ingredients), plant vigor (e. g. improved plant growth and/or greener leaves (“greening effect”)), quality (e. g. improved content or composition of certain ingredients), and tolerance to abiotic and/or biotic stress. The above identified indicators for the health condition of a plant may be interdependent or may result from each other.

The compounds of formula I can be present in different crystal modifications whose biological activity may differ. They are likewise subject matter of the present invention.

The compounds I are employed as such or in form of compositions by treating the fungi, the plants, plant propagation materials, such as seeds; soil, surfaces, materials, or rooms to be protected from fungal attack with a fungicidally effective amount of the active substances. The application can be carried out both before and after the infection of the plants, plant propagation materials, such as seeds; soil, surfaces, materials or rooms by the fungi.

Plant propagation materials may be treated with compounds I as such or a composition comprising at least one compound I prophylactically either at or before planting or transplanting.

The invention also relates to agrochemical compositions comprising an auxiliary and at least one compound I according to the invention.

An agrochemical composition comprises a fungicidally effective amount of a compound I.

The term “fungicidally effective amount” denotes an amount of the composition or of the compounds I, which is sufficient for controlling harmful fungi on cultivated plants or in the protection of stored products or harvest or of materials and which does not result in a substantial damage to the treated plants, the treated stored products or harvest, or to the treated materials. Such an amount can vary in a broad range and is dependent on various factors, such as the fungal species to be controlled, the treated cultivated plant, stored product, harvest or material, the climatic conditions and the specific compound I used.

The compounds I, their N-oxides and salts can be converted into customary types of agrochemical compositions, e. g. solutions, emulsions, suspensions, dusts, powders, pastes, granules, pressings, capsules, and mixtures thereof. Examples for composition types are suspensions (e. g. SC, OD, FS), emulsifiable concentrates (e. g. EC), emulsions (e. g. EW, EO, ES, ME), capsules (e. g. CS, ZC), pastes, pastilles, wettable powders or dusts (e. g. WP, SP, WS, DP, DS), pressings (e. g. BR, TB, DT), granules (e. g. WG, SG, GR, FG, GG, MG), insecticidal articles (e. g. LN), as well as gel formulations for the treatment of plant propagation materials, such as seeds (e. g. GF). These and further compositions types are defined in the “Catalogue of pesticide formulation types and international coding system”, Technical Monograph No. 2, 6^{th} Ed. May 2008, CropLife International.

The compositions are prepared in a known manner, such as described by Mollet and Grubemann, Formulation technology, Wiley VCH, Weinheim, 2001; or by Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers, and binders.

Suitable solvents and liquid carriers are water and organic solvents, such as mineral oil fractions of medium to high boiling point, e. g. kerosene, diesel oil; oils of vegetable or animal origin; aliphatic, cyclic and aromatic hydrocarbons, e. g. toluene, paraffin, tetrahydronaphthalene, and alkylated naphthalenes; alcohols, e. g. ethanol, propanol, butanol, benzyl alcohol, cyclohexanol, glycols; DMSO; ketones, e. g. cyclohexanone; esters, e. g. lactates, carbonates, fatty acid esters, gamma-butyrolactone; fatty acids; phosphonates; amines; amides, e. g. methyl pyrrolidone, fatty acid dimethyl amides; and mixtures thereof.

Suitable solid carriers or fillers are mineral earths, e. g. silicates, silica gels, talc, kaolins, limestone, lime, chalk, clays, dolomite, diatomaceous earth, bentonite, calcium sulfate, magnesium sulfate, magnesium oxide; polysaccharides, e. g. cellulose, starch; fertilizers, e. g. ammonium sulfate, ammonium phosphate, ammonium nitrate, ureas; products of vegetable origin, e. g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and of alkyl naphthalenes, sulfosuccinates, or sulfosuccinamates. Examples of sulfates are sulfates of fatty acids, of oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters, or monoglycerides. Examples of sugar-based surfactants are sorbitans, ethoxylated sorbitans, sucrose and glucose esters, or alkylpolyglucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide, and polypropylene oxide. Suitable polyelectrolytes are polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e. g. xanthan gum, carboxymethyl cellulose), inorganic clays (organically modified or unmodified), polycarboxylates, and silicates.

Suitable bactericides are bronopol and isothiazolinone derivatives, such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e. g. in red, blue, or green) are pigments of low water solubility and water-soluble dyes. Examples are inorganic colorants (e. g. iron oxide, titan oxide, iron hexacyanoferrate) and organic colorants (e. g. alizarin-, azo- and phthalocyanine colorants).

Suitable tackifiers or binders are polyvinyl pyrrolidones, polyvinyl acetates, polyvinyl alcohols, polyacrylates, biological or synthetic waxes, and cellulose ethers.

Examples for composition types and their preparation are:

i) Water-Soluble Concentrates (SL, LS)

10-60 wt % of a compound I and 5-15 wt % wetting agent (e. g. alcohol alkoxylates) are dissolved in water and/or in a water-soluble solvent (e. g. alcohols) ad 100 wt %. The active substance dissolves upon dilution with water.

ii) Dispersible Concentrates (DC)

5-25 wt % of a compound I and 1-10 wt % dispersant (e. g. polyvinyl pyrrolidone) are dissolved in organic solvent (e. g. cyclohexanone) ad 100 wt %. Dilution with water gives a dispersion.

iii) Emulsifiable Concentrates (EC)

15-70 wt % of a compound I and 5-10 wt % emulsifiers (e. g. calcium dodecylbenzenesulfonate and castor oil ethoxylate) are dissolved in water-insoluble organic solvent (e. g. aromatic hydrocarbon) ad 100 wt %. Dilution with water gives an emulsion.

iv) Emulsions (EW, EO, ES)

5-40 wt % of a compound I and 1-10 wt % emulsifiers (e. g. calcium dodecylbenzenesulfonate and castor oil ethoxylate) are dissolved in 20-40 wt % water-insoluble organic solvent (e. g. aromatic hydrocarbon). This mixture is introduced into water ad 100 wt % by means of an emulsifying machine and made into a homogeneous emulsion. Dilution with water gives an emulsion.

v) Suspensions (SC, OD, FS)

In an agitated ball mill, 20-60 wt % of a compound I are comminuted with addition of 2-10 wt % dispersants and wetting agents (e. g. sodium lignosulfonate and alcohol ethoxylate), 0.1-2 wt % thickener (e. g. xanthan gum) and water ad 100 wt % to give a fine active substance suspension. Dilution with water gives a stable suspension of the active substance. For FS type composition, up to 40 wt % binder (e. g. polyvinyl alcohol) is added.

vi) Water-Dispersible Granules and Water-Soluble Granules (WG, SG)

50-80 wt % of a compound I are ground finely with addition of dispersants and wetting agents (e. g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound I are ground in a rotor-stator mill with addition of 1-5 wt % dispersants (e. g. sodium lignosulfonate), 1-3 wt % wetting agents (e. g. alcohol ethoxylate) and solid carrier (e. g. silica gel) ad 100 wt %. Dilution with water gives a stable dispersion or solution of the active substance.

viii) Gel (GW, GF)

In an agitated ball mill, 5-25 wt % of a compound I are comminuted with addition of 3-10 wt % dispersants (e. g. sodium lignosulfonate), 1-5 wt % thickener (e. g. carboxymethyl cellulose) and water ad 100 wt % to give a fine suspension of the active substance. Dilution with water gives a stable suspension of the active substance.

ix) Microemulsion (ME)

5-20 wt % of a compound I are added to 5-30 wt % organic solvent blend (e. g. fatty acid dimethyl amide and cyclohexanone), 10-25 wt % surfactant blend (e. g. alcohol ethoxylate and arylphenol ethoxylate), and water ad 100%. This mixture is stirred for 1 h to produce spontaneously a thermodynamically stable microemulsion.

x) Microcapsules (CS)

An oil phase comprising 5-50 wt % of a compound I, 0-40 wt % water insoluble organic solvent (e. g. aromatic hydrocarbon), 2-15 wt % acrylic monomers (e. g. methylmethacrylate, methacrylic acid and a di- or triacrylate) are dispersed into an aqueous solution of a protective colloid (e. g. polyvinyl alcohol). Radical polymerization results in the formation of poly(meth)acrylate microcapsules. Alternatively, an oil phase comprising 5-50 wt % of a compound I according to the invention, 0-40 wt % water insoluble organic solvent (e. g. aromatic hydrocarbon), and an isocyanate monomer (e. g. diphenylmethene-4,4′-diisocyanate) are dispersed into an aqueous solution of a protective colloid (e. g. polyvinyl alcohol). The addition of a polyamine (e. g. hexamethylenediamine) results in the formation of polyurea microcapsules. The monomers amount to 1-10 wt %. The wt % relate to the total CS composition.

xi) Dustable Powders (DP, DS)

1-10 wt % of a compound I are ground finely and mixed intimately with solid carrier (e. g. finely divided kaolin) ad 100 wt %.

xii) Granules (GR, FG)

0.5-30 wt % of a compound I is ground finely and associated with solid carrier (e. g. silicate) ad 100 wt %. Granulation is achieved by extrusion, spray-drying or fluidized bed.

xiii) Ultra-Low Volume Liquids (UL)

1-50 wt % of a compound I are dissolved in organic solvent (e. g. aromatic hydrocarbon) ad 100 wt %.

The compositions types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, more preferably between 1 and 70%, and in particular between 10 and 60%, by weight of active substance. The active substances are employed in a purity of from 90% to 100%, preferably from 95-% to 100% (according to NMR spectrum).

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed. The compositions in question give, after two-to-tenfold dilution, active substance concentrations of from 0.01 to 60% by weight, preferably from 0.1 to 40%, in the ready-to-use preparations. Application can be carried out before or during sowing. Methods for applying compound I and compositions thereof, respectively, onto plant propagation material, especially seeds, include dressing, coating, pelleting, dusting, soaking, as well as in-furrow application methods. Preferably, compound I or the compositions thereof, respectively, are applied on to the plant propagation material by a method such that germination is not induced, e. g. by seed dressing, pelleting, coating, and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials, such as seeds, e. g. by dusting, coating, or drenching, amounts of active substance of from 0.1 to 1000 g, preferably from 1 to 1000 g, more preferably from 1 to 100 g and most preferably from 5 to 100 g, per 100 kg of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e. g. herbicides, insecticides, fungicides, growth regulators, safeners, biopesticides) may be added to the active substances or the compositions comprising them as premix, or, if appropriate, not until immediately prior to use (tank mix). These agents can be admixed with the compositions according to the invention in a weight ratio of 1:100 to 100:1, preferably 1:10 to 10:1.

A pesticide is generally a chemical or biological agent (such as pestidal active ingredient, compound, composition, virus, bacterium, antimicrobial, or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease. The term “pesticide” includes also plant growth regulators that alter the expected growth, flowering, or reproduction rate of plants; defoliants that cause leaves or other foliage to drop from a plant, usually to facilitate harvest; desiccants that promote drying of living tissues, such as unwanted plant tops; plant activators that activate plant physiology for defense of against certain pests; safeners that reduce unwanted herbicidal action of pesticides on crop plants; and plant growth promoters that affect plant physiology e.g. to increase plant growth, biomass, yield or any other quality parameter of the harvestable goods of a crop plant.

Biopesticides have been defined as a form of pesticides based on microorganisms (bacteria, fungi, viruses, nematodes, etc.) or natural products (compounds, such as metabolites, proteins, or extracts from biological or other natural sources) (U.S. Environmental Protection Agency: http://www.epa.gov/pesticides/biopesticides/). Biopesticides fall into two major classes, microbial and biochemical pesticides:
(1) Microbial pesticides consist of bacteria, fungi or viruses (and often include the metabolites that bacteria and fungi produce). Entomopathogenic nematodes are also classified as microbial pesticides, even though they are multi-cellular.
(2) Biochemical pesticides are naturally occurring substances that control pests or provide other crop protection uses as defined below, but are relatively non-toxic to mammals.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank or any other kind of vessel used for applications (e. g. seed treater drums, seed pelleting machinery, knapsack sprayer) and further auxiliaries may be added, if appropriate.

When living microorganisms, such as microbial pesticides from groups L1), L3) and L5), form part of such kit, it must be taken care that choice and amounts of the components (e. g. chemical pesticides) and of the further auxiliaries should not influence the viability of the microbial pesticides in the composition mixed by the user. Especially for bactericides and solvents, compatibility with the respective microbial pesticide has to be taken into account.

Consequently, one embodiment of the invention is a kit for preparing a usable pesticidal composition, the kit comprising a) a composition comprising component 1) as defined herein and at least one auxiliary; and b) a composition comprising component 2) as defined herein and at least one auxiliary; and optionally c) a composition comprising at least one auxiliary and optionally a further active component 3) as defined herein.

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of pesticides II (e. g. pesticidally-active substances and biopesticides), in conjunction with which the compounds I can be used, is intended to illustrate the possible combinations but does not limit them:

### A) Respiration Inhibitors

Inhibitors of complex III at Qₙ site: azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxystrobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylideneaminooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21), methyl-N-[2-[(1,4-dimethyl-5-phenyl-pyrazol-3-yl)oxylmethyl]phenyl]-N-methoxy-carbamate (A.1.22), metyltetrapole (A.1.25), (Z,2E)-5-[1-(2,4-dichlorophenyl)pyrazol-3-yl]-oxy-2-methoxyimino-N,3-dimethyl-pent-3-enamide (A.1.34), (Z,2E)-5-[1-(4-chlorophenyl)pyrazol-3-yl]oxy-2-methoxyimino-N,3-dimethylpent-3-enamide (A.1.35), pyriminostrobin (A.1.36), bifujunzhi (A.1.37), 2-(ortho-((2,5-dimethylphenyl-oxymethylen)phenyl)-3-methoxy-acrylic acid methylester (A.1.38);
inhibitors of complex III at Qᵢ site: cyazofamid (A.2.1), amisulbrom (A.2.2), [(6S,7R,8R)-8-benzyl-3-[(3-hydroxy-4-methoxy-pyridine-2-carbonyl)amino]-6-methyl-4,9-dioxo-1,5-dioxonan-7-yl] 2-methylpropanoate (A.2.3), fenpicoxamid (A.2.4), florylpicoxamid (A.2.5);
inhibitors of complex II: benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.15), penthiopyrad (A.3.16), pydiflumetofen (A.3.17), pyraziflumid (A.3.18), sedaxane (A.3.19), tecloftalam (A.3.20), thifluzamide (A.3.21), inpyrfluxam (A.3.22), pyrapropoyne (A.3.23), fluindapyr (A.3.28), methyl (E)-2-[2-[(5-cyano-2-methyl-phenoxy)methyl]phenyl]-3-methoxy-prop-2-enoate (A.3.30), isoflucypram (A.3.31), 2-(difluoromethyl)-N-(1,1,3-trimethyl-indan-4-yl)pyridine-3-carboxamide (A.3.32), 2-(difluoromethyl)-N-[(3R)-1,1,3-trimethylindan-4-yl]pyridine-3-carboxamide (A.3.33), 2-(difluoromethyl)-N-(3-ethyl-1,1-dimethyl-indan-4-yl)pyridine-3-carboxamide (A.3.34), 2-(difluoromethyl)-N-[(3R)-3-ethyl-1,1-dimethyl-indan-4-yl]pyridine-3-carboxamide (A.3.35), 2-(difluoromethyl)-N-(1,1-dimethyl-3-propyl-indan-4-yl)pyridine-3-carboxamide (A.3.36), 2-(difluoromethyl)-N-[(3R)-1,1-dimethyl-3-propyl-indan-4-yl]pyridine-3-carboxamide (A.3.37), 2-(difluoromethyl)-N-(3-isobutyl-1,1-dimethyl-indan-4-yl)pyridine-3-carboxamide (A.3.38), 2-(difluoromethyl)-N-[(3R)-3-isobutyl-1,1-dimethyl-indan-4-yl]pyridine-3-carboxamide (A.3.39);
other respiration inhibitors: diflumetorim (A.4.1); nitrophenyl derivates: binapacryl (A.4.2), dinobuton (A.4.3), dinocap (A.4.4), fluazinam (A.4.5), meptyldinocap (A.4.6), ferimzone (A.4.7); organometal compounds: fentin salts, e. g. fentin-acetate (A.4.8), fentin chloride (A.4.9) or fentin hydroxide (A.4.10); ametoctradin (A.4.11); silthiofam (A.4.12);

### B) Sterol Biosynthesis Inhibitors (SBI Fungicides)

C14 demethylase inhibitors: triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 2-(2,4-difluorophenyl)-1,1-difluoro-3-(tetrazol-1-yl)-1-[5-[4-(2,2,2-trifluoroethoxy)phenyl]-2-pyridyl]propan-2-ol (B.1.31), 2-(2,4-difluorophenyl)-1,1-difluoro-3-(tetrazol-1-yl)-1-[5-[4-(trifluoromethoxy)phenyl]-2-pyridyl]propan-2-ol (B.1.32), ipfentrifluconazole (B.1.37), mefentrifluconazole (B.1.38), 2-(chloromethyl)-2-methyl-5-(p-tolylmethyl)-1-(1,2,4-triazol-1-ylmethyl)cyclopentanol (B.1.43); imidazoles: imazalil (B.1.44), pefurazoate (B.1.45), prochloraz (B.1.46), triflumizol (B.1.47); pyrimidines, pyridines, piperazines: fenarimol (B.1.49), pyrifenox (B.1.50), triforine (B.1.51), [3-(4-chloro-2-fluoro-phenyl)-5-(2,4-difluorophenyl)isoxazol-4-yl]-(3-pyridyl)methanol (B.1.52);
Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);
Inhibitors of 3-keto reductase: fenhexamid (B.3.1);
Other Sterol biosynthesis inhibitors: chlorphenomizole (B.4.1);

### C) Nucleic Acid Synthesis Inhibitors

phenylamides or acyl amino acid fungicides: benalaxyl (C.1.1), benalaxyl-M (C.1.2), kiralaxyl (C.1.3), metalaxyl (C.1.4), metalaxyl-M (C.1.5), ofurace (C.1.6), oxadixyl (C.1.7);
other nucleic acid synthesis inhibitors: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7), 5-fluoro-2-(4-chlorophenylmethoxy)pyrimidin-4 amine (C.2.8);

### D) Inhibitors of Cell Division and Cytoskeleton

tubulin inhibitors: benomyl (D.1.1), carbendazim (D.1.2), fuberidazole (D1.3), thiabendazole (D.1.4),thiophanate-methyl(1.5), pyridachlometyl (D.1.6), (ethyl-2-[(3-ethynyl-8-methyl-6-quinolyl)oxy]butanamide (D.1.8), ethyl-2-[(3-ethynyl-8-methyl-6-quinolyl)oxy]-2-methylsulfanyl-acetamide (D.1.9), 2-[(3-ethynyl-8-methyl-6-quinolyl)oxy]-N-(2-fluoroethyl)butanamide (D.1.10), 2-[(3-ethynyl-8-methyl-6-quinolyl)oxy]-N-(2-fluoroethyl)-2-methoxy-acetamide (D.1.11), 2-[(3-ethynyl-8-methyl-6-quinolyl)oxy]-N-propyl-butanamide (D.1.12), 2-[(3-ethynyl-8-methyl-6-quinolyl)oxy]-2-methoxy-N-propyl-acetamide (D.1.13), 2-[(3-ethynyl-8-methyl-6-quinolyl)oxy]-2-methylsulfanyl-N-propyl-acetamide (D.1.14), 2-[(3-ethynyl-8-methyl-6-quinolyl)oxy]-N-(2-fluoroethyl)-2-methylsulfanyl-acetamide (D.1.15), 4-(2-bromo-4-fluorophenyl)-N-(2-chloro-6-fluoro-phenyl)-2,5-dimethyl-pyrazol-3-amine (D.1.16);
other cell division inhibitors: diethofencarb (D.2.1), ethaboxam (D.2.2), pencycuron (D.2.3), fluopicolide (D.2.4), zoxamide (D.2.5), metrafenone (D.2.6), pyriofenone (D.2.7);

### E) Inhibitors of Amino Acid and Protein Synthesis

methionine synthesis inhibitors: cyprodinil (E.1.1), mepanipyrim (E.1.2), pyrimethanil (E.1.3);
protein synthesis inhibitors: blasticidin-S (E.2.1), kasugamycin (E.2.2), kasugamycin hydrochloride-hydrate (E.2.3), mildiomycin (E.2.4), streptomycin (E.2.5), oxytetracyclin (E.2.6);

### F) Signal Transduction Inhibitors

MAP/histidine kinase inhibitors: fluoroimid (F.1.1), iprodione (F.1.2), procymidone (F.1.3), vinclozolin (F.1.4), fludioxonil (F.1.5);
G protein inhibitors: quinoxyfen (F.2.1);

### G) Lipid and Membrane Synthesis Inhibitors

Phospholipid biosynthesis inhibitors: edifenphos (G.1.1), iprobenfos (G.1.2), pyrazophos (G.1.3), isoprothiolane (G.1.4);
lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7);
phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7);
compounds affecting cell membrane permeability and fatty acides: propamocarb (G.4.1);
inhibitors of oxysterol binding protein: oxathiapiprolin (G.5.1), 2-{3-[2-(1-{[3,5-bis(difluoromethyl-1H-pyrazol-1-yl]acetyl}piperidin-4-yl)-1,3-thiazol-4-yl]-4,5-dihydro-1,2-oxazol-5-yl}phenyl methanesulfonate (G.5.2), 2-{3-[2-(1-{[3,5-bis(difluoromethyl)-1H-pyrazol-1-yl]acetyl}piperidin-4-yl) 1,3-thiazol-4-yl]-4,5-dihydro-1,2-oxazol-5-yl}-3-chlorophenyl methanesulfonate (G.5.3), 4-[1-[2-[3-(difluoromethyl)-5-methyl-pyrazol-1-yl]acetyl]-4-piperidyl]-N-tetralin-1-ylpyridine-2-carboxamide (G.5.4), 4-[1-[2-[3,5-bis(difluoromethyl)pyrazol-1-yl]acetyl]-4-piperidyl]-N-tetralin-1-yl-pyridine-2-carboxamide (G.5.5), 4-[1-[2-[3-(difluoromethyl)-5-(trifluoromethyl)pyrazol-1-yl]acetyl]-4-piperidyl]-N-tetralin-1-yl-pyridine-2-carboxamide (G.5.6), 4-[1-[2-[5-cyclopropyl-3-(difluoromethyl)pyrazol-1-yl]acetyl]-4-piperidyl]-N-tetralin-1-yl-pyridine-2-carboxamide (G.5.7), 4-[1-[2-[5-methyl-3-(trifluoromethyl)pyrazol-1-yl]acetyl]-4-piperidyl]-tetralin-1-yl-pyridine-2-carboxamide (G.5.8), 4-[1-[2-[5-(difluoromethyl)-3-(trifluoromethyl)pyrazol-1-yl]acetyl]-4-piperidyl]-N-tetralin-1-yl-pyridine-2-carboxamide (G.5.9), 4-[1-[2-[3,5-bis(trifluoromethyl)pyrazol-1-yl]acetyl]-4-piperidyl]-N-tetralin-1-yl-pyridine-2-carboxamide (G.5.10), (4-[1-[2-[5-cyclopropyl-3-(trifluoromethyl)pyrazol-1-yl]acetyl]-4-piperidyl]-N-tetralin-1-yl-pyridine-2-carboxamide (G.5.11);
   H) Inhibitors with Multi Site Action
inorganic active substances: Bordeaux mixture (H.1.1), copper (H.1.2), copper acetate (H.1.3), copper hydroxide (H.1.4), copper oxychloride (H.1.5), basic copper sulfate (H.1.6), sulfur (H.1.7);
thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
organochlorine compounds: anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11);
guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6)-tetraone (H.4.10);

### I) Cell Wall Synthesis Inhibitors

inhibitors of glucan synthesis: validamycin (1.1.1), polyoxin B (1.1.2);
melanin synthesis inhibitors: pyroquilon (1.2.1), tricyclazole (1.2.2), carpropamid (1.2.3), dicyclomet (1.2.4), fenoxanil (1.2.5);

### J) Plant Defence Inducers

acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), calcium phosphonate (J.1.11), potassium phosphonate (J.1.12), potassium or sodium bicarbonate (J.1.9), 4-cyclopropyl-N-(2,4-dimethoxyphenyl)thiadiazole-5-carboxamide (J.1.10);

### K) Unknown Mode of Action

bronopol (K.1.1), chinomethionat (K.1.2), cyflufenamid (K.1.3), cymoxanil (K.1.4), dazomet (K.1.5), debacarb (K.1.6), diclocymet (K.1.7), diclomezine (K.1.8), difenzoquat (K.1.9), difenzoquat-methylsulfate (K.1.10), diphenylamin (K.1.11), fenitropan (K.1.12), fenpyrazamine (K.1.13), flumetover (K.1.14), flusulfamide (K.1.15), flutianil (K.1.16), harpin (K.1.17), methasulfocarb (K.1.18), nitrapyrin (K.1.19), nitrothal-isopropyl (K.1.20), tolprocarb (K.1.21), oxincopper (K.1.22), proquinazid (K.1.23), tebufloquin (K.1.24), tecloftalam (K.1.25), triazoxide (K.1.26), N′-(4-(4-chloro-3-trifluoromethyl-phenoxy)-2,5-dimethyl-phenyl)-N-ethyl-N-methyl formamidine (K.1.27), N′-(4-(4-fluoro-3-trifluoromethyl-phenoxy)-2,5-dimethyl-phenyl)-N-ethyl-N-methyl formamidine (K.1.28), N′-[4-[[3-[(4-chlorophenyl)methyl]-1,2,4-thiadiazol-5-yl]-oxy]-2,5-dimethyl-phenyl]-N-ethyl-N-methyl-formamidine (K.1.29), N′-(5-bromo-6-indan-2-yloxy-2-methyl-3-pyridyl)-N-ethyl-N-methyl-formamidine (K.1.30), N′-[5-bromo-6-[1-(3,5-difluorophenyl)ethoxy]-2-methyl-3-pyridyl]-N-ethyl-N-methyl-formamidine (K.1.31), N′-[5-bromo6-(4-isopropylcyclohexoxy)-2-methyl-3-pyridyl]-N-ethyl-N-methyl-formamidine (K.1.32), N′-[5-bromo-2-methyl-6-(1-phenylethoxy)-3-pyridyl]-Methyl-N-methyl-formamidine (K.1.33), N′-(2-methyl-5-trifluoromethyl-4-(3-trimethylsilanyl-propoxy)-phenyl)-N-ethyl-N-methyl formamidine (K.1.34), N′-(5-difluoromethyl-2-methyl-4-(3-trimethylsilanyl-propoxy)-phenyl)-N-ethyl-N-methyl formamidine (K.1.35), 2-(4-chloro-phenyl)-N-[4-(3,4-dimethoxy-phenyl)-isoxazol-5-yl]-2-prop-2-ynyloxy-acetamide (K.1.36), 3-[5-(4-chloro-phenyl)-2,3-dimethyl-isoxazolidin-3-yl]-pyridine (pyrisoxazole) (K.1.37), 3-[5-(4-methylphenyl)-2,3-dimethyl-isoxazolidin-3 yl]-pyridine (K.1.38), 5-chloro-1-(4,6-dimethoxy-pyrimidin-2-yl)-2-methyl-1H-benzoimidazole (K.1.39), ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carbamate (K.1.42), but-3-ynyl N-[6-[[(Z)-[(1-methyltetrazol-5-yl)-phenyl-methylene]amino]oxymethyl]-2-pyridyl]carbamate (K.1.43), ipflufenoquin (K.1.44), quinofumelin (K.1.47), 2-(6-benzyl-2-pyridyl)quinazoline (K.1.50), 2-[6-(3-fluoro-4-methoxy-phenyl)-5-methyl-2-pyridyl]quinazoline (K.1.51), dichlobentiazox (K.1.52), N′-(2,5-dimethyl-4-phenoxy-phenyl)-N-ethyl-N-methyl-formamidine (K.1.53), pyrifenamine (K.1.54);

### L) Biopesticides

L1) Microbial pesticides with fungicidal, bactericidal, viricidal and/or plant defense activator activity: Ampelomyces quisqualis, Aspergillus flavus, Aureobasidium pullulans, Bacillus altitudinis, B. amyloliquefaciens, B. megaterium, B. mojavensis, B. mycoides, B. pumilus, B. simplex, B. solisalsi, B. subtilis, B. subtilis var. amyloliquefaciens, Candida oleophila, C. saitoana, Clavibacter michiganensis (bacteriophages), Coniothyrium minitans, Cryphonectria parasitica, Cryptococcus albidus, Dilophosphora alopecuri, Fusarium oxysporum, Clonostachys rosea f. catenulate (also named Gliocladium catenulatum), Gliocladium roseum, Lysobacter antibioticus, L. enzymogenes, Metschnikowia fructicola, Microdochium dimerum, Microsphaeropsis ochracea, Muscodor albus, Paenibacillus alvei, Paenibacillus epiphyticus, P. polymyxa, Pantoea vagans, Penicillium bilaiae, Phlebiopsis gigantea, Pseudomonas sp., Pseudomonas chloraphis, Pseudozyma flocculosa, Pichia anomala, Pythium oligandrum, Sphaerodes mycoparasitica, Streptomyces griseoviridis, S. lydicus, S. violaceusniger, Talaromyces flavus, Trichoderma asperelloides, T. asperellum, T. atroviride, T. fertile, T. gamsii, T. harmatum, T. harzianum, T. polysporum, T. stromaticum, T. virens, T. viride, Typhula phacorrhiza, Ulocladium oudemansii, Verticillium dahlia, zucchini yellow mosaic virus (avirulent strain);
L2) Biochemical pesticides with fungicidal, bactericidal, viricidal and/or plant defense activator activity: harpin protein, Reynoutria sachalinensis extract;
L3) Microbial pesticides with insecticidal, acaricidal, molluscidal and/or nematicidal activity: Agrobacterium radiobacter, Bacillus cereus, B. firmus, B. thuringiensis, B. thuringiensis ssp. aizawai, B. t. ssp. israelensis, B. t. ssp. galleriae, B. t. ssp. kurstaki, B. t. ssp. tenebrionis, Beauveria bassiana, B. brongniartii, Burkholderia spp., Chromobacterium subtsugae, Cydia pomonella granulovirus (CpGV), Cryptophlebia leucotreta granulovirus (CrleGV), Flavobacterium spp., Hellcoverpa armigera nucleopolyhedrovirus (HearNPV), Helicoverpazea nucleopolyhedrovirus (HzNPV), Helicoverpazea single capsid nucleopolyhedrovirus (HzSNPV), Heterorhabdtis bacteriophora, Isaria fumosorosea, Lecanicillium longisporum, L. muscarium, Metarhizium anisopliae, M. anisopliae var. anisopliae, M. anisopliae var. acridum, Nomuraea rileyi, Paecilomyces fumosoroseus, P. lilacinus, Paenibacillus popilliae, Pasteuria spp., P. nishizawae, P. penetrans, P. ramosa, P. thornea, P. usgae, Pseudomonas fluorescens, Spodoptera littoralis nucleopolyhedrovirus (SpliNPV), Steinernema carpocapsae, S. feltiae, S. kraussei, Streptomyces galbus, S. microflavus;
L4) Biochemical pesticides with insecticidal, acaricidal, molluscidal, pheromone and/or nematicidal activity: L-carvone, citral, (E,Z)-7,9-dodecadien-1-yl acetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, cis-jasmone, 2-methyl 1-butanol, methyl eugenol, methyl jasmonate, (E,Z)-2,13-octadecadien-1-ol, (E,Z)-2,13-octadecadien-1-ol acetate, (E,Z)-3,13-octadecadien-1-ol, (R)-1-octen-3-ol, pentatermanone, (E,Z,Z)-3,8,11-tetradecatrienyl acetate, (Z,E)-9,12-tetradecadien-1-yl acetate, (Z)-7-tetradecen-2-one, (Z)-9-tetradecen-1-yl acetate, (Z)-11-tetradecenal, (Z)-11-tetradecen-1-ol, extract of Chenopodium ambrosiodes, Neem oil, Quillay extract;
L5) Microbial pesticides with plant stress reducing, plant growth regulator, plant growth promoting and/or yield enhancing activity: Azospirillum amazonense, A. brasilense, A. lipoferum, A. irakense, A. halopraeferens, Bradyrhizobium spp., B. elkanii, B. japonicum, B. liaoningense, B. lupini, Delftia acidovorans, Glomus intraradices, Mesorhizobium spp., Rhizobium leguminosarum bv. phaseoli R. l. bv. trifolii R. l. bv. viciae, R. tropici, Sinorhizobium melilotr;

### M) Growth Regulators

abscisic acid (M.1.1), amidochlor, ancymidol, 6-benzylaminopurine, brassinolide, butralin, chlormequat, chlormequat chloride, choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat, mepiquat chloride, naphthaleneacetic acid, N-6-benzyladenine, paclobutrazol, prohexadione, prohexadione-calcium, prohydrojasmon, thidiazuron, triapenthenol, tributyl phosphorotrithioate, 2,3,5-tri-iodobenzoic acid, trinexapac-ethyl, uniconazole;
   N) Herbicides from Classes N.1 to N.15
N.1 Lipid biosynthesis inhibitors: alloxydim, alloxydim-sodium, butroxydim, clethodim, clodinafop, clodinafop-propargyl, cycloxydim, cyhalofop, cyhalofop-butyl, diclofop, diclofopmethyl, fenoxaprop, fenoxaprop-ethyl, fenoxaprop-P, fenoxaprop-P-ethyl, fluazifop, fluazifopbutyl, fluazifop-P, fluazifop-P-butyl, haloxyfop, haloxyfop-methyl, haloxyfop-P, haloxyfop-Pmethyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, 4-(4′-chloro-4-cyclo¬propyl-2′-fluoro[1,1′-biphenyl]-3-yl)-5-hydroxy-2,2,6,6-tetramethyl-2H-pyran-3(6H)-one (1312337-72-6); 4-(2′,4′-dichloro-4-cyclopropyl[1,1′-biphenyl]-3-yl)-5-hydroxy-2,2,6,6-tetramethyl-2H-pyran-3(6H)-one (1312337-45-3); 4-(4′-chloro-4-ethyl-2′-fluoro[1,1′-biphenyl]-3-yl)-5-hydroxy-2,2,6,6-tetramethyl-2H-pyran-3(6H)-one (1033757-93-5); 4-(2′,4′-dichloro-4-ethyl[1,1′-biphenyl]-3-yl)-2,2,6,6-tetramethyl-2H-pyran-3,5(4H,6H)-dione (1312340-84-3); 5-(acetyloxy)-4-(4′-chloro-4-cyclopropy-2′-fluoro[1,1′-biphenyl]-3-yl)-3,6-dihydro-2,2,6,6-tetramethyl-2H-pyran-3-one (1312337-48-6); 5-(acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(acetyloxy)-4-(4′-chloro-4-ethyl-2′-fluoro[1,1′-biphenyl]-3-yl)-3,6-dihydro-2,2,6,6-tetramethyl-2H-pyran-3-one (1312340-82-1); 5-(acetyloxy)-4-(2′,4′-dichloro-4-ethyl[1,1′-biphenyl]-3-yl)-3,6-dihydro-2,2,6,6-tetramethyl-2H-pyran-3-one (1033760-55-2); 4-(4′-chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (1312337-51-1); 4-(2′,4′-dichloro-4-cyclopropyl[1,1′-biphenyl]-3-yl)-5,6-dihydro-2,2,6,6-tetramethyl-5-oxo-2H-pyran-3-yl carbonic acid methyl ester; 4-(4′-chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (1312340-83-2); 4-(2′,4′-dichloro-4-ethyl,[1,1′-biphenyl]-3-yl)-5,6-dihydro-2,2,6,6-tetramethyl-5-oxo-2H-pyran-3-yl carbonic acid methyl ester (1033760-58-5); benfuresate, butylate, cycloate, dalapon, dimepiperate, EPTC, esprocarb, ethofumesate, flupropanate, molinate, orbencarb, pebulate, prosulfocarb, TCA, thiobencarb, tiocarbazil, triallate, vernolate;
N.2 ALS inhibitors: amidosulfuron, azimsulfuron, bensulfuron, bensulfuron-methyl, chlorimuron, chlorimuron-ethyl, chlorsulfuron, cinosulfuron, cyclosulfamuron, ethametsulfuron, ethametsulfuron-methyl, ethoxysulfuron, flazasulfuron, flucetosulfuron, flupyrsulfuron, flupyrsulfuronmethyl-sodium, foramsulfuron, halosulfuron, halosulfuron-methyl, imazosulfuron, iodosulfuron, iodosulfuron-methyl-sodium, iofensulfuron, iofensulfuron-sodium, mesosulfuron, metazosulfuron, metsulfuron, metsulfuron-methyl, nicosulfuron, orthosulfamuron, oxasulfuron, primisulfuron, primisulfuron-methyl, propyrisulfuron, prosulfuron, pyrazosulfuron, pyrazosulfuron-ethyl, rimsulfuron, sulfometuron, sulfometuron-methyl, sulfosulfuron, thifensulfuron, thifensulfuron-methyl, triasulfuron, tribenuron, tribenuron-methyl, trifloxysulfuron, triflusulfuron, triflusulfuron-methyl, tritosulfuron, imazamethabenz, imazamethabenz-methyl, imazamox, imazapic, imazapyr, imazaquin, imazethapyr; cloransulam, cloransulam-methyl, diclosulam, flumetsulam, florasulam, metosulam, penoxsulam, pyrimisulfan, pyroxsulam; bispyribac, bispyribac-sodium, pyribenzoxim, pyriftalid, pyriminobac, pyriminobac-methyl, pyrithiobac, pyrithiobac-sodium, 4-[[[2-[(4,6-dimethoxy-2-pyrimidinyl)oxy]phenyl]methyl]amino]-benzoic acid-1-methyl¬ethyl ester (420138-41-6), 4-[[[2-[(4,6-dimethoxy-2-pyrimidinyl)oxy]phenyl]¬methyl]amino]-benzoic acid propyl ester (420138-40-5), (4-bromophenyl)-2-[(4,6-dimethoxy-2-pyrimidinyl)oxy]benzenemethanamine (420138-01-8); flucarbazone, flucarbazone-sodium, propoxycarbazone, propoxycarbazone-sodium, thiencarbazone, thiencarbazone-methyl; triafamone;
N.3 Photosynthesis inhibitors: amicarbazone; chlorotriazine; ametryn, atrazine, chloridazone, cyanazine, desmetryn, dimethametryn, hexazinone, metribuzin, prometon, prometryn, propazine, simazine, simetryn, terbumeton, terbuthylazin, terbutryn, trietazin; chlorobromuron, chlorotoluron, chloroxuron, dimefuron, diuron, fluometuron, isoproturon, isouron, linuron, metamitron, methabenzthiazuron, metobenzuron, metoxuron, monolinuron, neburon, siduron, tebuthiuron, thiadiazuron, desmedipham, karbutilat, phenmedipham, phenmediphamethyl, bromofenoxim, bromoxynil and its salts and esters, ioxynil and its salts and esters, bromacil, lenacil, terbacil, bentazon, bentazon-sodium, pyridate, pyridafol, pentanochlor, propanil; diquat, diquat-dibromide, paraquat, paraquat-dichloride, paraquat-dimetilsulfate;
N.4 protoporphyrinogen-IX oxidase inhibitors: acifluorfen, acifluorfen-sodium, azafenidin, bencarbazone, benzfendizone, bifenox, butafenacil, carfentrazone, carfentrazone-ethyl, chlormethoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, tiafenacil, trifludimoxazin, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (353292-31-6), N-ethyl-3-(2,6-dichloro-4-trifluoromethylphenoxy)-5-methyl-1H-pyrazole-1-carboxamide (452098-92-9), N-tetrahydrofurfuryl-3-(2,6-dichloro-4-trifluoromethylphenoxy)-5-methyl-1H-pyrazole-1-carboxamide (915396-43-9), N-ethyl-3-(2-chloro-6-fluoro-4-trifluoromethyl¬phenoxy)-5-methyl-1H-pyrazole-1-carboxamide (452099-05-7), N-tetrahydro¬furfuryl-3-(2-chloro-6-fluoro-4-trifluoro¬methylphenoxy)-5-methyl-1H-pyrazole-1-carboxamide (452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (1300118-96-0), 1-methyl-6-trifluoro¬methyl-3-(2,2,7-tri-fluoro-3-oxo-4-prop-2-ynyl-3,4-dihydro-2H-benzo[1,4]oxazin-6-yl)-1H-pyrimidine-2,4-dione (1304113-05-0), methyl (E)-4-[2-chloro-5-[4-chloro-5-(difluoromethoxy)-1H-methylpyrazol-3-yl]-4-fluoro-phenoxy]-3-methoxy-but-2-enoate (948893-00-3), 3-[7-chloro-5-fluoro-2-(trifluoromethyl)-1H-benzimidazol-4-yl]-1-methyl-6-(trifluoromethyl)-1H-pyrimidine-2,4-dione (212754-02-4);
N.5 Bleacher herbicides: beflubutamid, diflufenican, fluridone, flurochloridone, flurtamone, norflurazon, picolinafen, 4-(3-trifluoromethyl-phenoxy)-2-(4-trifluoromethylphenyl)pyrimidine (180608-33-7); benzobicyclon, benzofenap, bicyclopyrone, clomazone, fenquintrione, isoxaflutole, mesotrione, pyrasulfotole, pyrazolynate, pyrazoxyfen, sulcotrione, tefuryltrione, tembotrione, tolpyralate, topramezone; aclonifen, amitrole, flumeturon;
N.6 EPSP synthase inhibitors: glyphosate, glyphosate-isopropylammonium, glyposate-potassium, glyphosate-trimesium (sulfosate);
N.7 Glutamine synthase inhibitors: bilanaphos (bialaphos), bilanaphos-sodium, glufosinate, glufosinate-P, glufosinate-ammonium;
N.8 DHP synthase inhibitors: asulam;
N.9 Mitosis inhibitors: benfluralin, butralin, dinitramine, ethalfluralin, fluchloralin, oryzalin, pendimethalin, prodiamine, trifluralin; amiprophos, amiprophos-methyl, butamiphos; chlorthal, chlorthal-dimethyl, dithiopyr, thiazopyr, propyzamide, tebutam; carbetamide, chlorpropham, flamprop, flamprop-isopropyl, flamprop-methyl, flamprop-M-isopropyl, flamprop-M-methyl, propham;
N.10 VLCFA inhibitors: acetochlor, alachlor, butachlor, dimethachlor, dimethenamid, dimethenamid-P, metazachlor, metolachlor, metolachlor-S, pethoxamid, pretilachlor, propachlor, propisochlor, thenylchlor, flufenacet, mefenacet, diphenamid, naproanilide, napropamide, napropamide-M, fentrazamide, anilofos, cafenstrole, fenoxasulfone, ipfencarbazone, piperophos, pyroxasulfone, isoxazoline compounds of the formulae II.1, II.2, II.3, II.4, II.5, II.6, II.7, II.8 and II.9

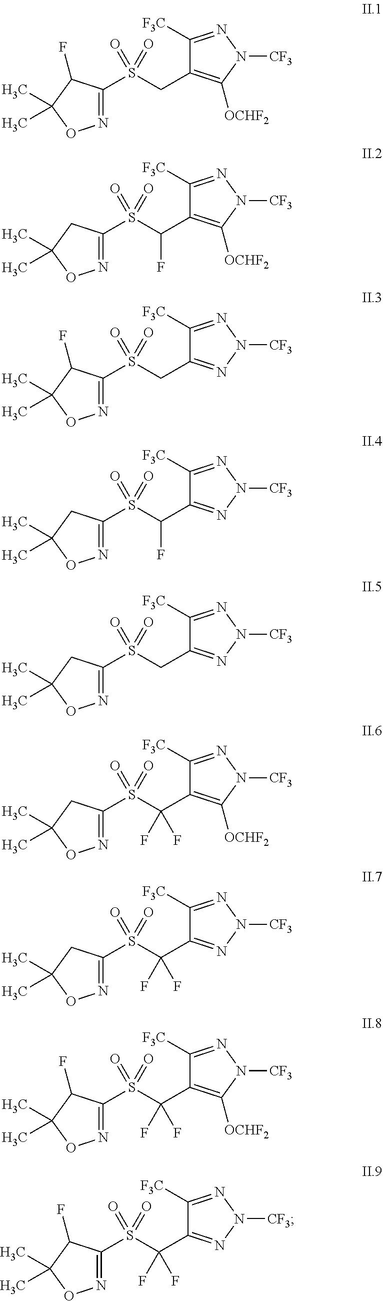

N.11 Cellulose biosynthesis inhibitors: chlorthiamid, dichlobenil, flupoxam, indaziflam, isoxaben, triaziflam, 1-cyclohexyl-5-pentafluorphenyloxy-14-[1,2,4,6]thiatriazin-3-ylamine (175899-01-1);
N.12 Decoupler herbicides: dinoseb, dinoterb, DNOC and its salts;
N.13 Auxinic herbicides: 2,4-D and its salts and esters, clacyfos, 2,4-DB and its salts and esters, aminocyclopyrachlor and its salts and esters, aminopyralid and its salts such as aminopyralid-dimethylammonium, aminopyralid-tris(2-hydroxypropyl)ammonium and its esters, benazolin, benazolin-ethyl, chloramben and its salts and esters, clomeprop, clopyralid and its salts and esters, dicamba and its salts and esters, dichlorprop and its salts and esters, dichlorprop-P and its salts and esters, fluroxypyr, fluroxypyr-butometyl, fluroxypyr-meptyl, halauxifen and its salts and esters (943832-60-8); MCPA and its salts and esters, MCPAthioethyl, MCPB and its salts and esters, mecoprop and its salts and esters, mecoprop-P and its salts and esters, picloram and its salts and esters, quinclorac, quinmerac, TBA (2,3,6) and its salts and esters, triclopyr and its salts and esters, 4-amino-3-chloro-6-(4-chloro-2-fluoro-3-methoxyphenyl)-5-fluoropyridine-2-carboxylic acid, benzyl 4-amino-3-chloro-6-(4-chloro-2-fluoro-3-methoxyphenyl)-5-fluoropyridine-2-carboxylate (1390661-72-9);
N.14 Auxin transport inhibitors: diflufenzopyr, diflufenzopyr-sodium, naptalam, naptalamsodium;
N.15 Other herbicides: bromobutide, chlorflurenol, chlorflurenol-methyl, cinmethylin, cumyluron, cyclopyrimorate (499223-49-3) and its salts and esters, dalapon, dazomet, difenzoquat, difenzoquat-metilsulfate, dimethipin, DSMA, dymron, endothal and its salts, etobenzanid, flurenol, flurenol-butyl, flurprimidol, fosamine, fosamine-ammonium, indanofan, maleic hydrazide, mefluidide, metam, methiozolin (403640-27-7), methyl azide, methyl bromide, methyldymron, methyl iodide, MSMA, oleic acid, oxaziclomefone, pelargonic acid, pyributicarb, quinoclamine, tridiphane;
   O) Insecticides from Classes O.1 to O.29
O.1 Acetylcholine esterase (AChE) inhibitors: aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb, triazamate; acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion;
O.2 GABA-gated chloride channel antagonists: endosulfan, chlordane; ethiprole, fipronil, flufiprole, pyrafluprole, pyriprole;
O.3 Sodium channel modulators: acrinathrin, allethrin, d-cis-trans allethrin, d-trans allethrin, bifenthrin, kappa-bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, heptafluthrin, imiprothrin, meperfluthrin, metofluthrin, momfluorothrin, epsilon-momfluorothrin, permethrin, phenothrin, prallethrin, profluthrin, pyrethrin (pyrethrum), resmethrin, silafluofen, tefluthrin, kappa-tefluthrin, tetramethylfluthrin, tetramethrin, tralomethrin, transfluthrin; DDT, methoxychlor;
O.4 Nicotinic acetylcholine receptor agonists (nAChR): acetamiprid, clothianidin, cycloxaprid, dinotefuran, imidacloprid, nitenpyram, thiacloprid, thiamethoxam; 4,5-dihydro-N-nitro-1-(2-oxiranylmethyl)-1H-imidazol-2-amine, (2E)-1-[(6-chloropyridin-3-yl)methyl]-N′-nitro-2-pentylidenehydrazinecarboximidamide; 1-[(6-chloropyridin-3-yl)methyl]-7-methyl-8-nitro-5-propoxy-1,2,3,5,6,7-hexahydroimidazo[1,2-a]pyridine; nicotine; sulfoxaflor, flupyradifurone, triflumezopyrim;
O.5 Nicotinic acetylcholine receptor allosteric activators: spinosad, spinetoram;
O.6 Chloride channel activators: abamectin, emamectin benzoate, ivermectin, lepimectin, milbemectin;
O.7 Juvenile hormone mimics: hydroprene, kinoprene, methoprene; fenoxycarb, pyriproxyfen;
O.8 miscellaneous non-specific (multi-site) inhibitors: methyl bromide and other alkyl halides; chloropicrin, sulfuryl fluoride, borax, tartar emetic;
O.9 Chordotonal organ TRPV channel modulators: pymetrozine, pyrifluquinazon; flonicamid;
O.10 Mite growth inhibitors: clofentezine, hexythiazox, diflovidazin; etoxazole;
O.11 Microbial disruptors of insect midgut membranes: Bacillus thuringiensis, Bacillus sphaericus and the insecticdal proteins they produce: Bacillus thuringiensis subsp. israelensis, Bacillus sphaericus, Bacillus thuringiensis subsp. alzawai, Bacillus thuringiensis subsp. kurstaki, Bacillus thuringiensis subsp. tenebrionis, the Bt crop proteins: Cry1Ab, Cry1Ac, Cry1Fa, Cry2Ab, mCry3A, Cry3Ab, Cry3Bb, Cry34/35Ab1;
O.12 Inhibitors of mitochondrial ATP synthase: diafenthiuron; azocyclotin, cyhexatin, fenbutatin oxide, propargite, tetradifon;
O.13 Uncouplers of oxidative phosphorylation via disruption of the proton gradient: chlorfenapyr, DNOC, sulfluramid;
O.14 Nicotinic acetylcholine receptor (nAChR) channel blockers: bensultap, cartap hydrochloride, thiocyclam, thiosultap sodium;
O.15 Inhibitors of the chitin biosynthesis type 0: bistrifluron, chlorfluazuron, diflubenzuron, flucycloxuron, flufenoxuron, hexaflumuron, lufenuron, novaluron, noviflumuron, teflubenzuron, triflumuron;
O.16 Inhibitors of the chitin biosynthesis type 1: buprofezin;
O.17 Moulting disruptors: cyromazine;
O.18 Ecdyson receptor agonists: methoxyfenozide, tebufenozide, halofenozide, fufenozide, chromafenozide;
O.19 Octopamin receptor agonists: amitraz;
O.20 Mitochondrial complex III electron transport inhibitors: hydramethylnon, acequinocyl, fluacrypyrim, bifenazate;
O.21 Mitochondrial complex I electron transport inhibitors: fenazaquin, fenpyroximate, pyrimidifen, pyridaben, tebufenpyrad, tolfenpyrad; rotenone;
O.22 Voltage-dependent sodium channel blockers: indoxacarb, metaflumizone, 2-[2-(4-cyanophenyl)-1-[3-(trifluoromethyl)phenyl]ethylidene]-N-[4-(difluoromethoxy)phenyl]-hydrazinecarboxamide, N-(3-chloro-2-methylphenyl)-2-[(4-chlorophenyl)-[4-[methyl(methylsulfonyl)amino]phenyl]methylene]-hydrazinecarboxamide;
O.23 Inhibitors of the of acetyl CoA carboxylase: spirodiclofen, spiromesifen, spirotetramat, spiropidion;
O.24 Mitochondrial complex IV electron transport inhibitors: aluminium phosphide, calcium phosphide, phosphine, zinc phosphide, cyanide;
O.25 Mitochondrial complex II electron transport inhibitors: cyenopyrafen, cyflumetofen;
O.26 Ryanodine receptor-modulators: flubendiamide, chlorantraniliprole, cyantraniliprole, cyclaniliprole, tetraniliprole; (R)-3-chloro-N′-{2-methyl-4-[1,2,2,2-tetrafluoro-1-(trifluoromethyl)ethyl]phenyl}-N²-(1-methyl-2-methylsulfonylethyl)phthalamide, (S)-3-chloro-N′-{2-methyl-4-[1,2,2,2-tetrafluoro-1-(trifluoromethyl)ethyl]phenyl}-N²-(1-methyl-2-methylsulfonylethyl)phthalamide, methyl-2-[3,5-dibromo-2-({[3-bromo-1-(3-chloropyridin-2-yl)-1H-pyrazol-5-yl]-carbonyl}amino)benzoyl]-1,2-dimethylhydrazinecarboxylate; N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide; N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide; N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide; -[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide; N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide; -[2-(5-amino-1,3,4-thiadiazol-2-yl)-4-chloro-6-methylphenyl]-3-bromo-1-(3-chloro-2-pyridinyl)-1H-pyrazole-5-carboxamide; 3-chloro-1-(3-chloro-2-pyridinyl)-N-[2,4-dichloro-6-[[(1-cyano-1-methylethyl)amino]carbonyl]phenyl]-1H-pyrazole-5-carboxamide; 3-bromo-N-[2,4-dichloro-6-(methylcarbamoyl)phenyl]-1-(3,5-dichloro-2-pyridyl)-1H-pyrazole-5-carboxamide; -[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide; cyhalodiamide;
O.27: Chordotonal organ Modulators—undefined target site: flonicamid;
O.28. insecticidal active compounds of unknown or uncertain mode of action: afidopyropen, afoxolaner, azadirachtin, amidoflumet, benzoximate, broflanilide, bromopropylate, chinomethionat, cryolite, dicloromezotiaz, dicofol, flufenerim, flometoquin, fluensulfone, fluhexafon, fluopyram, fluralaner, metoxadiazone, piperonyl butoxide, pyflubumide, pyridalyl, tioxazafen, 11-(4-chloro-2,6-dimethylphenyl)-12-hydroxy-1,4-dioxa-9-azadispiro[4.2.4.2]-tetradec-11-en-10-one, 3-(4′-fluoro-2,4-dimethylbiphenyl-3-yl)-4-hydroxy-8-oxa-1-azaspiro[4.5]dec-3-en2-one, 1-[2-fluoro-4-methyl-5-[(2,2,2-trifluoroethyl)sulfinyl]phenyl]-3-(trifluoromethyl)-1H-1,2,4-triazole-5-amine, Bacillus firmus I-1582; flupyrimin; fluazaindolizine; 4-[5-(3,5-dichlorophenyl)-5-(trifluoromethyl)-4H-isoxazol-3-yl]-2-methyl-N-(1-oxothietan-3-yl)benzamide; fluxametamide; 5-[3-[2,6-dichloro-4-(3,3-dichloroallyloxy)phenoxy]propoxy]-1H-pyrazole; 4-cyano-N-[2-cyano-5-[[2,6-dibromo-4-[1,2,2,3,3,3-hexafluoro-1-(trifluoromethyl)propyl]phenyl]carbamoyl]phenyl]-2-methyl-benzamide; 4-cyano-3-[(4-cyano-2-methyl-benzoyl)amino]-N-[2,6-dichloro-4-[1,2,2,3,3,3-hexafluoro-1-(trifluoromethyl)propyl]phenyl]-2-fluoro-benzamide; N-[5-[[2-chloro-6-cyano-4-[1,2,2,3,3,3-hexafluoro-1-(trifluoromethyl)propyl]phenyl]carbamoyl]-2-cyano-phenyl]-4-cyano-2-methyl-benzamide; N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide; N-[5-[[2-bromo-6-chloro-4-[1,2,2,3,3,3-hexafluoro-1-(trifluoromethyl)propyl]phenyl]carbamoyl]-2-cyano-phenyl]-4-cyano-2-methyl-benzamide; 4-cyano-N-[2-cyano-5-[[2,6-dichloro-4-[1,2,2,3,3,3-hexafluoro-1-(trifluoromethyl)propyl]phenyl]carbamoyl]phenyl]-2-methyl-benzamide; 4-cyano-N-[2-cyano-5-[[2,6-dichloro-4-[1,2,2,2-tetrafluoro-1-(trifluoromethyl)ethyl]phenyl]carbamoyl]phenyl]-2-methyl-benzamide; N-[5-[[2-bromo-6-chloro-4-[1,2,2,2-tetrafluoro-1-(trifluoromethyl)ethyl]phenyl]carbamoyl]-2-cyano-phenyl]-4-cyano-2-methyl-benzamide; 2-(1,3-dioxan-2-yl)-6-[2-(3-pyridinyl)-5-thiazolyl]-pyridine; 2-[6-[2-(5-fluoro-3-pyridinyl)-5-thiazolyl]-2-pyridinyl]-pyrimidine; 2-[6-[2-(3-pyridinyl)-5-thiazolyl]-2-pyridinyl]-pyrimidine; N-methylsulfonyl-6-[2-(3-pyridyl)thiazol-5-yl]pyridine-2-carboxamide; N-methylsulfonyl-6-[2-(3-pyridyl)thiazol-5-yl]pyridine-2-carboxamide; 1-[(6-chloro-3-pyridinyl)methyl]-1,2,3,5,6,7-hexahydro-5-methoxy-7-methyl-8-nitro-imidazo[1,2-a]pyridine; 1-[(6-chloropyridin-3-yl)methyl]-7-methyl-8-nitro-1,2,3,5,6,7-hexahydroimidazo[1,2-a]pyridin-5-ol; 1-isopropyl-N,5-dimethyl-N-pyridazin-4-yl-pyrazole-4-carboxamide; 1-(1,2-dimethylpropyl)-N-ethyl-5-methyl-N-pyridazin-4-yl-pyrazole-4-carboxamide; N,5-dimethyl-N-pyridazin-4-yl-1-(2,2,2-trifluoro-1-methyl-ethyl)pyrazole-4-carboxamide; 1-[1-(1-cyanocyclopropyl)ethyl]-N-ethyl-5-methyl-N-pyridazin-4-yl-pyrazole-4-carboxamide; N-ethyl-1-(2-fluoro-1-methyl-propyl)-5-methyl-N-pyridazin-4-yl-pyrazole-4-carboxamide; 1-(1,2-dimethylpropyl)-N,5-dimethyl-N-pyridazin-4-yl-pyrazole-4-carboxamide; 1-[1-(1-cyanocyclopropyl)ethyl]-N,5-dimethyl-N-pyridazin-4-yl-pyrazole-4-carboxamide; N-methyl-1-(2-fluoro-1-methyl-propyl]-5-methyl-1-pyridazin-4-yl-pyrazole-4-carboxamide; 1-(4,4-difluorocyclohexyl)-N-ethyl-5-methyl-N-pyridazin-4-yl-pyrazole-4-carboxamide; 1-(4,4-difluorocyclohexyl)-N,5-dimethyl-N-pyridazin-4-yl-pyrazole-4-carboxamide, N-(1-methylethyl)-2-(3-pyridinyl)-2H-indazole-4-carboxamide; N-cyclopropyl-2-(3-pyridinyl)-2H-indazole-4-carboxamide; N-cyclohexyl-2-(3-pyridinyl)-2H-indazole-4-carboxamide; 2-(3-pyridinyl)-N-(2,2,2-trifluoroethyl)-2H-indazole-4-carboxamide; 2-(3-pyridinyl)-N-[(tetrahydro-2-furanyl)methyl]-2H-indazole-5-carboxamide; methyl 2-[[2-(3-pyridinyl)-2H-indazol-5-yl]carbonyl]hydrazinecarboxylate; -[(2,2-difluorocyclopropyl)methyl]-2-(3-pyridinyl)-2H-indazole-5-carboxamide; N-(2,2-difluoropropyl)-2-(3-pyridinyl)-2H-indazole-5-carboxamide; 2-(3-pyridinyl)-N-(2-pyrimidinylmethyl)-2H-indazole-5-carboxamide; N-[(5-methyl-2-pyrazinyl)methyl]-2-(3-pyridinyl)-2H-indazole-5-carboxamide, tyclopyrazoflor; sarolaner, lotilaner, N-[4-chloro-3-[[(phenylmethyl)amino]carbonyl]phenyl]-1-methyl-3-(1,1,2,2,2-pentafluoroethyl)-4-(trifluoromethyl)-1H-pyrazole-5-carboxamide; M.UN.22a 2-(3-ethylsulfonyl-2-pyridyl)-3-methyl-6-(trifluoromethyl)imidazo[4,5-b]pyridine, 2-[3-ethylsulfonyl-5-(trifluoromethyl)-2-pyridyl]-3-methyl-6-(trifluoromethyl)imidazo[4,5-b]pyridine, 4-[5-(3,5-dichlorophenyl)-5-(trifluoromethyl)-4H-isoxazol-3-yl]-N-[(4R)-2-ethyl-3-oxo-isoxazolidin-4-yl]-2-methyl-benzamide, 4-[5-(3,5-dichloro-4-fluoro-phenyl)-5-(trifluoromethyl)-4H-isoxazol-3-yl]-MA[(4R)-2-ethyl-3-oxoisoxazolidin-4-yl]-2-methyl-benzamide; A[4-chloro-3-(cyclopropylcarbamoyl)phenyl]-2-methyl-5-(1,1,2,2,2-pentafluoroethyl)-4-(trifluoromethyl)pyrazole-3-carboxamide, N-[4-chloro-3-[(1-cyanocyclopropyl)carbamoyl]phenyl]-2-methyl-5-(1,1,2,2,2-pentafluoroethyl)-4-(trifluoromethyl)pyrazole-3-carboxamide; acynonapyr; benzpyrimoxan; chloro-N-(1-cyanocyclopropyl)-5-[1-[2-methyl-5-(1,1,2,2,2-pentafluoroethyl)-4-(trifluoromethyl)pyrazol-3-yl]pyrazol-4-yl]benzamide, oxazosulfyl, [(2S,3R,4R,5S,6S)-3,5-dimethoxy-6-methyl-4-propoxy-tetrahydropyran-2-yl]-MA[4-[1-[4-(trifluoromethoxy)phenyl]-1,2,4-triazol-3-yl]phenyl]carbamate, [(2S,3R,4R,5S,6S)-3,4,5-trimethoxy-6-methyl-tetrahydropyran-2-yl] N-[4-[1-[4-(trifluoromethoxy)phenyl]-1,2,4-triazol-3-yl]phenyl]carbamate, [(2S,3R,4R,5S,6S)-3,5-dimethoxy-6-methyl-4-propoxy-tetrahydropyran-2-yl]-N-[4-[1-[4-(1,1,2,2,2-pentafluoroethoxy)phenyl]-1,2,4-triazol-3-yl]phenyl]carbamate, [(2S,3R,4R,5S,6S)-3,4,5-trimethoxy-6-methyl-tetrahydropyran-2-yl]-A[4-[1-[4-(1,1,2,2,2-pentafluoroethoxy)phenyl]-1,2,4-triazol-3-yl]phenyl]carbamate, (22)-3-(2-isopropylphenyl)-2-[(E)-[4-[1-[4-(1,1,2,2,2-pentafluoroethoxy)phenyl]-1,2,4-triazol-3-yl]phenyl]methylenehydrazono]thiazolidin-4-one.

a

The active substances referred to as component 2, their preparation and their activity e. g. against harmful fungi is known (cf.: http://www.alanwood.net/pesticides/); these substances are commercially available. The compounds described by IUPAC nomenclature, their preparation and their pesticidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. Nos. 3,296,272; 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 10/139271, WO 11/028657, WO 12/168188, WO 07/006670, WO 11/77514; WO 13/047749, WO 10/069882, WO 13/047441, WO 03/16303, WO 09/90181, WO 13/007767, WO 13/010862, WO 13/127704, WO 13/024009, WO 13/24010, WO 13/047441, WO 13/162072, WO 13/092224, WO 11/135833, CN 1907024, CN 1456054, CN 103387541, CN 1309897, WO 12/84812, CN 1907024, WO 09094442, WO 14/60177, WO 13/116251, WO 08/013622, WO 15/65922, WO 94/01546, EP 2865265, WO 07/129454, WO 12/165511, WO 11/081174, WO 13/47441). Some compounds are identified by their CAS Registry Number which is separated by hyphens into three parts, the first consisting from two up to seven digits, the second consisting of two digits, and the third consisting of a single digit.

The present invention furthermore relates to agrochemical compositions comprising a mixture of at least one compound I (component 1) and at least one further active substance useful for plant protection, e. g. selected from the groups A) to O) (component 2), in particular one further fungicide, e. g. one or more fungicide from the groups A) to K), as described above, and if desired one suitable solvent or solid carrier. Those mixtures are of particular interest, since many of them at the same application rate show higher efficiencies against harmful fungi. Furthermore, combating harmful fungi with a mixture of compounds I and at least one fungicide from groups A) to K), as described above, is more efficient than combating those fungi with individual compounds I or individual fungicides from groups A) to K).

By applying compounds I together with at least one active substance from groups A) to O) a synergistic effect can be obtained, i.e. more then simple addition of the individual effects is obtained (synergistic mixtures).

This can be obtained by applying the compounds I and at least one further active substance simultaneously, either jointly (e. g. as tank-mix) or separately, or in succession, wherein the time interval between the individual applications is selected to ensure that the active substance applied first still occurs at the site of action in a sufficient amount at the time of application of the further active substance(s). The order of application is not essential for working of the present invention.

When applying compound I and a pesticide II sequentially the time between both applications may vary e. g. between 2 hours to 7 days. Also a broader range is possible ranging from 0.25 hour to 30 days, preferably from 0.5 hour to 14 days, particularly from 1 hour to 7 days or from 1.5 hours to 5 days, even more preferred from 2 hours to 1 day. In case of a mixture comprising a pesticide II selected from group L), it is preferred that the pesticide II is applied as last treatment.

According to the invention, the solid material (dry matter) of the biopesticides (with the exception of oils such as Neem oil) are considered as active components (e. g. to be obtained after drying or evaporation of the extraction or suspension medium in case of liquid formulations of the microbial pesticides).

In accordance with the present invention, the weight ratios and percentages used herein for a biological extract such as Quillay extract are based on the total weight of the dry content (solid material) of the respective extract(s).

The total weight ratios of compositions comprising at least one microbial pesticide in the form of viable microbial cells including dormant forms, can be determined using the amount of CFU of the respective microorganism to calculate the total weight of the respective active component with the following equation that 1×10¹⁰ CFU equals one gram of total weight of the respective active component. Colony forming unit is measure of viable microbial cells, in particular fungal and bacterial cells. In addition, here “CFU” may also be understood as the number of (juvenile) individual nematodes in case of (entomopathogenic) nematode biopesticides, such as Steinernema fetiae.

In the binary mixtures and compositions according to the invention the weight ratio of the component 1) and the component 2) generally depends from the properties of the active components used, usually it is in the range of from 1:10,000 to 10,000:1, often it is in the range of from 1:100 to 100:1, regularly in the range of from 1:50 to 50:1, preferably in the range of from 1:20 to 20:1, more preferably in the range of from 1:10 to 10:1, even more preferably in the range of from 1:4 to 4:1 and in particular in the range of from 1:2 to 2:1.

According to further embodiments of the binary mixtures and compositions, the weight ratio of the component 1) and the component 2) usually is in the range of from 1000:1 to 1:1, often in the range of from 100:1 to 1:1, regularly in the range of from 50:1 to 1:1, preferably in the range of from 20:1 to 1:1, more preferably in the range of from 10:1 to 1:1, even more preferably in the range of from 4:1 to 1:1 and in particular in the range of from 2:1 to 1:1.

According to further embodiments of the mixtures and compositions, the weight ratio of the component 1) and the component 2) usually is in the range of from 20,000:1 to 1:10, often in the range of from 10,000:1 to 1:1, regularly in the range of from 5,000:1 to 5:1, preferably in the range of from 5,000:1 to 10:1, more preferably in the range of from 2,000:1 to 30:1, even more preferably in the range of from 2,000:1 to 100:1 and in particular in the range of from 1,000:1 to 100:1.

According to a further embodiments of the binary mixtures and compositions, the weight ratio of the component 1) and the component 2) usually is in the range of from 1:1 to 1:1000, often in the range of from 1:1 to 1:100, regularly in the range of from 1:1 to 1:50, preferably in the range of from 1:1 to 1:20, more preferably in the range of from 1:1 to 1:10, even more preferably in the range of from 1:1 to 1:4 and in particular in the range of from 1:1 to 1:2.

According to further embodiments of the mixtures and compositions, the weight ratio of the component 1) and the component 2) usually is in the range of from 10:1 to 1:20,000, often in the range of from 1:1 to 1:10,000, regularly in the range of from 1:5 to 1:5,000, preferably in the range of from 1:10 to 1:5,000, more preferably in the range of from 1:30 to 1:2,000, even more preferably in the range of from 1:100 to 1:2,000 to and in particular in the range of from 1:100 to 1:1,000.

In the ternary mixtures, i.e. compositions according to the invention comprising the component 1) and component 2) and a compound III (component 3), the weight ratio of component 1) and component 2) depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1, and the weight ratio of component 1) and component 3) usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1.

Any further active components are, if desired, added in a ratio of from 20:1 to 1:20 to the component 1).

These ratios are also suitable for inventive mixtures applied by seed treatment.

When mixtures comprising microbial pesticides are employed in crop protection, the application rates preferably range from about 1×10⁶ to 5×10¹⁶ (or more) CFU/ha, preferably from about 1×10⁸ to about 1×10¹³ CFU/ha, and even more preferably from about 1×10⁹ to 5×10¹⁵ CFU/ha and particularly preferred even more preferably from 1×10¹² to 5×10¹⁴ CFU/ha. In the case of (entomopathogenic) nematodes as microbial pesticides (e. g. Steinernema feltiae), the application rates preferably range inform about 1×10⁵ to 1×10¹² (or more), more preferably from 1×10⁸ to 1×10¹¹, even more preferably from 5×10⁸ to 1×10¹⁰ individuals (e. g. in the form of eggs, juvenile or any other live stages, preferably in an infective juvenile stage) per ha.

When mixtures comprising microbial pesticides are employed in seed treatment, the application rates with respect to plant propagation material preferably range from about 1×10⁶ to 1×10¹² (or more) CFU/seed. Preferably, the concentration is about 1×10⁶ to about 1×10⁹ CFU/seed. In the case of the microbial pesticides II, the application rates with respect to plant propagation material also preferably range from about 1×10⁷ to 1×10¹⁴ (or more) CFU per 100 kg of seed, preferably from 1×10⁹ to about 1×10¹² CFU per 100 kg of seed.

Preference is also given to mixtures comprising as component 2) at least one active substance selected from inhibitors of complex III at Q₀ site in group A), more preferably selected from compounds (A.1.1), (A.1.4), (A.1.8), (A.1.9), (A.1.10), (A.1.12), (A.1.13), (A.1.14), (A.1.17), (A.1.21), (A.1.25), (A.1.34) and (A.1.35); particularly selected from (A.1.1), (A.1.4), (A.1.8), (A.1.9), (A.1.13), (A.1.14), (A.1.17), (A.1.25), (A.1.34) and (A.1.35).

Preference is also given to mixtures comprising as component 2) at least one active substance selected from inhibitors of complex III at Qᵢ site in group A), more preferably selected from compounds (A.2.1), (A.2.3) and (A.2.4); particularly selected from (A.2.3) and (A.2.4).

Preference is also given to mixtures comprising as component 2) at least one active substance selected from inhibitors of complex II in group A), more preferably selected from compounds (A.3.2), (A.3.3), (A.3.4), (A.3.7), (A.3.9), (A.3.11), (A.3.12), (A.3.15), (A.3.16), (A.3.17), (A.3.18), (A.3.19), (A.3.20), (A.3.21), (A.3.22), (A.3.23), (A.3.28), (A.3.31), (A.3.32), (A.3.33), (A.3.34), (A.3.35), (A.3.36), (A.3.37), (A.3.38) and (A.3.39); particularly selected from (A.3.2), (A.3.3), (A.3.4), (A.3.7), (A.3.9), (A.3.12), (A.3.15), (A.3.17), (A.3.19), (A.3.22), (A.3.23), (A.3.31), (A.3.32), (A.3.33), (A.3.34), (A.3.35), (A.3.36), (A.3.37), (A.3.38) and (A.3.39).

Preference is also given to mixtures comprising as component 2) at least one active substance selected from other respiration inhibitors in group A), more preferably selected from compounds (A.4.5) and (A.4.11); in particular (A.4.11).

Preference is also given to mixtures comprising as component 2) at least one active substance selected from C14 demethylase inhibitors in group B), more preferably selected from compounds (B.1.4), (B.1.5), (B.1.8), (B.1.10), (B.1.11), (B.1.12), (B.1.13), (B.1.17), (B.1.18), (B.1.21), (B.1.22), (B.1.23), (B.1.25), (B.1.26), (B.1.29), (B.1.34), (B.1.37), (B.1.38), (B.1.43) and (B.1.46); particularly selected from (B.1.5), (B.1.8), (B.1.10), (B.1.17), (B.1.22), (B.1.23), (B.1.25), (B.1.33), (B.1.34), (B.1.37), (B.138), (B.1.43) and (B.1.46).

Preference is also given to mixtures comprising as component 2) at least one active substance selected from Delta14-reductase inhibitors in group B), more preferably selected from compounds (B.2.4), (B.2.5), (B.2.6) and (B.2.8); in particular (B.2.4).

Preference is also given to mixtures comprising as component 2) at least one active substance selected from phenylamides and acyl amino acid fungicides in group C), more preferably selected from compounds (C.1.1), (C.1.2), (C.1.4) and (C.1.5); particularly selected from (C.1.1) and (C.1.4).

Preference is also given to mixtures comprising as component 2) at least one active substance selected from other nucleic acid synthesis inhibitors in group C), more preferably selected from compounds (C.2.6), (C.2.7) and (C.2.8).

Preference is also given to mixtures comprising as component 2) at least one active substance selected from group D), more preferably selected from compounds (D.1.1), (D.1.2), (D.1.5), (D.2.4) and (D.2.6); particularly selected from (D.1.2), (D.1.5) and (D.2.6).

Preference is also given to mixtures comprising as component 2) at least one active substance selected from group E), more preferably selected from compounds (E.1.1), (E.1.3), (E.2.2) and (E.2.3); in particular (E.1.3).

Preference is also given to mixtures comprising as component 2) at least one active substance selected from group F), more preferably selected from compounds (F.1.2), (F.1.4) and (F.1.5).

Preference is also given to mixtures comprising as component 2) at least one active substance selected from group G), more preferably selected from compounds (G.3.1), (G.3.3), (G.3.6), (G.5.1), (G.5.2), (G.5.3), (G.5.4), (G.5.5), G.5.6), G.5.7), (G.5.8), (G.5.9), (G.5.10) and (G.5.11); particularly selected from (G.3.1), (G.5.1), (G.5.2) and (G.5.3).

Preference is also given to mixtures comprising as component 2) at least one active substance selected from group H), more preferably selected from compounds (H.2.2), (H.2.3), (H.2.5), (H.2.7), (H.2.8), (H.3.2), (H.3.4), (H.3.5), (H.4.9) and (H.4.10); particularly selected from (H.2.2), (H.2.5), (H.3.2), (H.4.9) and (H.4.10).

Preference is also given to mixtures comprising as component 2) at least one active substance selected from group 1), more preferably selected from compounds (1.2.2) and (1.2.5).

Preference is also given to mixtures comprising as component 2) at least one active substance selected from group J), more preferably selected from compounds (J.1.2), (J.1.5), (J.1.8), (J.1.11) and (J.1.12); in particular (J.1.5).

Preference is also given to mixtures comprising as component 2) at least one active substance selected from group K), more preferably selected from compounds (K.1.41), (K.1.42), (K.1.44) and (K.1.47); particularly selected from (K.1.41), (K.1.44) and (K.1.47).

### SYNTHESIS EXAMPLE

With due modification of the starting compounds, the procedures shown in the synthesis examples below were used to obtain further compounds I. The resulting compounds, together with physical data, are listed in Table I below.

HPLC-MS: HPLC-column Kinetex XB C18 1.7μ (50×2.1 mm); eluent: acetonitrile/water+0.1% TFA (5 gradient from 5:95 to 100:0 in 1.5 min at 60° C., flow gradient from 0.8 to 1.0 ml/min in 1.5 min). MS: Quadrupol Electrospray Ionisation, 80 V (positive mode).

### 1. Synthesis of Intermediate Ethyl 2-benzyl-4-methyl-pentanoate

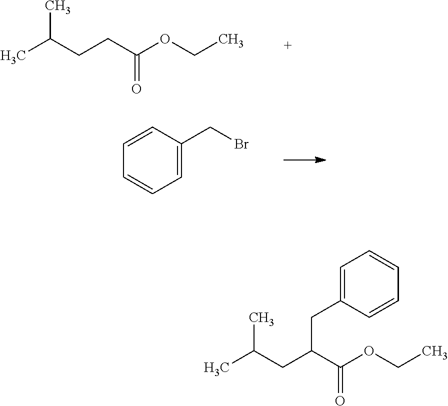

Under inert atmosphere, lithium diisopropylamide (4.457 g, 32 mmol) was dissolved in THF (250 mL) and cooled to −78° C. Ethyl-4-methylvalerate (5.000 g, 35 mmol) was added dropwise and stirred at −78° C. for 2 h. Benzyl bromide (7.116 g, 41.6 mmol) was added dropwise, stirred at −78° C. for 1 h and subsequently for 2 h at 25° C. Aq. sat. NH₄Cl solution was added and the organic phase was extracted with MTBE, washed with water and brine, dried over MgSO₄, and evaporated. Column chromatography (SiO₂; cyclohexane/ethyl acetate 2:1) yielded ethyl 2-benzyl-4-methyl-pentanoate (8.100 g, quant.) as colorless powder.

### 2. Synthesis of Intermediate Ethyl 2-benzyl-2,4-dimethyl-pentanoate

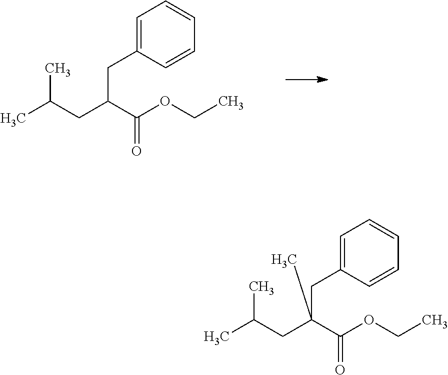 [SMILES: CCOC(=O)C(Cc1ccccc1)CC(C)C ; CCOC(=O)C(C)(Cc1ccccc1)CC(C)C]

Under inert atmosphere, lithium diisopropylamide (3.456 g, 42 mmol) was dissolved in THF (40 mL) and cooled to −78° C. Ethyl 2-benzyl-4-methyl-pentanoate (6.300 g, 26.9 mmol) was dissolved in THF (10 mL), added dropwise, and stirred at −78° C. for 2 h. Methyl iodide (4.579 g, 32 mmol) was added dropwise, stirred at −78° C. for 10 min and subsequently for 2 h at 25° C. Aq. sat. NH₄Cl solution was added and the organic phase was extracted with MTBE, washed with water and brine, dried over MgSO₄, and evaporated. Column chromatography (SiO₂; cyclohexane/ethyl acetate 2:1) yielded Intermediate ethyl 2-benzyl-2,4-dimethyl-pentanoate (6.300 g, 94%) as colorless powder.

### 3. Synthesis of Intermediate 2-benzyl-2,4-dimethyl-pentanoic acid

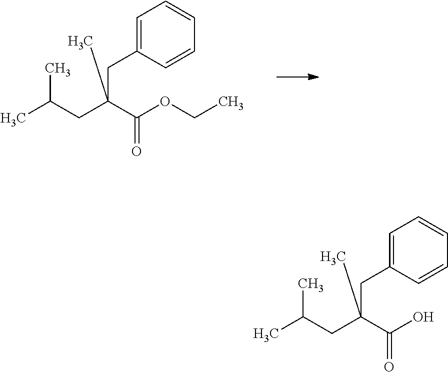 [SMILES: CCOC(=O)C(C)(Cc1ccccc1)CC(C)C ; CC(C)CC(C)(Cc1ccccc1)C(=O)O]

Ethyl 2-benzyl-2,4-dimethyl-pentanoate (6.300 g, 25.4 mmol) was dissolved in dioxane/EtOH 1:1 (30 mL). Aq. 2 M NaOH solution (25 mL) was added and the mixture was stirred at 120° C. over night. The organic solvents were evaporated, the mixture was taken up in water and washed with cyclohexane. The aq. phase was adjusted to pH=1 with HCl and extracted with dichloromethane. The combined organic phases were washed with water and brine, dried over MgSO₄, and evaporated to yield 2-benzyl-2,4-dimethyl-pentanoic acid (650 mg, 12%) as colorless powder.

### Synthesis of Intermediate 2-benzyl-2,4-dimethyl-pentanoyl Chloride

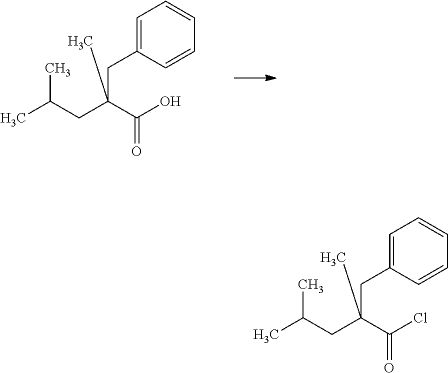 [SMILES: CC(C)CC(C)(Cc1ccccc1)C(=O)O ; CC(C)CC(C)(Cc1ccccc1)C(=O)Cl]

Under inert atmosphere, 2-benzyl-2,4-dimethyl-pentanoic acid (650 mg, 3 mmol) was dissolved in thionyl chloride (6 mL), stirred at 95° C. for 3 h, and evaporated to yield 2-benzyl-2,4-dimethyl-pentanoyl chloride (690 mg, 98%) as a brown oil.

### 4. Synthesis of Ex-80

Under inert atmosphere, 5,6-dimethyl-3-amino-pyridine (512 mg, 4.2 mmol) was dissolved in dichloromethane (15 mL) and treated with NEt₃ (424 mg, 4.2 mmol). The mixture was cooled to 0° C., treated with a solution of 2-benzyl-2,4-dimethyl-pentanoyl chloride (553 mg, 2.3 mmol) in dichloromethane (3 mL), stirred at 0° C. for 20 min and subsequently at 25° C. over night. Water was added and phases were separated. The aq. phase was extracted with dichloromethane and the combined organic phases were washed with aq. sat. NH₄Cl solution, aq. sat. NaHCO₃ solution, and brine, dried over MgSO₄, and evaporated. Column chromatography (SiO₂; cyclohexane/ethyl acetate 2:1) yielded Target Molecule Ex-80 (550 mg, 73%) as colorless powder.

TABLE I
Compounds Ex-1 to Ex-81 of the formula I.A-1

| | |
|---|---|
| | I.A-1 |
| 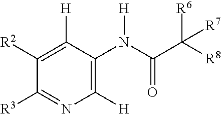 | |

| | | | | | | Melting | HPLC |
|---|---|---|---|---|---|---|---|
| Ex. | | | | | | Point | Rt |
| no | R² | R³ | R⁶ | R⁷ | R⁸ | (° C.) | (min)*/** |
| Ex-1 | —CH₃ | —CH₃ | ^{t-}butyl | H | —CH₂Ph | 169 | 0.91* |
| Ex-2 | —CH₃ | —CH₃ | —CH₃ | H | —CH₂Ph | 103 | 0.79** |
| Ex-3 | —CH₃ | —CH₃ | —CH₃ | H | —Ph | 168 | 0.85* |
| Ex-4 | —CH₃ | —CH₃ | —CH₂CH₃ | H | —Ph | 126 | 0.84* |
| Ex-5 | —CH₃ | —CH₃ | —CH(CH₃)₂ | H | —Ph | 160 | 0.87** |
| Ex-6 | —CH₃ | —CH₃ | —CH₂CH₂CH₃ | H | —Ph | 130 | 0.87** |
| Ex-7 | —CH₃ | —CH₃ | —CH₂CH(CH₃)₂ | H | —Ph | 136 | 0.94** |
| Ex-8 | —CH₃ | —CH₃ | —CH₂CH₃ | H | —CH₂Ph | nd | 0.84** |
| Ex-9 | —CH₃ | —CH₃ | —CH(CH₃)₂ | H | —CH₂Ph | nd | 0.88** |
| Ex-10 | —CH₃ | —CH₃ | —CH₂CH₂CH₃ | H | —CH₂Ph | nd | 0.91** |
| Ex-11 | —CH₃ | —CH₃ | —O—^{t-}butyl | H | —Ph | 142 | 0.90** |
| Ex-12 | —CH₃ | —CH₃ | ^{t-}butyl | H | —Ph | 159 | 0.98** |
| Ex-13 | —CH₃ | —CH₃ | —CH₂CH(CH₃)₂ | H | —CH₂Ph | 121 | 0.98** |
| Ex-14 | —CH₃ | —CH₃ | —CH(CH₃)₂ | H | —CH₂CH₂Ph | nd | 0.97** |
| Ex-15 | —CH₃ | —CH₃ | —CH₂CH(CH₃)₂ | H | —CH₂CH₂Ph | 83 | 1.03** |
| Ex-16 | —CH₃ | —CH₃ | ^{t-}butyl | H | —CH₂CH₂Ph | 174 | 0.98** |
| Ex-17 | —CH₃ | —CH₃ | —CH₂CH₂CH₃ | H | —CH₂CH₂Ph | nd | 0.94** |
| Ex-18 | —CH₃ | —CH₃ | —CH₂CH₃ | H | —CH₂CH₂Ph | 150 | 0.92** |
| Ex-19 | —CH₃ | —CH₃ | —CH₃ | OH | —Ph | 158 | 0.72** |
| Ex-20 | —CH₃ | —CH₃ | —CH₂CH(CH₃)₂ | OH | —Ph | nd | 0.91** |
| Ex-21 | —CH₃ | —CH₃ | —CH₂CH₂CH₃ | OH | —Ph | 145 | 0.85** |
| Ex-22 | —CH₃ | —CH₃ | —CH(CH₃)₂ | OH | —Ph | nd | 0.85** |
| Ex-23 | —CH₃ | —CH₃ | —CH₂CH₃ | OH | —CH₂Ph | nd | 0.79** |
| Ex-24 | —CH₃ | —CH₃ | H | H | —Ph | 113 | 0.70* |
| Ex-25 | —CH₃ | —CH₃ | F | F | —Ph | 143 | 0.78* |
| Ex-26 | —CH₃ | —CH₃ | —CH₃ | —CH₃ | —Ph | 144 | 0.81* |
| Ex-27 | —CH₃ | —CHF₂ | H | H | —Ph | 113 | 1.01* |
| Ex-28 | —CH₃ | —CH₃ | —CH₃ | —CH₃ | —CH₂CH₃ | nd | 0.70* |
| Ex-29 | —CH₃ | —CH₃ | —CH₃ | —CH₃ | —CH₂CH₂CH₃ | nd | 0.78* |
| Ex-30 | —CH₃ | —CH₃ | —CH₃ | —CH₃ | —CH₂CH₂CH₂CH₃ | nd | 0.85* |
| Ex-31 | —CH₃ | —CH₃ | —CH₃ | —CH₃ | —CH₂Ph | 98 | 0.83* |
| Ex-32 | —CH₃ | —CH₃ | —CH₃ | —CH₃ | —CH(CH₃)₂ | nd | 0.79* |
| Ex-33 | —CH₃ | —CH₃ | —CH₃ | —CH₃ | ^{t-}butyl | 110 | 0.82* |
| Ex-34 | —CH₃ | —CH₃ | H | H | —CH₂Ph | 129 | 0.74* |
| Ex-35 | —CH₃ | —CH₃ | H | H | —CH₂CH₂Ph | nd | 0.79* |
| Ex-36 | —CH₃ | —CH₃ | —CH₃ | —CH₃ | cyclopropyl | nd | 0.73* |
| Ex-37 | —CH₃ | —CH₃ | F | F | —Ph | 117 | 1.08* |
| Ex-38 | —CH₃ | —CH₃ | ^{t-}butyl | H | —CH₂Ph | 169 | 0.91* |
| Ex-39 | —CH₃ | —CH₃ | —CH₃ | H | —CH₂Ph | 103 | 0.79* |
| Ex-40 | —CH₃ | —CHF₂ | —CH₃ | —CH₃ | —Ph | 108 | 1.13* |
| Ex-41 | —CH₃ | —CH₃ | —CH₃ | H | —Ph | 168 | 0.84* |
| Ex-42 | —CH₃ | —CHF₂ | ^{t-}butyl | H | —CH₂Ph | nd | 1.25* |
| Ex-43 | —CH₃ | —CH₃ | H | H | —CH₂—cyclohexyl | nd | 0.94* |
| Ex-44 | —CH₃ | —CH₃ | —CH(CH₃)₂ | H | Br | nd | 0.82* |
| Ex-45 | —CH₃ | —CH₃ | —CH₂CH₂CH₂CH₃ | H | Phenyl | nd | 0.99* |
| Ex-46 | —CH₃ | —CH₃ | cyclopentyl | H | Phenyl | nd | 0.99* |
| Ex-47 | —CH₃ | —CH₃ | H | H | | nd | 0.89* |
| Ex-48 | —CH₃ | —CH₃ | H | H | | nd | 0.89* |
| Ex-49 | —CH₃ | —CH₃ | H | H | | nd | 0.91* |
| Ex-50 | —CH₃ | —CH₃ | —CH₃ | H | —CH₂CH₂CH₂CH₂CH₃ | nd | 0.93* |
| Ex-51 | —CH₃ | —CH₃ | H | H | 3,4-dichlorophenyl | nd | 0.92* |
| Ex-52 | —CH₃ | —CH₃ | H | H | | nd | 0.87* |
| Ex-53 | —CH₃ | —CH₃ | —CH₂CH₂CH₃ | H | —CH₂CH₂CH₃ | nd | 0.91* |
| Ex-54 | —CH₃ | —CH₃ | —CH₂Ph | H | | nd | 0.93* |
| Ex-55 | —CH₃ | —CH₃ | —CN | H | | nd | 0.81* |
| Ex-56 | —CH₃ | —CH₃ | —CH(CH₃)₂ | H | —CH₂CH₂CH(CH₃)₂ | nd | 1.00* |
| Ex-57 | —CH₃ | —CH₃ | H | H | —CH₂—cyclopentyl | nd | 0.90* |
| Ex-58 | —CH₃ | —CH₃ | H | H | | nd | 0.88* |
| Ex-59 | —CH₃ | —CH₃ | H | H | | nd | 0.99* |
| Ex-60 | —CH₃ | —CH₃ | H | H | | nd | 0.88* |
| Ex-61 | —CH₃ | —CH₃ | H | H | | nd | 0.74* |
| Ex-62 | —CH₃ | —CH₃ | H | H | | nd | 0.84* |
| Ex-63 | —CH₃ | —CH₃ | —CF₃ | —OCH₃ | phenyl | nd | 0.94* |
| Ex-64 | —CH₃ | —CH₃ | H | H | -cyclohexyl | nd | 0.87* |
| Ex-65 | —CH₃ | —CH₃ | H | Cl | —CH₂Cl | nd | 0.70* |
| Ex-66 | —CH₃ | —CH₃ | —CH₃ | —CH₃ | | nd | 0.77* |
| Ex-67 | —Cl | —Cl | H | H | 4-trifluoromethylphenyl | nd | 1.24* |
| Ex-68 | H | —N(CH₃)₂ | H | H | 4-trifluoromethylphenyl | nd | 0.82* |
| Ex-69 | —CH₃ | —CHF₂ | cyclopropyl | H | —CH₂Ph | nd | 1.18* |
| Ex-70 | —CH₃ | —CH₃ | cyclopropyl | H | —CH₂Ph | nd | 0.84* |
| Ex-71 | H | —N(CH₃)₂ | —CH(CH₃)₂ | H | 4-methoxyphenyl | nd | 0.91* |
| Ex-72 | H | —OCH₃ | —CH(CH₃)₂ | H | 4-methoxyphenyl | nd | 1.05* |
| Ex-73 | H | —SCH₃ | —CH(CH₃)₂ | H | 4-methoxyphenyl | nd | 1.10* |
| Ex-74 | —Cl | —Cl | —CH(CH₃)₂ | H | 4-methoxyphenyl | nd | 1.31* |
| Ex-75 | —CH₃ | —CH₃ | —CH(CH₃)₂ | H | 4-methoxyphenyl | nd | 0.90* |
| Ex-76 | —CH₃ | —CHF₂ | —CH(CH₃)₂ | H | 4-methoxyphenyl | nd | 1.24* |
| Ex-77 | —CH₃ | —CF₃ | —CH(CH₃)₂ | H | 4-methoxyphenyl | nd | 1.32* |
| Ex-78 | H | —Cl | —CH(CH₃)₂ | H | 4-methoxyphenyl | nd | 1.21* |
| Ex-79 | —OCH₃ | —OCH₃ | —CH(CH₃)₂ | H | 4-methoxyphenyl | nd | 1.13* |
| Ex-80 | —CH₃ | —CH₃ | —CH₂CH(CH₃)₂ | —CH₃ | —CH₂Ph | 123 | 1.01* |
| Ex-81 | —CH₃ | —CHF₂ | —CH₂CH(CH₃)₂ | —CH₃ | —CH₂Ph | 112 | 1.32* |

TABLE 2
Compounds Ex-82 to Ex-96 of the formula I.A-2

| | | | | | | Melting | HPLC |
|---|---|---|---|---|---|---|---|
| | | | | | | Point | Rₜ |
| Ex. no. | R² | R³ | R⁶ | R⁷ | R⁸ | (° C.) | (min)*/** |
| Ex-82 | —CH₃ | —CH₃ | —CH₃ | —CH₃ | —CH₃ | 104 | 0.66* |
| Ex-83 | —CH₃ | —CH₃ | H | H | —Ph | 172 | 0.69* |
| Ex-84 | —CH₃ | —CH₃ | —CH₃ | —CH₃ | —CH₂CH₃ | 98 | 0.69* |
| Ex-85 | —CH₃ | —CH₃ | F | F | —Ph | 99 | 0.76* |
| Ex-86 | —CH₃ | —CH₃ | —CH₃ | —CH₃ | —CH₂CH₂CH₃ | nd | 0.79* |
| Ex-87 | —CH₃ | —CH₃ | —CH₃ | —CH₃ | —Ph | 104 | 0.79* |
| Ex-88 | —CH₃ | —CH₃ | —CH₃ | —CH₃ | —CH₂CH₂CH₂CH₃ | nd | 0.86* |
| Ex-89 | —CH₃ | —CH₃ | —CH₃ | —CH₃ | —CH₂Ph | nd | 0.85* |
| Ex-90 | —CH₃ | —CH₃ | —CH₃ | —CH₃ | —CH(CH₃)₂ | 131 | 0.77* |
| Ex-91 | —CH₃ | —CH₃ | H | H | —CH₂Ph | 184 | 0.74* |
| Ex-92 | —CH₃ | —CH₃ | H | H | —CH₂CH₂Ph | nd | 0.83* |
| Ex-93 | —CH₃ | —CH₃ | —CH₃ | —CH₃ | ^{t-}Butyl | 156 | 0.82* |
| Ex-94 | —CH₃ | —CH₃ | H | ^{t-}Butyl | —CH₂Ph | 149 | 0.91* |
| Ex-95 | —CH₃ | —CH₃ | —CH₃ | —CH₃ | cyclopropyl | nd | 0.71* |
| Ex-96 | —CH₃ | —CH₃ | H | —CH₃ | —CH₂Ph | 218 | 0.73* |

*Standart 100_700
**pos_Standard
HPLC: High Performance Liquid Chromatography; HPLC-column Kinetex XB C18 1.7μ (50 × 2.1 mm); eluent: acetonitrile/water + 0.1% trifluoroacetic acid (gradient from 5:95 to 100:0 in 1.5 min at 60° C., flow gradient from 0.8 to 1.0 ml/min in 1.5 min). MS: Quadrupol Electrospray lonisation, 80 V (positive mode). Rt: retention time i

II. Biological Trials

Microtest

The active compounds were formulated separately as a stock solution having a concentration of 10000 ppm in dimethyl sulfoxide.

### EXAMPLE 1

### Activity Against the Grey Mold Botrytis cinerea in the Microtiterplate Test

The stock solutions were mixed according to the ratio, pipetted onto a micro titer plate (MTP) and diluted with water to the stated concentrations. A spore suspension of Botrci cinerea in an aqueous biomalt or yeast-bactopeptone-sodiumacetate or DOB solution was then added. The plates were placed in a water vapor-saturated chamber at a temperature of 18° C. Using an absorption photometer, the MTPs were measured at 405 nm 7 days after the inoculation.

In this test, the samples which had been treated with 31 ppm of the active substance from examples Ex-12, Ex-14, Ex-16, Ex-33, Ex-38, Ex-42, Ex-56, Ex-63, Ex-70, Ex-74, Ex-76, Ex-77, Ex-80 and Ex-88 respectively, showed up to at most 11% growth of the pathogen.

### EXAMPLE 2

### Activity Against Fusarium culmorum in the Microtiterplate Test

The stock solutions were mixed according to the ratio, pipetted onto a micro titer plate (MTP) and diluted with water to the stated concentrations. A spore suspension of Fusarium culmorum in an aqueous biomalt or yeast-bactopeptone-glycerine or DOB solution was then added. The plates were placed in a water vapor-saturated chamber at a temperature of 18° C. Using an absorption photometer, the MTPs were measured at 405 nm 7 days after the inoculation.

In this test, the samples which had been treated with 31 ppm of the active substance from examples Ex-4, Ex-5, Ex-6, Ex-7, Ex-12, Ex-12, Ex-13, Ex-14, Ex-15, Ex-16, Ex-17, Ex-18, Ex-25, Ex-33, Ex-35, Ex-41, Ex-43, Ex-44, Ex-45, Ex-46, Ex-47, Ex-49, Ex-50, Ex-53, Ex-56, Ex-57, Ex-59, Ex-63, Ex64, Ex-70, Ex-74, Ex-80 and Ex-88 respectively, showed up to at most 15% growth of the pathogen.

### EXAMPLE-3

### Activity Against Rice Blast Pyricularia oryzae in the Microtiterplate Test

The stock solutions were mixed according to the ratio, pipetted onto a micro titer plate (MTP) and diluted with water to the stated concentrations. A spore suspension of Pyricularia oryzae in an aqueous biomalt or yeast-bactopeptone-glycerine or DOB solution was then added. The plates were placed in a water vapor-saturated chamber at a temperature of 18° C. Using an absorption photometer, the MTPs were measured at 405 nm 7 days after the inoculation.

The measured parameters were compared to the growth of the active compound-free control variant (100%) and the fungus-free and active compound-free blank value to determine the relative growth in % of the pathogens in the respective active compounds.

In this test, the samples which had been treated with 31 ppm of the active substance from examples Ex-30, Ex-31, Ex-32, Ex-33, Ex-35, Ex-38, Ex-42, Ex-49, Ex-50, Ex-51, Ex-52, Ex-56, Ex-59, Ex-63, Ex-80, Ex-81, Ex-84, Ex-85, Ex-87, Ex-88, Ex-90, Ex-93, Ex-94, Ex-95 and Ex-96 respectively, showed up to at most 15% growth of the pathogen.

Green House

The Spray Solutions were Prepared in Several Steps:

The stock solution was prepared: a mixture of acetone and/or dimethylsulfoxide and the wetting agent/emulsifier Wettol, which is based on ethoxylated alkylphenoles, in a relation (volume) solvent-emulsifier of 99 to 1 was added to the initial weight of the compound to give a total of 5 ml. Water was then added to total volume of 100 ml.

This stock solution was diluted with the described solvent-emulsifier-water mixture to the given concentration.

Preventative Fungicidal Control of Botrytis cinerea on Leaves of Green Pepper

Young seedlings of green pepper were grown in pots to the 4 to 5 leaf stage. These plants were sprayed to run-off with an aqueous suspension, containing the concentration of active ingredient or their mixture mentioned in the table below. The next day the plants were inoculated with an aqueous biomalt or DOB solution containing the spore suspension of Botrytis cinerea. Then the plants were immediately transferred to a humid chamber. After 5 days at 22 to 24⋅ C and a relative humidity close to 100% the extent of fungal attack on the leaves was visually assessed as % diseased leaf area.

In this test, the samples which had been treated with 250 ppm of the active substance from examples from Ex-33, Ex-36, Ex-38, Ex-42, Ex-80 and Ex-94 respectively, showed up to at most 15% growth of the pathogen whereas the untreated plants were 90% infected.

1. A compound of formula I 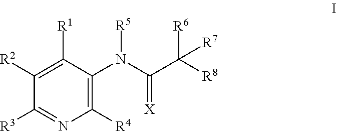
wherein
X is O, S, NH
R¹ is in each case independently selected from hydrogen, halogen, OH, CN, COOH, CONH₂, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
R^{x} is C₁-C₄-alkyl, C₁-C₄-halogenalkyl, unsubstituted aryl or aryl that is substituted by 1, 2, 3, 4 or 5 substituents R′ independently selected from C₁-C₄-alkyl, halogen, OH, CN, C₁-C₄-halogenalkyl, C₁-C₄-alkoxy and C₁-C₄-halogenalkoxy;
wherein the acyclic moieties of R¹ are unsubstituted or substituted with identical or different groups R^{1a} which independently of one another are selected from:
R^{1a} halogen, OH, CN, COOH, CONH₂, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{11a} selected from the group consisting of halogen, OH, C₁-C₄-alkyl, C₁-C₄-halogenalkyl, C₁-C₄-alkoxy and C₁-C₄-halogenalkoxy;
wherein the carbocyclic, heteroaryl and aryl moieties of R¹ are not further substituted or carry 1, 2, 3, 4, 5 or up to the maximum number of identical or different groups R^{1b} which independently of one another are selected from:
R^{1b} halogen, OH, CN, COOH, CONH₂, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R² is in each case independently selected from halogen, OH, CN, COOH, CONH₂, NO₂, SH, NH₂, NH(C₁-C₄-alkyl), N(C₁-C₄-alkyl)₂, NH(C₂-C₄-alkenyl), N(C₂-C₄-alkenyl)₂, NH(C₂-C₄-alkynyl), N(C₂-C₄-alkynyl)₂, NH(C₃-C₆-cycloalkyl), N(C₃-C₆-cycloalkyl)₂, N(C₁-C₄-alkyl)(C₂-C₄-alkenyl), N(C₁-C₄-alkyl)(C₂-C₄-alkynyl), N(C₁-C₄-alkyl)(C₃-C₆-cycloalkyl), N(C₂-C₄-alkenyl)(C₂-C₄-alkynyl), N(C₂-C₄-alkenyl)(C₃-C₆-cycloalkyl), N(C₂-C₄-alkynyl)(C₃-C₆-cycloalkyl), NH(C(═O)C₁-C₄-alkyl), N(C(═O)C₁-C₄-alkyl)₂, NH—SO₂—R^{x}, S(O)ₙ—C₁-C₆-alkyl, S(O)ₙ-aryl, C₁-C₆-cycloalkylthio, S(O)ₙ—C₂-C₆-alkenyl, S(O)ₙ—C₂-C₆-alkynyl, CH(═O), C(═O)C₁-C₆-alkyl, C(═O)C₂-C₆-alkenyl, C(═O)C₂-C₆-alkynyl, C(═O)C₃-C₆-cycloalkyl, C(═O)NH(C₁-C₆-alkyl), CH(═S), C(═S)C₁-C₆-alkyl, C(═S)C₂-C₆-alkenyl, C(═S)C₂-C₆-alkynyl, C(═S)C₃-C₆-cycloalkyl, C(═S)O(C₂-C₆-alkenyl), C(═S)O(C₂-C₆-alkynyl), C(═S)O(C₃-C₇-cycloalkyl), C(═S)NH(C₁-C₆-alkyl), C(═S)NH(C₂-C₆-alkenyl), C(═S)NH(C₂-C₆-alkynyl), C(═S)NH(C₃-C₇-cycloalkyl), C(═S)N(C₁-C₆-alkyl)₂, C(═S)N(C₂-C₆-alkenyl)₂, C(═S)N(C₂-C₆-alkynyl)₂, C(═S)N(C₃-C₇-cycloalkyl)₂, C₁-C₆-alkyl, C₁-C₆-halogenalkyl, C₂-C₆-alkenyl, C₂-C₆-alkynyl, OR′, C₃-C₆-cycloalkyl, five- or six-membered heteroaryl and aryl; wherein the heteroaryl contains one, two or three heteroatoms selected from N, O and S; wherein
R^{x} is as defined above;
R^{Y} is C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₃-C₆-cycloalkyl, C₃-C₆-halogencycloalkyl, phenyl and phenyl-C₁-C₆-alkyl; wherein the phenyl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
wherein the acyclic moieties of R² are unsubstituted or substituted by groups R^{2a} which independently of one another are selected from:
R^{2a} halogen, OH, CN, COOH, CONH₂, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or substituted by substituents R^{91a} selected from the group consisting of halogen, OH, C₁-C₄-alkyl, C₁-C₄-halogenalkyl, C₁-C₄-alkoxy and C₁-C₄-halogenalkoxy;
wherein the carbocyclic, heteroaryl and aryl moieties of R² are unsubstituted or substituted by groups R^{3b} which independently of one another are selected from:
R^{2b} halogen, OH, CN, COOH, CONH₂, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
and wherein n is defined as above;
R³ is in each case independently selected from the substituents as defined for R², wherein the possible substituents for R³ are R^{3a} and R^{3b}, respectively, which correspond to R^{2a} and R^{2b}, respectively;
R², R³ together with the carbon atoms to which they are bound form a five-, six-, or seven-membered carbo-, heterocyclic or heteroaromatic ring; wherein the heterocyclic or heteroaromatic ring contains 1, 2, 3 or 4 heteroatoms selected from N, O and S, wherein N may carry one substituent R^{N} selected from C₁-C₄-alkyl, C₁-C₄-halogenalkyl and SO₂Ph, wherein Ph is unsubstituted or substituted by substituents selected from C₁-C₄-alkyl, halogen, C₁-C₄-halogenalkyl, C₁-C₄-alkoxy, C₁-C₄-halogenalkoxy, and CN; and wherein S may be in the form of its oxide SO or SO₂; and wherein in each case one or two CH₂ groups of the carbo- or heterocycle may be replaced by a group independently selected from C(═O) and C(═S); and wherein the carbo-, heterocyclic or heteroaromatic ring is substituent by (R²³)ₘ, wherein m is 0, 1, 2, 3 or 4;
R²³ is in each case independently selected from halogen, OH, CN, COOH, CONH₂, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, saturated or partially unsaturated three-, four-, five-, six-, seven-, eight-, nine-, or ten-membered carbo- and heterocycle, five- or six-membered heteroaryl and aryl; wherein the heterocycle and heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
R^{x} is as defined above;
wherein the acyclic moieties of R²³ are unsubstituted or carry 1, 2, 3 or up to the maximum possible number of identical or different groups R^{23a} which independently of one another are selected from:
R^{23a} halogen, OH, CN, COOH, CONH₂, C₁-C₆-alkoxy, C₃-C₆-cycloalkyl, C₃-C₆-halogencycloalkyl, C₁-C₄-halogenalkoxy, C₁-C₆-alkylthio and phenoxy, wherein the phenyl group is unsubstituted or unsubstituted or substituted with R^{91a} selected from the group consisting of halogen, OH, C₁-C₄-alkyl, C₁-C₄-halogenalkyl, C₁-C₄-alkoxy, C₁-C₄-halogenalkoxy, CN, C₃-C₆-cycloalkyl, C₃-C₆-halogencycloalkyl, C₁-C₄-alkylthio;
wherein the carbocyclic, heterocyclic, heteroaryl and aryl moieties of R²³ are unsubstituted or substituted with identical or different groups R²³ which independently of one another are selected from:
R^{23b} halogen, OH, CN, COOH, CONH₂, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, and C₁-C₆-alkylthio;
R⁴ is in each case independently selected from hydrogen, halogen, OH, CN, COOH, CONH₂, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
R^{x} is as defined above
wherein the aliphatic moieties of R⁴ are unsubstituted or substituted with identical or different groups R^{2a} which independently of one another are selected from:
R^{4a} halogen, OH, CN, COOH, CONH₂, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{41a} selected from the group consisting of halogen, OH, C₁-C₄-alkyl, C₁-C₄-halogenalkyl, C₁-C₄-alkoxy and C₁-C₄-halogenalkoxy;
wherein the cycloalkyl, heteroaryl and aryl moieties of R⁴ are not further substituted or carry 1, 2, 3, 4, 5 or up to the maximum number of identical or different groups R⁴ which independently of one another are selected from:
R^{4b} halogen, OH, CN, COOH, CONH₂, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R⁵ is in each case independently selected from hydrogen, OH, CH(═O), C(═O)C₁-C₆-alkyl, C(═O)C₂-C₆-alkenyl, C(═O)C₂-C₆-alkynyl, C(═O)C₃-C₆-cycloalkyl, C₁-C₆-alkyl, C₁-C₄-halogenalkyl, C₃-C₆-cycloalkyl, C₃-C₆-halogencycloalkyl, C₁-C₄-alkoxy, C₁-C₄-halogenalkoxy, OR′, C₂-C₆-alkenyl, C₂-C₆-halogenalkenyl C₂-C₆-alkynyl, C₂-C₆-halogenalkynyl, five- or six-membered heteroaryl and aryl; wherein the heteroaryl contains one, two or three heteroatoms selected from N, O and S; wherein the aryl groups are unsubstituted or carry one, two, three, four or five substituents selected from the group consisting of CN, halogen, OH, C₁-C₄-alkyl, C₁-C₄-halogenalkyl, C₁-C₄-alkoxy and C₁-C₄-halogenalkoxy;
R^{Y} is defined as above;
wherein the acyclic moieties of R⁵ are not further substituted or carry one, two, three or up to the maximum possible number of identical or different groups R^{5a} which independently of one another are selected from:
R^{5a} halogen, OH, CN, COOH, CONH₂, C₁-C₆-alkoxy, C₃-C₆-cycloalkyl, C₃-C₆-cycloalkenyl, C₃-C₆-halogencycloalkyl, C₃-C₆-halogencycloalkenyl, C₁-C₄-halogenalkoxy, C₁-C₆-alkylthio, five- or six-membered heteroaryl, phenyl and phenoxy, wherein the heteroaryl, phenyl and phenoxy group is unsubstituted or carries one, two, three, four or five substituents R^{78a′} selected from the group consisting of halogen, OH, C₁-C₄-alkyl, C₁-C₄-halogenalkyl, C₁-C₄-alkoxy and C₁-C₄-halogenalkoxy;
wherein the alicyclic, phenyl, heterocyclic and heteroaryl moieties of R¹² are not further substituted or carry one, two, three, four, five or up to the maximum number of identical or different groups R^{12b} which independently of one another are selected from:
R^{5b} halogen, OH, CN, COOH, CONH₂, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R⁶ is independently selected from hydrogen, halogen, OH, CN, COOH, CONH₂, NO₂, SH, C₁-C₆-alkylthio, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, aryloxy, heteroaryloxy, arylamino, heteroarylamino, arylthio, heteroarylthio, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or
wherein the aliphatic moieties of and R⁶ are independently not further substituted or carry 1, 2, 3 or up to the maximum possible number of identical or different groups R^{6a}, respectively, which independently of one another are selected from:
R^{6a} halogen, OH, CN, COOH, CONH₂, NO₂, SH, NH₂, NH(C₁-C₄-alkyl), N(C₁-C₄-alkyl)₂, NH-aryl, N(aryl)₂, NH(C(═O)C₁-C₄-alkyl), N(C(═O)C₁-C₄-alkyl)₂, NHSO₂—R^{x}, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S), five- or six-membered heteroaryl and aryl; wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′, R″ and R″ are as defined above
wherein the carbocyclic, heterocyclic, heteroaryl and aryl moieties of R⁶ are independently not further substituted or carry 1, 2, 3, 4, 5 or up to the maximum number of identical or different groups R^{6b}, respectively, which independently of one another are selected from:
R^{6b} halogen, OH, CN, COOH, CONH₂, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy;
and wherein R^{x} is as defined above; or
n is 0, 1, 2
R⁷ is independently selected from
halogen, OH, CN, COOH, CONH₂, NO₂, SH, C₁-C₆-alkylthio, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₂-C₆-alkenyloxy, C₂-C₆-alkynyloxy, aryloxy, heteroaryloxy, arylamino, heteroarylamino, arylthio, heteroarylthio, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or
wherein the aliphatic moieties of and R⁷ are independently not further substituted or carry 1, 2, 3 or up to the maximum possible number of identical or different groups R^{7a}, respectively, which independently of one another are selected from:
R^{7a} halogen, OH, CN, COOH, CONH₂, NO₂, SH, NH₂, NH(C₁-C₄-alkyl), N(C₁-C₄-alkyl)₂, NH-aryl, N(aryl)₂, NH(C(═O)C₁-C₄-alkyl), N(C(═O)C₁-C₄-alkyl)₂, NHSO₂—R^{x}, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S), five- or six-membered heteroaryl and aryl; wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′, R″ and R″ are as defined above
wherein the carbocyclic, heterocyclic, heteroaryl and aryl moieties of R⁷ are independently not further substituted or carry 1, 2, 3, 4, 5 or up to the maximum number of identical or different groups R^{7b}, respectively, which independently of one another are selected from:
R^{7b} halogen, OH, CN, COOH, CONH₂, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy;
and wherein R^{x} is as defined above;
n is 0, 1, 2
R⁸ is independently selected from CN, COOH, CONH₂, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight, nine-, or ten-membered carbocycle or heterocycle, a five- or six-membered heteroaryl or aryl; wherein in each case one or two CH₂ groups of the carbo- and heterocycle may be replaced by a group independently selected from C(═O) and C(═S), and wherein the heterocycle and the heteroaryl contain independently one, two, three or four heteroatoms selected from N, O and S, and wherein heterocycle and heteroaryl are connected via C atom; and wherein R′ and R″ are independently selected from H, C₁-C₄-alkyl, C₂-C₆-alkenyl, C₂-C₆-alkynyl, saturated or partially unsaturated three-, four-, five-, six-, seven-, eight-, nine-, or ten-membered carbo- and heterocycle, five- or six-membered heteroaryl or aryl; wherein the heterocycle or heteroaryl contains one, two or three heteroatoms selected from N, O and S, and wherein R′ and R″ are independently unsubstituted or substituted by R′″ which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy, C₃-C₆-cycloalkyl, C₃-C₆-halogencycloalkyl and phenyl; or
wherein the aliphatic moieties of and R⁸ are independently not further substituted or carry 1, 2, 3 or up to the maximum possible number of identical or different groups R^{8a}, respectively, which independently of one another are selected from:
R^{8a} halogen, OH, CN, COOH, CONH₂, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkoxy, C₃-C₆-halogencycloalkyl, C₁-C₄-halogenalkoxy, C₁-C₆-alkylthio, C₁-C₆-halogenalkylthio, S(O)ₙ—C₁-C₆-alkyl, S(O)ₙ-aryl, CH(═O), C(═O)C₁-C₆-alkyl, C(═O)O(C₁-C₆-alkyl), C(═O)NH(C₁-C₆-alkyl), C(═O)N(C₁-C₆-alkyl)₂, CR′═NOR″, a saturated or partially unsaturated three-, four-, five-, six-, seven-, eight-, nine-, or ten-membered carbocycle or heterocycle, aryl, phenoxy, a five-, six- or ten-membered heteroaryl; wherein in each case one or two CH₂ groups of the carbo- and heterocycle may be replaced by a group independently selected from C(═O) and C(═S), five- or six-membered heteroaryl and aryl; wherein the heterocycle and the heteroaryl contain independently 1, 2, 3 or 4 heteroatoms selected from N, O and S; wherein in each case one or two CH₂ groups of the carbo- and heterocycle may be replaced by a group independently selected from C(═O) and C(═S); wherein the carbocyclic, heterocyclic, aryl and phenyl groups are independently unsubstituted or carry one, two, three, four or five substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-halogenalkyl, C₁-C₄-alkoxy, C₁-C₄-halogenalkoxy, and S(O)ₙ—C₁-C₆-alkyl; and wherein R^{x}, R′, R″ and R″ are as defined above
wherein the carbocyclic, heterocyclic, heteroaryl and aryl moieties of R⁸ are independently not further substituted or carry 1, 2, 3, 4, 5 or up to the maximum number of identical or different groups R^{8b}, respectively, which independently of one another are selected from:
R^{8b} halogen, OH, CN, COOH, CONH₂, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy, C₁-C₆-alkylthio, C₁-C₆-halogenalkylthio, S(O)ₙ—C₁-C₆-alkyl, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-halogenalkyl, C₁-C₄-alkoxy and C₁-C₄-halogenalkoxy;
and wherein R^{x} is as defined above;
n is 0, 1, 2
and the N-oxides and the agriculturally acceptable salts thereof for use as a fungicide.

2. The compound of claim 1, wherein R¹ is H, F, Cl, Br, CN, C₁-C₆-alkyl, C₂-C₆-alkenyl, C₂-C₆-alkynyl, C₁-C₆-alkoxy, C₃-C₆-cycloalkyl, wherein the acyclic moieties of R¹ are unsubstituted or substituted by halogen.

3. The compound of claim 1, wherein R² is selected from CN, halogen, C₁-C₆-alkyl, C₁-C₆-halogenalkyl, C₂-C₆-alkynyl, OR^{Y}, C₃-C₆-cycloalkyl, wherein
R^{Y} is C₁-C₆-alkyl, C₁-C₆-halogenalkyl, C₂-C₆-alkenyl or C₂-C₆-alkynyl.

4. The compound of claim 1, wherein R³ is selected from CN, halogen, C₁-C₆-alkyl, C₁-C₆-halogenalkyl, C₂-C₆-alkynyl, OR^{Y}, C₃-C₆-cycloalkyl, wherein
R^{Y} is C₁-C₆-alkyl, C₁-C₆-halogenalkyl, C₂-C₆-alkenyl or C₂-C₆-alkynyl.

5. The compound of claim 1, wherein R⁴ is H, F, Cl, Br, CN, C₁-C₆-alkyl, C₂-C₆-alkenyl, C₂-C₆-alkynyl, C₁-C₆-alkoxy, C₃-C₆-cycloalkyl, wherein the acyclic moieties of R¹ are unsubstituted or substituted by halogen.

6. The compound of claim 1, wherein R⁵ is H.

7. The compound of claim 1, wherein R⁶ is selected from CN, C₁-C₆-alkyl, C₁-C₆-halogenalkyl, C₂-C₆-alkenyl, C₂-C₆-halogenalkenyl, C₃-C₆-cycloalkenyl, C₂-C₆-alkynyl, C₂-C₆-halogenalkynyl, C₁-C₆-alkoxy, C₃-C₆-cycloalkynyl, C(═O)C₁-C₆-alkyl, C(═O)O(C₁-C₆-alkyl), CR′═NOR″, a saturated three-, four-, five-, six-, membered carbocycle or heterocycle, a five- or six-membered heteroaryl, benzyl or aryl.

8. The compound of claim 1, wherein R⁷ and R⁸ are independently selected from CN, C₁-C₆-alkyl, C₁-C₆-halogenalkyl, C₂-C₆-alkenyl, C₂-C₆-halogenalkenyl, C₃-C₆-cycloalkenyl, C₂-C₆-alkynyl, C₂-C₆-halogenalkynyl, C₁-C₆-alkoxy, C₃-C₆-cycloalkynyl, C(═O)C₁-C₆-alkyl, C(═O)O(C₁-C₆-alkyl), CR′═NOR″, a saturated three-, four-, five-, six-, membered carbocycle or heterocycle, a five- or six-membered heteroaryl, benzyl or aryl.

9. The compound of formula I as defined in claim 1,
wherein
X is O,
R¹ is H,
R² is C₁-C₆-alkyl and C₁-C₆-halogenalkyl,
R³ is C₁-C₆-alkyl and C₁-C₆-halogenalkyl,
R⁴ is H, and
R⁵ is H.

10. The compound of formula I as defined in claim 1,
wherein
X is O,
R¹ is in each case independently selected from CN, COOH, CONH₂, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
R^{x} is C₁-C₄-alkyl, C₁-C₄-halogenalkyl, unsubstituted aryl or aryl that is substituted by 1, 2, 3, 4 or 5 substituents R′ independently selected from C₁-C₄-alkyl, halogen, OH, CN, C₁-C₄-halogenalkyl, C₁-C₄-alkoxy and C₁-C₄-halogenalkoxy;
wherein the acyclic moieties of R¹ are unsubstituted or substituted with identical or different groups R^{1a} which independently of one another are selected from:
R^{1a} halogen, OH, CN, COOH, CONH₂, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{11a} selected from the group consisting of halogen, OH, C₁-C₄-alkyl, C₁-C₄-halogenalkyl, C₁-C₄-alkoxy and C₁-C₄-halogenalkoxy;
wherein the carbocyclic, heteroaryl and aryl moieties of R¹ are not further substituted or carry 1, 2, 3, 4, 5 or up to the maximum number of identical or different groups R^{1b} which independently of one another are selected from:
R^{1b} halogen, OH, CN, COOH, CONH₂, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio;
R² is C₁-C₆-alkyl and C₁-C₆-halogenalkyl,
R³ is C₁-C₆-alkyl and C₁-C₆-halogenalkyl,
R⁴ is is in each case independently selected from CN, COOH, CONH₂, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
R^{x} is C₁-C₄-alkyl, C₁-C₄-halogenalkyl, unsubstituted aryl or aryl that is substituted by 1, 2, 3, 4 or 5 substituents R^{x}4 independently selected from C₁-C₄-alkyl, halogen, OH, CN, C₁-C₄-halogenalkyl, C₁-C₄-alkoxy and C₁-C₄-halogenalkoxy;
wherein the acyclic moieties of R⁴ are unsubstituted or substituted with identical or different groups R^{4a} which independently of one another are selected from:
R^{4a} halogen, OH, CN, COOH, CONH₂, C₁-C₆-alkoxy, C₃-C₆-cycloalkyl, C₃-C₆-halogencycloalkyl, C₁-C₄-halogenalkoxy, C₁-C₆-alkylthio, aryl and phenoxy, wherein the aryl group is unsubstituted or carries 1, 2, 3, 4 or 5 substituents R^{41a} selected from the group consisting of halogen, OH, C₁-C₄-alkyl, C₁-C₄-halogenalkyl, C₁-C₄-alkoxy and C₁-C₄-halogenalkoxy;
wherein the carbocyclic, heteroaryl and aryl moieties of R⁴ are not further substituted or carry 1, 2, 3, 4, 5 or up to the maximum number of identical or different groups R^{4b} which independently of one another are selected from:
R^{4b} halogen, OH, CN, COOH, CONH₂, C₁-C₄-alkyl, C₁-C₄-alkoxy, C₁-C₄-halogenalkyl, C₃-C₆-cycloalkyl, C₃-C₆-halogencycloalkyl, C₁-C₄-halogenalkoxy and C₁-C₆-alkylthio; and
R⁵ is H.

11. A composition, comprising one compound of formula I, as defined in claim 1, an N-oxide or an agriculturally acceptable salt thereof.

12. A compound of formula I, as defined in claim 1, and of an agriculturally acceptable salt thereof for use in combating phytopathogenic fungi.

13. A method for combating phytopathogenic fungi,
comprising treating the fungi or the materials, plants, the soil or seeds to be protected against fungal attack with an effective amount of at least one compound of formula I, as defined in claim 1.

14. A seed coated with at least one compound of the formula I, as defined in claim 1 or an agriculturally acceptable salt thereof, in an amount of from 0.1 to 10 kg per 100 kg of seed.
